(19) United States
(12) Patent Application Publication
(10) Pub. No.: US 2022/0298135 A1
(43) Pub. Date: Sep. 22, 2022

(54) 5-HETEROARYL-PYRIDIN-2-AMINE COMPOUNDS AS NEUROPEPTIDE FF RECEPTOR ANTAGONISTS

(71) Applicant: Domain Therapeutics, Illkirch-Graffenstaden (FR)

(72) Inventors: Stephan SCHANN, Illkirch-Graffenstaden (FR); Mickaël FER, Illkirch-Graffenstaden (FR); Stanislas MAYER, Illkirch-Graffenstaden (FR); Christelle DOEBELIN, Illkirch-Graffenstaden (FR)

(73) Assignee: Domain Therapeutics, Illkirch-Graffenstaden (FR)

(21) Appl. No.: 17/633,209

(22) PCT Filed: Aug. 6, 2020
(86) PCT No.: PCT/EP2020/072128

§ 371 Date: Feb. 6, 2022

(30) Foreign Application Priority Data
Aug. 6, 2019 (EP) 19315085.1

(51) Int. Cl. C07D 401/04; A61K 31/4709; A61K 31/4436; C07D 409/04; C07D 471/04; A61K 31/437; C07D 405/04; C07D 417/04; C07D 401/10; C07D 401/14; A61K 31/498; A61K 31/506; C07D 413/14; A61K 31/553; A61K 31/55; C07D 498/08; C07D 405/14; A61P 25/36; A61P 29/02; A61K 45/06

(52) U.S. Cl. CPC C07D 401/04; A61K 31/4709; A61K 31/4436; C07D 409/04; C07D 471/04; A61K 31/437; C07D 405/04; C07D 417/04; C07D 401/10; C07D 401/14; A61K 31/498; A61K 31/506; C07D 413/14; A61K 31/553; A61K 31/55; C07D 498/08; C07D 405/14; A61P 25/36; A61P 29/02; A61K 45/06

(57) ABSTRACT

The present invention relates to novel aminopyridine derivatives of the general formula (I) and pharmaceutical compositions comprising these compounds, as well as their therapeutic use, particularly as neuropeptide FF (NPFF) receptor antagonists, including, e.g., for the treatment or prevention of pain, opioid-induced hyperalgesia, or addiction.

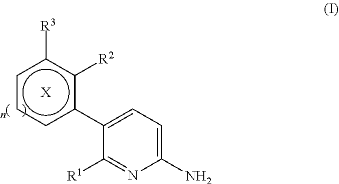

The present invention relates to novel aminopyridine derivatives of the general formula (I), as defined further below, and pharmaceutical compositions comprising these compounds, as well as their therapeutic use, particularly as neuropeptide FF (NPFF) receptor antagonists, including, e.g., for the treatment or prevention of pain, opioid-induced hyperalgesia, or addiction.

The treatment of pain, in particular chronic pain, is a major public health issue. Opiate analgesics are at present the treatment of choice for moderate or severe pain. For many patients, notably those suffering from advanced cancer, the treatment of pain requires strong, repeated doses of opiates such as morphine or fentanyl. The clinical effectiveness and tolerability of such treatments are, however, qualified by two phenomena induced by the use of opiates. The first is the tolerance effect, which is characterized by a shortening of action duration and a reduction in analgesia intensity. The clinical result is a growing need to increase the doses of opiates in order to maintain the same analgesic effect, uncorrelated with a progression of the disease. The second problem, related to repeated administration of strong doses of opiates, is known as opioid-induced hyperalgesia (OIH). Indeed, prolonged administration of opiates leads to a paradoxical increase in pain, unrelated to the initial nociceptive stimulus.

It has been suggested that such hyperalgesia would be the cause of tolerance. Tolerance would indeed be apparent since the analgesic effect characteristic of each daily dose remains constant; it would thus be the development of hypersensitivity to pain which would give the impression of a decrease in the effects of the opiate. It would thus not be the opiate that would have lost its effectiveness, but the individual who would have become hypersensitive to pain.

These two phenomena have been widely documented in both animal and human studies. Overall, they have been observed after administration of all types of opiates, regardless of the routes of administration or the doses used. Furthermore, the administration of high doses of opiates leads to a certain number of side effects such as nausea, constipation, sedation and respiratory deficiencies (e.g., delayed respiratory depression).

Currently, several strategies for mitigating these opiate-induced effects of tolerance and hyperalgesia are under investigation:

1) One of the most commonly used clinical strategies consists of combining opiates with adjuvants such as anticonvulsants or antidepressants, particularly in the treatment of neuropathic pain. In spite of some effectiveness, these additives involve numerous side effects, notably cardiac risks.
2) The rotation of opiates is also used as an alternative strategy, supported by the fact that different opiates have different affinities for each receptor, and that tolerance develops independently for each receptor. However, very few results have been described, and this strategy is the subject of much discussion.
3) NMDA receptor antagonists are known to block calcium channels, which leads in man or animals to a reduction in opiate-induced hyperalgesia as well as to a delay in tolerance effects. However, the clinical use of ketamine as an NMDA receptor antagonist involves a broad spectrum of side effects in man, notably hallucinations.

Although a certain amount of success has been reported, no strategy at present effectively blocks the effects of hyperalgesia and tolerance related to the repeated use of opiates. Consequently, the search for alternative strategies is necessary, notably in the field of neuropathic or cancer pain. Indeed, in the context of these pathologies, the treatments currently used are relatively ineffective and involve the use of high doses of opiates, leading to many particularly disabling side effects. Consequently, a major therapeutic issue relates to the development of novel drugs that act on novel therapeutic targets involved in the modulation of pain.

Research is currently under way for therapies that improve the use of opioid analgesics in mammals, particularly humans, in particular during the long-term use or the single administration of high doses, as is the case during surgical procedures.

Among the anti-opiate systems responsible for the loss of effectiveness of opiate analgesics and the appearance of hyperalgesia, neuropeptide FF (NPFF) receptors are considered to be relevant targets. The design of drugs that inhibit the action of these receptors will make it possible to restore the long-term effectiveness of opiate analgesics while preventing the appearance of opiate-induced hyperalgesia.

WO 02/24192 described Arg-Phe dipeptide derivatives which provided proof of this concept in vivo. In particular, a single administration of Arg-Phe dipeptide derivatives in the rat blocks hyperalgesia induced by administration of fentanyl, an opiate analgesic that acts as a p receptor agonist and is typically used in a hospital setting.

In Simonin et al., PNAS, 2006, 103(2):466-71, the dipeptide RF9 (referred to as Na-adamantan-1-yl-L-Arg-L-Phe-NH₂ acetate in WO 02/24192) was described as being the first nanomolar NPFF receptor antagonist. Administered in vivo in the rat, RF9 showed antihyperalgesic activity, reversing hyperalgesia induced by the repeated administration of opiate analgesics. Similar results were later observed in mice (Elhabazi, K. et al. British Journal of Pharmacology, 2012, 165, 2, 424-35).

WO 03/084303, WO 2004/083218 and WO 2018/152134 describe various further compounds as NPFF receptor antagonists.

Sampirtine and derivatives thereof have been described in U.S. Pat. No. 4,851,420. They are described as analgesic and antipyretic agents. WO 94/14780 describes pyridine derivatives as NO synthase inhibitors for use as analgesics and for the treatment of chronic neurodegenerative diseases and chronic pain. WO 2008/135826 discloses certain pyridine derivatives as Na_{v1.8} sodium channel modulators for the treatment of pain.

Yet, there is an ongoing need for novel and/or improved NPFF receptor antagonists that can be used in therapy, including in the treatment or prevention of pain.

The present invention addresses this need and provides novel aminopyridine derivatives which, surprisingly, have been found to be highly effective NPFF receptor antagonists, particularly antagonists of NPFF receptor 1 (NPFFR1), which is involved in the modulation of nociceptive signals. Moreover, these compounds have been found to prevent opioid-induced hyperalgesia, as demonstrated in a mouse model. The compounds provided herein are therefore particularly well suited for the treatment or prevention of pain, opioid-induced hyperalgesia, and other conditions in which NPFF receptors are implicated, including addiction (as further discussed herein below).

Accordingly, the present invention provides a compound of the following formula (I)

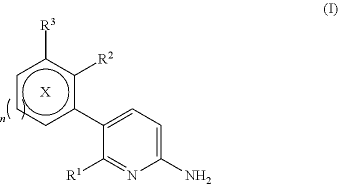

or a pharmaceutically acceptable salt or solvate thereof.

In formula (I), R¹ is selected from C₁₋₅ alkyl, C₂₋₅ alkenyl, C₂₋₅ alkynyl, halogen, C₁₋₅ haloalkyl, —CN, —NH₂, —NH(C₁₋₅ alkyl), —N(C₁₋₅ alkyl)(C₁₋₅ alkyl), —(C₀₋₃ alkylene)-cycloalkyl, and —(C₀₋₃ alkylene)-heterocycloalkyl, wherein the cycloalkyl moiety in said —(C₀₋₃ alkylene)-cycloalkyl and the heterocycloalkyl moiety in said —(C_{0.3} alkylene)-heterocycloalkyl are each optionally substituted with one or more groups R^{A}.

Ring X is phenyl or a monocyclic heteroaryl having 5+n ring members, wherein said phenyl or said heteroaryl is optionally substituted with one or more groups R^{X}.

n is 0 or 1.

It will be understood that the index n specifies the presence or absence of the corresponding ring atom of ring X (which is marked by a parenthesis). Accordingly, if n is 1, then this ring atom is present, and ring X is thus a phenyl or a heteroaryl ring having 6 ring members; conversely, if n is 0, then the corresponding ring atom is absent, and ring X is consequently a heteroaryl having 5 ring members. It will further be understood that each of the two ring atoms (of ring X) carrying the groups R² and R³ may be a carbon ring atom or a nitrogen ring atom.

As depicted in formula (I), ring X is attached to the pyridinyl ring (which carries R¹) in a specific position, such that R² is bound to ring X in 2-position and R³ is bound to ring X in 3-position in relation to said pyridinyl ring.

R² and R³ are mutually joined to form, together with ring X, a bicyclic or tricyclic heteroaryl, wherein said heteroaryl is optionally substituted with one or more groups R^{X}; or alternatively, R² is ring Y, and R³ is hydrogen or R^{X}.

Ring Y is phenyl or a monocyclic heteroaryl, wherein said phenyl or said monocyclic heteroaryl is optionally substituted with one or more groups R^{Y}, and further wherein ring X and ring Y are not both phenyl.

Each R^{A}, each R^{X}, and each R^{Y} is independently selected from C₁₋₅ alkyl, C₂₋₅ alkenyl, C₂₋₅ alkynyl, —(C₀₋₃ alkylene)-O—R^{B}, —(C₀₋₃ alkylene)-O—(C₁₋₅ alkylene)-O—R^{B}, —(C₀₋₃ alkylene)-S—R^{B}, —(C₀₋₃ alkylene)-S—(C₁₋₅ alkylene)-S—R^{B}, —(C₀₋₃ alkylene)-N(R^{B})—R^{B}, —(C₀₋₃ alkylene)-N(R^{B})—O—R^{B}, halogen, C₁₋₅ haloalkyl, —(C₀₋₃ alkylene)-O—(C₁₋₅ haloalkyl), —(C₀₋₃ alkylene)-CN, —(C₀₋₃ alkylene)-CO—R^{B}, —(C₀₋₃ alkylene)-CO—O—R^{B}, —(C₀₋₃ alkylene)-O—CO—R^{B}, —(C₀₋₃ alkylene)-CO—N(R^{B})—R^{B}, —(C₀₋₃ alkylene)-N(R^{B})—CO—R^{B}, —(C₀₋₃ alkylene)-N(R^{B})—CO—O—R^{B}, —(C₀₋₃ alkylene)-O—CO—N(R^{B})—R^{B}, —(C₀₋₃ alkylene)-SO₂—N(R^{B})—R^{B}, —(C₀₋₃ alkylene)-N(R^{B})—SO₂—(C₁₋₅ alkyl), —(C₀₋₃ alkylene)-SO₂—(C₁₋₅ alkyl), —(C₀₋₃ alkylene)-SO—(C₁₋₅ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more groups independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂—N(R^{B})—R^{B}, —N(R^{B})—SO₂—(C₁₋₅ alkyl), —SO₂—(C₁₋₅ alkyl), and —SO—(C₁₋₅ alkyl), wherein each L is independently a covalent bond or C₁₋₅ alkylene, wherein one or more —CH₂— units comprised in said C₁₋₅ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl.

The present invention further relates to a pharmaceutical composition comprising a compound of formula (I) or a pharmaceutically acceptable salt or solvate thereof, in combination with a pharmaceutically acceptable excipient. Accordingly, the invention relates to a compound of formula (I) or a pharmaceutically acceptable salt or solvate thereof, or a pharmaceutical composition comprising any of the aforementioned entities and a pharmaceutically acceptable excipient, for use as a medicament.

The invention also relates to a compound of formula (I) or a pharmaceutically acceptable salt or solvate thereof, or a pharmaceutical composition comprising any of the aforementioned entities and a pharmaceutically acceptable excipient, for use in the treatment or prevention of a disease/disorder selected from: pain, including, e.g., acute pain, chronic pain, postsurgical pain (or postoperative pain or incisional pain), cancer pain, inflammatory pain (or pain associated with an inflammatory disease/disorder), rheumatoid arthritis-associated pain, neuropathic pain, or diabetes-associated pain; opioid-induced hyperalgesia (e.g., morphine-induced hyperalgesia or fentanyl-induced hyperalgesia) or analgesic tolerance associated with chronic opioid administration; or addiction, including, e.g., substance addiction (or drug addiction), particularly alcohol addiction, amphetamine addiction, cocaine addiction, methamphetamine addiction, methylphenidate addiction, nicotine addiction, or opioid addiction, behavioral addiction (or a compulsive control disease/disorder), particularly pathological forms of any one of gambling addiction, food or overeating addiction (or compulsive overeating), sex or sexual intercourse addiction, pornography addiction, electronic communication devices addiction, mobile phone addiction, computer addiction, internet addiction, videogames addiction, internet gaming addiction, digital media addiction, physical exercise addiction (or compulsive overexercising), shopping addiction (or compulsive spending), or work addiction (or compulsive overworking), or an obsessive-compulsive spectrum disorder, particularly obsessive-compulsive disorder, anorexia (or anorexia nervosa), bulimia (or bulimia nervosa), binge eating disorder, impulse control disorder, intermittent explosive disorder, kleptomania, pyromania, compulsive hoarding, a body-focused repetitive behavior disorder, or trichotillomania.

As described above, the compounds of formula (I) can be used in the treatment or prevention of addiction. The NPFF system involvement in opioid rewarding effect or withdrawal symptoms and thus addiction is supported by several facts. Intra-cerebro-ventricular (i.c.v.) injection of NPFF produced an abstinence syndrome in morphine-dependent rats (Malin D H et al., Pharmacology Biochemistry and Behavior, 1996, 54(3), 581-585), whereas IgG i.c.v. injection from an antiserum against NPFF attenuated naloxone-induced withdrawal syndrome (Lake J R et al., Neuroscience Letters, 1991, 132(1), 29-32; Malin D H et al., Peptides, 1990, 11(5), 969-972). NPFF analogue (dNPA) i.c.v. injection in mice blocks the c-Fos expression induced by morphine in the shell of nucleus accumbens (Mouledous L et al., Synapse (New York, N.Y.), 2010, 64(9), 672-681). This data supports NPFF involvement in opioid rewarding effect since the shell of nucleus accumbens is known to be required for the acquisition of morphine-conditioned place preference (CPP) (Tolliver B K et al., The European Journal of Neuroscience, 2000, 12(9), 3399-3406). Moreover, pharmacological blockade of NPFFR1/R2 using an antagonist treatment is able to increase morphine-induced CPP and decreased naltrexone-precipitated withdrawal syndrome (Elhabazi K et al., British Journal of Pharmacology, 2012, 165(2), 424-435). In accordance with these data, injection of the NPFF analogue (1DMe)-NPYF inhibited the rewarding effect of morphine (Marchand S et al., Peptides, 2006, 27(5), 964-972). In addition, the NPFF system has been described as involved in the neuronal process of amphetamine addiction since NPFF chronic administration potentiates the behavioural sensitization to amphetamine in one study (Chen J C et al., Brain Research, 1999, 816(1), 220-224) but i.c.v. administration of NPFF inhibited the expression of amphetamine CPP in another study (Kotlinska J H et al., Peptides, 2012, 33(1), 156-163). NPFF i.c.v. injection inhibited the expression of cocaine-induced CPP (Kotlinska J et al., Peptides, 2008, 29(6), 933-939). NPFF administration is also able to inhibit ethanol-induced sensitization (Kotlinska J et al., Neuropeptides, 2007, 41(1), 51-58). The NPFF system seems to also be implicated in nicotine addiction since NPFF or analogue precipitate abstinence syndrome in nicotine dependent rats (Malin D H et al., Peptides, 1990, 11(2), 277-280). Altogether these data highlights NPFF system involvement in addiction. The compounds of formula (I) have been found to be potent NPFF receptor antagonists and can thus be used in the therapy of addiction.

Moreover, the present invention relates to the use of a compound of formula (I) or a pharmaceutically acceptable salt or solvate thereof in the preparation of a medicament for the treatment or prevention of a disease/disorder selected from: pain, including, e.g., acute pain, chronic pain, postsurgical pain (or postoperative pain or incisional pain), cancer pain, inflammatory pain (or pain associated with an inflammatory disease/disorder), rheumatoid arthritis-associated pain, neuropathic pain, or diabetes-associated pain; opioid-induced hyperalgesia (e.g., morphine-induced hyperalgesia or fentanyl-induced hyperalgesia) or analgesic tolerance associated with chronic opioid administration; or addiction, including, e.g., substance addiction (or drug addiction), particularly alcohol addiction, amphetamine addiction, cocaine addiction, methamphetamine addiction, methylphenidate addiction, nicotine addiction, or opioid addiction, behavioral addiction (or a compulsive control disease/disorder), particularly pathological forms of any one of gambling addiction, food or overeating addiction (or compulsive overeating), sex or sexual intercourse addiction, pornography addiction, electronic communication devices addiction, mobile phone addiction, computer addiction, internet addiction, videogames addiction, internet gaming addiction, digital media addiction, physical exercise addiction (or compulsive overexercising), shopping addiction (or compulsive spending), or work addiction (or compulsive overworking), or an obsessive-compulsive spectrum disorder, particularly obsessive-compulsive disorder, anorexia (or anorexia nervosa), bulimia (or bulimia nervosa), binge eating disorder, impulse control disorder, intermittent explosive disorder, kleptomania, pyromania, compulsive hoarding, a body-focused repetitive behavior disorder, or trichotillomania.

The invention likewise relates to a method of treating or preventing a disease/disorder, the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof, wherein the disease/disorder is selected from: pain, including, e.g., acute pain, chronic pain, postsurgical pain (or postoperative pain or incisional pain), cancer pain, inflammatory pain (or pain associated with an inflammatory disease/disorder), rheumatoid arthritis-associated pain, neuropathic pain, or diabetes-associated pain; opioid-induced hyperalgesia (e.g., morphine-induced hyperalgesia or fentanyl-induced hyperalgesia) or analgesic tolerance associated with chronic opioid administration; or addiction, including, e.g., substance addiction (or drug addiction), particularly alcohol addiction, amphetamine addiction, cocaine addiction, methamphetamine addiction, methylphenidate addiction, nicotine addiction, or opioid addiction, behavioral addiction (or a compulsive control disease/disorder), particularly pathological forms of any one of gambling addiction, food or overeating addiction (or compulsive overeating), sex or sexual intercourse addiction, pornography addiction, electronic communication devices addiction, mobile phone addiction, computer addiction, internet addiction, videogames addiction, internet gaming addiction, digital media addiction, physical exercise addiction (or compulsive overexercising), shopping addiction (or compulsive spending), or work addiction (or compulsive overworking), or an obsessive-compulsive spectrum disorder, particularly obsessive-compulsive disorder, anorexia (or anorexia nervosa), bulimia (or bulimia nervosa), binge eating disorder, impulse control disorder, intermittent explosive disorder, kleptomania, pyromania, compulsive hoarding, a body-focused repetitive behavior disorder, or trichotillomania. It will be understood that a therapeutically effective amount of the compound of formula (I) or the pharmaceutically acceptable salt or solvate thereof, or of the pharmaceutical composition, is to be administered in accordance with this method.

The present invention furthermore relates to the use of a compound of formula (I) or a pharmaceutically acceptable salt or solvate thereof as a neuropeptide FF (NPFF) receptor antagonist in research, particularly as a research tool compound for antagonizing NPFF receptors. Accordingly, the invention refers to the in vitro use of a compound of formula (I) or a pharmaceutically acceptable salt or solvate thereof as an NPFF receptor antagonist and, in particular, to the in vitro use of a compound of formula (I) or a pharmaceutically acceptable salt or solvate thereof as a research tool compound acting as an NPFF receptor antagonist. The invention likewise relates to a method, particularly an in vitro method, of antagonizing an NPFF receptor, the method comprising the application of a compound of formula (I) or a pharmaceutically acceptable salt or solvate thereof. The invention further relates to a method of antagonizing an NPFF receptor, the method comprising applying a compound of formula (I) or a pharmaceutically acceptable salt or solvate thereof to a test sample (e.g., a biological sample) or a test animal (i.e., a non-human test animal). The invention also refers to a method, particularly an in vitro method, of antagonizing an NPFF receptor in a sample (e.g., a biological sample), the method comprising applying a compound of formula (I) or a pharmaceutically acceptable salt or solvate thereof to said sample. The present invention further provides a method of antagonizing an NPFF receptor, the method comprising contacting a test sample (e.g., a biological sample) or a test animal (i.e., a non-human test animal) with a compound of formula (I) or a pharmaceutically acceptable salt or solvate thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compounds of formula (I) and the pharmaceutically acceptable salts and solvates thereof will be described in more detail in the following.

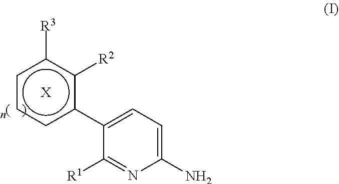

In formula (I), R¹ is selected from C₁₋₅ alkyl, C₂₋₅ alkenyl, C₂₋₅ alkynyl, halogen, C₁₋₅ haloalkyl, —CN, —NH₂, —NH(C₁₋₅ alkyl), —N(C₁₋₅ alkyl)(C₁₋₅ alkyl), —(C₀₋₃ alkylene)-cycloalkyl, and —(C₀₋₃ alkylene)-heterocycloalkyl, wherein the cycloalkyl moiety in said —(C₀₋₃ alkylene)-cycloalkyl and the heterocycloalkyl moiety in said —(C₀₋₃ alkylene)-heterocycloalkyl are each optionally substituted with one or more (e.g., one, two or three) groups R^{A}.

Preferably, R¹ is selected from C₁₋₅ alkyl, halogen (e.g., —F), C₁₋₅ haloalkyl, —CN, —NH₂, —NH(C₁₋₅ alkyl), —N(C₁₋₅ alkyl)(C₁₋₅ alkyl), —(C₀₋₃ alkylene)-(C₃₋₆ cycloalkyl), and a —(C₀₋₃ alkylene)-heterocycloalkyl having 3 to 6 ring atoms. More preferably, R¹ is selected from C₁₋₅ alkyl, C₁₋₃ haloalkyl (e.g., —CF₃), —NH₂, —NH(C₁₋₃ alkyl), —N(C₁₋₃ alkyl)(C₁₋₃ alkyl), and —(C₀₋₃ alkylene)-cyclopropyl. Even more preferably, R¹ is selected from —NH₂, C₁₋₅ alkyl, cyclopropyl, and —(CH₂)₁₋₃-cyclopropyl. Yet even more preferably, R¹ is —NH₂ or C₁₋₅ alkyl (e.g., methyl, ethyl, n-propyl, isopropyl, n-butyl, isobutyl, sec-butyl, or tert-butyl). Still more preferably, R¹ is —NH₂, ethyl or n-propyl.

Ring X is phenyl or a monocyclic heteroaryl having 5+n ring members, wherein said phenyl or said heteroaryl is optionally substituted with one or more (e.g., one or two) groups R^{X}.

n is 0 or 1.

It will be understood that the index n specifies the presence or absence of the corresponding ring atom of ring X (which is marked by a parenthesis). Accordingly, if n is 1, then this ring atom is present, and ring X is thus a phenyl or a heteroaryl ring having 6 ring members; conversely, if n is 0, then the corresponding ring atom is absent, and ring X is consequently a heteroaryl having 5 ring members. It will further be understood that each of the two ring atoms (of ring X) carrying the groups R² and R³ may be a carbon ring atom or a nitrogen ring atom.

As depicted in formula (I), ring X is attached to the pyridinyl ring (which carries R¹) in a specific position, such that R² is bound to ring X in 2-position and R³ is bound to ring X in 3-position in relation to said pyridinyl ring.

R² and R³ are mutually joined to form, together with ring X, a bicyclic or tricyclic heteroaryl, wherein said heteroaryl is optionally substituted with one or more groups R^{X}; or alternatively, R² is ring Y, and R³ is hydrogen or R^{X}.

It is preferred that R² and R³ are mutually joined to form, together with ring X, a bicyclic or tricyclic heteroaryl, wherein said bicyclic or tricyclic heteroaryl is optionally substituted with one or more (e.g., one, two, three or four) groups R^{X}. Said bicyclic or tricyclic heteroaryl may be, for example, a quinolinyl (e.g., quinolin-4-yl, quinolin-5-yl or quinolin-8-yl), isoquinolinyl (e.g., isoquinolin-4-yl, isoquinolin-5-yl or isoquinolin-8-yl), benzo[b]thiophenyl (e.g., benzo[b]thiophen-3-yl, benzo[b]thiophen-4-yl or benzo[b]thiophen-7-yl), pyrazolo[1,5-a]pyridinyl (e.g., pyrazolo[1,5-a]pyridin-3-yl, pyrazolo[1,5-a]pyridin-4-yl or pyrazolo[1,5-a]pyridin-7-yl), benzofuranyl (e.g., benzofuran-3-yl, benzofuran-4-yl or benzofuran-7-yl), 2,3-dihydrobenzofuranyl (e.g., 2,3-dihydrobenzofuran-4-yl or 2,3-dihydrobenzofuran-7-yl), indolyl (e.g., 1H-indol-1-yl, 1H-indol-3-yl, 1H-indol-4-yl or 1H-indol-7-yl), isoindolyl (e.g., 2H-isoindol-1-yl or 2H-isoindol-7-yl), isoindolin-1-onyl (e.g., isoindolin-1-on-4-yl or isoindolin-1-on-7-yl), indazolyl (e.g., 1H-indazol-1-yl, 1H-indazol-3-yl, 1H-indazol-4-yl or 1H-indazol-7-yl), 1,3-benzothiazolyl (e.g., 1,3-benzothiazol-4-yl or 1,3-benzothiazol-7-yl), chromanyl (e.g., chroman-5-yl or chroman-8-yl), 1,4-benzodioxanyl (e.g., 1,4-benzodioxan-5-yl), dibenzothiophenyl (e.g., dibenzothiophen-1-yl or dibenzothiophen-4-yl), dibenzofuranyl (e.g., dibenzofuran-1-yl or dibenzofuran-4-yl), 1,7-naphthyridinyl (e.g., 1,7-naphthyridin-8-yl), quinoxalinyl (e.g., quinoxalin-5-yl), imidazo[1,2-a]pyridinyl (e.g., imidazo[1,2-a]pyridin-5-yl or imidazo[1,2-a]pyridin-8-yl), 1,2,3,4-tetrahydroquinolinyl (e.g., 1,2,3,4-tetrahydroquinolin-8-yl), 5,6,7,8-tetrahydroacridinyl (e.g., 5,6,7,8-tetrahydroacridin-4-yl), 1,2,3,4-tetrahydrobenzo[b][1,6]naphthyridinyl (e.g., 1,2,3,4-tetrahydrobenzo[b][1,6]naphthyridin-6-yl), 2,3-dihydro-1H-cyclopenta[b]quinolinyl (e.g., 2,3-dihydro-1H-cyclopenta[b]quinolin-5-yl), quinolin-2(1H)-onyl (e.g., quinolin-2(1H)-on-8-yl), 3,4-dihydroquinolin-2(1H)-onyl (e.g., 3,4-dihydroquinolin-2(1H)-on-8-yl), indolinyl (e.g., indolin-7-yl), indolin-2-onyl (e.g., indolin-2-on-7-yl), 2,5-dihydrobenzo[b]oxepinyl (e.g., 2,5-dihydrobenzo[b]oxepin-9-yl), or 2,3,4,5-tetrahydrobenzo[b]oxepinyl (e.g., 2,3,4,5-tetrahydrobenzo[b]oxepin-9-yl). While ring X comprised in the bicyclic or tricyclic ring system is aromatic, the remaining ring(s) in this ring system may be aromatic, partially unsaturated (non-aromatic), or saturated. It is preferred that each individual ring comprised in the bicyclic or tricyclic heteroaryl ring system (which is formed from ring X, R² and R³) is aromatic, i.e. that this bicyclic or tricyclic heteroaryl ring system is fully aromatic. Moreover, it is preferred that said ring system is a bicyclic ring system, particularly a bicyclic ring system different from (other than) benzofuranyl. Corresponding examples of a bicyclic heteroaryl ring system formed from R², R³ and ring X include, in particular, quinolin-4-yl, quinolin-5-yl, quinolin-8-yl, isoquinolin-4-yl, isoquinolin-5-yl, isoquinolin-8-yl, benzo[b]thiophen-3-yl, benzo[b]thiophen-4-yl, benzo[b]thiophen-7-yl, pyrazolo[1,5-a]pyridin-3-yl, pyrazolo[1,5-a]pyridin-4-yl, pyrazolo[1,5-a]pyridin-7-yl, benzofuran-3-yl, benzofuran-4-yl, benzofuran-7-yl, 2,3-dihydrobenzofuran-4-yl, 2,3-dihydrobenzofuran-7-yl, 1H-indol-1-yl, 1H-indol-3-yl, 1H-indol-4-yl, 1H-indol-7-yl, 2H-isoindol-1-yl, 2H-isoindol-7-yl, 1H-indazol-1-yl, 1H-indazol-3-yl, 1H-indazol-4-yl, 1H-indazol-7-yl, 1,3-benzothiazol-4-yl, 1,3-benzothiazol-7-yl, chroman-5-yl, chroman-8-yl, or 1,4-benzodioxan-5-yl, wherein each of the aforementioned bicyclic ring systems is optionally substituted with one or more groups R^{X}. It is particularly preferred that R² and R³ are mutually joined to form, together with ring X, a quinolinyl or isoquinolinyl group (e.g., quinolin-8-yl or isoquinolin-8-yl), wherein said quinolinyl or isoquinolinyl group is optionally substituted with one or more (e.g., one, two, three or four) groups R^{X}. Even more preferably, R² and R³ are mutually joined to form, together with ring X, a quinolin-8-yl which is optionally substituted with one or more groups R^{X}. Accordingly, it is particularly preferred that the compound of formula (I) has the following structure:

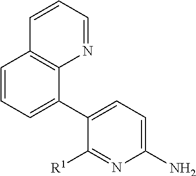

wherein the quinolin-8-yl group comprised in the above-depicted compound is optionally substituted with one or more (e.g., one, two, three or four) groups R^{X}. It will be understood that the optional substituent(s) R^{X} may be attached to any available attachment site(s) of the quinolinyl ring, i.e. on the phenyl moiety and/or the pyridinyl moiety comprised in said quinolinyl ring.

As described above, R² may also be ring Y, and R³ may be hydrogen or R^{X}. In this case, it is preferred that R³ is hydrogen.

Ring Y is phenyl or a monocyclic heteroaryl, wherein said phenyl or said monocyclic heteroaryl is optionally substituted with one or more (e.g., one, two or three) groups R^{Y}, and further wherein ring X and ring Y are not both phenyl. In other words, if ring X is phenyl (which is optionally substituted with one or more R^{X}, as defined above) and R² is ring Y, then ring Y must be a monocyclic heteroaryl (which is optionally substituted with one or more R^{Y}, as defined above).

Ring Y is preferably a monocyclic heteroaryl which is optionally substituted with one or more groups R^{Y}. Said monocyclic heteroaryl is preferably attached via a ring carbon atom to the remainder of the compound of formula (I). A particularly preferred example of ring Y is pyridinyl (e.g., pyridin-2-yl, pyridin-3-yl, or pyridin-4-yl; particularly pyridin-3-yl), wherein said pyridinyl is optionally substituted with one or more groups R^{Y}.

Each R^{A}, each R^{X}, and each R is independently selected from C₁₋₅ alkyl, C₂₋₅ alkenyl, C₂₋₅ alkynyl, —(C₀₋₃ alkylene)-O—R^{B}, —(C₀₋₃ alkylene)-O—(C₁₋₅ alkylene)-O—R^{B}, —(C₀₋₃ alkylene)-S—R^{B}, —(C₀₋₃ alkylene)-S—(C₁₋₅ alkylene)-S—R^{B}, —(C₀₋₃ alkylene)-N(R^{B})—R^{B}, —(C₀₋₃ alkylene)-N(R^{B})—O—R^{B}, halogen, C₁₋₅ haloalkyl, —(C₀₋₃ alkylene)-O—(C₁₋₅ haloalkyl), —(C₀₋₃ alkylene)-CN, —(C₀₋₃ alkylene)-CO—R^{B}, —(C₀₋₃ alkylene)-CO—O—R^{B}, —(C₀₋₃ alkylene)-O—CO—R^{B}, —(C₀₋₃ alkylene)-CO—N(R^{B})—R^{B}, —(C₀₋₃ alkylene)-N(R^{B})—CO—R^{B}, —(C₀₋₃ alkylene)-N(R^{B})—CO—O—R, —(C₀₋₃ alkylene)-O—CO—N(R^{B})—R^{B}, —(C₀₋₃ alkylene)-SO₂—N(R^{B})—R^{B}, —(C₀₋₃ alkylene)-N(R^{B})—SO₂—(C₁₋₅ alkyl), —(C₀₋₃ alkylene)-SO₂—(C₁₋₅ alkyl), —(C₀₋₃ alkylene)-SO—(C₁₋₅ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more (e.g., one, two or three) groups independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—O—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂—N(R^{B})—R^{B}, —N(R^{B})—SO₂—(C₁₋₅ alkyl), —SO₂—(C₁₋₅ alkyl), and —SO—(C₁₋₅ alkyl), wherein each L is independently a covalent bond or C₁₋₅ alkylene, wherein one or more (e.g., one, two or three) —CH₂— units comprised in said C₁₋₅ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl.

Preferably, each R^{A} is independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—O—R^{B}, —O—CO—R^{B}, —CO—N(R^{B})—R^{B}, —N(R^{B})—O—R^{B}, —N(R^{B})—CO—O—R^{B}, —O—CO—N(R^{B})—R^{B}, —SO₂—N(R^{B})—R^{B}, —N(R^{B})—SO₂—(C₁₋₅ alkyl), —SO₂—(C₁₋₅ alkyl), —SO—(CO₁ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more groups independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—O—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂—N(R^{B})—R^{B}, —N(R^{B})—SO₂—(C₁₋₅ alkyl), —SO₂—(C₁₋₅ alkyl), and —SO—(C₁₋₅ alkyl), wherein each L is independently a covalent bond or C₁₋₄ alkylene, wherein one or two —CH₂— units comprised in said C₁₋₄ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl (e.g., —CF₃ or —CH₂—CF₃). More preferably, each R^{A} is independently selected from C₁₋₅ alkyl, C₂₋₅ alkenyl, C₂₋₅ alkynyl, —OH, —O(CO₁ alkyl), —O(C₁₋₅ alkylene)-OH, —O(CO₁₋₅ alkylene)-O(C₁₋₅ alkyl), —SH, —S(CO₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—CO—O—(C₁₋₅ alkyl), —N(C₁₋₅ alkyl)-CO—O—(C₁₋₅ alkyl), —O—CO—NH—(C₁₋₅ alkyl), —O—CO—N(C₁₋₅ alkyl)-(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, —(C₁₋₄ alkylene)-aryl, —(C₁₋₄ alkylene)-heteroaryl, —(C₁₋₄ alkylene)-cycloalkyl, and —(C₁₋₄ alkylene)-heterocycloalkyl, wherein one or two —CH₂— units comprised in the C₁₋₄ alkylene moiety of said —(C₁₋₄ alkylene)-aryl, said —(C₁₋₄ alkylene)-heteroaryl, said —(C₁₋₄ alkylene)-cycloalkyl or said —(C₁₋₄ alkylene)-heterocycloalkyl are each optionally replaced by a group independently selected from —O—, —NH—, —N(C₁₋₅ alkyl)-, —CO—, and —SO₂—, and further wherein said aryl, said heteroaryl, said cycloalkyl, said heterocycloalkyl, the aryl moiety of said —(C₁₋₄ alkylene)-aryl, the heteroaryl moiety of said —(C₁₋₄ alkylene)-heteroaryl, the cycloalkyl moiety of said —(C₁₋₄ alkylene)-cycloalkyl, and the heterocycloalkyl moiety of said —(C₁₋₄ alkylene)-heterocycloalkyl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁— haloalkyl, and —CN. Even more preferably, each R^{A} is independently selected from C₁₋₅ alkyl (e.g., methyl), —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN.

Preferably, each R^{X} is independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—R^{B}, —O—CO—R^{B}, —CO—N(R^{B})—R B, —N(R^{B})—CO—R B, —N(R^{B})—CO—O—R^{B}, —O—CO—N(R^{B})—R^{B}, —SO₂N(R^{B})—R^{B}, —N(R^{B})—SO₂(C₁₋₅ alkyl), —SO₂—(C₁₋₅ alkyl), —SO—(CO₁ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more groups independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(CO₁ haloalkyl), —CN, —CO—R^{B}, —CO—O—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂N(R^{B})—R^{B}, —N(R^{B})—SO₂(C₁₋₅ alkyl), —SO₂—(C₁₋₅ alkyl), and —SO—(C₁₋₅ alkyl), wherein each L is independently a covalent bond or C₁₋₄ alkylene, wherein one or two —CH₂— units comprised in said C₁₋₄ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl (e.g., —CF₃ or —CH₂—CF₃). More preferably, each R^{X} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₅ alkyl), —NH—CO—(C₁₋₅ alkyl), —N(C₁₋₅ alkyl)-CO—(C₁₋₅ alkyl), —NH—CO—O—(C₁₋₅ alkyl), —N(C₁₋₅ alkyl)-CO—O—(C₁₋₅ alkyl), —O—CO—NH—(C₁₋₅ alkyl), —O—CO—N(C₁₋₅ alkyl)-(C₁₋₅ alkyl), —SO₂—NH₂, —SO₂—NH(C₁₋₅ alkyl), —SO₂—N(C₁₋₅ alkyl)(C₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, —(C₁₋₄ alkylene)-aryl, —(C₁₋₄ alkylene)-heteroaryl, —(C₁₋₄ alkylene)-cycloalkyl, and —(C₁₋₄ alkylene)-heterocycloalkyl, wherein one or two —CH₂— units comprised in the C₁₋₄ alkylene moiety of said —(C₁₋₄ alkylene)-aryl, said —(C₁₋₄ alkylene)-heteroaryl, said —(C₁₋₄ alkylene)-cycloalkyl or said —(C₁₋₄ alkylene)-heterocycloalkyl are each optionally replaced by a group independently selected from —O—, —NH—, —N(C₁₋₅ alkyl)-, —CO—, and —SO₂—, and further wherein said aryl, said heteroaryl, said cycloalkyl, said heterocycloalkyl, the aryl moiety of said —(C₁₋₄ alkylene)-aryl, the heteroaryl moiety of said —(C₁₋₄ alkylene)-heteroaryl, the cycloalkyl moiety of said —(C₁₋₄ alkylene)-cycloalkyl, and the heterocycloalkyl moiety of said —(C₁₋₄ alkylene)-heterocycloalkyl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN. Even more preferably, each R^{X} is independently selected from C₁₋₅ alkyl (e.g., methyl), —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN. Particularly preferred examples of R^{X} include C₁₋₅ alkyl (e.g., methyl), —OH, or halogen (e.g., —F or —Cl).

Preferably, each R^{Y} is independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—O—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂—N(R^{B})—R^{B}, —N(R^{B})—SO₂—(C₁₋₅ alkyl), —SO₂—(C₁₋₅ alkyl), —SO—(C₁₋₅ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more groups independently selected from C₁₋₅ alkyl, C₂₋₅ alkenyl, C₂₋₅ alkynyl, —O—R^{B}, —S—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—O—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂N(R^{B})—R^{B}, —N(R^{B})—SO₂(C₁₋₅ alkyl), —SO₂—(C₁₋₅ alkyl), and —SO—(C₁₋₅ alkyl), wherein each L is independently a covalent bond or C₁₋₄ alkylene, wherein one or two —CH₂— units comprised in said C₄ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl (e.g., —CF₃ or —CH₂—CF₃). More preferably, each R^{Y} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂(C₁₋₅ alkyl), —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—CO—O—(C₁₋₅ alkyl), —N(C₁₋₅ alkyl)-CO—O—(C₁₋₅ alkyl), —O—CO—NH—(C₁₋₅ alkyl), —O—CO—N(C₁₋₅ alkyl)-(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, —(C₁₋₄ alkylene)-aryl, —(C₁₋₄ alkylene)-heteroaryl, —(C₁₋₄ alkylene)-cycloalkyl, and —(C₁₋₄ alkylene)-heterocycloalkyl, wherein one or two —CH₂— units comprised in the C₁₋₄ alkylene moiety of said —(C₁₋₄ alkylene)-aryl, said —(C₁₋₄ alkylene)-heteroaryl, said —(C₁₋₄ alkylene)-cycloalkyl or said —(C₁₋₄ alkylene)-heterocycloalkyl are each optionally replaced by a group independently selected from —O—, —NH—, —N(C₁₋₅ alkyl)-, —CO—, and —SO₂—, and further wherein said aryl, said heteroaryl, said cycloalkyl, said heterocycloalkyl, the aryl moiety of said —(C₁₋₄ alkylene)-aryl, the heteroaryl moiety of said —(C₁₋₄ alkylene)-heteroaryl, the cycloalkyl moiety of said —(C₁₋₄ alkylene)-cycloalkyl, and the heterocycloalkyl moiety of said —(C₁₋₄ alkylene)-heterocycloalkyl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN. Even more preferably, each R^{Y} is independently selected from C₁₋₅ alkyl (e.g., methyl), —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN.

It will be understood that the number of optional substituents R^{A}, R^{X} and R^{Y} is limited by the number of available attachment sites (i.e., hydrogen atoms) on the respective ring group. In particular, it will be understood that the number of optional substituents R^{X} is limited by the number of available attachment sites (hydrogen atoms) on ring X or on the bicyclic or tricyclic heteroaryl ring system formed from ring X, R² and R³; preferably, the total number of substituents R^{X} in the compound of formula (I) is 0, 1, 2, 3 or 4, more preferably 0, 1, 2 or 3. While the present invention also relates to compounds of formula (I) which do not contain any substituent R^{X}, it is particularly preferred that the total number of substituents R^{X} in the compound of formula (I) is 1, 2 or 3. Even more preferably, the total number of substituents R^{X} in the compound of formula (I) is 2 or 3.

In accordance with the above, it is particularly preferred that the number of substituents R^{X} in the compound of formula (I) is 1, 2 or 3 (more preferably 2 or 3), and that each R^{X} is independently selected from C₁₋₅ alkyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN, more preferably from C₁₋₅ alkyl (e.g., methyl), —OH, and halogen (e.g., —F or —Cl). Still more preferably, the number of substituents R^{X} in the compound of formula (I) is 2 or 3, and each R^{X} is independently selected from C₁₋₅ alkyl (e.g., methyl), —OH, and halogen (e.g., —F or —Cl).

It is preferred that the compound of formula (I) is one of the specific compounds described in the examples section of this specification, including any one of the compounds of Examples 1 to 179 described further below, either in non-salt form (e.g., in free base/acid form) or as a pharmaceutically acceptable salt or solvate of the respective compound.

Accordingly, it is particularly preferred that the compound of formula (I) is selected from:
6-ethyl-5-(5-fluoroquinolin-8-yl)pyridin-2-amine;
6-fluoro-5-quinolin-8-yl-pyridin-2-ylamine;
6-methyl-5-quinolin-8-yl-pyridin-2-ylamine;
5-benzo[b]thiophen-3-yl-6-ethyl-pyridin-2-ylamine;
6-ethyl-5-(6-methoxybenzothiophen-3-yl)pyridin-2-amine;
6-ethyl-5-(8-isoquinolyl)pyridin-2-amine;
5-benzo[b]thiophen-3-yl-6-propyl-pyridin-2-ylamine;
6-propyl-5-(8-quinolyl)pyridin-2-amine;
5-(8-isoquinolyl)-6-propyl-pyridin-2-amine;
5-benzo[b]thiophen-3-yl-6-isopropyl-pyridin-2-ylamine;
6-isopropyl-5-(8-quinolyl)pyridin-2-amine;
6-isopropyl-5-(8-isoquinolyl)pyridin-2-amine;
5-benzo[b]thiophen-3-yl-6-cyclopropyl-pyridin-2-ylamine;
6-cyclopropyl-5-(8-quinolyl)pyridin-2-amine;
6-cyclopropyl-5-(8-isoquinolyl)pyridin-2-amine;
3-(1-methylindol-3-yl)pyridine-2,6-diamine;
tert-butyl 3-(2,6-diamino-3-pyridyl)indole-1-carboxylate;
3-(1H-indol-3-yl)pyridine-2,6-diamine;
3-pyrazolo[1,5-a]pyridin-3-ylpyridine-2,6-diamine;
3-(benzofuran-3-yl)pyridine-2,6-diamine;
3-(benzothiophen-3-yl)pyridine-2,6-diamine;
3-(5-fluoro-benzo[b]thiophen-3-yl)pyridine-2,6-diamine;
3-(7-fluoro-2-methylquinolin-8-yl)pyridine-2,6-diamine;
3-(1H-indol-4-yl)pyridine-2,6-diamine;
3-(1H-indol-7-yl)pyridine-2,6-diamine;
3-(1-methylindazol-7-yl)pyridine-2,6-diamine;
4-(2,6-diamino-3-pyridyl)-2-methyl-isoindolin-1-one;
3-(2,3-dihydrobenzofuran-7-yl)pyridine-2,6-diamine;
3-(benzothiophen-7-yl)pyridine-2,6-diamine;
3-(1,3-benzothiazol-4-yl)pyridine-2,6-diamine;
3-(8-quinolyl)pyridine-2,6-diamine;
3-isoquinolin-8-yl-pyridine-2,6-diamine;
3-(5-isoquinolyl)pyridine-2,6-diamine;
3-quinolin-5-yl-pyridine-2,6-diamine;
3-quinolin-4-yl-pyridine-2,6-diamine;
3-isoquinolin-4-yl-pyridine-2,6-diamine;
3-chroman-8-yl-pyridine-2,6-diamine;
3-(2,3-dihydro-benzo[1,4]dioxin-5-yl)-pyridine-2,6-diamine;
3-dibenzothiophen-4-ylpyridine-2,6-diamine;
3-dibenzofuran-4-ylpyridine-2,6-diamine;
6-ethyl-5-(2-methylbenzothiophen-3-yl)pyridin-2-amine;
6-ethyl-5-(5-methylbenzothiophen-3-yl)pyridin-2-amine;
6-ethyl-5-(5-fluorobenzothiophen-3-yl)pyridin-2-amine;
6-ethyl-5-[2-(3-pyridyl)phenyl]pyridin-2-amine;
3-[2-(3-pyridyl)phenyl]pyridine-2,6-diamine;
3-[2-(6-morpholino-3-pyridyl)phenyl]pyridine-2,6-diamine;
6-ethyl-5-(quinolin-8-yl)pyridin-2-amine;
3-(2-(1-methyl-1H-pyrazol-5-yl)phenyl)pyridine-2,6-diamine;
3-(1-methyl-1H-indol-7-yl)pyridine-2,6-diamine;
3-(benzofuran-7-yl)pyridine-2,6-diamine;
3-(benzo[b]thiophen-4-yl)pyridine-2,6-diamine;
3-(6-fluoroquinolin-8-yl)pyridine-2,6-diamine;
3-(6-methylquinolin-8-yl)pyridine-2,6-diamine;
3-(5-(trifluoromethyl)quinolin-8-yl)pyridine-2,6-diamine;
3-(5-fluoroquinolin-8-yl)pyridine-2,6-diamine;
8-(2,6-diaminopyridin-3-yl)quinolin-2(1H)-one;
3-(7-fluoroquinolin-8-yl)pyridine-2,6-diamine;
3-(3-fluoroquinolin-8-yl)pyridine-2,6-diamine;
3-(5,7-difluoroquinolin-8-yl)pyridine-2,6-diamine;
3-(3-chloro-7-fluoroquinolin-8-yl)pyridine-2,6-diamine;
3-(3,5,7-trifluoroquinolin-8-yl)pyridine-2,6-diamine;
8-(2,6-diaminopyridin-3-yl)-7-fluoroquinolin-2-ol;
8-(2,6-diaminopyridin-3-yl)-7-chloroquinolin-2-ol;
8-(2,6-diaminopyridin-3-yl)-6,7-difluoroquinolin-2-ol;
6-ethyl-5-(7-fluoroquinolin-8-yl)pyridin-2-amine;
5-(chroman-8-yl)-6-ethylpyridin-2-amine;
6-isobutyl-5-(quinolin-8-yl)pyridin-2-amine;
6-(cyclobutylmethyl)-5-(quinolin-8-yl)pyridin-2-amine;
5-(7-fluoroquinolin-8-yl)-6-(3,3,3-trifluoropropyl)pyridin-2-amine;
5-(7-fluoroquinolin-8-yl)-6-isobutylpyridin-2-amine;
5-(7-fluoroquinolin-8-yl)-6-(4,4,4-trifluorobutyl)pyridin-2-amine;
6-(cyclopropylmethyl)-5-(7-fluoroquinolin-8-yl)pyridin-2-amine;
5-(7-fluoroquinolin-8-yl)-6-isopentylpyridin-2-amine;
6-ethyl-5-(6-fluoroquinolin-8-yl)pyridin-2-amine;
6-ethyl-5-(5-(trifluoromethyl)quinolin-8-yl)pyridin-2-amine;
6-ethyl-5-(7-fluoro-2-methylquinolin-8-yl)pyridin-2-amine;
6-ethyl-5-(6-methylquinolin-8-yl)pyridin-2-amine;
5-(benzo[b]thiophen-4-yl)-6-ethylpyridin-2-amine;
5-(benzofuran-7-yl)-6-ethylpyridin-2-amine;
6-ethyl-5-(2-(6-(piperidin-1-yl)pyridin-3-yl)phenyl)pyridin-2-amine;
6-ethyl-5-(2-(6-(trifluoromethyl)pyridin-3-yl)phenyl)pyridin-2-amine;
6-ethyl-5-(4-fluoro-2-(6-morpholinopyridin-3-yl)phenyl)pyridin-2-amine;
6-ethyl-5-(5-fluoro-2-(6-morpholinopyridin-3-yl)phenyl)pyridin-2-amine;
6-ethyl-5-(2-(6-morpholinopyridin-3-yl)phenyl)pyridin-2-amine;
6-ethyl-5-(2-(5-methylpyridin-3-yl)phenyl)pyridin-2-amine;
6-ethyl-5-(2-(5-methylpyridin-3-yl)phenyl)pyridin-2-amine;
6-ethyl-5-(2-(6-fluoropyridin-3-yl)phenyl)pyridin-2-amine;
5-(2-(6-amino-2-ethylpyridin-3-yl)phenyl)pyridin-2-ol;
6-ethyl-5-(2-(6-methoxypyridin-3-yl)phenyl)pyridin-2-amine;
6-ethyl-5-(2-methylquinolin-8-yl)pyridin-2-amine;
6-ethyl-5-(4-methylquinolin-8-yl)pyridin-2-amine;
8-(6-amino-2-ethylpyridin-3-yl)quinolin-2-amine;
6-ethyl-5-(7-methylquinolin-8-yl)pyridin-2-amine;
5-(2-ethoxyquinolin-8-yl)-6-ethylpyridin-2-amine;
6-ethyl-5-(3-methylquinolin-8-yl)pyridin-2-amine;
6-ethyl-5-(5-methylquinolin-8-yl)pyridin-2-amine;
6-ethyl-5-(3-fluoroquinolin-8-yl)pyridin-2-amine;
6-ethyl-5-(7-methoxyquinolin-8-yl)pyridin-2-amine;
6-ethyl-5-(2-(trifluoromethyl)quinolin-8-yl)pyridin-2-amine;
6-ethyl-5-(1,7-naphthyridin-8-yl)pyridin-2-amine;
6-ethyl-5-(quinoxalin-5-yl)pyridin-2-amine;
6-ethyl-5-(imidazo[1,2-a]pyridin-8-yl)pyridin-2-amine;
6-ethyl-5-(imidazo[1,2-a]pyridin-5-yl)pyridin-2-amine;
6-ethyl-5-(pyrazolo[1,5-a]pyridin-7-yl)pyridin-2-amine:
5-(7-(difluoromethoxy)quinolin-8-yl)-6-ethylpyridin-2-amine;
6-ethyl-5-(1,2,3,4-tetrahydroquinolin-8-yl)pyridin-2-amine;
6-ethyl-5-(7-fluoro-3-phenylquinolin-8-yl)pyridin-2-amine;
5-(5,7-difluoroquinolin-8-yl)-6-ethylpyridin-2-amine;
6-ethyl-5-(7-(trifluoromethyl)quinolin-8-yl)pyridin-2-amine;
5-(7-chloroquinolin-8-yl)-6-ethylpyridin-2-amine;
5-(6,7-difluoroquinolin-8-yl)-6-ethylpyridin-2-amine;
8-(6-amino-2-ethylpyridin-3-yl)-6,7-difluoroquinolin-3-ol;
6-ethyl-5-(5,6,7,8-tetrahydroacridin-4-yl)pyridin-2-amine;
6-ethyl-5-(2-methyl-1,2,3,4-tetrahydrobenzo[b][1,6]naphthyridin-6-yl)pyridin-2-amine,
5-(2,3-dihydro-1H-cyclopenta[b]quinolin-5-yl)-6-ethylpyridin-2-amine;
6-ethyl-5-(2-phenylquinolin-8-yl)pyridin-2-amine;
6-ethyl-5-(2-(pyridin-3-yl)quinolin-8-yl)pyridin-2-amine;
5-(2-cyclohexylquinolin-8-yl)-6-ethylpyridin-2-amine;
6-ethyl-5-(2-(pyridin-2-yl)quinolin-8-yl)pyridin-2-amine;
6-ethyl-5-(2-(1-methylcyclopropyl)quinolin-8-yl)pyridin-2-amine;
6-ethyl-5-(2-(tetrahydro-2H-pyran-4-yl)quinolin-8-yl)pyridin-2-amine;
6-ethyl-5-(2-(pyridin-4-yl)quinolin-8-yl)pyridin-2-amine;
6-ethyl-5-(2-(imidazo[1,2-a]pyridin-6-yl)quinolin-8-yl)pyridin-2-amine;
6-ethyl-5-(2-(pyrimidin-5-yl)quinolin-8-yl)pyridin-2-amine;
6-ethyl-5-(2-(isoxazol-4-yl)quinolin-8-yl)pyridin-2-amine;
6-ethyl-5-(2-(pyrazin-2-yl)quinolin-8-yl)pyridin-2-amine;
6-ethyl-5-(2-(4-methylpyridin-3-yl)quinolin-8-yl)pyridin-2-amine;
6-ethyl-5-(2-(2-methylpyridin-3-yl)quinolin-8-yl)pyridin-2-amine;
6-ethyl-5-(2-morpholinoquinolin-8-yl)pyridin-2-amine;
6-ethyl-5-(2-(2-morpholinoethoxy)quinolin-8-yl)pyridin-2-amine;
6-ethyl-5-(2-(pyrrolidin-1-yl)quinolin-8-yl)pyridin-2-amine;
5-(2-(4,4-difluoropiperidin-1-yl)quinolin-8-yl)-6-ethylpyridin-2-amine;
5-(2-(1,4-oxazepan-4-yl)quinolin-8-yl)-6-ethylpyridin-2-amine;
6-ethyl-5-(7-fluoro-2-(1,4-oxazepan-4-yl)quinolin-8-yl)pyridin-2-amine;
6-ethyl-5-(7-fluoro-2-morpholinoquinolin-8-yl)pyridin-2-amine;
5-(2-(8-oxa-3-azabicyclo[3.2.1]octan-3-yl)-7-fluoroquinolin-8-yl)-6-ethylpyridin-2-amine;
5-(2-(azepan-1-yl)-7-fluoroquinolin-8-yl)-6-ethylpyridin-2-amine;
8-(6-amino-2-ethylpyridin-3-yl)-N-cyclohexyl-N-ethyl-7-fluoroquinolin-2-amine;
8-(6-amino-2-ethylpyridin-3-yl)-N-ethyl-7-fluoro-N-isopropylquinolin-2-amine;
8-(6-amino-2-ethylpyridin-3-yl)-N,N-dimethylquinoline-2-carboxamide;
(8-(6-amino-2-ethylpyridin-3-yl)quinolin-2-yl)(pyrrolidin-1-yl)methanone;
6-ethyl-5-(2-(methoxymethyl)quinolin-8-yl)pyridin-2-amine;
5-(3,7-difluoroquinolin-8-yl)-6-ethylpyridin-2-amine;
5-(7-chloro-3-fluoroquinolin-8-yl)-6-ethylpyridin-2-amine;
6-ethyl-5-(3,5,7-trifluoroquinolin-8-yl)pyridin-2-amine;
5-(3-chloro-7-fluoroquinolin-8-yl)-6-ethylpyridin-2-amine;
5-(3,7-dichloroquinolin-8-yl)-6-ethylpyridin-2-amine;
5-(3-chloro-5,7-difluoroquinolin-8-yl)-6-ethylpyridin-2-amine;
5-(3-chloro-6,7-difluoroquinolin-8-yl)-6-ethylpyridin-2-amine;
6-ethyl-5-(3,6,7-trifluoroquinolin-8-yl)pyridin-2-amine;
5-(3-bromo-7-fluoroquinolin-8-yl)-6-ethylpyridin-2-amine;
8-(6-amino-2-ethylpyridin-3-yl)quinoline-7-carboxamide;
8-(6-amino-2-ethylpyridin-3-yl)quinoline-7-carbonitrile;
8-(6-amino-2-ethylpyridin-3-yl)quinolin-2(1H)-one;
8-(6-amino-2-ethylpyridin-3-yl)-3,4-dihydroquinolin-2(1H)-one;
8-(6-amino-2-ethylpyridin-3-yl)-1-methylquinolin-2(1H)-one;
8-(6-amino-2-ethylpyridin-3-yl)-1-methyl-3,4-dihydroquinolin-2(1H)-one;
8-(6-amino-2-ethylpyridin-3-yl)-7-fluoroquinolin-2(1H)-one;
8-(6-amino-2-ethylpyridin-3-yl)-5,7-difluoroquinolin-2(1H)-one;
8-(6-amino-2-ethylpyridin-3-yl)-7-chloroquinolin-2(1H)-one;
8-(6-amino-2-ethylpyridin-3-yl)-6,7-difluoroquinolin-2(1H)-one;
6-ethyl-5-(1-methylindolin-7-yl)pyridin-2-amine;
7-(6-amino-2-ethylpyridin-3-yl)indolin-2-one;
6-ethyl-5-(indolin-7-yl)pyridin-2-amine;
6-ethyl-5-(1-methyl-1,2,3,4-tetrahydroquinolin-8-yl)pyridin-2-amine;
(8-(6-amino-2-ethylpyridin-3-yl)quinolin-2-yl)(morpholino)methanone;
(8-(6-amino-2-ethylpyridin-3-yl)quinolin-2-yl)(1,4-oxazepan-4-yl)methanone;
8-(6-amino-2-ethylpyridin-3-yl)-N-cyclohexyl-N-ethylquinoline-2-carboxamide;
(8-(6-amino-2-ethylpyridin-3-yl)quinolin-2-yl)(azepan-1-yl)methanone;
8-(6-amino-2-ethylpyridin-3-yl)-N-ethyl-N-isopropylquinoline-2-carboxamide;
(8-(6-amino-2-ethylpyridin-3-yl)quinolin-2-yl)(8-oxa-3-azabicyclo[3.2.1]octan-3-yl)methanone
(8-(6-amino-2-ethylpyridin-3-yl)quinolin-2-yl)(4-phenylpiperidin-1-yl)methanone;
8-(6-amino-2-ethylpyridin-3-yl)-N-(tetrahydro-2H-pyran-4-yl)quinoline-2-carboxamide;
8-(6-amino-2-ethylpyridin-3-yl)-N-benzylquinoline-2-carboxamide;
8-(6-amino-2-ethylpyridin-3-yl)-N-(oxetan-3-yl)quinoline-2-carboxamide;
6-ethyl-5-(7-fluorochroman-8-yl)pyridin-2-amine;
6-ethyl-5-(7-fluoro-2,2-dimethylchroman-8-yl)pyridin-2-amine;
6-ethyl-5-(8-fluoro-2,5-dihydrobenzo[b]oxepin-9-yl)pyridin-2-amine; or
   6-ethyl-5-(8-fluoro-2,3,4,5-tetrahydrobenzo[b]oxepin-9-yl)pyridin-2-amine;
   or a pharmaceutically acceptable salt (e.g., a hydrochloride salt) or solvate of any one of the above-mentioned compounds.

The present invention also relates to each of the intermediates described in the examples section of this specification, including any one of these intermediates in non-salt form or in the form of a salt or solvate (e.g., a pharmaceutically acceptable salt or solvate) of the respective compound. Such intermediates can be used, in particular, in the synthesis of the compounds of formula (I).

It is particularly preferred that the compound of formula (I) is a compound of the following formula or a pharmaceutically acceptable salt or solvate thereof:

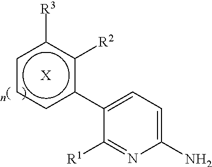

wherein:
R¹ is selected from C₁₋₅ alkyl, C₂₋₅ alkenyl, C₂₋₅ alkynyl, —Br, —I, C₁₋₅ haloalkyl, —CN, —NH₂, —NH(C₁₋₅ alkyl), —N(C₁₋₅ alkyl)(C₁₋₅ alkyl), —(C₀₋₃ alkylene)-cycloalkyl, and —(C₀₋₃ alkylene)-heterocycloalkyl, wherein the cycloalkyl moiety in said —(C₀₋₃ alkylene)-cycloalkyl and the heterocycloalkyl moiety in said —(C₀₋₃ alkylene)-heterocycloalkyl are each optionally substituted with one or more groups R^{A};
ring X is phenyl or a monocyclic heteroaryl having 5+n ring members, wherein said phenyl or said heteroaryl is optionally substituted with one or more groups R^{X};
n is 0 or 1;
R² and R³ are mutually joined to form, together with ring X, a bicyclic or tricyclic heteroaryl, wherein said heteroaryl is optionally substituted with one or more groups R^{X}, and wherein said heteroaryl is not 1H-indazol-4-yl or benzimidazolyl; or alternatively, R² is ring Y, and R³ is hydrogen or R^{X};
ring Y is phenyl or a monocyclic heteroaryl, wherein said phenyl or said monocyclic heteroaryl is optionally substituted with one or more groups R^{Y}, and further wherein ring X and ring Y are not both phenyl; and
each R^{A}, each R^{X}, and each R^{Y} is independently selected from C₁₋₅ alkyl, C₂₋₅ alkenyl, C₂₋₅ alkynyl, —(C₀₋₃ alkylene)-O—R^{B}, —(C₀₋₃ alkylene)-O—(C₁₋₅ alkylene)-O—R^{B}, —(C₀₋₃ alkylene)-S—R^{B}, —(C₀₋₃ alkylene)-S—(C₁₋₅ alkylene)-S—R^{B}, —(C₀₋₃ alkylene)-N(R^{B})R^{B}, —(C₀₋₃ alkylene)-N(R^{B})—O—R^{B}, halogen, C₁₋₅ haloalkyl, —(C₀₋₃ alkylene)-O—(C₁₋₅ haloalkyl), —(C₀₋₃ alkylene)-CN, —(C₀₋₃ alkylene)-CO—R^{B}, —(C₁₋₃ alkylene)-COOH, —(C₀₋₃ alkylene)-CO—O—(C₁₋₅ alkyl), —(C₀₋₃ alkylene)-CO—O—(C₁₋₅ haloalkyl), —(C₀₋₃ alkylene)-O—CO—R^{B}, —(C₀₋₃ alkylene)-CO—N(R^{B})—R^{B}, —(C₀₋₃ alkylene)-N(R^{B})—CO—R^{B}, —(C₀₋₃ alkylene)-N(R^{B})—CO—O—R^{B}, —(C₀₋₃ alkylene)-O—CO—N(R^{B})—R^{B}, —(C₀₋₃ alkylene)-SO₂—N(R^{B})—R^{B}, —(C₀₋₃ alkylene)-N(R^{B})—SO₂—(C₁₋₅ alkyl), —(C₀₋₃ alkylene)-SO₂—(C₁₋₅ alkyl), —(C₀₋₃ alkylene)-SO—(C₁₋₅ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more groups independently selected from C₁₋₅ alkyl, C₂₋₅ alkenyl, C₂₋₅ alkynyl, —O—R^{B}, —O—(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(C₁₋₅ haloalkyl), —CN, —CO—R^{B}, —CO—O—R^{B}, —O—CO—R^{B}, —CO—N(R^{B})—R^{B}, —N(R^{B})—CO—R^{B}, —N(R^{B})—CO—O—R^{B}, —O—CO—N(R^{B})—R^{B}, —SO₂—N(R^{B})—R^{B}, —N(R^{B})—SO₂—(C₁₋₅ alkyl), —SO₂—(C₁₋₅ alkyl), and —SO—(C₁₋₅ alkyl), wherein each L is independently a covalent bond or C₁₋₅ alkylene, wherein one or more —CH₂— units comprised in said C₁₋₅ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl.

The preferred definitions for the groups/variables in the compound of formula (I), as described and defined herein above, likewise apply to the respective groups/variables in this compound.

In a first specific embodiment, the compound of formula (I) is a compound of the following formula (Ia) or a pharmaceutically acceptable salt or solvate thereof:

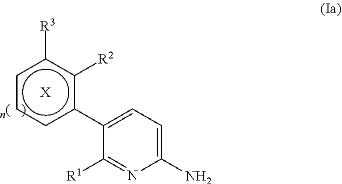

wherein R¹ is —NH₂, and wherein the further groups/variables in formula (Ia), including in particular ring X, n, R² and R³, have the same meanings, including the same preferred meanings, as described and defined herein above for the compound of formula (I).

In a second specific embodiment, the compound of formula (I) is a compound of formula (Ia), as depicted above, or a pharmaceutically acceptable salt or solvate thereof, wherein R¹ is C₁₋₅ alkyl, and wherein the further groups/variables in formula (Ia), including in particular ring X, n, R² and R³, have the same meanings, including the same preferred meanings, as described and defined herein above for the compound of formula (I).

In a third specific embodiment, the compound of formula (I) is a compound of formula (Ia), as depicted above, or a pharmaceutically acceptable salt or solvate thereof, wherein R¹ is methyl, and wherein the further groups/variables in formula (Ia), including in particular ring X, n, R² and R³, have the same meanings, including the same preferred meanings, as described and defined herein above for the compound of formula (I).

In a fourth specific embodiment, the compound of formula (I) is a compound of formula (Ia), as depicted above, or a pharmaceutically acceptable salt or solvate thereof, wherein R¹ is ethyl, and wherein the further groups/variables in formula (Ia), including in particular ring X, n, R² and R³, have the same meanings, including the same preferred meanings, as described and defined herein above for the compound of formula (I).

In a fifth specific embodiment, the compound of formula (I) is a compound of formula (Ia), as depicted above, or a pharmaceutically acceptable salt or solvate thereof, wherein R⁴ is n-propyl, and wherein the further groups/variables in formula (Ia), including in particular ring X, n, R² and R³, have the same meanings, including the same preferred meanings, as described and defined herein above for the compound of formula (I).

In a sixth specific embodiment, the compound of formula (I) is a compound of formula (Ia), as depicted above, or a pharmaceutically acceptable salt or solvate thereof, wherein R¹ is isopropyl, and wherein the further groups/variables in formula (Ia), including in particular ring X, n, R² and R³, have the same meanings, including the same preferred meanings, as described and defined herein above for the compound of formula (I).

In a seventh specific embodiment, the compound of formula (I) is a compound of the following formula (Ib) or a pharmaceutically acceptable salt or solvate thereof:

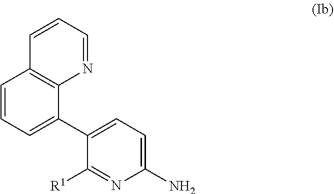

wherein R¹ is —NH₂, wherein the quinolin-8-yl group comprised in the compound of formula (Ib) is optionally substituted with one or more (e.g., one, two, three or four) groups R^{X}, and wherein R^{X} has the same meaning, including the same preferred meaning, as described and defined herein above for the compound of formula (I).

In an eighth specific embodiment, the compound of formula (I) is a compound of formula (Ib), as depicted above, or a pharmaceutically acceptable salt or solvate thereof, wherein R¹ is C₁₋₅ alkyl, wherein the quinolin-8-yl group comprised in the compound of formula (Ib) is optionally substituted with one or more (e.g., one, two, three or four) groups R^{X}, and wherein R^{X} has the same meaning, including the same preferred meaning, as described and defined herein above for the compound of formula (I).

In a ninth specific embodiment, the compound of formula (I) is a compound of formula (Ib), as depicted above, or a pharmaceutically acceptable salt or solvate thereof, wherein R¹ is methyl, wherein the quinolin-8-yl group comprised in the compound of formula (Ib) is optionally substituted with one or more (e.g., one, two, three or four) groups R^{X}, and wherein R^{X} has the same meaning, including the same preferred meaning, as described and defined herein above for the compound of formula (I).

In a tenth specific embodiment, the compound of formula (I) is a compound of formula (Ib), as depicted above, or a pharmaceutically acceptable salt or solvate thereof, wherein R¹ is ethyl, wherein the quinolin-8-yl group comprised in the compound of formula (Ib) is optionally substituted with one or more (e.g., one, two, three or four) groups R^{X}, and wherein R^{X} has the same meaning, including the same preferred meaning, as described and defined herein above for the compound of formula (I).

In an 11^{th} specific embodiment, the compound of formula (I) is a compound of formula (Ib), as depicted above, or a pharmaceutically acceptable salt or solvate thereof, wherein R¹ is n-propyl, wherein the quinolin-8-yl group comprised in the compound of formula (Ib) is optionally substituted with one or more (e.g., one, two, three or four) groups R^{X}, and wherein R^{X} has the same meaning, including the same preferred meaning, as described and defined herein above for the compound of formula (I).

In a 12^{th} specific embodiment, the compound of formula (I) is a compound of formula (Ib), as depicted above, or a pharmaceutically acceptable salt or solvate thereof, wherein R¹ is isopropyl, wherein the quinolin-8-yl group comprised in the compound of formula (Ib) is optionally substituted with one or more (e.g., one, two, three or four) groups R^{X}, and wherein R^{X} has the same meaning, including the same preferred meaning, as described and defined herein above for the compound of formula (I).

In a 13^{th} specific embodiment, the compound of formula (I) is a compound of the following formula (Ic) or a pharmaceutically acceptable salt or solvate thereof:

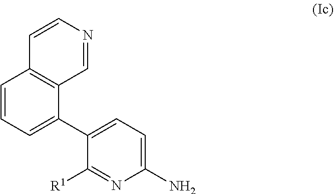

wherein R¹ is —NH₂, wherein the isoquinolin-8-yl group comprised in the compound of formula (Ic) is optionally substituted with one or more (e.g., one, two, three or four) groups R^{X}, and wherein R^{X} has the same meaning, including the same preferred meaning, as described and defined herein above for the compound of formula (I).

In a 14^{th} specific embodiment, the compound of formula (I) is a compound of formula (Ic), as depicted above, or a pharmaceutically acceptable salt or solvate thereof, wherein R¹ is C₁₋₅ alkyl, wherein the isoquinolin-8-yl group comprised in the compound of formula (Ic) is optionally substituted with one or more (e.g., one, two, three or four) groups R^{X}, and wherein R^{X} has the same meaning, including the same preferred meaning, as described and defined herein above for the compound of formula (I).

In a 15^{th} specific embodiment, the compound of formula (I) is a compound of formula (Ic), as depicted above, or a pharmaceutically acceptable salt or solvate thereof, wherein R¹ is methyl, wherein the isoquinolin-8-yl group comprised in the compound of formula (Ic) is optionally substituted with one or more (e.g., one, two, three or four) groups R^{X}, and wherein R^{X} has the same meaning, including the same preferred meaning, as described and defined herein above for the compound of formula (I).

In a 16^{th} specific embodiment, the compound of formula (I) is a compound of formula (Ic), as depicted above, or a pharmaceutically acceptable salt or solvate thereof, wherein R¹ is ethyl, wherein the isoquinolin-8-yl group comprised in the compound of formula (Ic) is optionally substituted with one or more (e.g., one, two, three or four) groups R^{X}, and wherein R^{X} has the same meaning, including the same preferred meaning, as described and defined herein above for the compound of formula (I).

In a 17^{th} specific embodiment, the compound of formula (I) is a compound of formula (Ic), as depicted above, or a pharmaceutically acceptable salt or solvate thereof, wherein R¹ is n-propyl, wherein the isoquinolin-8-yl group comprised in the compound of formula (Ic) is optionally substituted with one or more (e.g., one, two, three or four) groups R^{X}, and wherein R^{X} has the same meaning, including the same preferred meaning, as described and defined herein above for the compound of formula (I).

In an 18^{th} specific embodiment, the compound of formula (I) is a compound of formula (Ic), as depicted above, or a pharmaceutically acceptable salt or solvate thereof, wherein R¹ is isopropyl, wherein the isoquinolin-8-yl group comprised in the compound of formula (Ic) is optionally substituted with one or more (e.g., one, two, three or four) groups R^{X}, and wherein R^{X} has the same meaning, including the same preferred meaning, as described and defined herein above for the compound of formula (I).

In a 19^{th} specific embodiment, the compound of formula (I) is a compound of the following formula (Id) or a pharmaceutically acceptable salt or solvate thereof:

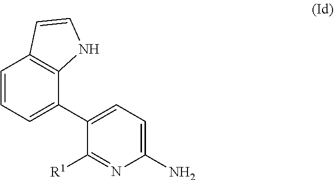

wherein R¹ is —NH₂, wherein the 1H-indol-7-yl group comprised in the compound of formula (Id) is optionally substituted with one or more (e.g., one, two, three or four) groups R^{X}, and wherein R^{X} has the same meaning, including the same preferred meaning, as described and defined herein above for the compound of formula (I).

In a 20^{th} specific embodiment, the compound of formula (I) is a compound of formula (Id), as depicted above, or a pharmaceutically acceptable salt or solvate thereof, wherein R¹ is C₁₋₅ alkyl, wherein the 1H-indol-7-yl group comprised in the compound of formula (Id) is optionally substituted with one or more (e.g., one, two, three or four) groups R^{X}, and wherein R^{X} has the same meaning, including the same preferred meaning, as described and defined herein above for the compound of formula (I).

In a 21^{st} specific embodiment, the compound of formula (I) is a compound of formula (Id), as depicted above, or a pharmaceutically acceptable salt or solvate thereof, wherein R¹ is methyl, wherein the 1H-indol-7-yl group comprised in the compound of formula (Id) is optionally substituted with one or more (e.g., one, two, three or four) groups R^{X}, and wherein R^{X} has the same meaning, including the same preferred meaning, as described and defined herein above for the compound of formula (I).

In a 22^{nd} specific embodiment, the compound of formula (I) is a compound of formula (Id), as depicted above, or a pharmaceutically acceptable salt or solvate thereof, wherein R¹ is ethyl, wherein the 1H-indol-7-yl group comprised in the compound of formula (Id) is optionally substituted with one or more (e.g., one, two, three or four) groups R^{X}, and wherein R^{X} has the same meaning, including the same preferred meaning, as described and defined herein above for the compound of formula (I).

In a 23^{rd} specific embodiment, the compound of formula (I) is a compound of formula (Id), as depicted above, or a pharmaceutically acceptable salt or solvate thereof, wherein R¹ is n-propyl, wherein the 1H-indol-7-yl group comprised in the compound of formula (Id) is optionally substituted with one or more (e.g., one, two, three or four) groups R^{X}, and wherein R^{X} has the same meaning, including the same preferred meaning, as described and defined herein above for the compound of formula (I).

In a 24^{th} specific embodiment, the compound of formula (I) is a compound of formula (Id), as depicted above, or a pharmaceutically acceptable salt or solvate thereof, wherein R¹ is isopropyl, wherein the 1H-indol-7-yl group comprised in the compound of formula (Id) is optionally substituted with one or more (e.g., one, two, three or four) groups R^{X}, and wherein R^{X} has the same meaning, including the same preferred meaning, as described and defined herein above for the compound of formula (I).

In a 25^{th} specific embodiment, the compound of formula (I) is a compound of the following formula (Ie) or a pharmaceutically acceptable salt or solvate thereof:

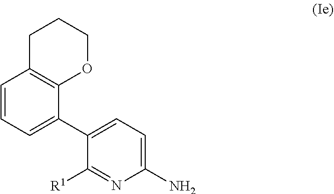

wherein R¹ is —NH₂, wherein the chroman-8-yl group comprised in the compound of formula (Ie) is optionally substituted with one or more (e.g., one, two, three or four) groups R^{X}, and wherein R^{X} has the same meaning, including the same preferred meaning, as described and defined herein above for the compound of formula (I).

In a 26^{th} specific embodiment, the compound of formula (I) is a compound of formula (Ie), as depicted above, or a pharmaceutically acceptable salt or solvate thereof, wherein R¹ is C₁₋₅ alkyl, wherein the chroman-8-yl group comprised in the compound of formula (Ie) is optionally substituted with one or more (e.g., one, two, three or four) groups R^{X}, and wherein R^{X} has the same meaning, including the same preferred meaning, as described and defined herein above for the compound of formula (I).

In a 27^{th} specific embodiment, the compound of formula (I) is a compound of formula (Ie), as depicted above, or a pharmaceutically acceptable salt or solvate thereof, wherein R¹ is methyl, wherein the chroman-8-yl group comprised in the compound of formula (Ie) is optionally substituted with one or more (e.g., one, two, three or four) groups R^{X}, and wherein R^{X} has the same meaning, including the same preferred meaning, as described and defined herein above for the compound of formula (I).

In a 28^{th} specific embodiment, the compound of formula (I) is a compound of formula (Ie), as depicted above, or a pharmaceutically acceptable salt or solvate thereof, wherein R¹ is ethyl, wherein the chroman-8-yl group comprised in the compound of formula (Ie) is optionally substituted with one or more (e.g., one, two, three or four) groups R^{X}, and wherein R^{X} has the same meaning, including the same preferred meaning, as described and defined herein above for the compound of formula (I).

In a 29^{th} specific embodiment, the compound of formula (I) is a compound of formula (Ie), as depicted above, or a pharmaceutically acceptable salt or solvate thereof, wherein R¹ is n-propyl, wherein the chroman-8-yl group comprised in the compound of formula (Ie) is optionally substituted with one or more (e.g., one, two, three or four) groups R^{X}, and wherein R^{X} has the same meaning, including the same preferred meaning, as described and defined herein above for the compound of formula (I).

In a 30^{th} specific embodiment, the compound of formula (I) is a compound of formula (Ie), as depicted above, or a pharmaceutically acceptable salt or solvate thereof, wherein R¹ is isopropyl, wherein the chroman-8-yl group comprised in the compound of formula (Ie) is optionally substituted with one or more (e.g., one, two, three or four) groups R^{X}, and wherein R^{X} has the same meaning, including the same preferred meaning, as described and defined herein above for the compound of formula (I).

For a person skilled in the field of synthetic chemistry, various ways for the preparation of the compounds of formula (I) will be readily apparent. For example, the compounds of formula (I) can be prepared as described in the following and, in particular, they can be prepared in accordance with or in analogy to the synthetic routes described in the examples section.

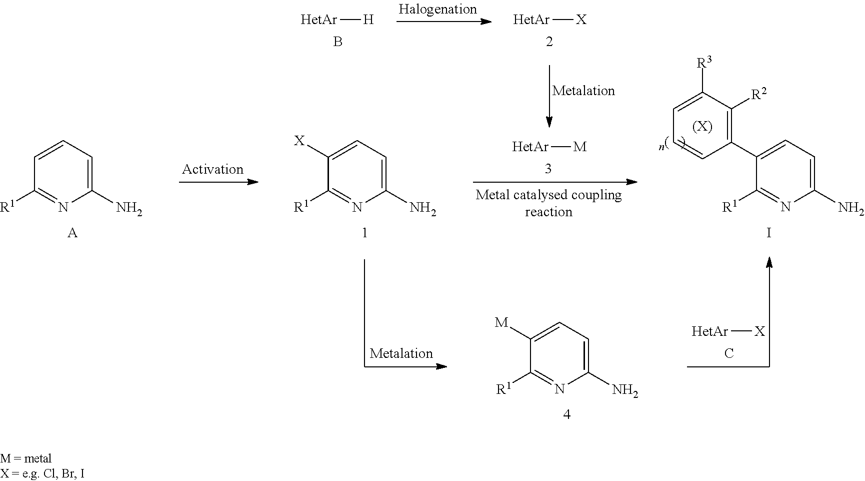

When R² and R³ are mutually joined to form, together with ring X, a bicyclic or tricyclic heteroaryl, the corresponding compounds of general formula (I) can be synthesized by metal catalysed cross coupling reactions such as Stille or Suzuki coupling reactions (Jana et al., Chem. Rev., 2011, 111, 1417). These reactions can be performed from the relevant activated compounds 1 and various metallic partners 3 like stannanes or boronates. The activated compounds 1 can be prepared by regioselective halogenation from the corresponding 2-amino-6-substituted pyridines A typically using N-bromosuccinimide, bromine or iodine as a halogenation reagent in an appropriate solvent such dichloromethane or tetrahydrofuran. The metallic partners 3 can be prepared, for example, from the corresponding halides 2 by metalation, typically using halogen-exchange conditions or performing a palladium-catalysed Miyaura borylation (Miyaura et al., J. Org. Chem., 1995, 60, 7508). The halides 2 can be prepared by halogenation of the corresponding hetero-aromatic B using similar conditions as for the preparation of halides 1. The halides 2, especially quinoline derivates could be prepared by a Skraup reaction (Dennmark et al. J. Org. Chem., 2006, 71, 1668) or by a Friedlander reaction (Marco-Contelles et al., Chem. Rev.; 2006, 22, 3825) from the corresponding haloaniline. Quinolone could be synthesized by condensation of methyl 3,3-dimethyoxypropionate from the corresponding haloaniline (Zaugg et al., Org. Process. Res. Dev., 2017, 7, 1003). Other heterocycles could be prepared using classical organic chemistry method.

Alternatively, the compounds of general formula (I) can be synthesized by these cross-coupling reactions from 2-amino-6-substituted-pyridines 4 bearing a metal such as tin or boron in position 5 and various activated heteroaryl compounds C.

Alternatively, the 2-aminopyridine A can be protected to improve the chemistry.

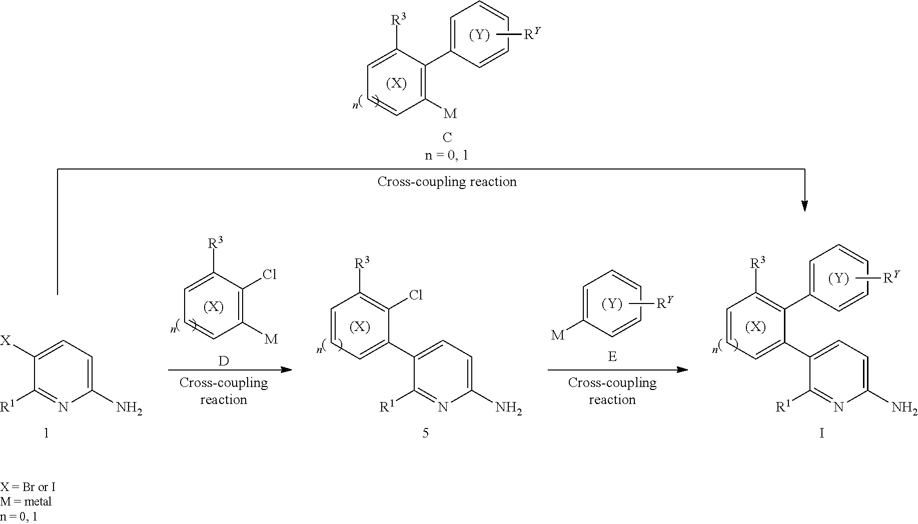

When R² is ring Y, and R³ is hydrogen or R^{X}, the corresponding compounds of general formula (I) can be synthesized from the 2-amino-6-substituted pyridines 1 by cross-coupling reactions, such as Stille or Suzuki coupling, with the 2-metalo-heterobiphenyl derivatives C.

Alternatively, the compounds of general formula (I) can be synthesized from the same 2-amino-6-substituted pyridines 1 by cross-coupling reaction, such as Stille or Suzuki coupling, with the metalo-derivatives D, followed by a second cross-coupling reaction, such as Suzuki coupling, with metalo-derivatives E.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, p-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I). As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint. If the term “about” is used in connection with a parameter that is quantified in integers, such as the number of nucleotides in a given nucleic acid, the numbers corresponding to ±10% or ±5% of the indicated numerical value are to be rounded to the nearest integer (using the tie-breaking rule “round half up”).

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the present invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts include, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts include, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

Moreover, the scope of the invention embraces the compounds of formula (I) in any solvated form, including, e.g., solvates with water (i.e., as a hydrate) or solvates with organic solvents such as, e.g., methanol, ethanol or acetonitrile (i.e., as a methanolate, ethanolate or acetonitrilate). All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments (pharmaceutical compositions). The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-γ-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, sufficient salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration. Even more preferably, the compounds or pharmaceutical compositions provided herein are to be administered orally.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, particularly 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease or condition that is to be treated or prevented with the compound of formula (I)). The invention particularly relates to a compound of formula (I) or a pharmaceutically acceptable salt or solvate thereof, or a pharmaceutical composition comprising said compound, for use in the treatment or prevention of pain without concomitantly administering any further analgesic.

However, the compound of formula (I) or a pharmaceutical composition comprising the compound of formula (I) can also be administered in combination with one or more further therapeutic agents. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in two or more different (separate) pharmaceutical formulations.

In particular, the one or more further therapeutic agents to be administered in combination with a compound of formula (I) may be an analgesic, preferably an opioid analgesic. It has been found that the compounds of formula (I) can improve the analgesic effect of opioids and can prevent, reduce or delay the development of opioid-induced hyperalgesia, which renders the combined use of a compound of formula (I) and an opioid analgesic particularly advantageous, including for the treatment or prevention of pain, but also for other therapeutic approaches in which opioid analgesics are used. The opioid analgesic(s) to be administered in combination with a compound of formula (I) according to the present invention are preferably selected from codeine, morphine, opium, laudanum, paregoric, acetyldihydrocodeine, benzylmorphine, buprenorphine, desomorphine, diamorphine, dihydrocodeine, dihydromorphine, ethylmorphine, hydrocodone, hydromorphinol, hydromorphone, nicocodeine, nicodicodine, nicomorphine, oxycodone, oxymorphone, thebacon, alfentanil, alphaprodine, anileridine, butorphanol, carfentanil, dextromoramide, dextropropoxyphene, dezocine, fentanyl, ketobemidone, levorphanol, lofentanil, meptazinol, methadone, nalbuphine, NFEPP (i.e., N-(3-fluoro-1-phenethylpiperidin-4-yl)-N-phenylpropionamide), pentazocine, pethidine (or meperidine), phenadoxone, phenazocine, piminodine, piritramide, propiram, remifentanil, sufentanil, tapentadol, tilidine, tramadol, and pharmaceutically acceptable salts and solvates of any of the aforementioned agents.

The present invention thus relates to a compound of formula (I) or a pharmaceutically acceptable salt or solvate thereof, or a pharmaceutical composition comprising any of the aforementioned entities in combination with a pharmaceutically acceptable excipient, for use in therapy, particularly for use in the treatment or prevention of pain, wherein the compound or the pharmaceutical composition is to be administered in combination with one or more further analgesics, preferably in combination with one or more opioid analgesics. The opioid analgesics may be, for example, selected from codeine, morphine, opium, laudanum, paregoric, acetyldihydrocodeine, benzylmorphine, buprenorphine, desomorphine, diamorphine, dihydrocodeine, dihydromorphine, ethylmorphine, hydrocodone, hydromorphinol, hydromorphone, nicocodeine, nicodicodine, nicomorphine, oxycodone, oxymorphone, thebacon, alfentanil, alphaprodine, anileridine, butorphanol, carfentanil, dextromoramide, dextropropoxyphene, dezocine, fentanyl, ketobemidone, levorphanol, lofentanil, meptazinol, methadone, nalbuphine, NFEPP, pentazocine, pethidine, phenadoxone, phenazocine, piminodine, piritramide, propiram, remifentanil, sufentanil, tapentadol, tilidine, and tramadol. The combined administration of a compound of formula (I) or a pharmaceutical composition comprising a compound of formula (I) according to the present invention with one or more analgesics, particularly one or more opioid analgesics, may be effected, e.g., by simultaneous/concomitant administration (either in a single pharmaceutical formulation or in separate pharmaceutical formulations) or by sequential/separate administration.

Moreover, the compound of formula (I) according to the invention may also be administered in combination with a standard of care treatment of addiction. For example, the compound of formula (I) may be administered in combination with naltrexone or naloxone. The present invention thus relates to a compound of formula (I) or a pharmaceutically acceptable salt or solvate thereof, or a pharmaceutical composition comprising any of the aforementioned entities in combination with a pharmaceutically acceptable excipient, for use in the treatment or prevention of addiction, wherein the compound or the pharmaceutical composition is to be administered in combination with standard of care treatment of addiction (e.g., in combination with naltrexone or naloxone).

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all documents mentioned herein are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The present invention is also described by the appended illustrative figure.

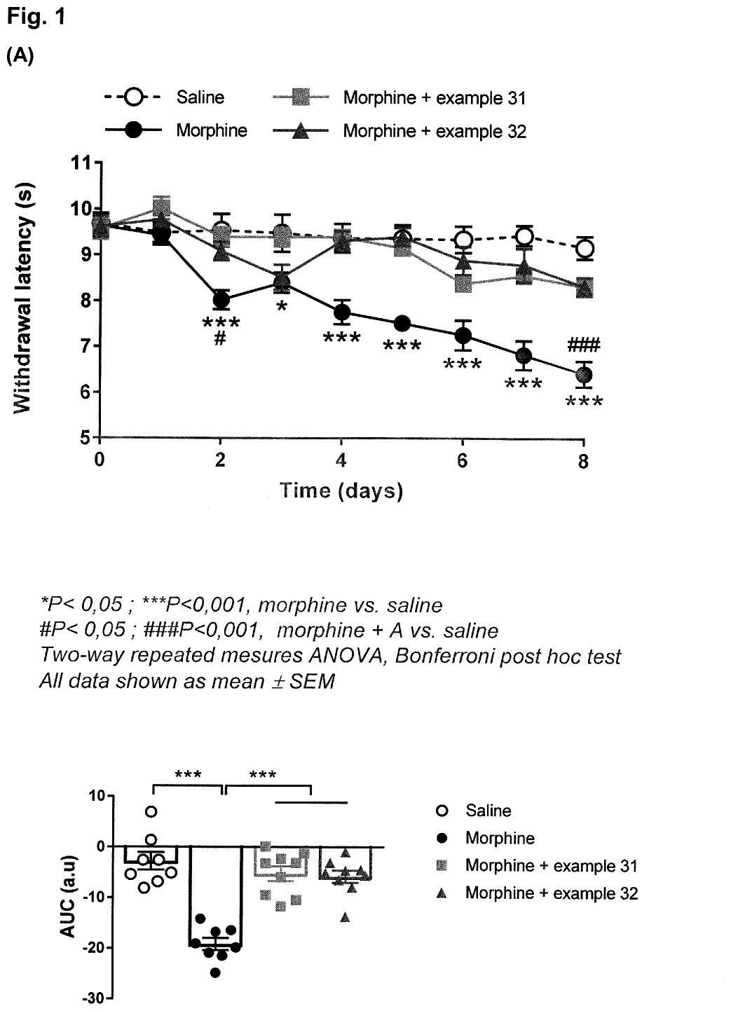

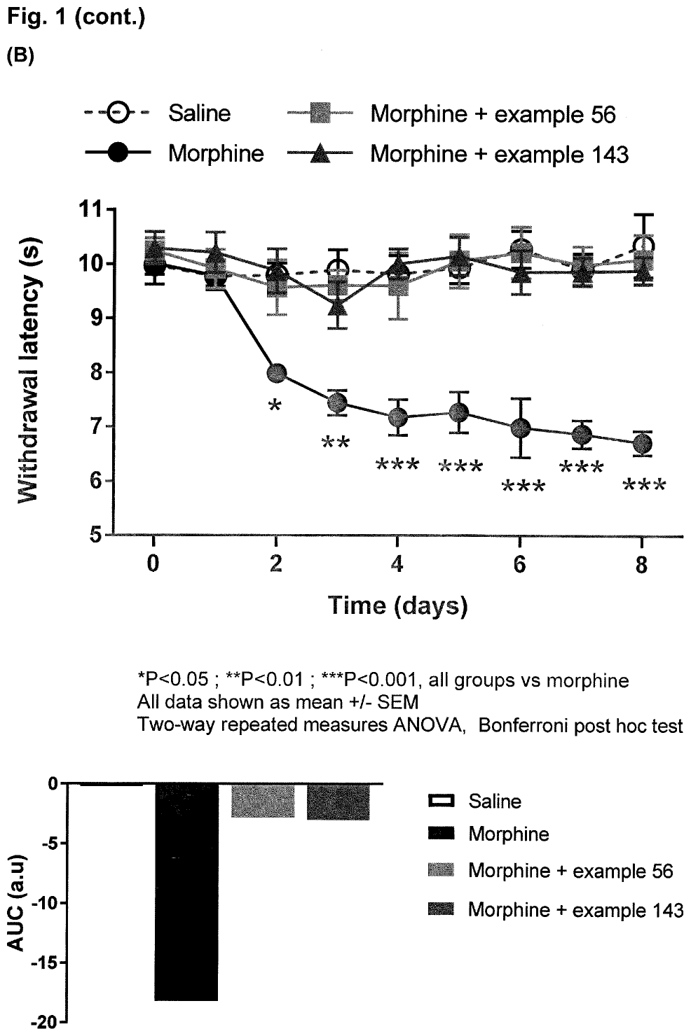

FIG. 1: Effect of exemplary compounds of formula (I) on morphine-induced hyperalgesia in a mouse model (see Example 181). The tested compounds are (A) Examples 31 and 32 as well as (B) Examples 56 and 143. The figure shows the mean time of tail withdrawal latency in each group of animals. The anti-hyperalgesia effect of the tested compounds was compared to vehicle-treated group using ANOVA test followed by the Bonferroni's test. The insert at the bottom shows the comparison between groups of the global Area Under Curve (AUC) over D0 to D8 period.

The invention will now be described by reference to the following examples which are merely illustrative and are not to be construed as a limitation of the scope of the present invention.

The compounds described in the following examples section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### EXAMPLES

All reagents were commercial grade and used without further purification. Reactions were typically run using anhydrous solvents under argon atmosphere. Organic layers were usually dried over sodium or magnesium sulphate or filtered through an Isolute® SPE Single Fritted column. Thin layer chromatography was carried out using pre-coated silica gel F-254 plate. Flash column chromatography was performed using a Biotage® isolera 4 system, with the Biotage® SNAP cartridge KP-Sil (50 μm) if not specified. In specific cases, a Biotage® SNAP cartridge KP-NH or Interchim PF-15SIHP-F0025 (15 μm or 20 μm) could be used.

Reactions were monitored and compounds were characterized using a Waters Acquity UPLC H-class system with a photodiode array detector (190-400 nm). An Acquity CSH C18 1.7 μM 2.1×30 mm column was used. The mobile phase consisted in a gradient of A and B: A was water with 0.025% of trifluoroacetic acid and B was acetonitrile with 0.025% of trifluoroacetic acid. Flow rate was 0.8 mL per min. All analyses were performed at 55° C. The UPLC system was coupled to a Waters SQD2 platform. All mass spectra were full-scan experiments (mass range 100-800 amu). Mass spectra were obtained using positive electrospray ionization.

Preparative LC-MS were performed using a Waters HPLC system with a 2767 sample manager, a 2525 pump, a photodiode array detector (190-400 nm) enabling analytical and preparative modes. An Xselect CSH C18 3.5 μM 4.6×50 mm column was used in analytical mode and an Xselect CSH C18 5 μM 19×100 mm column in preparative mode. The mobile phase consisted in both cases in a gradient of A and B: A was water with 0.1% of formic acid and B was acetonitrile with 0.1% of formic acid. Flow rate was 1 mL per min in analytical mode and 25 mL per min in preparative mode. All LCMS analyses/purifications were performed at room temperature. The HPLC system was coupled with a Waters Acquity QDa detector. All mass spectra were full-scan experiments (mass range 100-800 amu). Mass spectra were obtained using positive electrospray ionization.

All NMR experiments were recorded on a Bruker AMX-400 spectrometer. Proton chemical shifts are listed relative to residual DMSO (2.50 ppm). Splitting patterns are designated as s (singlet); d (doublet); dd (doublet of doublet); t (triplet); dt (doublet of triplet); td (triplet of doublet); tt (triplet of triplet); q (quartet); quint (quintuplet); m (multiplet); bs (broad singlet).

### I. Preparation of Synthetic Intermediates

### General Methods

### Method a: Heteroaromatic Bromination

At 0° C., to a solution of heteroaromatic (1.0 eq.) in DCM (C=0.2 M), N-bromosuccinimide (1.0 eq.) was added portion-wise. The mixture was stirred at rt until no more evolution was noticed by UPLC-MS (1 h, unless mentioned otherwise). The reaction mixture was concentrated and the residue was purified by flash chromatography.

### Method b: Heteroaromatic Iodination

To a suspension of heteroaromatic (1.0 eq.) and solid K₂CO₃ (1.0 eq.) in THE (C=0.125 M), at 0° C., a solution of iodine (1.0 eq.) in THF (C=0.125 M) was added dropwise over the course of one hour. The mixture was stirred at rt until no more evolution was noticed by UPLC-MS (2 h, unless mentioned otherwise). The reaction mixture was hydrolysed, extracted thrice with EtOAc and the organic layer was dried and concentrated.

### Method c: Heteroaromatic Halogenation/Miyaura Borylation

### Step 1: Heteroaryl Halogenation

Under argon, at 0° C., to a solution of heteroaryl (1.0 eq.) in chloroform (C=0.2 M), bromine (1.1 eq.) was added dropwise. The mixture was stirred from 0° C. to rt for 2 h. The reaction mixture was diluted with a saturated aqueous Na₂S₂O₃ solution and the aqueous layer was extracted with DCM. Combined organic layers were washed with brine, dried over MgSO₄, filtered and concentrated.

### Step 2: Miyaura Borylation

In a sealed vial, a suspension of heteroaryl halide (1.0 eq.), bis(pinacolato)diboron (1.5 eq.) and KOAc (2.0 eq.) in dioxane (0.2 M) was degassed with argon bubbling for 15 min and P(tBu)₃ Pd G2 (10 mol %) or Xphos (20 mol %) followed by Pd₂dba₃ (10 mol %) were added in one portion. The reaction mixture was stirred for 17 h at 90° C., cooled to rt, filtered through a Celite® pad and the cake was washed with DCM. The filtrate was concentrated and the crude was purified by flash chromatography.

### Method d: Heteroaromatic Miyaura Borylation

In a sealed vial, a suspension of heteroaromatic halide (1.0 eq.), bis(pinacolato)diboron (1.2 eq.), KOAc (2.0 eq.), in dioxane (C=0.2 M) was degassed with argon bubbling for 15 min and PdCl₂dppf (5 mol %) was then added. The reaction mixture was stirred for 17 h at 90° C., cooled to rt, hydrolysed with NH₄Cl and extracted with DCM thrice. The combined organic layers were dried. The crude was purified by flash chromatography.

### Method e: Suzuki Coupling

In a sealed vial, to a solution of heteroaromatic halide (1.0 eq.) and heteroaryl boronic derivative (1.2-1.5 eq.) in dioxane (C=0.2 M), an aqueous solution of K₂CO₃ (1.2 M, 2.0 eq.) was added dropwise. The resulting suspension was degassed with argon bubbling for 15 min and PdCl₂(dppf).CH₂Cl₂ (5 mol %) was then added in one portion. The vial was sealed and the mixture was stirred at 80° C. until no more evolution was noticed by UPLC-MS (overnight, unless mentioned otherwise). The reaction mixture was cooled to rt, hydrolysed and then extracted thrice with EtOAc. The combined organic layers were washed with brine, dried over MgSO₄, filtered and concentrated. The residue was purified by flash chromatography.

### Method f: Negishi Coupling/Heteroaromatic Halogenation

### Step 1: Negishi Coupling

In a sealed vial under Argon, to a solution of 6-bromopyridin-2-amine (1.0 eq.) in THF (C=0.2 mL) was added Alkylzinc(II) bromide (0.5M in THF) (1.4 eq.) and Pd(dppf)Cl₂ (0.1 eq.). The reaction mixture was heated at 90° C. until no more evolution was noticed by UPLC-MS (4 h, unless mentioned otherwise). The reaction mixture was filtered through a pad of Celite® and the cake washed with EtOAc. Filtrated was hydrolyzed with NaHCO₃ sat. and extracted twice with EtOAc. Organic layers were washed with brine, dried over magnesium sulfate and concentrated. The crude was purified by flash chromatography.

### Step 2: Heteroaromatic Bromination

At 0° C., to a solution of heteroaromatic (1.0 eq.) in DCM (C=0.2 M), N-bromosuccinimide (1.0 eq.) was added portion-wise. The mixture was stirred at rt until no more evolution was noticed by UPLC-MS (1 h, unless mentioned otherwise). The reaction mixture was concentrated, and the residue was purified by flash chromatography.

Method g: Skraup reaction

Under Argon, to a suspension of aniline derivative (1.0 eq.), sodium 3-nitrobenzenesulfonate (2.0 eq.) and propane-1,2,3-triol (4.0 eq.) was added H₂SO₄ 70% (C=0.70 M). The reaction mixture was heated at 135° C. for 2 h. After cooling to rt, the reaction mixture was poured on ice and neutralized with NaOH 6N. The obtained suspension was filtered over Celite® and washed with EtOAc. The filtrate was extracted with EtOAc. Combined organic layers were washed with brine, dried over sodium sulfate and concentrated. The crude was purified by flash chromatography.

### Method h: SNAr

In a sealed vial under Argon, to a solution of 2-chloroquinoline derivate (1.0 eq.) in DMA (C=0.2 M) was added amine (3.0 eq.). The reaction mixture was subjected to microwave irradiation at 150° C. for 15 min. A mixture 50/50 of NH₄Cl sat/H₂O was added to the reaction mixture and was extracted twice with EtOAc. Combined organic layers were washed with brine, dried over magnesium sulfate and concentrated. The crude was purified by flash chromatography to afford the corresponding 8-bromo-2-aminoquinoline.

### Method i; Quinolone Formation

Under Argon, at 0° C., to a solution of aniline derivative (1.0 eq.) in THF (C=0.2 M) was added methyl 3,3-dimethoxypropanoate (1.2 eq.) followed by the addition dropwise of LiHMDS (1 M in THF, 2.5 eq.). The reaction mixture was stirred overnight allowing the ice bath coming back at rt. The reaction mixture was hydrolyzed at 0° C. with aqueous citric acid solution (20 wt. %) then extracted twice with DCM. Combined organic layers were washed with brine, dried over sodium sulfate and concentrated.

To the obtained crude in DCM (C=0.2 M) at 0° C. was added H₂SO₄ conc. (15 eq.). The reaction mixture was stirred 2 h at rt. The reaction mixture was poured on an ice-water mixture, then extracted twice with DCM. Combined organic layers were washed with brine, dried over sodium sulfate and concentrated. The crude was purified by flash chromatography.

### Method j: Peptidic Couplinq

Under Argon, to a solution of 2-carboxylic acid (1.0 eq.) in DCM (C=0.2 M) was added BOP (1.3-1.5 eq.), DIPEA (1.5-3 eq.) and amine (1.3-1.5 eq.). The reaction mixture was stirred at rt for 2 h, then was diluted with DCM, washed with NaHCO₃ sat., brine, dried over sodium sulfate and concentrated. The crude was purified by flash chromatography.

### Method k: Nitration of 8-Bromo Quinoline

Under Argon, at 0° C., to a solution of 8-bromoquinoline derivative (1.0 eq.) in DCM (C=0.6 M) was added tetrabutylammonium nitrate (1.5 eq.) and dropwise trifluoroacetic anhydride (15 eq.). The reaction mixture was stirred at 0° C. for 3 h. The reaction mixture was hydrolyzed with NaHCO₃ sat. then extracted thrice with EtOAc. The organic layer was washed with brine, dried over sodium sulfate and concentrated. The crude was purified by flash chromatography.

### Method l: Reduction of Nitro

Under Argon, to a suspension of nitro heteroaromatic derivate (1.0 eq.) in a mixture EtOH/THF/H₂O (2/2/1, C=0.05 M) were added ammonium chloride (6.4 eq) and iron (6.0 eq.). The reaction mixture was stirred at 80° C. for 2 h. After cooling to rt, the reaction mixture was filtered over a Celite® pad and the cake was washed with THF and EtOH. The filtrate was concentrated. The obtained residue was diluted with EtOAc and washed with water, brine, dried over sodium sulfate and concentrated. The crude was purified by flash chromatography.

### Method m: Fluorination

Under Argon, at 0° C., to a suspension of 3-aminoheteroaryl derivative (1.0 eq.) in H₂O (C=0.35 M) was added tetrafluoroboric acid (48 wt. % in H₂O, 4.0 eq.) followed by the addition dropwise of a solution of sodium nitrite (1.5 eq.) in H₂O (C=0.81 M). The reaction mixture was stirred 3 h allowing the ice bath coming back to rt. The obtained solid was filtered and washed with iPrOH/Et₂O (2/8) and Et₂O (4 times). The obtained light yellow solid was suspended in trifluorotoluene (C=0.35 M) and the suspension was heated at 120° C. for 1 h. The reaction mixture was hydrolyzed with NaHCO₃ sat. then extracted thrice with EtOAc. Combined organic layers were washed with brine, dried over sodium sulfate and concentrated. The crude was purified by flash chromatography to obtain the corresponding fluoro-heteroaromatic derivative.

### Method n: Chlorination

Under Argon, at −5° C. to a solution of amino-heteroaromatic derivate (1.0 eq.) in HCl 37% (C=0.2 M) was added dropwise a solution of sodium nitrite (1.5 eq.) in H₂O (C=0.65 M). The reaction mixture was stirred at −5° C. for 10 min then copper(I) chloride (4.0 eq.) was added. 5 min later, the ice bath was removed, and the reaction mixture was stirred 2 h at rt. The reaction mixture was diluted with H₂O and basified with NaOH 1N until pH-7. The aqueous layer was extracted thrice with EtOAc. Combined organic layers were washed with brine, dried over sodium sulfate and concentrated. The crude was purified by flash chromatography to obtain the corresponding chloro-heteroaromatic derivative.

### Method o: Friedlander 1^{st} Method

In a sealed vial under Argon, to a solution of (2-aminophenyl)methanol derivative (1.0 eq.) in dioxane (C=0.3 M) was added ketone (1.0-1.5 eq.), KOtBu (1.0-1.5 eq.) and benzophenone (1.0 eq.). The reaction mixture was heated at 90° C. for 1 h. The reaction mixture was diluted with EtOAc, washed with water, brine, dried over sodium sulfate and concentrated. The crude was purified by flash chromatography to obtain the corresponding quinoline derivative

### Method P: Friedlander 2^{nd} Method

In a sealed vial under Argon, to a solution of (2-amino-3-bromophenyl)methanol (1.0 eq.) in EtOH (C=0.3 M) was added MnO₂ (5 eq.). The reaction mixture was heated at 80° C. for 1 h. The reaction mixture was cooled to 0° C. followed by the addition of ketone (1.2 eq.) and dropwise a solution of KOH (1.4 eq.) in EtOH (C=1 M). The reaction mixture was stirred at 0° C. until no more evolution was noticed by UPLC-MS (1 h, unless mentioned otherwise). The reaction mixture was filtered through a pad of Celite® and the cake was washed with EtOAc. The organic layer was washed with NH₄Cl sat., brine, dried over sodium sulfate and concentrated. The crude was purified by flash chromatography to obtain the corresponding 8-bromo-2-alkyl/arylquinoline.

### Synthetic Intermediate Syntheses

### Compound Id: 5-bromo-6-ethyl-pyridin-2-amine

Compound 1d was prepared according to method a, starting from 6-ethylpyridin-2-amine (5.00 g, 41.0 mmol). The crude was purified by flash chromatography (SiO₂, CyHex/EtOAc: 100/0 to 60/40) to afford compound 1d as a brown solid (6.87 g, 83%).

¹H NMR (400 MHz, DMSO-d₆) δ: 1.13 (t, J 7.6 Hz, 3H, CH₂—CH₃); 2.63 (q, J 7.6 Hz, 2H, CH₂—CH₃); 6.03 (bs, 2H, NH₂); 6.23 (d, J 8.8 Hz, 1H, Ar); 7.44 (d, J 8.8 Hz, 1H, Ar). M/Z (M[⁸′Br]+H)+: 202.8.

### Compound Le: 5-bromo-6-propyl-pyridin-2-amine

Compound 1e was prepared according to method a starting from 6-propylpyridin-2-amine (500 mg, 3.67 mmol). The crude was purified by flash chromatography (SiO₂, DCM/MeOH: 100/0 to 96/4) to afford compound 1e as an orange solid (511 mg, 64%).

¹H NMR (400 MHz, DMSO-d₆) δ: 0.91 (t, J 7.6 Hz, 3H, CH₂—CH₃); 1.61 (sex, J 7.6 Hz, 2H, CH₂—CH₂—CH₃); 2.60 (t, J 7.6 Hz, 2H, CH₂—CH₂—CH₃); 6.01 (bs, 2H, NH₂); 6.23 (d, J 8.8 Hz, 1H, Ar); 7.44 (d, J 8.8 Hz, 1H, Ar). M/Z (M[⁷⁹Br]+H)+: 217.6.

### Compound 1f: 5-bromo-6-isopropyl-pyridin-2-amine

Compound 1f was prepared according to method a starting from 6-isopropylpyridin-2-amine (500 mg, 3.67 mmol). The crude was purified by flash chromatography (SiO₂, CyHex/EtOAc: 100/0 to 75/25) to afford compound 1f (568 mg, 71%).

¹H NMR (400 MHz, DMSO-d₆) δ: 1.12 (d, J 6.8 Hz, 6H, CH—(CH₃)₂); 3.26 (sep, J 6.8 Hz, 1H CH—(CH₃)₂); 5.99 (bs, 2H, NH₂); 6.23 (d, J 8.4 Hz, 1H, Ar); 7.44 (d, J 8.4 Hz, 1H, Ar). M/Z (M[⁷⁹Br]+H)+: 217.6.

Compound 1g: 5-bromo-6-cyclopropyl-pyridin-2-amine Compound 1g was prepared according to method a starting from 6-cyclopropylpyridin-2-amine (500 mg, 3.67 mmol). The crude was purified by flash chromatography (SiO₂, DCM/MeOH: 100/0 to 97/3) to afford compound 1g as an orange oil (618 mg, 78%).

¹H NMR (400 MHz, DMSO-d₆) δ: 0.86 (d, J 6.4 Hz, 4H, CH—CH_{2CyPr}); 2.25 (quint, J 6.4 Hz, 1H CH—CH_{2CyPr}); 5.92 (bs, 2H, NH₂); 6.16 (d, J 8.4 Hz, 1H, Ar); 7.42 (d, J 8.4 Hz, 1H, Ar). M/Z (M[⁷⁹Br]+H)+: 215.6.

### Compound 1h: 3-iodopyridine-2,6-diamine

Compound 1h was prepared according to method b starting from 2,6-diaminopyridine (4.5 g, 41.2 mmol). The residue was triturated in MeOH. The precipitate was filtered, and the filtrate was concentrated. The resulting foam was purified by flash chromatography (SiO₂, CyHex/EtOAc: 80/20 to 0/100) to afford compound 1h as a pink solid (7.7 g, 79%).

¹H NMR (400 MHz, DMSO-d₆) δ: 5.40 (bs, 2H, NH₂); 5.58 (d, J 8.4 Hz, 1H, Ar); 5.63 (bs, 2H, NH₂); 7.37 (d, J 8.4 Hz, 1H, Ar). M/Z (M+H)⁺: 236.5.

### Compound 2a: 3-bromo-2-methyl-benzothiophene

Compound 2a was prepared according to method c step 1 starting from 2-methyl-benzothiophene (500 mg, 3.37 mmol) and was obtained without further purification as a beige oil (838 mg, quant. yield).

¹H NMR (400 MHz, DMSO-d₆) δ: 2.54 (s, 3H, CH₃); 7.41 (dt, J 8.4, 1.2 Hz, 1H, Ar); 7.48 (dt, J 8.4, 1.2 Hz, 1H, Ar); 7.66 (d, J 8.0 Hz, 1H, Ar); 7.96 (d, J 8.0 Hz, 1H, Ar).

### Compound 2b: 3-bromo-5-methyl-benzothiophene

Compound 2b was prepared according to method c step 1 starting from 5-methyl-benzothiophene (337 mg, 2.68 mmol). The crude was purified by flash chromatography (SiO₂, CyHex/EtOAc: 100/0 to 90/10) to afford compound 2b as a light-yellow oil (537 mg, 88%).

¹H NMR (400 MHz, DMSO-d₆) δ: 2.48 (s, 3H, CH₃); 7.29-7.34 (m, 1H, Ar); 7.55-7.58 (m, 1H, Ar); 7.93-7.98 (m, 2H, Ar).

### Compound 2c: 3-bromo-5-fluoro-benzothiophene

Compound 2c was prepared according to method c step 1 starting from 5-fluorobenzothiophene (500 mg, 3.25 mmol) and was obtained without further purification as a beige solid (789 mg, quant. yield).

¹H NMR (400 MHz, DMSO-d₆) δ: 7.39 (dt, J 8.8, 2.4 Hz, 1H, Ar); 7.52 (dd, J 9.2, 2.4 Hz, 1H, Ar); 8.12-8.18 (m, 2H, Ar).

### Compound 2d: 3-bromo-6-methoxy-benzothiophene

Compound 2d was prepared according to method c step 1 starting from 6-methoxybenzothiophene (400 mg, 2.44 mmol). The crude was purified by flash chromatography (SiO₂, CyHex/EtOAc, 100/0 to 90/10) to afford compound 2d as a light-yellow oil (492 mg, 83%).

¹H NMR (400 MHz, DMSO-d₆) δ: 3.8 (s, 3H, CH₃); 7.00 (dd, J 8.8, 2.4 Hz, 1H, Ar); 7.51 (s, 1H, Ar); 7.54 (d, J 2.4 Hz, 1H, Ar); 7.68 (d, J 8.8 Hz, 1H, Ar).

### Compound 3a: 2-(5-fluorobenzothiophen-3-yl)-4,4,5,5-tetramethyl-1,3,2-dioxaborolane

Compound 3a was prepared according to method c step 2 starting from 2-bromo-5-fluorobenzothiophene 2c (200 mg, 0.87 mmol), using P(tBu)₃ Pd G2 (45 mg, 0.09 mmol, 10 mol %) as catalyst. The crude was purified by flash chromatography (SiO₂, DCM: 100%) to afford compound 3a as a brown oil (59 mg, 25%, contamination with the corresponding boronic acid, 20% by NMR).

### Compound 3b: 2-(6-methoxybenzothiophen-3-yl)-4,4,5,5-tetramethyl-1,3,2-dioxaborolane

Compound 3b was prepared according to method c step 2 starting from 2-bromo-6-methoxybenzothiophene 2d (393 mg, 1.62 mmol), using Pd₂dba₃ and XPhos as catalyst. The crude was purified by flash chromatography (SiO₂, DCM: 100%) to afford compound 3b as a yellow solid (252 mg, 54%).

¹H NMR (400 MHz, DMSO-d₆) δ: 1.32 (s, 12H, 2 O—(CH₃)₂); 3.84 (s, 3H, Ar—O—CH₃); 7.01 (dd, J 8.4, 2.4 Hz, 1H, Ar); 7.56 (d, J 2.4 Hz, 1H, Ar); 7.75-7.78 (m, 2H, Ar).

### Compound 4a: 6-ethyl-5-(4,4,5,5-tetramethyl-1,3,2-dioxaborolan-2-yl)pyridin-2-amine

Compound 4a was prepared according to method d starting from 5-bromo-6-ethylpyridin-2-amine 1d (1.5 g, 7.46 mmol). The crude was purified by flash chromatography (SiO₂, DCM/MeOH: 100/0 to 90/10) to afford compound 4a as a brown solid (570 mg, 31%).

¹H NMR (400 MHz, DMSO-d₆) δ: 1.06-1.21 (m, 3H, CH₂.CH₃); 1.26 (s, 12H, 2 O—C(CH₃)₂); 2.77 (q, J 7.6 Hz, 2H, CH₂CH₃); 6.31 (d, J 8.4 Hz, 1H, Ar); 6.50 (bs, 2H, NH₂); 7.62 (d, J 8.4 Hz, 1H, Ar). M/Z (M+H)+: 249.5.

### Compound 5A: 5-(2-chlorophenyl)-6-ethyl-pyridin-2-amine

Compound 5a was prepared according to method e from 5-bromo-6-ethylpyridin-2-amine 1d (1.00 g, 4.97 mmol) and 2-chlorophenylboronic acid (1.16 g, 7.46 mmol, 1.5 eq.). The crude was purified by flash chromatography (SiO₂, DCM/MeOH: 100/0 to 95/5) to afford compound 5a as an orange solid (1.00 g, 86%).

¹H NMR (400 MHz, DMSO-d₆) δ: 1.00 (t, J 7.6 Hz, 3H, CH₃); 2.19-3.38 (m, 2H, CH₂); 5.92 (s, 1H, NH₂); 6.33 (d, J 8.4 Hz, 1H, Ar); 7.08 (d, J 8.4 Hz, 1H, Ar); 7.25-7.30 (m, 1H, Ar); 7.34-7.39 (m, 2H, Ar); 7.50-7.55 (m, 1H, Ar). M/Z (M[³⁵Cl]+H)⁺: 233.1.

### Compound 5b: 3-(2-chlorophenyl)pyridine-2,6-diamine

Compound 5b was prepared according to method e from 2,6-diamino-3-iodopyridine 1h (1.0 g, 4.25 mmol) and 2-chlorophenylboronic acid (0.99 g, 6.38 mmol, 1.5 eq.). The crude was purified by flash chromatography (SiO₂, DCM/MeOH: 100/0 to 96/4) to afford compound 5b as a smoothy brown solid (897 mg, 96%).

¹H NMR (400 MHz, DMSO-d₆) δ: 4.83 (s, 2H, NH₂); 5.54 (s, 2H, 2 NH₂); 5.79 (d, J 8.0 Hz, 1H, Ar); 6.91 (d, J 8.0 Hz, 1H, Ar); 7.26-7.38 (m, 3H, Ar); 7.48-7.53 (m, 1H, Ar). M/Z (M[³⁵Cl]+H)⁺: 220.6.

### Compound 6: 3-bromo-6-(2,5-dimethyl-1H-pyrrol-1-yl)-2-ethylpyridine

Under Argon, to a solution of 5-bromo-6-ethylpyridin-2-amine 1d (5.30 g, 26.4 mmol, 1.0 eq.) in toluene (26 mL) was added hexane-2,5-dione (3.16 g, 3.25 mL, 27.7 mmol, 1.05 eq.) and 4-methylbenzenesulfonic acid (45.4 mg, 264 μmol, 0.01 eq.). The reaction mixture was heated at 135° C. for 18 h. The reaction mixture was concentrated, dissolved in toluene (26 mL) and heated at 135° C. for 20 h. The reaction mixture was concentrated to obtain a brown oil. The crude was purified by flash chromatography (SiO₂, CyHex/EtOAc: 100/0 to 95/5) to afford compound 6 (7.09 g, 96%) as an orange oil. M/Z (M[⁷⁹Br]+H)⁺: 281.0.

### Compound 7: 6-(2,5-dimethyl-1H-pyrrol-1-yl)-2-ethyl-3-(4,4,5,5-tetramethyl-1,3,2-dioxaborolan-2-yl)pyridine

Under Argon, to a solution of compound 6 (7.09 g, 25.4 mmol, 1.0 eq.) in dioxane (63.5 mL) was added Bis(pinacolato)diboron (12.9 g, 50.8 mmol, 2.0 eq.) and potassium acetate (4.98 g, 50.8 mmol, 2.0 eq.). The reaction mixture was sparged with argon for 10 min then Pd(dppf)C₂ (557 mg, 0.76 mmol, 0.03 eq.) was added. The reaction mixture was heated at 110° C. for 3 h. The reaction mixture was filtered through a pad of Celite® and the cake was washed with EtOAc (200 mL). Filtrate was hydrolyzed with NH₄Cl sat. (200 mL) and extracted twice with EtOAc (200 mL). The organic layers were washed with brine (200 mL), dried over magnesium sulfate and concentrated. The crude was purified by flash chromatography (SiO₂, CyHex/EtOAc: 100/0 to 95/5) to afford to afford compound 7 (6.66 g, 80%) as a yellow oil. M/Z (M+H)⁺: 327.0.

### Compound 8: N-(6-amino-5-bromopyridin-2-yl)pivalamide

Under Argon, at −78° C., to a solution of 3-bromopyridine-2,6-diamine (1.20 g, 6.42 mmol, 1.0 eq.) in a mixture THE (9 mL)/DCM (9 mL) at −78° C. was added Et₃N (1.16 mL, 8.35 mmol, 1.3 eq.) and pivaloyl chloride (833 μL, 6.74 mmol, 1.05 eq.) in solution in DCM (5 mL) over a period of 10 min. The reaction mixture was stirred 18 h allowing the bath coming back to room temperature. The reaction mixture was hydrolyzed with NaHCO₃ sat. (100 mL) and extracted twice with EtOAc (100 mL). Combined organic layers were washed with brine (50 mL), dried over sodium sulfate and concentrated. The crude was purified by flash chromatography (SiO₂, CyHex/EtAOc: 100/0 to 50/50) to afford compound 8 (1.26 g, 4.65 mmol, 72%) as a light yellow solid. M/Z (M[⁸¹Br]+H)⁺: 273.9.

### Compound 9: N-(6-amino-5-(4,4,5,5-tetramethyl-1,3,2-dioxaborolan-2-yl)pyridin-2-yl)pivalamide

In a sealed tube under Argon, to a solution of N-(6-amino-5-bromopyridin-2-yl)pivalamide 8 (860 mg, 3.17 mmol, 1.0 eq.) in dioxane (6 mL), bis(pinacolato)diboron (1.61 g, 6.34 mmol, 2.0 eq.) and KOAc (622 mg, 6.34 mmol, 2.0 eq.) were added. The reaction mixture was sparged with Ar for 10 min then Pd(dppf)Cl₂.CH₂Cl₂ (129 mg, 159 μmol, 0.05 eq.) was added. The reaction mixture was heated at 110° C. for 4 h. The reaction mixture was filtered through a pad of Celite® and the cake was washed with EtOAc (100 mL). Filtrate was hydrolyzed with NH₄Cl sat. (100 mL) and extracted twice with EtOAc (100 mL). The organic layers were washed with brine (200 mL), dried over magnesium sulfate and concentrated. The crude was purified by flash chromatography (SiO₂, CyHex/EtOAc: 100/0 to 50/50) to afford compound 9 (760 mg, 2.38 mmol, 75%) as a light yellow solid. M/Z (M+H)⁺: 238.0.

### Compound 10: 8-(6-(2,5-dimethyl-1H-pyrrol-1-yl)-2-ethylpyridin-3-yl)quinoline-2-carboxylic Acid

In a sealed tube under Argon, to a solution of 8-bromoquinoline-2-carboxylic acid (600 mg, 2.38 mmol) and compound 7 (1.16 g, 3.57 mmol, 1.5 eq.) in DME (24 mL) was added a solution of K₂CO₃ 1.2M in H₂O (3.97 mL, 4.76 mmol, 2.0 eq.). The reaction mixture was sparged with argon for 10 min then SPhos Pd G2 (85.8 mg, 119 μmol, 0.05 eq.) was added. The reaction mixture was heated at 80° C. for 1.5 h. The reaction mixture was filtered through a pad of Celite® and the cake was washed with EtOAc (125 mL). Filtrate was hydrolyzed with HCl 0.05 N. (120 mL) until pH˜4-5 and extracted twice with EtOAc (100 mL). The organic layers were dried over magnesium sulfate and concentrated. The crude was purified by flash chromatography (SiO₂, DCM/MeOH: 100/0 to 96/4) to afford compound 10 (870 mg, 98%) as an orange gum. M/Z (M+H)⁺: 372.1.

### Compound 11: 6-isobutylpyridin-2-amine

Compound 11 was prepared according to method f step 1 starting from 6-bromopyrind-2-amine (2.00 g, 12.0 mmol) and isobutylzinc bromide (0.5 M in THF, 32 mL, 16 mmol, 1.4 eq.). The crude was purified by flash chromatography (SiO₂, DCM/MeOH 100/0 to 95/5) to afford compound 11 (1.10 g, 63%) as a brown oil. M/Z (M+H)⁺: 151.2.

### Compound 12: 5-bromo-6-isobutylpyridin-2-amine

Compound 12 was prepared according to method f step 2 starting from compound 11 (1.10 g, 7.30 mmol) and NBS (0.95 g, 7.0 mmol, 0.95 eq.). The crude was purified by flash chromatography (SiO₂, DCM/MeOH: 100/0 to 96/4) to afford compound 12 (1.21 g, 75%) as a brown solid. M/Z (M[⁸¹Br]+H)⁺: 231.0.

### Compound 13: 6-(cyclobutylmethyl)pyridin-2-amine

Compound 13 was prepared according to method f step 1 starting from 6-bromopyrind-2-amine (1.00 g, 5.80 mmol) and (cyclobutylmethyl)zinc bromide (0.5 M in THF, 25 mL, 12.5 mmol, 2.2 eq.). The crude was purified by flash chromatography (SiO₂, DCM/MeOH 100/0 to 95/5) to afford compound 13 (622 mg, 66%) as a brown oil. M/Z (M+H)⁺: 163.1

### Compound 14: 5-bromo-6-(cyclobutylmethyl)pyridin-2-amine

Compound 14 was prepared according to method f step 2 starting compound 13 (622 mg, 3.83 mmol) and NBS (682 mg, 3.83 mmol, 1.0 eq.). The crude was purified by flash chromatography (SiO₂, DCM/MeOH: 100/0 to 96/4) to afford compound 14 (100 mg, 11%) as a brown solid. M/Z (M[¹Br]+H)⁺: 243.0.

### Compound 15: 6-(3,3,3-trifluoropropyl)pyridin-2-amine

Compound 15 was prepared according to method f step 1 starting from 6-bromopyrind-2-amine (1.00 g, 5.80 mmol) and (3,3,3-trifluoropropyl)zinc bromide (0.5 M in THF, 25 mL, 12.5 mmol, 2.2 eq.). The crude was purified by flash chromatography (SiO₂, DCM/MeOH 100/0 to 95/5) to afford compound 15 (417 mg, 38%) as an orange oil. M/Z (M+H)⁺: 191.0

### Compound 16: 5-bromo-6-(3,3,3-trifluoropropyl)pyridin-2-amine

Compound 16 was prepared according to method f step 2 starting from compound 14 (417 mg, 2.19 mmol) and NBS (371 mg, 2.08 mmol, 0.95 eq.). The crude was purified by flash chromatography (SiO₂, DCM/MeOH: 100/0 to 98/2) to afford compound 16 (467 mg, 84%) as a yellow solid. M/Z (M[⁷⁹Br]+H)⁺: 268.9.

### Compound 17: 6-(4,4,4-trifluorobutyl)pyridin-2-amine

Compound 17 was prepared according to method f step 2 starting from 6-bromopyrind-2-amine (1.10 g, 6.40 mmol) and 4,4,4-trifluorobutyl)zinc bromide (0.5 M in THF, 25 mL, 12.5 mmol, 2 eq.). The crude was purified by flash chromatography (SiO₂, DCM/MeOH 100/0 to 95/5) to afford compound 17 (1.05 g) as an orange oil. M/Z (M+H)⁺: 191.0

### Compound 18: 5-bromo-6-(4,4,4-trifluorobutyl)pyridin-2-amine

Compound 18 was prepared according to method f step 2 starting from compound 17 (1.05 g, 5.14 mmol) and NBS (824 mg, 4.63 mmol, 0.9 eq.). The crude was purified by flash chromatography (SiO₂, DCM/MeOH: 100/0 to 98/2) to afford compound 18 (914 mg, 51% over 2 steps) as a brown oil. M/Z (M[⁷⁹Br]+H)⁺: 284.9.

### Compound 19: 6-(cyclopropylmethyl)pyridin-2-amine

Compound 19 was prepared according to method f step 1 starting from 6-bromopyrind-2-amine (1.00 g, 5.8 mmol) and (cyclopropylmethyl)zinc bromide (0.5 M in THF, 25 mL, 12.5 mmol, 2.2 eq.). The crude was purified by flash chromatography (SiO₂, DCM/MeOH: 100/0 to 95/5) to afford compound 19 (420 mg) as an orange oil. M/Z (M+H)⁺: 149.2.

### Compound 20: 5-bromo-6-(cyclopropylmethyl)pyridin-2-amine

Compound 20 was prepared according to method f step 2 starting from compound 19 (420 mg, 2.83 mmol, 1.0 eq.) and NBS (479 mg, 2.69 mmol, 0.95 eq.). The crude was purified by flash chromatography (SiO₂, DCM/MeOH: 100/0 to 98/2) to afford compound 20 (286 mg, 22% over 2 steps) as a red oil. M/Z (M[⁷⁹Br]+H)⁺: 227.0

### Compound 21: 6-isopentylpyridin-2-amine

Compound 21 was prepared according to method f step1 starting from 6-bromopyrind-2-amine (1.00 g, 5.8 mmol) and isopentylzinc bromide (0.5 M in THF, 25 mL, 12.5 mmol, 2.2 eq.). The crude was purified by flash chromatography (SiO₂, DCM/MeOH 100/0 to 95/5) to afford compound 21 (620 mg, 65%) as a light orange oil. M/Z (M+H)⁺: 165.2

### Compound 22: 5-bromo-6-isopentylpyridin-2-amine

Compound 22 was prepared according to method f step1 starting from compound 21 (620 mg, 3.77 mmol) and NBS (707 mg, 3.97 mmol, 1.05 eq.). The crude was purified by flash chromatography (SiO₂, DCM/MeOH: 100/0 to 98/2) to afford compound 22 (807 mg, 88%) as an orange solid. M/Z (M[⁹Br]+H)⁺: 243.0

### Compound 23: 8-bromo-7-fluoroquinoline

Compound 23 was prepared according to method g starting from 2-bromo-3-fluoroaniline (4.00 g, 21.1 mmol). The crude was purified by flash chromatography (SiO₂, CyHex/EtOAc, 100/0 to 70/30) to afford compound 23 as a white solid (4.20 g, 88%). M/Z ([⁸¹Br]+H)⁺: 228.0

### Compound 24: 8-bromo-5,7-difluoroquinoline

Compound 24 was prepared according to method g starting from 2-bromo-3,5-difluoroaniline (2.50 g, 12.0 mmol). The crude was purified by flash chromatography (SiO₂, CyHex/EtOAc: 100/0 to 75/25) to afford compound 24 as a white solid (2.40 g, 82%). M/Z ([⁷⁹Br]+H)⁺: 243.8

### Compound 25: 8-bromo-7-(trifluoromethyl)quinoline

Compound 25 was prepared according to method g starting from 2-bromo-3-(trifluoromethyl)aniline (500 mg, 2.08 mmol). The crude was purified by flash chromatography (SiO₂, CyHex/EtOAc: 100/0 to 70/30) to afford compound 25 as a beige solid (428 mg, 80%). M/Z ([⁷⁹Br]+H)⁺: 275.9

### Compound 26: 8-bromo-7-chloroquinoline

Compound 26 was prepared according to method g starting from 2-bromo-3-chloroaniline (2.50 g, 12.0 mmol). The crude was purified by flash chromatography (SiO₂, CyHex/EtOAc: 100/0 to 75/25) to afford compound 26 as a white solid (2.42 g, 82%). M/Z ([⁸¹Br][³⁷Cl]+H)⁺: 245.8

### Compound 27: 8-bromo-6,7-difluoroquinoline

Compound 27 was prepared according to method g starting from 2-bromo-3,4-difluoroaniline (3.00 g, 14.4 mmol). The crude was purified by flash chromatography (SiO₂, CyHex/EtOAc, 100/0 to 75/25) to afford compound 27 as a beige solid (3.36 g, 95%). M/Z ([⁸¹Br]+H)⁺: 245.8

### Compound 28: 5-bromo-1,2,3,4-tetrahydroacridine

Compound 28 was prepared according to method o starting from (2-amino-3-bromophenyl)methanol (100 mg, 0.50 mmol) and cyclohexanone (51 μL, 0.50 mmol). The crude was purified by flash chromatography (15 μm Interchim® SiO₂, CyHex/EtOAc: 100/0 to 80/20) to afford compound 28 (84 mg, 65%) as a yellow oil. M/Z (M[⁸¹Br]+H)⁺: 261.9.

### Compound 29: 6-bromo-2-methyl-1,2,3,4-tetrahydrobenzo[b][1,6]naphthyridine

Compound 29 was prepared according to method o starting from (2-amino-3-bromophenyl)methanol (150 mg, 0.74 mmol) and 1-methylpiperidin-4-one (91 μL, 0.74 mmol). The crude was purified by flash chromatography (15 μm Interchim® SiO₂, CyHex/EtOAc: 100/0 to 0/100) to afford compound 29 (115 mg, 56%) as a yellow oil. M/Z (M[⁷⁹Br]+H)⁺: 276.9.

### Compound 30: 5-bromo-2,3-dihydro-1H-cyclopenta[b]quinoline

In a sealed vial under Argon, to a solution of (2-amino-3-bromophenyl)methanol (150 mg, 0.74 mmol, 1.0 eq.) in dioxane (1.5 mL) was added cyclopentanol (67 μL, 0.74 mmol, 1.0 eq.), potassium tert-butoxide (167 mg, 1.48 mmol, 2.0 eq.) and benzophenone (271 mg, 1.48 mmol, 2.0 eq.). The reaction mixture was heated at 90° C. for 1 h. The reaction mixture was diluted with EtOAc (50 mL), washed with water (50 mL), brine (50 mL), dried over sodium sulfate and concentrated. The crude was purified by flash chromatography (15 μm Interchim® SiO₂, CyHex/EtOAc: 100/0 to 80/20) to afford compound 30 (77 mg, 42%) as an orange solid. M/Z (M[⁸¹Br]+H)⁺: 249.9.

### Compound 31: 8-bromo-2-phenylquinoline

Compound 31 was prepared according to method o starting from (2-amino-3-bromophenyl)methanol (202 mg, 1.00 mmol) and acetophenone (120 mg, 1.00 mmol). The crude was purified by flash chromatography (SiO₂, CyHex/EtOAc: 100/0 to 70/30) to afford compound 31 (162 mg, 57%) as a yellow oil. M/Z (M[⁷⁹Br]+H)⁺: 284.0.

### Compound 32: 8-bromo-2-(pyridin-3-yl)quinoline

Compound 32 was prepared according to method o starting from (2-amino-3-bromophenyl)methanol (202 mg, 1.00 mmol) and 3-acetylpyridine (110 μL, 1.00 mmol). The crude was purified by flash chromatography (SiO₂, CyHex/EtOAc: 100/0 to 50/50) to afford compound 32 (156 mg, 55%) as a colorless oil. M/Z (M[79Br]+H)⁺: 285.0.

### Compound 33: 8-bromo-2-cyclohexylquinoline

Compound 33 was prepared according to method o starting from (2-amino-3-bromophenyl)methanol (200 mg, 0.99 mmol) and 1-cyclohexylethan-1-one (125 mg, 0.99 mmol). The crude was purified by flash chromatography (SiO₂, CyHex/EtOAc: 100/0 to 70/30) to afford compound 33 (162 mg, 56%) as a colorless oil. M/Z (M[⁷⁹Br]+H)⁺: 290.0.

### Compound 34: 8-bromo-2-(1-methylcyclopropyl)quinoline

Compound 34 was prepared according to method o starting from (2-amino-3-bromophenyl)methanol (200 mg, 0.99 mmol) and 1-(1-methylcyclopropyl)ethan-1-one (97 mg, 0.99 mmol). The crude was purified by flash chromatography (SiO₂, CyHex/EtOAc: 100/0 to 70/30) to afford compound 34 (145 mg, 53%) as a colorless oil. M/Z (M[⁷⁹Br]+H)⁺: 262.0

### Compound 35: 8-bromo-2-(pyridin-2-yl)quinoline

Compound 35 was prepared according to method o starting from (2-amino-3-bromophenyl)methanol (202 mg, 1.00 mmol) and 2-acetylpyridine (118 μL, 1.00 mmol). The crude was purified by flash chromatography (SiO₂, CyHex/EtOAc: 100/0 to 80/80) to afford compound 35 (190 mg, 67%) as a brown solid. M/Z (M[⁷⁹Br]+H)⁺: 285.0

### Compound 36: 8-bromo-2-(tetrahydro-2H-pyran-4-yl)quinoline

Compound 36 was prepared according to method o starting from (2-amino-3-bromophenyl)methanol (200 mg, 0.99 mmol) and 1-(tetrahydro-2H-pyran-4-yl)ethan-1-one (127 mg, 0.99 mmol). The crude was purified by flash chromatography (SiO₂, CyHex/EtOAc: 100/0 to 70/30) to afford compound 36 (100 mg, 34%) as a colorless oil. M/Z (M[⁸¹Br]+H)⁺: 294.0.

### Compound 37: 8-bromo-2-(pyridin-4-yl)quinoline

Compound 37 was prepared according to method o starting from (2-amino-3-bromophenyl)methanol (250 mg, 1.24 mmol) and 1-(pyridin-4-yl)ethan-1-one (225 mg, 1.86 mmol). The crude was purified by flash chromatography (SiO₂, CyHex/DCM: 20/80 to 0/100) to afford compound 37 (220 mg, 62%) as a white solid. M/Z (M[⁸¹Br]+H)⁺: 286.9.

### Compound 38: 8-bromo-2-(imidazo[1,2-a]pyridin-6-yl)quinoline

Compound 38 was prepared according to method o starting from (2-amino-3-bromophenyl)methanol (200 mg, 0.99 mmol) and 1-(imidazo[1,2-a]pyridin-6-yl)ethan-1-one (237 mg, 1.48 mmol). The crude was purified by flash chromatography (SiO₂, DCM/MeOH: 100/0 to 95/5) to afford compound 38 (100 mg, 31%) as a yellow solid. M/Z (M[⁶¹Br]+H)⁺: 326.0.

### Compound 39: 8-bromo-2-(pyrimidin-5-yl)quinoline

Compound 39 was prepared according to method p starting from (2-amino-3-bromophenyl)methanol (200 mg, 0.99 mmol) and 1-(pyrimidin-5-yl)ethan-1-one (142 mg, 1.16 mmol). The crude was purified by flash chromatography (SiO₂, CyHex/EtOAc: 100/0 to 50/50) to afford compound 39 (250 mg, 88%) as a white solid. M/Z (M[⁸¹Br]+H)⁺: 287.9.

### Compound 40: 8-bromo-2-(pyrazin-2-yl)quinoline

Compound 40 was prepared according to method p starting from (2-amino-3-bromophenyl)methanol (200 mg, 0.99 mmol) and 1-(pyrazin-2-yl)ethan-1-one (133 mg, 1.08 mmol). The crude was purified by flash chromatography (SiO₂, CyHex/EtOac: 100/0 to 70/30) to afford compound 40 (210 mg, 74%) as a white solid. M/Z (M[⁸¹Br]+H)⁺: 287.9.

### Compound 41: 8-bromo-2-(4-methylpyridin-3-yl)quinoline

Compound 41 was prepared according to method p starting from (2-amino-3-bromophenyl)methanol (150 mg, 0.74 mmol) and 1-(4-methylpyridin-3-yl)ethan-1-one (120 mg, 0.89 mmol). The reaction mixture was stirred at 0° C. for 2 h and then heated at 80° C. for 20 h. The crude was purified by flash chromatography (SiO₂, CyHex/EtOAc: 100/0 to 50/50) to afford compound 41 (150 mg, 68%) as a white solid. M/Z (M[⁸¹Br]+H)⁺: 301.0.

### Compound 42: 8-bromo-2-(2-methylpyridin-3-yl)quinoline

Compound 42 was prepared according to method p starting from (2-amino-3-bromophenyl)methanol (150 mg, 0.74 mmol) and 1-(2-methylpyridin-3-yl)ethan-1-one (120 mg, 0.89 mmol). The reaction mixture was stirred at 0° C. for 2 h and then heated at 80° C. for 20 h. The crude was purified by flash chromatography (SiO₂, CyHex/EtOAc: 100/0 to 50/50) to afford compound 42 (170 mg, 77%) as a white solid. M/Z (M[⁸¹Br]+H)⁺: 301.0.

### Compound 43: 4-(8-bromoquinolin-2-yl)morpholine

In a sealed vial under Argon, to a solution of 8-bromo-2-chloroquinoline (100 mg, 0.41 mmol, 1.0 eq.) in DMA (2.0 mL) was added morpholine (72 μL, 0.83 mmol, 2.0 eq.). The reaction mixture was subjected to microwave irradiation at 150° C. for 15 min and was heated at 80° C. for 3 days. The reaction mixture was hydrolyzed with water (50 mL) and extracted twice with EtOAc (30 mL). The organic layers were washed brine (40 mL), dried over magnesium sulfate and concentrated. The crude was purified by flash chromatography (SiO₂, CyHex/EtOAc: 100/0 to 70/30) to afford compound 43 (103 mg, 85%) as a pink oil. M/Z (M[⁷⁹Br]+H)⁺: 293.1.

### Compound 44: 4-(2-((8-bromoquinolin-2-yl)oxy)ethyl)morpholine

In a sealed vial under Argon, to a solution of 8-bromo-2-chloroquinoline (500 mg, 2.06 mmol, 1.0 eq.) in THF (9 mL) was added 2-morpholinoethan-1-ol (541 mg, 4.12 mmol, 2.0 eq.) and potassium tert-butoxide (347 mg, 3.09 mmol, 1.5 eq.). The reaction mixture was heated at 80° C. for 16 h. The reaction mixture was hydrolyzed with NH₄Cl sat. (100 mL) and extracted twice with EtOAc (80 mL). The organic layers were washed with brine (150 mL), dried over magnesium sulfate and concentrated. The crude was purified by flash chromatography (SiO₂, DCM/MeOH: 100/0 to 97/3) to afford compound 44 (600 mg, 86%) as a light brown oil. M/Z (M[⁷⁹Br]+H)⁺: 337.1.

### Compound 45: 8-bromo-2-(pyrrolidin-1-yl)quinoline

Compound 45 was prepared according to method h starting from 8-bromo-2-chloroquinoline (500 mg, 2.06 mmol) and pyrrolidine (440 mg, 6.19 mmol). The crude was purified by flash chromatography (SiO₂, CyHex/EtOAc: 100/0 to 80/20) to afford compound 45 as a pink solid (482 mg, 84%). M/Z ([⁸¹Br]+H)⁺: 229.0

### Compound 46: 8-bromo-2-(4,4-difluoropiperidin-1-yl)quinoline

In a sealed vial under Argon, to a suspension of 8-bromo-2-chloroquinoline (373 mg, 1.54 mmol, 1.0 eq.) in DMA (9 mL) was added 4,4-difluoropiperidine hydrochloride (364 mg, 2.31 mmol, 1.5 eq.) and Et₃N (729 μL, 5.23 mmol, 3.4 eq.). The reaction mixture was subjected thrice to microwave irradiation at 150° C. for 25 min. The reaction mixture was hydrolyzed with water (50 mL) then extracted twice with EtOAc (50 mL). Organic layers were combined, washed with brine (50 mL), dried over magnesium sulfate and concentrated. The crude was purified by flash chromatography (SiO₂, CyHex/EtAOc: 100/0 to 80/20) to afford compound 46 (150 mg, 30%) as a red oil. M/Z (M[⁷⁹Br]+H)⁺: 327.0.

### Compound 47: 4-(8-bromoquinolin-2-yl)-1,4-oxazepane

Compound 47 was prepared according to method h starting from 8-bromo-2-chloroquinoline (500 mg, 2.06 mmol) and 1,4-oxazepane (626 mg, 6.19 mmol). The crude was purified by flash chromatography (SiO₂, CyHex/EtOAc: 100/0 to 70/30) to afford compound 47 as a purple oil (588 mg, 93%). M/Z ([⁷⁹Br]+H)⁺: 307.0

### Compound 48: 8-bromo-7-fluoroquinolin-2(1H)-one

Compound 48 was prepared according to method i starting from 2-bromo-3-fluoroaniline (600 mg, 3.16 mmol). The crude was purified by flash chromatography (SiO₂, DCM/MeOH: 100/0 to 97/3) to afford compound 48 as an orange solid (540 mg, 71%). M/Z ([⁸¹Br]+H)⁺: 243.8

### Compound 49: 8-bromo-5,7-difluoroquinolin-2(1H)-one

Compound 49 was prepared according to method i starting from 2-bromo-3,5-difluoroaniline (600 mg, 2.88 mmol). The crude was purified by flash chromatography (SiO₂, DCM/MeOH: 100/0 to 97/3) to afford compound 49 as an orange solid (347 mg, 46%). M/Z ([⁸¹Br]+H)⁺: 261.8

### Compound 50: 8-bromo-7-chloroquinolin-2(1H)-one

Compound 50 was prepared according to method i starting from 2-bromo-3-chloroaniline (600 mg, 2.91 mmol). The crude was purified by flash chromatography (SiO₂, DCM/MeOH: 100/0 to 98/2) to afford compound 50 as an orange solid (347 mg, 46%). M/Z ([⁷⁹Br][³⁵Cl]+H)⁺: 258.8

### Compound 51: 8-bromo-5,6-difluoroquinolin-2(1H)-one

Compound 51 was prepared according to method i starting from 2-bromo-3,4-difluoroaniline (600 mg, 2.88 mmol). The crude was purified by flash chromatography (SiO₂, DCM/MeOH: 100/0 to 98/2) to afford compound 51 as an orange solid (297 mg, 40%). M/Z ([⁷⁹Br]+H)⁺: 259.8

### Compound 52: 8-bromo-2-chloro-7-fluoroquinoline

Under Argon, to a suspension of 8-bromo-7-fluoroquinolin-2(1H)-one 48 (1.17 g, 4.83 mmol, 1.0 eq.) in toluene (23 mL) was added DMF (0.56 mL, 7.25 mmol, 1.5 eq.). The reaction mixture was heated at 90° C. then POCl₃ (451 μL, 4.83 mmol, 1.0 eq.) was added. The reaction mixture was stirred at 90° C. for 1 h. The reaction mixture was hydrolyzed at 0° C. with NaOH 1N (100 mL) then extracted twice with EtOAc (150 mL). The organic layers were washed thrice with brine (150 mL), dried over magnesium sulfate, concentrated to afford compound 52 (1.11 g, 88%) as a brown solid and was used without further purification. M/Z (M[⁷⁹Br][³⁵Cl]+H)⁺: 260.9.

### Compound 53: 4-(8-bromo-7-fluoroquinolin-2-yl)-1,4-oxazepane

Compound 53 was prepared according to method h starting from 8-bromo-2-chloro-7-fluoroquinoline 52 (150 mg, 0.58 mmol) and 1,4-oxazepane (175 mg, 1.73 mmol, 3.0 eq.). The crude was purified by flash chromatography (SiO₂, CyHex/EtOAc: 100/0 to 70/30) to afford compound 53 as a white solid (162 mg, 87%). M/Z ([⁷⁹Br]+H)⁺: 326.9

### Compound 54: 4-(8-bromo-7-fluoroquinolin-2-yl)morpholine

Compound 54 was prepared according to method h starting from 8-bromo-2-chloro-7-fluoroquinoline 52 (150 mg, 0.58 mmol) and morpholine (151 mg, 1.73 mmol). The crude was purified by flash chromatography (SiO₂, DCM/MeOH: 100/0 to 98/02) to afford compound 54 as a white solid (150 mg, 83%). M/Z ([⁷⁹Br]+H)⁺: 310.9

### Compound 55: 3-(8-bromo-7-fluoroquinolin-2-yl)-8-oxa-3-azabicyclo[3.2.1]octane

Compound 55 was prepared according to method h starting from 8-bromo-2-chloro-7-fluoroquinoline 52 (150 mg, 0.58 mmol) and 8-oxa-3-azabicyclo[3.2.1]octane hydrochloride (151 mg, 1.73 mmol). Starting from a chlorhydrate, Et₃N (241 μL, 1.73 mmol) was added. The crude was purified by flash chromatography (SiO₂, CyHex/EtOAc: 100/0 to 70/30) to afford compound 55 as a beige solid (165 mg, 85%). M/Z ([⁸¹Br]+H)⁺: 339.0

### Compound 56: 2-(azepan-1-yl)-8-bromo-7-fluoroquinoline

Compound 56 was prepared according to method h starting from 8-bromo-2-chloro-7-fluoroquinoline 52 (150 mg, 0.58 mmol) and azepane (171 mg, 1.73 mmol). The crude was purified by flash chromatography (SiO₂, CyHex/EtOAc: 100/0 to 80/20) to afford compound 56 as a beige solid (144 mg, 77%). M/Z ([⁸¹Br]+H)⁺: 325.0

### Compound 57: 8-bromo-N-cyclohexyl-N-ethyl-7-fluoroquinolin-2-amine

Compound 57 was prepared according to method h starting from 8-bromo-2-chloro-7-fluoroquinoline 52 (150 mg, 0.58 mmol) and N-ethylcyclohexanamine (220 mg, 1.73 mmol). The crude was purified by flash chromatography (SiO₂, CyHex/EtOAc: 100/0 to 80/20) to afford compound 57 as a yellow oil (132 mg, 65%). M/Z ([⁸¹Br]+H)⁺: 353.0

### Compound 58: 8-bromo-N-ethyl-7-fluoro-N-isopropylquinolin-2-amine

Compound 58 was prepared according to method h starting from 8-bromo-2-chloro-7-fluoroquinoline 52 (150 mg, 0.58 mmol) and N-ethylpropan-2-amine (151 mg, 1.73 mmol). The crude was purified by flash chromatography (SiO₂, CyHex/EtOAc: 100/0 to 80/20) to afford compound 58 as a yellow oil (132 mg, 74%). M/Z ([⁸¹Br]+H)⁺: 313.0.

### Compound 59: 8-bromo-N,N-dimethylquinoline-2-carboxamide

Compound 59 was prepared according to method j starting from 8-bromo-2-carboxylic acid (500 mg, 1.98 mmol) and dimethylamine (2M in THF, 1.50 mL, 3.00 mmol). The crude was purified by flash chromatography (SiO₂, CyHex/EtOAc: 100/0 to 60/40) to afford compound 59 as a white solid (540 mg, 71%). M/Z ([⁸¹Br]+H)⁺: 281.0

### Compound 60: (8-bromoquinolin-2-yl)(pyrrolidin-1-yl)methanone

Compound 60 was prepared according to method j starting from 8-bromo-2-carboxylic acid (500 mg, 1.98 mmol) and pyrrolidine (0.34 mL, 4.20 mmol). The crude was purified by flash chromatography (SiO₂, CyHex/EtOAc: 100/0 to 60/40) to afford compound 60 as a light yellow solid (550 mg, 91%). M/Z ([⁷⁹Br]+H)⁺: 304.9

### Compound 61: 8-bromo-N-(oxetan-3-yl)quinoline-2-carboxamide

Compound 61 was prepared according to method j starting from 8-bromo-2-carboxylic acid (125 mg, 0.50 mmol) and oxetan-3-amine (47 mg, 0.64 mmol). The crude was purified by flash chromatography (SiO₂, CyHex/EtOAc: 100/0 to 50/50) to afford compound 61 as a yellow oil (96 mg, 63%). M/Z ([⁷⁹Br]+H)⁺: 306.9

### Compound 62: (8-bromoquinolin-2-yl)methanol

Under Argon, at 0° C., to a solution of 8-bromoquinoline-2-carboxylic acid (500 mg, 1.98 mmol, 1.0 eq.) in THF (15 mL) was added Et₃N (332 μL, 2.38 mmol, 1.2 eq.) and dropwise isobutyl chloroformate (309 μL, 2.38 mmol, 1.2 eq.). The reaction mixture was stirred at 0° C. for 1 h. The formed precipitated was filtered off and washed with anhydrous THF (5 mL). Under Argon, at 0° C., to the filtrate was added dropwise a lithium borohydride solution (2M in THF, 2.58 mL, 5.16 mmol, 2.6 eq.). The reaction mixture was stirred at 0° C. for 1 h. The reaction mixture was hydrolyzed at 0° C. with HCl 1N (20 mL) then extracted thrice with DCM (30 mL). Combined organic layers were washed with brine (30 mL), dried over sodium sulfate and concentrated. The crude was purified by flash chromatography (SiO₂, DCM/MeOH: 100/0 to 97/3) to afford compound 62 (300 mg, 64%) as a colorless oil. M/Z (M[⁸′Br]+H)⁺: 240.0.

### Compound 63: 8-bromo-2-(methoxymethyl)quinoline

Under Argon, to a solution of (8-bromoquinolin-2-yl)methanol 62 (300 mg, 1.26 mmol, 1.0 eq.) in DCM (12 mL) were added DMAN (948 mg, 4.42 mmol, 3.5 eq.) and trimethyloxonium tetrafluoroborate (559 mg, 3.84 mmol, 3.0 eq.). The reaction mixture was stirred at 25° C. for 3 h. The reaction mixture was hydrolyzed with HCl 1N (40 mL), then extracted twice with DCM (30 mL). The organic layers were washed with brine (20 mL), dried over sodium sulfate and concentrated. The crude was purified by flash chromatography (SiO₂, DCM/MeOH: 100/0 to 90/10) to afford compound 63 (178 mg, 56%) as a yellow oil. M/Z (M[⁸¹Br]+H)⁺: 254.0

### Compound 64: 8-bromo-7-(difluoromethoxy)quinoline

In a sealed tube under Argon, to a solution of 8-bromoquinolin-7-ol (100 mg, 0.45 mmol, 1 eq.) in DMF (2.2 mL) was added Cs₂CO₃ (291 mg, 0.89 mmol, 2.0 eq.) and sodium 2-chloro-2,2-difluoroacetate (170 mg, 1.12 mmol, 2.5 eq.). The reaction mixture was heated at 120° C. for 24 h. The reaction mixture was diluted with EtOAc (30 mL), washed with water (20 mL), dried over magnesium sulfate and concentrated. The crude was purified by flash chromatography (SiO₂, DCM/MeOH: 100/0 to 95/5) to afford compound 64 (73 mg, 60%) as a white solid. M/Z (M[⁸¹Br]+H)⁺: 275.8.

### Compound 65: 8-bromo-7-fluoro-3-iodoquinoline

In a sealed tube under Argon, to a solution of 8-bromo-7-fluoroquinoline 23 (1.00 g, 4.42 mmol, 1.0 eq.) in acetic acid (13 mL) was added NIS (1.09 g, 4.87 mmol, 1.1 eq.). The reaction mixture was heated at 100° C. for 1 hour, then NIS (498 mg, 2.21 mmol, 0.5 eq.) was added and heating at 100° C. was maintains for 1 h. The reaction mixture was poured over ice and extracted twice with EtOAc (100 mL). Combined organic layers were washed with brine (100 mL), dried and concentrated. The crude was purified by flash chromatography (SiO₂, CyHex/EtOAc: 100/0 to 80/20) to afford compound 65 (720 mg 46%) as a white solid M/Z (M[⁸¹Br]+H)⁺: 353.7.

### Compound 66: 8-bromo-7-fluoro-3-phenylquinoline

In a sealed tube under Argon, to a suspension of 8-bromo-7-fluoro-3-iodoquinoline 65 (170 mg 0.48 mmol, 1.0 eq.) and phenylboronic acid (59 mg, 0.48 mmol, 1.0 eq.) in toluene (1.0 mL), EtOH (0.17 mL) and H₂O (0.17 mL) was added Na₂CO₃ (102 mg, 0.96 mmol, 2.0 eq.). The reaction mixture was sparged with argon for 10 min before addition of Pd(PPh₃)₄ (28 mg, 24 μmol, 0.05 eq.). The reaction mixture was heated at 90° C. for 5 h. The reaction mixture was hydrolyzed with water (50 mL) and extracted twice EtOAc (50 mL). The organic layers were washed with brine (80 mL), dried over sodium sulfate and concentrated. The crude residue was purified by flash chromatography (SiO₂, CyHex/EtOAc: 100/0 to 80/20) to afford compound 66 (109 mg, 75%) as a yellow solid. M/Z (M[¹Br]+H)⁺: 303.9.

### Compound 67: 7-bromo-1-methylindoline

Under Argon at 0° C., to a solution of 7-bromoindoline (150 mg, 0.76 mmol, 1.0 eq.) in THF (5 mL) was added tBuOK (127 mg, 1.14 mmol, 1.5 eq.). The reaction mixture was stirred at 0° C. for 1 h then iodomethane (161 mg, 1.14 mmol, 1.5 eq.) was added. The reaction mixture was stirred at 0° C. for 1 h. The reaction mixture was hydrolyzed with water (20 mL) then extracted twice with EtOAc (20 mL). The organic layers were filtered through an hydrophobic cartridge and concentrated. The crude was purified by flash chromatography (SiO₂, CyHex/EtOAc: 100/0 to 90/10) to afford compound 67 (105 mg, 65%) as a yellow liquid. M/Z (M[⁸¹Br]+H)⁺: 213.9

### Compound 68: 8-bromo-7-fluoro-3-nitroquinoline

Compound 68 was prepared according to method k starting from compound 23 (1.00 g, 4.40 mmol). The crude was purified by flash chromatography (15 μm Interchim® SiO₂, CyHex/EtOAc: 100/0 to 80/20) to afford compound 68 as a yellow solid (650 mg). M/Z ([⁸¹Br]+H)⁺: 272.1

### Compound 69: 8-bromo-7-fluoroquinolin-3-amine

Compound 69 was prepared according to method l starting from 68 (650 mg). The crude was purified by flash chromatography (SiO₂, CyHex/EtOAc: 100/0 to 20/80) to afford compound 69 (330 mg, 31% over 2 steps) as an orange solid. M/Z (M[⁸¹Br]+H)⁺: 242.8.

### Compound 70: 8-bromo-3,7-difluoroquinoline

Compound 70 was prepared according to method m starting from 8-bromo-7-fluoroquinolin-3-amine 69 (330 mg, 1.37 mmol). The crude was purified by flash chromatography (SiO₂, CyHex/EtOAc: 100/0 to 80/20) to afford compound 70 as a white solid (286 mg, 80%). M/Z ([⁷⁹Br]+H)⁺: 243.8

### Compound 71: 8-bromo-3-chloro-7-fluoroquinoline

Compound 71 was prepared according to method n starting from 8-bromo-7-fluoroquinolin-3-amine 69 (390 mg, 1.62 mmol). The crude was purified by flash chromatography (SiO₂, CyHex/EtOAc: 100/0 to 80/20) to afford compound 71 as a light yellow solid (286 mg, 80%). M/Z ([⁸¹Br][³⁷Cl]+H)⁺: 263.9

### Compound 72: 8-bromo-7-chloro-3-nitroquinoline

Compound 72 was prepared according to method k starting from compound 26 (1.00 g, 4.12 mmol). The crude was purified by flash chromatography (SiO₂, CyHex/EtOAc: 100/0 to 70/30) to afford compound 72 as a yellow solid (504 mg, 43%). M/Z ([⁸¹Br][³⁷Cl]+H)⁺: 290.8.

### Compound 73: 8-bromo-7-chloroquinolin-3-amine

Compound 73 was prepared according to method l starting from compound 72 (500 mg, 1.74 mmol). The crude was purified by flash chromatography (SiO₂, CyHex/EtOAc: 100/0 to 10/90) to afford compound 73 (373 mg, 95%) as a beige solid. M/Z ([⁸¹Br][³⁷Cl]+H)⁺: 260.8.

### Compound 74: 8-bromo-7-chloro-3-fluoroquinoline

Compound 74 was prepared according to method m starting from 8-bromo-7-chloroquinolin-3-amine 73 (290 mg, 1.13 mmol). The crude was purified by flash chromatography (15 μm Interchim® SiO₂, CyHex/DCM: 100/0 to 30/70) to afford compound 74 as a white solid (166 mg, 57%). M/Z ([⁸¹Br][³⁷Cl]+H)⁺: 263.7.

### Compound 75: 8-bromo-3,7-dichloroquinoline

Compound 75 was prepared according to method n starting from 8-bromo-7-chloroquinolin-3-amine 73 (370 mg, 1.44 mmol). The crude was purified by flash chromatography (SiO₂, CyHex/EtOAc: 100/0 to 75/25) to afford compound 75 as a white solid (307 mg, 77%). M/Z ([⁸¹Br][³⁷Cl]₂+H)⁺: 279.8

### Compound 76: 8-bromo-5,7-difluoro-3-nitroquinoline

Compound 76 was prepared according to method k starting from compound 24 (640 mg, 2.62 mmol). The crude was purified by flash chromatography (SiO₂, CyHex/EtOAc: 100/0 to 75/25) to afford compound 76 as a white solid (295 mg, 39%). M/Z ([⁸¹Br]+H)⁺: 290.8.

### Compound 77: 8-bromo-5,7-difluoroquinolin-3-amine

Compound 77 was prepared according to method l starting from compound 76 (368 mg, 1.27 mmol). The crude was purified by flash chromatography (SiO₂, CyHex/EtOAc: 100/0 to 10/90) to afford compound 77 (293 mg, 89%) as a light yellow solid. M/Z ([⁸¹Br]+H)⁺: 260.9.

### Compound 78: 8-bromo-3,5,7-trifluoroquinoline

Compound 78 was prepared according to method m starting from 8-bromo-5,7-difluoroquinolin-3-amine 77 (400 mg, 1.54 mmol). The crude was purified by flash chromatography (SiO₂, CyHex/EtOAc: 100/0 to 80/20) to afford compound 78 as a white solid (166 mg, 57%). M/Z ([⁷⁹Br]+H)⁺: 261.9.

### Compound 79: 8-bromo-3-chloro-5,7-difluoroquinoline

Compound 79 was prepared according to method n starting from 8-bromo-5,7-difluoroquinolin-3-amine 77 (290 mg, 1.12 mmol). The crude was purified by flash chromatography (SiO₂, CyHex/EtOAc: 100/0 to 75/25) to afford compound 79 as a white solid (243 mg, 78%). M/Z ([⁸¹Br][³⁷Cl]+H)⁺: 281.7.

### Compound 80: 8-bromo-6,7-difluoro-3-nitroquinoline & Compound 81: 8-bromo-6,7-difluoroquinolin-3-ol

Compound 80 & compound 81 were prepared according to method k starting from compound 27 (1.50 g, 6.15 mmol). The crude was purified by flash chromatography (SiO₂, CyHex/EtOAc: 100/0 to 70/30) to afford compound 80 as a white solid (660 mg, 37%) M/Z ([⁸¹Br]+H)⁺: 290.8 and compound 81 as a yellow solid (144 mg, 9%) M/Z ([⁸¹Br]+H)⁺: 261.9.

### Compound 82: 8-bromo-6,7-difluoroquinolin-3-amine

Compound 82 was prepared according to method starting from compound 80 (660 mg, 2.28 mmol). The crude was purified by flash chromatography (SiO₂, CyHex/EtOAc: 100/0 to 10/90) to afford compound 82 (576 mg, 98%) as a light yellow solid. M/Z ([⁸¹Br]+H)⁺: 260.8.

### Compound 83: 8-bromo-3,6,7-trifluoroquinoline

Compound 83 was prepared according to method m starting from 8-bromo-6,7-difluoroquinolin-3-amine 82 (280 mg, 1.08 mmol). The crude was purified by flash chromatography (15 μm Interchim® SiO₂, CyHex/EtOAc: 100/0 to 80/20) to afford compound 83 as a white solid (163 mg, 58%). M/Z ([⁸¹Br]+H)⁺: 263.9.

### Compound 84: 8-bromo-3-chloro-6,7-difluoroquinoline

Compound 84 was prepared according to method n starting from 8-bromo-6,7-difluoroquinolin-3-amine 82 (280 mg, 1.08 mmol). The crude was purified by flash chromatography (15 μm Interchim® SiO₂, CyHex/EtOAc: 100/0 to 85/15) to afford compound 84 as a white solid (233 mg, 77%). M/Z ([⁸¹Br][³⁷Cl]+H)⁺: 281.8

### Compound 85: 8-(6-(2,5-dimethyl-1H-pyrrol-1-yl)-2-ethylpyridin-3-yl)-7-fluoroquinolin-3-amine

In a seal tube under Argon, to a solution of compound 7 (284 mg, 0.87 mmol, 1.5 eq.) and 8-bromo-7-fluoroquinolin-3-amine 69 (140 mg, 0.58 mmol, 1.0 eq.) in dioxane (2 mL) was added a solution of K₂CO₃ 1.2M in water (968 μL, 1.16 mmol, 2.0 eq.). The reaction mixture was vacuum purged with argon (3 times) before addition of SPhos Pd G2 (21 mg, 0.03 mmol, 0.05 eq.). The reaction mixture was heated at 80° C. for 16 h. The reaction mixture hydrolyzed with water (40 mL) and extracted twice with EtOAc (50 mL). The organic layers were washed brine (40 mL), dried over sodium sulfate and concentrated. The crude was purified by flash chromatography (20 μm Interchim® SiO₂, DCM/MeOH: 100/0 to 95/5) to afford compound 85 (189 mg, 90%) as a brown solid. M/Z (M+H)⁺: 361.2.

### Compound 86: 3-bromo-8-(6-(2,5-dimethyl-1H-pyrrol-1-yl)-2-ethylpyridin-3-yl)-7-fluoroquinoline

Under Argon, at 0° C., to a suspension of copper(I) bromide (147 mg, 1.03 mmol, 2.0 eq.) in acetonitrile (2.0 mL) was added tert-butyl nitrite (136 μL, 1.03 mmol, 2.0 eq.) and a solution of compound 85 (185 mg, 0.51 mmol, 1.0 eq.) in acetonitrile (4.0 mL). The reaction mixture was heated at 60° C. for 1 h. The reaction mixture was hydrolyzed with NaHCO₃ sat. (50 mL) then extracted thrice with EtOAc (50 mL). The organic layers were washed with brine (100 mL), dried over sodium sulfate and concentrated. The crude was purified by flash chromatography (20 μm Interchim® SiO₂, CyHex/EtOAc: 100/0 to 80/20) to afford compound 86 (47 mg, 19%) as a colorless oil. M/Z (M[⁸¹Br]+H)⁺: 426.0

### Compound 87: 8-bromo-1-methyl-1,2,3,4-tetrahydroquinoline

Under Argon, to a suspension of 8-bromo-1,2,3,4-tetrahydroquinoline hydrochloride (300 mg, 1.21 mmol, 1 eq.) in THE (12 mL) was added formaldehyde 40 wt % in water (831 μL, 12.1 mmol, 10 eq.) and NaBH₃CN (758 mg, 12.1 mmol, 10 eq.). The reaction mixture was stirred at 25° C. for 40 h. The reaction mixture was hydrolyzed with NaOH 1N (100 mL) then extracted with EtOAc (100 mL). The organic layer was washed with brine (100 mL), dried over magnesium sulfate and concentrated. The crude was purified by flash chromatography (SiO₂, CyHex/DCM: 100/0 to 70/30) to obtain compound 87 (149 mg, 55%) as a colorless oil. M/Z (M[⁷⁹Br]+H)⁺: 225.9.

### Compound 88: (7-fluoroquinolin-8-yl)boronic Acid

Under Argon and anhydrous atmosphere, at −78° C., to a solution of 8-bromo-7-fluoroquinoline (1.0 g, 4.4 mmol, 1.0 eq.) in THE (12 mL) was added dropwise n-butyllithium (1.6 M in hexanes, 3.0 mL, 4.90 mmol, 1.1 eq.). The reaction mixture was stirred at −78° C. for 30 min then isopropoxyboronic acid (2.7 mL, 13 mmol, 3.0 eq.) was added in one portion. The reaction mixture was stirred 45 min at −78° C. The reaction mixture was hydrolyzed with water (20 mL) and EtOAc (10 mL) was added. The obtained white solid was filtered and washed with water (30 mL) and iPr₂O (15 mL) to afford compound 88 after an overnight drying under vacuum at 50° C. with P₂O₅ (618 mg, 73%) as a white solid. M/Z (M+H)⁺: 192.0.

### Compound 89: 8-(4,4,5,5-tetramethyl-1,3,2-dioxaborolan-2-yl)quinolin-2(1H)-one

In a sealed tube under Argon, to a solution of 8-bromoquinolin-2(1H)-one (200 mg, 0.89 mmol, 1.0 eq.) and Bis(pinacolato)diboron (453 mg, 1.79 mmol, 2.0 eq.) in 1,4-dioxane (4 mL) was added KOAc (175 mg, 1.79 mmol, 2.0 eq.). The reaction mixture was sparged with argon for 10 min before addition of Pd(dppf)Cl₂ (33 mg, 0.05 Eq, 44.6 μmol). The reaction mixture was heated at 110° C. for 2 h. The reaction mixture was filtered through a pad of Celite® and the cake was washed with EtOAc (30 mL). Filtrate was hydrolyzed NH₄Cl sat. (40 mL), then extracted twice with EtOAc (40 mL). Combined organic layers were washed with brine (50 mL), dried over magnesium sulfate and concentrated. The crude was purified by flash chromatography (SiO₂, DCM/MeOH: 100/0 to 95/5) to afford compound 89 (200 mg, 82%) as an orange solid. M/Z (M+H)⁺: 272.1.

### Compound 90: 5-(2-chloro-4-fluorophenyl)-6-ethylpyridin-2-amine

Compound 90 was prepared according to method e from 5-bromo-6-ethylpyridin-2-amine 1d (308 mg, 1.53 mmol) and (2-chloro-4-fluorophenyl)boronic acid (400 mg, 2.29 mmol, 1.5 eq.). The crude was purified by flash chromatography (SiO₂, DCM/MeOH: 100/0 to 96/4) to afford compound 90 as a beige solid (195 mg, 51%). M/Z (M[³⁵Cl]+H)⁺: 251.1.

### Compound 91: 5-(2-chloro-5-fluorophenyl)-6-ethylpyridin-2-amine

Compound 91 was prepared according to method e from 5-bromo-6-ethylpyridin-2-amine 1d (500 mg, 2.49 mmol) and (2-chloro-5-fluorophenyl)boronic acid (650 mg, 3.73 mmol, 1.5 eq.). The crude was purified by flash chromatography (SiO₂, DCM/MeOH: 100/0 to 97/3) to afford compound 91 as a beige solid (393 mg, 63%). M/Z (M[³⁵Cl]+H)⁺: 251.1.

### Compound 92: 1-(allyloxy)-2-bromo-3-fluorobenzene

Under Argon, to a solution of 2-bromo-3-fluorophenol (1.13 mL, 10.5 mmol, 1.0 eq.) in Acetonitrile (27 mL) was added K₂CO₃ (1.74 g, 12.6 mmol, 1.2 eq.). The reaction mixture was heated at 80° C. then a solution of allyl bromide (1.45 mL, 16.8 mmol, 1.6 eq.) in acetonitrile (3.3 mL) was added. The reaction mixture was heated at 80° C. for 18 h. The reaction mixture was hydrolyzed with water (300 mL) and extracted twice with EtOAc (300 mL). The organic layers were washed with brine (300 mL), dried over sodium sulfate and concentrated. The crude was purified by flash chromatography (SiO₂, CyHex/EtOAc: 100/0 to 80/20) to afford compound 92 as a colorless oil (2.25 g, 93%). ¹H-NMR (DMSO-d₆, 400 MHz) δ: 4.69 (dt, J 5.0, 1.6 Hz, 2H, O—CH₂); 5.30 (dq, J 10.4, 1.6 Hz, 1H, CH═CH₂); 5.46 (dq, J 17.2, 1.6 Hz, 1H, CH═CH₂); 6.05 (ddt, J 17.2, 10.4, 5.0 Hz, 1H, CH═CH₂); 6.94-6.98 (m, 2H, Ar); 7.34-7.40 (m, 1H, Ar).

### Compound 93: 6-allyl-2-bromo-3-fluorophenol

In a MW vial under Argon, compound 92 (2.22 g, 9.60 mmol, 1.0 eq.) was stirred neat under microwave irradiation at 200° C. for 20 min. The reaction was further subjected to microwave irradiation at 200° C. for 15 min. The crude was purified by flash chromatography (SiO₂, CyHex/DCM: 100/0 to 70/30) to afford compound 93 as a yellow oil (1.80 g). ¹H-NMR (DMSO-d₆, 400 MHz) δ: 3.36 (d, J 6.4 Hz, 2H, Ph-CH₂); 4.99-5.05 (m, 2H, CH═CH₂); 5.87-5.97 (m, 1H, CH═CH₂); 6.80 (t, J, 8.4 Hz, 1H, Ar); 7.07 (dd, J 8.4, 6.8 Hz, 1H, Ar), 9.52 (s, 1H, OH).

### Compound 94: 2-bromo-3-fluoro-6-(3-hydroxypropyl)phenol

Under Argon, at 0° C., to a solution of compound 93 (500 mg, 2.16 mmol, 1.0 eq.) in THF (22 mL) was added dropwise borane dimethyl sulfide complex (0.82 mL, 8.66 mmol, 4.0 eq.). The reaction was stirred at 0° C. for 2 h. When borylation was complete (full conversion of the starting material was noticed by UPLC-MS), NaOH 2N (2.2 mL) was added dropwise at 0° C. then hydrogen peroxide (30 wt. % in water, 17.7 mL, 173 mmol, 80 eq.) was added dropwise. The reaction mixture was stirred at 25° C. for 45 min. The reaction mixture was hydrolyzed with HCl 1N (200 mL) until pH ˜1, then extracted twice with DCM (200 mL). The organic layer were washed with brine (200 mL), dried over magnesium sulfate and concentrated. The crude was purified by flash chromatography (SiO₂, DCM/EtOAc: 100/0 to 95/5) to afford compound 94 (520 mg, 97%) as a colorless oil. ¹H-NMR (DMSO-d₆, 400 MHz) δ: 1.65 (tt, J 7.6, 6.4 Hz, 2H, Ph-CH₂—CH₂); 2.61 (t, J 7.6 Hz, 2H, Ph-CH₂—CH₂); 3.40 (t, J 6.4 Hz, 2H, O—CH₂); 4.59 (bs, 1H, OH); 6.77 (t, J, 8.4 Hz, 1H, Ar); 7.10 (dd, J 8.4, 6.6 Hz, 1H, Ar), 9.44 (bs, 1H, Ph-OH).

### Compound 95: 8-bromo-7-fluorochromane

Under Argon, at 0° C., to a solution of 2-bromo-3-fluoro-6-(3-hydroxypropyl)phenol 94 (250 mg, 1.00 mmol, 1.0 eq.) in THF (5 mL), diisopropyl-diazene-1,2-dicarboxylate (217 μL, 1.10 mmol, 1.1 eq.) and triphenylphosphine (290 mg, 1.10 mmol, 1.1 eq.) were added. The reaction mixture was stirred at 25° C. for 3 h. The reaction mixture was hydrolyzed with NaOH 1N (10 mL), then extracted twice with EtOAc (10 mL). The organic layers were washed with HCl 1N (10 mL), brine (10 mL), dried over magnesium sulfate and concentrated. The crude was purified by flash chromatography (SiO₂, CyHex/EtOAc: 100/0 to 70/30) to afford compound 95 (166 mg, 72%) as a colorless oil. ¹H-NMR (DMSO-d₆, 400 MHz) δ: 1.92 (tt, J 6.4, 5.2 Hz, 2H, O—CH₂—CH₂); 2.75 (td, J 6.4, 0.8 Hz, 2H, Ph-CH₂); 4.26 (t, J 5.2 Hz, 2H, O—CH₂—CH₂); 6.82 (t, J, 8.4 Hz, 1H, Ar); 7.10-7.13 (m, 1H, Ar).

### Compound 96: 8-bromo-7-fluoro-2,2-dimethylchromane

Under Argon, to a solution of 2-bromo-3-fluorophenol (0.57 mL, 5.2 mmol, 10.0 eq.) in DCM (2.6 mL), 3-methylbut-2-en-1-yl acetate (73 μL, 0.52 mmol, 1.0 eq.) and indium(III) trifluoromethanesulfonate (29 mg, 0.52 mmol, 0.1 eq.) were added. The reaction mixture was stirred at 25° C. for 2 h. The reaction mixture was hydrolyzed with NaOH 1N (50 mL) then extracted twice with Et₂O (25 mL). The organic layers were washed twice with NaOH 1N (50 mL), brine (50 mL), dried over magnesium sulfate and concentrated to afford compound 96 (88 mg, 65%) as a colorless oil which was used without further purification. ¹H-NMR (DMSO-d₆, 400 MHz) δ: 1.31 (s, 6H, 2*CH₃); 1.78 (t, J 6.8 Hz, 2H, Ph-CH₂—CH₂); 2.75 (t, J 6.8 Hz, 2H, Ph-CH₂); 6.80 (t, J, 8.4 Hz, 1H, Ar); 7.11-7.15 (m, 1H, Ar).

### Compound 97: 1-allyl-2-(allyloxy)-3-bromo-4-fluorobenzene

Under Argon, to a solution of 6-allyl-2-bromo-3-fluorophenol 93 (1.18 g, 5.11 mmol, 1.0 eq.) in acetonitrile (25 mL), K₂CO₃ (847 mg, 5.35 mmol, 1.2 eq.) was added. The reaction mixture was heated at 80° C., then a solution of allyl bromide (707 μL, 7.13 mmol, 1.6 eq.) in acetonitrile (4 mL) was added. The reaction mixture was stirred at 80° C. for 18 h. The reaction mixture was hydrolyzed with water (40 mL) and extracted twice with EtOAc (40 mL). The organic layers were washed with brine (40 mL), dried over sodium sulfate and concentrated. The crude was purified by flash chromatography (SiO₂, CyHex/EtOAc: 100/0 to 80/20) to afford compound 97 as a colorless oil (1.45 g). ¹H-NMR (DMSO-d₆, 400 MHz) δ: 3.39 (d, J 6.4 Hz, 2H, Ph-CH₂); 4.46 (ddd, J 5.6, 1.6, 1.2 Hz, 2H, O—CH₂); 5.02-5.10 (m, 2H, CH═CH₂); 5.28 (m, 1H, CH═CH₂); 5.44 (dq, J 17.2, 1.6 Hz, 1H, CH═CH₂); 5.94 (ddt, J 16.8, 10.4, 6.4 Hz, 1H, CH═CH₂); 6.11 (ddt, J 17.2, 10.4, 5.8 Hz, 1H, CH═CH₂); 7.14 (t, J, 8.4 Hz, 1H, Ar); 7.25 (dd, J 8.4, 6.8 Hz, 1H, Ar).

### Compound 98: 9-bromo-8-fluoro-2,5-dihydrobenzo[b]oxepine

Under Argon, to a solution of 1-allyl-2-(allyloxy)-3-bromo-4-fluorobenzene 97 (876 mg, 3.23 mmol, 1.0 eq.) in DCM (16 mL), Grubbs II catalyst (15 mg, 0.02 mmol, 0.006 eq.) was added. The reaction mixture was stirred at 25° C. for 22 h. The reaction mixture was filtered through a Celite® pad and the cake washed with DCM (100 mL). The organic layer was hydrolyzed with NaHCO₃ sat. (100 mL) and extracted twice with DCM (100 mL). The organic layers were washed with brine (100 mL), dried over magnesium sulfate and concentrated. The crude was purified by flash chromatography (20 μm Interchim® SiO₂, CyHex/EtOAc: 100/0 to 90/10) to afford compound 98 (734 mg, 94% over 2 steps) as a colorless oil. ¹H-NMR (DMSO-d₆, 400 MHz) δ: 3.44-3.46 (m, 2H, Ph-CH₂); 4.47-4.60 (m, 2H, O—CH₂); 5.49-5.53 (m, 1H, CH═CH); 5.80-5.86 (m, 1H, CH═CH); 7.05 (t, J, 8.4 Hz, 1H, Ar); 7.23 (dd, J 8.4, 6.4 Hz, 1H, Ar).

### II. Preparation of Selected Examples of the Invention

### General Methods

### Method 1: Suzuki Coupling

In a sealed vial, to a solution of aminopyridine halide 1 (1.0 eq.) and heteroaryl boronic derivative (1.2-1.5 eq.) in dioxane (C=0.2 M), an aqueous solution of K₂CO₃ (1.2 M, 2.0 eq.) was added dropwise. The resulting suspension was degassed with argon bubbling for 15 min and PdCl₂(dppf).CH₂Cl₂ (5 mol %) was then added in one portion. The vial was sealed and the mixture was stirred at 90° C. until no more evolution was noticed by UPLC-MS (overnight, unless mentioned otherwise). The reaction mixture was cooled to rt, hydrolysed and then extracted thrice with EtOAc. The combined organic layers were washed with brine, dried over MgSO₄, filtered and concentrated. For specific examples, the corresponding hydrochloride salt was prepared.

### Method 2: Suzuki Couplinq

In a sealed vial, to a solution of aminopyridine halide 1 (1.0 eq.) and heteroaryl boronic derivative (1.2-1.5 eq.) in dioxane (C=0.2 M) an aqueous solution of K₂CO₃ (1.2 M, 2.0 eq.) was added dropwise. The resulting suspension was degassed with argon bubbling for 15 min and SPhos Pd G2 (5 mol %) was then added in one portion. The vial was sealed and the mixture was stirred at 80° C. until no more evolution was noticed by UPLC-MS (overnight, unless mentioned otherwise). The reaction mixture was cooled to rt, hydrolysed and then extracted thrice with EtOAc. The combined organic layers were washed with brine, dried over MgSO₄, filtered and concentrated. The residue was purified by flash chromatography. For specific examples, the corresponding hydrochloride salt was prepared.

### Method 3: Suzuki Coupling

In a sealed vial, to a solution of aminopyridine halide 1 (1.0 eq.) and heteroaryl boronic derivative (1.2-1.5 eq.) in dioxane (C=0.2 M), an aqueous solution of K₂CO₃ (1.2 M, 2.0 eq.) was added dropwise. The resulting suspension was degassed with argon bubbling for 15 min and P(tBu)₃ Pd G2 (7 mol %) was then added in one portion. The vial was sealed and the mixture was stirred at 90° C. until no more evolution was noticed by UPLC-MS (overnight, unless mentioned otherwise). The reaction mixture was cooled to rt, filtered on a Celite® pad and the cake was washed with MeOH. The filtrate was concentrated and purified by flash chromatography. For specific examples, the corresponding hydrochloride salt was prepared.

### Method 4: Suzuki Coupling

In a sealed vial, to a solution of aminopyridine halide 1 (1.0 eq.) and heteroaryl boronic derivative (1.2-1.5 eq.) in ethanol (C=0.2 M) an aqueous solution of Na₂CO₃ (1.2 M, 1.5 eq.) was added dropwise. The resulting suspension was degassed with argon bubbling for 15 min and XPhos Pd G2 (5 mol %) was then added in one portion. The vial was sealed and the mixture was stirred at 90° C. until no more evolution was noticed by UPLC-MS (overnight, unless mentioned otherwise). The reaction mixture was cooled to rt, filtered on a Celite® pad and the cake was washed with DCM/MeOH 9/1. The filtrate was concentrated and purified by flash chromatography. For specific examples, the corresponding hydrochloride salt was prepared.

### Method 5: Suzuki Coupling

In a sealed vial, to a solution of aminopyridine halide 1 (1.0 eq.), heteroaryl boronic derivative 3 (1.2-2.5 eq.) and CataCXium HI (0.1 eq.) in dioxane (C=0.2 M), an aqueous solution of K₂CO₃ (1.2 M, 2.5 eq.) was added dropwise. The resulting suspension was degassed with argon bubbling for 15 min and Pd(OAc)₂ (5 mol %) was then added in one portion. The vial was sealed and the mixture was stirred at 120° C. until no more evolution was noticed by UPLC-MS (overnight, unless mentioned otherwise). The reaction mixture was cooled to rt, filtered on a Celite® pad and the cake was washed with DCM/MeOH 9/1. The organic layer was washed with NH₄Cl sat., the aqueous layer was extracted with DCM and two organic layers was washed with brine, dried over magnesium sulfate and concentrated. The crude residue was purified by flash chromatography. For specific examples, the corresponding hydrochloride salt was prepared.

### Method 6: Suzuki Coupling

In a sealed vial, to a suspension of heteroarylbromide (1.0 eq.) and boronate (1.5 eq.) in dioxane (C=0.2 M), an aqueous solution of K₂CO₃ (1.2 M, 2.0 eq.) was added dropwise. The resulting suspension was degassed with argon bubbling for 15 min and SPhos Pd G2 (5 mol %) was then added in one portion. The vial was sealed and the mixture was stirred at 80° C. until no more evolution was noticed by UPLC-MS (overnight, unless mentioned otherwise). The reaction mixture was cooled to rt, filtered on a Celite® pad and the cake was washed with DCM/MeOH 9/1. The filtrate was concentrated and purified by flash chromatography. For specific examples, the corresponding hydrochloride salt was prepared.

### Method 7: Suzuki Coupling

In a sealed vial, to a suspension of heteroarylbromide (1.0 eq.) and boronate (1.5 eq.) in dioxane (C=0.2 M), an aqueous solution of K₂CO₃ (1.2 M, 2.0 eq.) was added dropwise. The resulting suspension was degassed with argon bubbling for 15 min and PdCl₂ dppf (5 mol %) was then added in one portion. The vial was sealed and the mixture was stirred at 90° C. until no more evolution was noticed by UPLC-MS (overnight, unless mentioned otherwise). The reaction mixture was cooled to rt, filtered on a Celite® pad and the cake was washed with DCM/MeOH 9/1. The filtrate was concentrated and purified by flash chromatography. For specific examples, the corresponding hydrochloride salt was prepared.

### Method 8: Suzuki Coupling

In a sealed vial, to a solution of halide 5b (1.0 eq.), (hetero)aryl boronic derivative (1.2-1.5 eq.) and P(Cy)₃ (20 mol %) in dioxane (C=0.2 M), a solution of TBAF (1.0 M in THF, 2.0 eq.) was added dropwise. The resulting solution was degassed with argon bubbling for 15 min and Pd₂(dba)₃ (10 mol %) was then added in one portion. The vial was sealed, and the mixture was stirred at 100° C. until no more evolution was noticed by UPLC-MS (overnight, unless mentioned otherwise). The reaction mixture was cooled to rt, hydrolysed and then extracted thrice with EtOAc. The combined organic layers were washed with brine, dried over MgSO₄, filtered and concentrated. The residue was purified by flash chromatography. For specific examples, the corresponding hydrochloride salt was prepared.

### Method 9: Suzuki Coupling/Dimethylpyrrole Cleavage

Step 1: In a sealed vial, to a suspension of bromo (heteroaryl) (1.0 eq.) and boronic ester (1.1-1.5 eq.) in dioxane (C=0.2 M) was added dropwise an aqueous solution of K₂CO₃ (1.2 M, 2.0 eq.). The resulting suspension was sparged with argon for 10 min and SPhos Pd G2 (5 mol %) was added. The vial was sealed, and the reaction mixture was stirred at 80° C. until no more evolution was noticed by UPLC-MS (overnight, unless mentioned otherwise). The reaction mixture was hydrolysed and then extracted thrice with EtOAc. The combined organic layers were washed with brine, dried over MgSO₄, filtered and concentrated. The residue was purified by flash chromatography to afford the protected intermediate.

Step 2: Under Argon, to a suspension of the protected intermediate (1.0 eq.) in a mixture of EtOH/H₂O: 2/1 (C=0.1 M) was added hydroxylamine hydrochloride (20.0 eq.) and triethylamine (3.6 eq.). The reaction mixture was heated at 90° C. until no more evolution was noticed by UPLC-MS (4 h, unless mentioned otherwise). The reaction mixture was hydrolyzed with HCl 1 M and extracted twice with Et₂O. The aqueous layer was basified with NaOH 6N, and extracted thrice with DCM. Combined DCM layers were dried over sodium sulfate and concentrated. The crude was purified by flash chromatography.

### Method 10 Peptidic Coupling/Dimethylpyrrole Cleavage

Step 1: Under Argon, to a solution of quinoline-2-carboxylic acid derivative (1.00 eq.) in DMF (C=0.1 M), BOP (1.3 eq.), N-ethyl-N-isopropylpropan-2-amine (3.0 eq.) and amine (1.1 eq.) were added. The reaction mixture was stirred at rt for 1 h. The reaction mixture was hydrolyzed with NH₄Cl sat. and extracted twice with EtOAc. The organic layers were washed with brine, dried over magnesium sulfate and concentrated. The residue was purified by flash chromatography to afford the protected intermediate.

Step 2: Under Argon, to a suspension of the protected intermediate (1 eq.) in a mixture of EtOH/H₂O: 2/1 (C=0.1 M) was added hydroxylamine hydrochloride (20.0 eq.) and triethylamine (3.6 eq.). The reaction mixture was heated at 90° C. until no more evolution was noticed by UPLC-MS (4 h, unless mentioned otherwise). The reaction mixture was hydrolyzed with HCl 1 M and extracted twice with Et₂O. The aqueous layer was basified with NaOH 6N and extracted thrice with DCM. Combined DCM layers were dried over sodium sulfate and concentrated. The crude was purified by flash chromatography.

### Method 11: Suzuki Coupling/Pivaloyl Deprotection

Step 1: In a sealed vial, to a suspension of bromo (heteroaryl) (1.0 eq.) and boronic ester (1.1-1.5 eq.) in dioxane (C=0.2 M), an aqueous solution of K₂CO₃ (1.2 M, 2.0 eq.) was added. The resulting suspension was sparged with argon for 10 min and SPhos Pd G2 (5 mol %) was added. The vial was sealed, and the reaction mixture was stirred at 80° C. until no more evolution was noticed by UPLC-MS (overnight, unless mentioned otherwise). The reaction mixture hydrolysed and then extracted thrice with EtOAc. The combined organic layers were washed with brine, dried over MgSO₄, filtered and concentrated. The residue was purified by flash chromatography to afford the protected intermediate.

Step 2: In a MW vial under Argon, a solution of the protected intermediate (1 eq.) in a mixture dioxane/aqueous HCl 3N: 1/1 (C=0.1 M) was heated at 150° C. under MW irradiation for 30 min. The reaction mixture was neutralized with K₂CO₃ sat. until pH-9 and extracted twice with EtOAc. Combined organic layers were washed with brine, dried over sodium sulfate and concentrated. The crude was purified by flash chromatography.

### Method 12: Suzuki Coupling/Dimethylpyrrole Cleavage

Step 1: In a sealed vial, to a suspension of bromo (heteroaryl) (1.0 eq.) and boronic ester (1.0-1.1 eq.) in toluene/EtOH/H₂O: 6/1/1 (C=0.2 M), K₂CO₃ (2.0 eq.) was added. The resulting suspension was sparged with argon for 10 min and Pd(PPh₃)₄ (5 mol %) was added. The vial was sealed, and the reaction mixture was stirred at 80° C. until no more evolution was noticed by UPLC-MS (overnight, unless mentioned otherwise). The reaction mixture was filtered through a pad of Celite® and the cake was washed with DCM. The reaction mixture was hydrolysed with NH₄Cl sat. and then extracted thrice with DCM. The combined organic layers were washed with brine, dried over MgSO₄, filtered and concentrated. The residue was purified by flash chromatography to afford the protected intermediate.

Step 2: Under Argon, to a suspension of the protected intermediate (1.0 eq.) in a mixture of EtOH/H₂O: 2/1 (C=0.1 M) was added hydroxylamine hydrochloride (20.0 eq.) and triethylamine (3.6 eq.). The reaction mixture was heated at 90° C. until no more evolution was noticed by UPLC-MS (4 h, unless mentioned otherwise). The reaction mixture was hydrolyzed with HCl 1 M and extracted twice with Et₂O. The aqueous layer was basified with NaOH 6N and extracted thrice with DCM. Combined DCM layers were dried over sodium sulfate and concentrated. The crude was purified by flash chromatography.

### Example Syntheses

### Example 1: 6-ethyl-5-(5-fluoroquinolin-8-yl)pyridin-2-amine (hydrochloride)

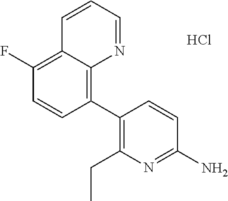 [SMILES: CCc1nc(N)ccc1-c1ccc(F)c2cccnc12 ; Cl]

Example 1 was prepared according to method 2 starting from 2-amino-5-bromo-6-ethylpyridine 1d (60 mg, 0.30 mmol) and (5-fluoroquinolin-8-yl)boronic acid (87 mg, 0.46 mmol, 1.5 eq.). The crude was purified by flash chromatography (SiO₂, DCM/MeOH: 100/0 to 95/5). The resulting foam was further purified by flash chromatography (15 μm Interchim® SiO₂, DCM/MeOH: 100/0 to 96/4). The obtained foam was dissolved in a mixture of aqueous 1N HCl/ACN and the resulting solution was freeze dried to afford Example 1 as a white solid (24 mg, 26%).

¹H-NMR (DMSO-d₆, 400 MHz) δ: 1.06 (t, J 7.6 Hz, 3H, CH₂—CH₃); 2.37-2.46 (m, 2H, CH₂—CH₃); 6.94 (d, J 9.0 Hz, 1H, Ar); 7.58 (dd, J 9.9, 8.1 Hz, 1H, Ar); 7.71 (dd, J 8.5, 4.2 Hz, 1H, Ar); 7.77 (dd, J 8.1, 6.1 Hz, 1H, Ar); 7.80 (d, J 9.0 Hz, 1H, Ar); 8.00 (bs, 2H, NH₂); 8.59 (dd, J 8.5, 1.7 Hz, 1H, Ar); 8.97 (dd, J 4.2, 1.7 Hz, 1H, Ar); 14.20 (bs, 1H, HCl salt). M/Z (M+H)⁺: 268.1. Mp: 120-140° C.

### Example 2: 6-Fluoro-5-quinolin-8-yl-pyridin-2-ylamine (hydrochloride)

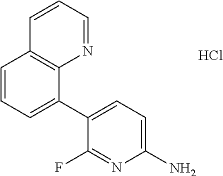 [SMILES: Nc1ccc(-c2cccc3cccnc23)c(F)n1 ; Cl]

Example 2 was prepared according to method 1 starting from 2-amino-5-bromo-6-fluoropyridine (100 mg, 0.58 mmol) and 8-quinolinyl boronic acid (150 mg, 0.87 mmol, 1.5 eq.). The crude was purified by flash chromatography (SiO₂, DCM/MeOH: 100/0 to 95/5). The obtained foam was dissolved in a mixture of aqueous 1 N HCl/ACN and the resulting solution was freeze dried to afford Example 2 as a yellow solid (93 mg, 58%).

¹H NMR (400 MHz, DMSO-d₆) δ: 6.50 (dd, J 8.2, 1.9 Hz, 1H, Ar); 7.64 (dd, J 10.2, 8.2 Hz, 1H, Ar); 7.79-7.89 (m, 3H, Ar); 8.19 (dd, J 7.9, 1.3 Hz, 1H, Ar); 8.84 (d, J 8.2 Hz, 1H, Ar); 9.02 (dd, J 4.7, 1.3 Hz, 1H, Ar) HCl salt signal not observed. M/Z (M+H)⁺: 240.0. Mp>250° C.

### Example 3: 6-Methyl-5-quinolin-8-yl-pyridin-2-ylamine (hydrochloride)

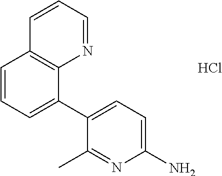 [SMILES: Cc1nc(N)ccc1-c1cccc2cccnc12 ; Cl]

Example 3 was prepared according to method 1 starting from 2-amino-5-bromo-6-methylpyridine (100 mg, 0.58 mmol) and 8-quinolinyl boronic acid (150 mg, 0.87 mmol, 1.5 eq.). The crude was purified by flash chromatography (SiO₂, DCM/MeOH: 100/0 to 95/5). The obtained foam was dissolved in a mixture of aqueous 1 N HCl/ACN and the resulting solution was freeze dried to afford Example 3 as a brown solid (81 mg, 51%).

¹H NMR (400 MHz, DMSO-d₆) δ: 2.18 (s, 3H, CH₃); 6.95 (d, J 9.0 Hz, 1H, Ar); 7.71 (dd, J 8.3, 4.4 Hz, 1H, Ar); 7.75-7.83 (m, 2H, Ar); 7.84 (d, J 9.0 Hz, 1H, Ar); 7.97 (bs, 2H, NH₂); 8.17 (dd, J 7.7, 1.8 Hz, 1H, Ar); 8.63 (dd, J 8.2, 1.3 Hz, 1H, Ar); 8.94 (dd, J 4.4, 1.8 Hz, 1H, Ar); 14.47 (s, 1H, HCl salt). M/Z (M+H)⁺: 236.1. Mp>250° C.

### Example 4: 5-Benzo[b]thiophen-3-yl-6-ethyl-pyridin-2-ylamine

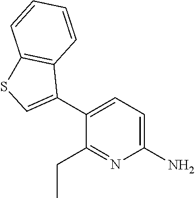 [SMILES: CCc1nc(N)ccc1-c1csc2ccccc12]

Example 4 was prepared according to method 1 starting from 2-amino-5-bromo-6-ethylpyridine 1d (75 mg, 0.37 mmol) and benzo[b]thien-3-yl boronic acid (100 mg, 0.56 mmol, 1.5 eq.). The crude was purified by flash chromatography (SiO₂, DCM/MeOH: 100/0 to 97/3). The obtained foam was triturated in pentane and the collected precipitate was dried under high vacuum at 70° C. overnight to afford Example 4 as a brown solid (53 mg, 56%).

¹H NMR (400 MHz, DMSO-d₆) δ: 1.01 (t, J 7.5 Hz, 3H, CH₂—CH₃); 2.36 (q, J 7.5 Hz, 2H, CH₂—CH₃); 5.98 (s, 2H, NH₂); 6.39 (d, J 8.4 Hz, 1H, Ar); 7.23 (d, J 8.4 Hz, 1H, Ar); 7.36-7.41 (m, 3H, Ar); 7.57 (s, 1H, Ar); 8.01-8.04 (m, 1H, Ar). M/Z (M+H)⁺: 255.7. Mp: 108-120° C.

### Example 5: 6-Ethyl-5-(6-methoxybenzothiophen-3-yl)pyridin-2-amine

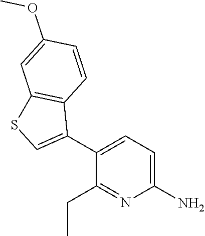 [SMILES: CCc1nc(N)ccc1-c1csc2cc(OC)ccc12]

Example 5 was prepared according to method 1 starting from 2-amino-5-bromo-6-ethylpyridine 1d (100 mg, 0.50 mmol) and 2-(6-methoxybenzothiophen-3-yl)-4,4,5,5-tetramethyl-1,3,2-dioxaborolane 3b (217 mg, 0.75 mmol, 1.5 eq.). The crude was purified by flash chromatography (SiO₂, CyHex/EtOAc: 100/0 to 50/50). The obtained foam was further purified by flash chromatography (15 μm Interchim® SiO₂, CyHex/EtOAc, 100/0 to 50/50). The obtained foam was triturated in pentane and the collected precipitate was dried under high vacuum at 70° C. overnight to afford Example 5 as a brown solid (49 mg, 34%).

¹H NMR (400 MHz, DMSO-d₆) δ: 1.16 (t, J 7.5 Hz, 3H, CH₂—CH₃); 2.70 (q, J 7.5 Hz, 2H, CH₂—CH₃); 3.82 (s, 3H, O—CH₃); 6.10 (bs, 2H, NH₂); 6.35 (d, J 8.4 Hz, 1H, Ar); 6.99 (dd, J 8.7, 2.4 Hz, 1H, Ar); 7.18 (s, 1H, Ar); 7.38 (d, J 8.4 Hz, 1H, Ar); 7.51 (d, J 2.4 Hz, 1H, Ar); 7.70 (d, J 8.7 Hz, 1H, Ar). M/Z (M+H)⁺: 285.7. Mp: 151-155° C.

### Example 6: 6-Ethyl-5-(8-isoquinolyl)pyridin-2-amine (hydrochloride)

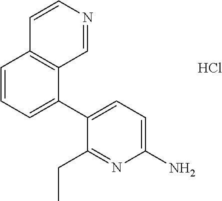 [SMILES: CCc1nc(N)ccc1-c1cccc2ccncc12 ; Cl]

Example 6 was prepared according to method 2 starting from 2-amino-5-bromo-6-ethylpyridine 1d (100 mg, 0.47 mmol) and 8-isoquinolinyl boronic acid (130 mg, 0.75 mmol, 1.5 eq.). The crude was purified by flash chromatography (SiO₂, DCM/MeOH: 100/0 to 95/5). The obtained foam was dissolved in a mixture of aqueous 1N HCl/ACN and the resulting solution was freeze dried to afford Example 6 as a white solid (116 mg, 81%).

¹H NMR (400 MHz, DMSO-d₆) δ: 1.08 (t, J 7.6 Hz, 3H, CHₐH_{b}—CH₃); 2.32-2.40 (m, 1H, CHₐH_{b}—CH₃); 2.52-2.59 (m, 1H, CHₐH_{b}—CH₃); 7.00 (d, J 9.0 Hz, 1H, Ar); 7.81-7.84 (m, 2H, Ar); 8.12-8.29 (m, 3H, Ar+NH₂); 8.32 (d, J 8.3 Hz, 1H, Ar); 8.40 (d, J 6.2 Hz, 1H, Ar); 8.69 (d, J 6.2 Hz, 1H, Ar); 9.33 (s, 1H, Ar); 14.56 (bs, 1H, HCl salt). M/Z (M+H)⁺: 250.8. Mp>250° C.

### Example 7: 5-Benzo[b]thiophen-3-yl-6-propyl-pyridin-2-ylamine (hydrochloride)

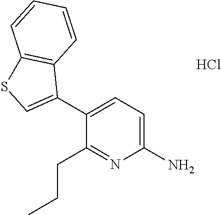 [SMILES: CCCc1nc(N)ccc1-c1csc2ccccc12 ; Cl]

Example 7 was prepared according to method 2 starting from 2-amino-5-bromo-6-propylpyridine 1e (93 mg, 0.43 mmol) and benzo[b]thien-3-yl boronic acid (129 mg, 0.65 mmol, 1.5 eq.). The crude was purified by flash chromatography (SiO₂, DCM/MeOH, 100/0 to 97/3). The obtained foam was further purified by flash chromatography (15 μm Interchim® SiO₂, DCM/MeOH: 100/0 to 97/3). The resulting solid was dissolved in a mixture of aqueous 1N HCl/ACN and the resulting solution was freeze dried to afford Example 7 as a white solid (81 mg, 62%).

¹H NMR (400 MHz, DMSO-d₆) δ: 0.70 (t, J 7.4 Hz, 3H, CH₂—CH₂—CH₃); 1.50-1.59 (m, 2H, CH₂—CH₂—CH₃); 2.52-2.56 (m, 2H, CH₂—CH₂—CH₃); 6.96 (d, J 9.0 Hz, 1H, Ar); 7.40-7.50 (m, 3H, Ar); 7.83 (d, J 9.0 Hz, 1H, Ar); 7.83 (s, 1H, Ar); 8.03 (bs, 2H, NH₂); 8.08-8.10 (m, 1H, Ar); 14.24 (bs, 1H, HCl salt). M/Z (M+H)⁺: 269.7. Mp: 175-190° C.

### Example 8: 6-Propyl-5-(8-quinolyl)pyridin-2-amine (hydrochloride)

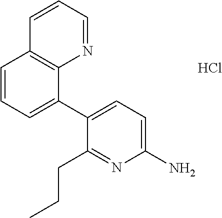 [SMILES: CCCc1nc(N)ccc1-c1cccc2cccnc12 ; Cl]

Example 8 was prepared according to method 2 starting from 2-amino-5-bromo-6-propylpyridine 1e (100 mg, 0.43 mmol) and 8-quinolinyl boronic acid (112 mg, 0.65 mmol, 1.5 eq.). The crude was purified by flash chromatography (SiO₂, DCM/MeOH, 100/0 to 95/5). The obtained foam was dissolved in a mixture of aqueous 1N HCl/ACN and the resulting solution was freeze dried to afford Example 8 as a yellow solid (124 mg, 96%).

¹H NMR (400 MHz, DMSO-d₆) δ: 0.63 (t, J 7.4 Hz, 3H, CHₐH_{b}—CH₂—CH₃); 1.46-1.55 (m, 2H, CHₐH_{b}—CH₂—CH₃); 2.28-2.40 (m, 1H, CHₐH_{b}—CH₂CH₃); 2.52-2.56 (m, 1H, CHₐH_{b}—CH₂CH₃); 6.96 (d, J 9.0 Hz, 1H, Ar); 7.69 (dd, J 8.4, 4.2 Hz, 1H, Ar); 7.74-7.81 (m, 2H, Ar); 7.81 (d, J 9.0 Hz, 1H, Ar); 8.06 (bs, 2H, NH₂); 8.16 (dd, J 7.4, 2.4 Hz, 1H, Ar); 8.60 (dd, J 8.4, 1.6 Hz, 1H, Ar); 8.92 (dd, J 4.3, 1.6 Hz, 1H, Ar); 14.43 (bs, 1H, HCl salt). M/Z (M+H)⁺: 264.5. Mp: 80-120° C.

### Example 9: 5-(8-Isoquinolyl)-6-propyl-pyridin-2-amine (hydrochloride)

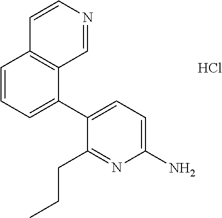 [SMILES: CCCc1nc(N)ccc1-c1cccc2ccncc12 ; Cl]

Example 9 was prepared according to method 2 starting from 2-amino-5-bromo-6-propylpyridine 1e (100 mg, 0.43 mmol) and 8-isoquinolinyl boronic acid (112 mg, 0.65 mmol, 1.5 eq.). The crude was purified by flash chromatography (SiO₂, DCM/MeOH: 100/0 to 90/10). The obtained foam was further purified by flash chromatography (15 μm Interchim® SiO₂, DCM/MeOH, 100/0 to 90/10). The obtained solid was dissolved in a mixture of aqueous 1N HCl/ACN and the resulting solution was freeze dried to afford Example 9 as a yellow solid (97 mg, 75%).

¹H NMR (400 MHz, DMSO-d₆) δ: 0.67 (t, J 7.4 Hz, 3H, CHₐH_{b}—CH₂—CH₃); 1.46-1.60 (m, 2H, CHₐH_{b}—CH₂—CH₃); 2.26-2.33 (m, 1H, CHₐH_{b}—CH₂—CH₃); 2.52-2.56 (m, 1H, CHₐH_{b}—CH₂—CH₃); 7.01 (d, J 9.0 Hz, 1H, Ar); 7.12-7.15 (m, 2H, Ar); 7.24-7.34 (m, 4H, Ar); 7.46-7.57 (m, 3H, Ar); 7.67 (d, J 9.0 Hz, 1H, Ar); 7.84 (bs, 2H, NH₂); 13.74 (bs, 1H, HCl salt). M/Z (M+H)⁺: 264.8. Mp: 100-117° C.

### Example 10: 5-Benzo[b]thiophen-3-yl-6-isopropyl-pyridin-2-ylamine (hydrochloride)

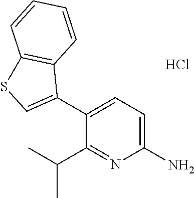 [SMILES: CC(C)c1nc(N)ccc1-c1csc2ccccc12 ; Cl]

Example 10 was prepared according to method 1 starting from 2-amino-5-bromo-6-isopropylpyridine 1f (100 mg, 0.46 mmol) and benzo[b]thien-3-yl boronic acid (123 mg, 0.65 mmol, 1.5 eq.). The crude was purified by flash chromatography (SiO₂, CyHex/EtOAc: 100/0 to 50/50). The obtained foam was triturated in pentane and the collected precipitate was dissolved in a mixture of aqueous 1N HCl/ACN. The resulting solution was freeze dried to afford Example 10 as a white solid (65 mg, 46%).

¹H NMR (400 MHz, DMSO-d₆) δ: 1.27 (d, J 6.9 Hz, 6H, CH(CH₃)₂); 2.84 (sep, J 6.9 Hz, 1H, CH(CH₃)₂); 6.95 (d, J 8.5 Hz, 1H, Ar); 7.38-7.52 (m, 3H, Ar); 7.74-7.85 (m, 2H, Ar); 8.06-8.12 (m, 1H, Ar); 8.37 (bs, 2H, NH₂); 14.02 (s, 1H, HCl salt). M/Z (M+H)⁺: 269.7. Mp>250° C.

### Example 11: 6-Isopropyl-5-(8-quinolyl)pyridin-2-amine (hydrochloride)

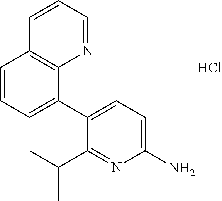 [SMILES: CC(C)c1nc(N)ccc1-c1cccc2cccnc12 ; Cl]

Example 11 was prepared according to method 2 starting from 2-amino-5-bromo-6-isopropylpyridine if (100 mg, 0.46 mmol) and 8-quinolinyl boronic acid (112 mg, 0.65 mmol, 1.5 eq.). The hydrolysis induced the precipitation of the product which was collected by filtration. The resulting powder was further purified by flash chromatography (SiO₂, DCM/MeOH: 100/0 to 95/5). The obtained foam was triturated in pentane and the collected precipitate was dissolved in a mixture of aqueous 1N HCl/ACN. The solution was freeze dried to afford Example 11 as a yellow solid (70 mg, 51%).

¹H NMR (400 MHz, DMSO-d₆) δ: 1.24 (d, J 7.0 Hz, 3H, CH(CH₃)₂); 1.25 (d, J 7.0 Hz, 3H, CH(CH₃)₂); 2.62 (sep, J 7.0 Hz, 1H, CH(CH₃)₂); 6.95 (d, J 9.0 Hz, 1H, Ar); 7.70 (dd, J 8.1, 4.2 Hz, 1H, Ar); 7.74-7.81 (m, 3H, Ar); 8.14-8.21 (m, 1H, Ar); 8.43 (bs, 2H, NH₂); 8.62 (d, J 8.1 Hz, 1H, Ar); 8.94 (dd, J 4.2, 1.6 Hz, 1H, Ar); 14.21 (s, 1H, HCl). M/Z (M+H)⁺: 264.8. Mp>250° C.

### Example 12: 6-Isopropyl-5-(8-isoquinolyl)pyridin-2-amine (hydrochloride)

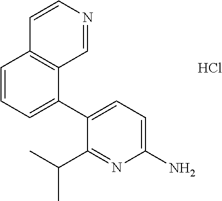 [SMILES: CC(C)c1nc(N)ccc1-c1cccc2ccncc12 ; Cl]

Example 12 was prepared according to method 2 starting from 2-amino-5-bromo-6-isopropylpyridine 1f (100 mg, 0.46 mmol) and 8-isoquinolinyl boronic acid (112 mg, 0.65 mmol, 1.5 eq.). The crude was purified by flash chromatography (SiO₂, DCM/MeOH: 100/0 to 95/5). The obtained foam was triturated in pentane and the collected precipitate was dissolved in a mixture of aqueous 1N HCl/ACN. The solution was freeze dried to afford Example 12 as a yellow solid (88 mg, 64%).

¹H NMR (400 MHz, DMSO-d₆) δ: 1.16 (d, J 6.9 Hz, 3H, CH(CH₃)₂); 1.21 (d, J 6.9 Hz, 3H, CH(CH₃)₂); 2.52 (sep, J 6.9 Hz, 1H, CH(CH₃)₂); 6.93 (d, J 9.0 Hz, 1H, Ar); 7.73 (d, J 9.0 Hz, 1H, Ar); 7.77 (dd, J 7.1, 0.7 Hz, 1H, Ar); 8.10 (dd, J 8.2, 7.1 Hz, 1H, Ar); 8.27 (dt, J 8.2, 0.7 Hz, 1H, Ar); 8.37 (d, J 6.2 Hz, 1H, Ar); 8.50 (bs, 2H, NH₂); 8.63 (d, J 6.2 Hz, 1H, Ar); 9.34 (s, 1H, Ar); 14.24 (s, 1H, HCl salt). M/Z (M+H)⁺: 264.9. Mp>250° C.

### Example 13: 5-Benzo[b]thiophen-3-yl-6-cyclopropyl-pyridin-2-ylamine (hydrochloride)

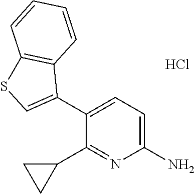 [SMILES: Nc1ccc(-c2csc3ccccc23)c(C2CC2)n1 ; Cl]

Example 13 was prepared according to method 2 starting from 2-amino-5-bromo-6-cyclopropylpyridine 1 g (100 mg, 0.47 mmol) and benzo[b]thien-3-yl boronic acid (126 mg, 0.71 mmol, 1.5 eq.). The crude was purified by flash chromatography (SiO₂, DCM/MeOH: 100/0 to 98/2). The obtained foam was triturated in pentane and the collected precipitate was dissolved in a mixture of aqueous 1N HCl/ACN. The resulting solution was freeze dried to afford Example 13 as a beige solid (78 mg, 55%).

¹H NMR (400 MHz, DMSO-d₆) δ: 0.95 (bs, 2H, 2 CHₐCH_{b}CyPr); 1.20 (bs, 1H, 2 CHₐCH_{b}CyPr); 1.79-1.87 (m, 1H, CHCyPr); 6.85 (bs, 1H, Ar); 7.40-7.46 (m, 1H, Ar); 7.55-7.59 (m, 2H, Ar); 7.77 (bs, 1H, Ar); 7.86 (s, 1H, Ar); 8.06-8.40 (bs, 3H, Ar+NH₂); 13.05 (bs, 1H, HCl salt). M/Z (M+H)⁺: 267.0. Mp: 210-220° C.

### Example 14: 6-Cyclopropyl-5-(8-quinolyl)pyridin-2-amine (hydrochloride)

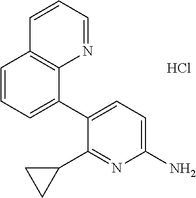 [SMILES: Nc1ccc(-c2cccc3cccnc23)c(C2CC2)n1 ; Cl]

Example 14 was prepared according to method 2 starting from 2-amino-5-bromo-6-cyclopropylpyridine 1 g (100 mg, 0.47 mmol) and 8-quinolinyl boronic acid (115 mg, 0.69 mmol, 1.5 eq.). The crude was purified by flash chromatography (SiO₂, DCM/MeOH: 100/0 to 96/4). The obtained foam was dissolved in a mixture of aqueous 1N HCl/ACN. The resulting solution was freeze dried to afford Example 14 as a beige solid (76 mg, 54%).

¹H NMR (400 MHz, DMSO-d₆) δ: 0.82 (bs, 2H, 2 CHₐCH_{b}CyPr); 1.14 (bs, 2H, 2 CHₐCH_{b}CyPr); 2.1.66-1.74 (m, 1H, 2 CHCyPr); 6.89 (d, J 9.0 Hz, 1H, Ar); 7.70 (dd, J 8.3, 4.4 Hz, 1H, Ar); 7.78 (d, J 7.2 Hz, 1H, Ar); 7.79 (d, J 9.0 Hz, 1H, Ar); 7.86 (dd, J 7.2, 1.4 Hz, 1H, Ar); 8.17 (dd, J 8.2, 1.4 Hz, 1H, Ar); 8.28 (bs, 2H, NH₂); 8.63 (d, J 8.2 Hz, 1H, Ar); 8.93 (dd, J 4.4, 1.7 Hz, 1H, Ar); 13.24 (bs, 1H, HCl salt). M/Z (M+H)⁺: 262.0. Mp>250° C.

### Example 15: 6-Cyclopropyl-5-(8-isoquinolyl)pyridin-2-amine (hydrochloride)

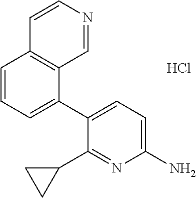 [SMILES: Nc1ccc(-c2cccc3ccncc23)c(C2CC2)n1 ; Cl]

Example 15 was prepared according to method 1 starting from 2-amino-5-bromo-6-cyclopropylpyridine 1 g (100 mg, 0.47 mmol) and 8-isoquinolinyl boronic acid (112 mg, 0.71 mmol, 1.5 eq.). The crude was purified by flash chromatography (SiO₂, DCM/MeOH: 100/0 to 97/3). The obtained foam was dissolved in a mixture of aqueous 1N HCl/ACN and the resulting solution was freeze dried to afford Example 15 as a beige solid (141 mg, 95%).

¹H NMR (400 MHz, DMSO-d₆) δ: 0.75-0.91 (m, 2H, 2 CHₐH_{b}CyPr); 1.20-1.34 (m, 2H, 2 CHₐH_{b}CyPr); 1.56-1.63 (m, 1H, CHCyPr); 6.93 (d, J 9.0 Hz, 1H, Ar); 7.80 (d, J 9.0 Hz, 1H, Ar); 7.90 (dd, J 7.2, 0.8 Hz, 1H, Ar); 8.19 (dd, J 7.2, 0.8 Hz, 1H, Ar); 8.34 (d, J 8.3 Hz, 1H, Ar); 8.46 (bd, J 6.3 Hz, 3H, Ar+NH₂); 8.70 (d, J 6.3 Hz, 1H, Ar); 9.45 (s, 1H, Ar); 13.38 (bs, 1H, HCl salt). M/Z (M+H)⁺: 262.0. Mp: 200-215° C.

### Example 16: 3-(1-Methylindol-3-yl)pyridine-2,6-diamine

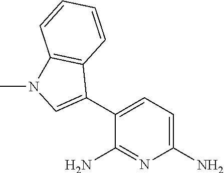 [SMILES: Cn1cc(-c2ccc(N)nc2N)c2ccccc21]

Example 16 was prepared according to method 3 starting from 2,6-diamino-3-iodopyridine 1h (100 mg, 0.43 mmol) and 1-methylindol-3-yl boronic pinacol ester (167 mg, 0.65 mmol, 1.5 eq.). The crude was purified by flash chromatography (SiO₂, CyHex/EtOAc: 100/0 to 0/100). The obtained foam was triturated in pentane and the collected precipitate was dissolved in a mixture of H₂O/ACN. The suspension was freeze dried to afford Example 16 as a white solid (38 mg, 37%).

¹H NMR (400 MHz, DMSO-d₆) δ: 3.80 (s, 3H, N—CH₃); 4.99 (bs, 2H, NH₂); 5.42 (bs, 2H, NH₂); 5.83 (d, J 8.0 Hz, 1H, Ar); 7.04 (ddd, J 7.8, 6.9, 0.9 Hz, 1H, Ar); 7.14 (d, J 8.0 Hz, 1H, Ar); 7.16-7.19 (m, 1H, Ar); 7.32 (s, 1H, Ar); 7.41-7.47 (m, 2H, Ar). M/Z (M+H)⁺: 239.8. Mp: 47-55° C.

### Example 17: Tert-butyl 3-(2,6-diamino-3-pyridyl)indole-1-carboxylate

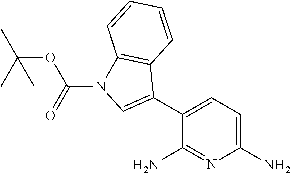 [SMILES: CC(C)(C)OC(=O)n1cc(-c2ccc(N)nc2N)c2ccccc21]

Example 17 was prepared according to method 4 starting from 2,6-diamino-3-iodopyridine 1h (200 mg, 0.85 mmol) and 1-N-Boc-indol-3-yl boronic pinacol ester (167 mg, 0.65 mmol, 1.5 eq.). The crude was purified by flash chromatography (SiO₂, DCM/MeOH: 100/0 to 90/10). The obtained foam was triturated in Et₂O and then in pentane and the collected precipitate was dried under high vacuum at 70° C. overnight to afford Example 17 as a yellow solid (35 mg, 12%).

¹H NMR (400 MHz, DMSO-d₆) δ:1.64 (s, 9H, tBu); 5.11 (bs, 2H, NH₂); 5.56 (bs, 2H, NH₂); 5.83 (d, J 8.0 Hz, 1H, Ar); 7.15 (d, J 8.0 Hz, 1H, Ar); 7.25 (ddd, J 7.9, 7.5, 1.0 Hz, 1H, Ar); 7.35 (ddd, J 7.9, 7.5, 1.0 Hz, 1H, Ar); 7.45 (dd, J 7.5, 1.0 Hz, 1H, Ar); 7.57 (s, 1H, Ar); 8.11 (dd, J 7.9, 1.0 Hz, 1H, Ar). M/Z (M+H)⁺: 325.6. Mp: 150-154° C.

### Example 18: 3-(1H-indol-3-yl)pyridine-2,6-diamine

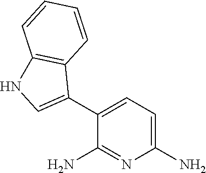 [SMILES: Nc1ccc(-c2c[nH]c3ccccc23)c(N)n1]

Example 18 was prepared according to method 4 starting from 2,6-diamino-3-iodopyridine 1h (200 mg, 0.85 mmol) and 1-N-Boc-indol-3-yl boronic pinacol ester (167 mg, 0.65 mmol, 1.5 eq.) and was obtained concomitantly with example 17. The obtained foam was triturated in Et₂O and then in pentane and the collected precipitate was dried under high vacuum at 70° C. overnight to afford Example 18 as a white solid (83 mg, 43%).

¹H NMR (400 MHz, DMSO-d₆) δ: 4.94 (bs, 2H, NH₂); 5.40 (bs, 2H, NH₂); 5.83 (d, J 7.8 Hz, 1H, Ar); 7.00 (ddd, J 7.7, 7.3, 1.0 Hz, 1H, Ar); 7.10 (ddd, J 7.7, 7.3, 1.0 Hz, 1H, Ar); 7.15 (d, J 7.8 Hz, 1H, Ar); 7.32 (d, J 72.4 Hz, 1H, Ar); 7.38-7.46 (m, 2H, Ar); 11.13 (bs, 1H, NH). M/Z (M+H)⁺: 225.7. Mp: 152-155° C.

### Example 19: 3-Pyrazolo[1,5-a]pyridin-3-ylpyridine-2,6-diamine

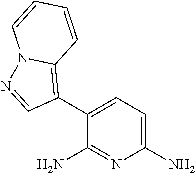 [SMILES: Nc1ccc(-c2cnn3ccccc23)c(N)n1]

Example 19 was prepared according to method 4 starting from 2,6-diamino-3-iodopyridine 1h (100 mg, 0.43 mmol) and 3-pyrazolo[1,5-a]pyridin-3-yl boronic pinacol ester (124 mg, 0.51 mmol, 1.2 eq.). The crude was purified by flash chromatography (SiO₂, DCM/MeOH: 100/0 to 95/5). The obtained foam was triturated in pentane and the collected precipitate was dried under high vacuum at 70° C. overnight to afford Example 19 as a white solid (44 mg, 45%).

¹H NMR (400 MHz, DMSO-d₆) δ: 5.02 (bs, 2H, NH₂); 5.51 (bs, 2H, NH₂); 5.84 (d, J 8.0 Hz, 1H, Ar); 6.88 (td, J 6.8, 1.1 Hz, 1H, Ar); 7.11 (d, J 8.0 Hz, 1H, Ar); 7.15-7.22 (ddd, J 8.9, 6.8, 1.1 Hz, 1H, Ar); 7.46 (dt, J 8.9, 1.1 Hz, 1H, Ar); 7.99 (s, 1H, Ar); 8.65 (dt, J 6.8, 1.1 Hz, 1H, Ar). M/Z (M+H)⁺: 225.7. Mp: 195-200° C.

### Example 20: 3-(Benzofuran-3-yl)pyridine-2,6-diamine

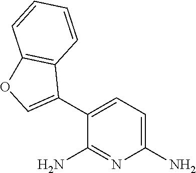 [SMILES: Nc1ccc(-c2coc3ccccc23)c(N)n1]

Example 20 was prepared according to method 4 starting from 2,6-diamino-3-iodopyridine 1h (100 mg, 0.43 mmol) and 3-benzofuran-3-yl boronic pinacol ester (124 mg, 0.51 mmol, 1.2 eq.). The crude was purified by flash chromatography (SiO₂, DCM/MeOH: 100/0 to 95/5). The obtained foam was triturated in pentane and the collected precipitate was dried under high vacuum at 70° C. overnight to afford Example 20 as a white solid (53 mg, 56%).

¹H NMR (400 MHz, DMSO-d₆) δ: 5.17 (bs, 2H, NH₂); 5.59 (bs, 2H, NH₂); 5.85 (d, J 8.0 Hz, 1H, Ar); 7.22 (d, J 8.0 Hz, 1H, Ar); 7.27 (td, J 8.0, 1.2 Hz, 1H, Ar); 7.35 (ddd, J 8.0, 7.2, 1.2 Hz, 1H, Ar); 7.53-7.55 (m, 1H, Ar); 7.61 (dt, J 8.0, 1.2 Hz, 1H, Ar); 7.98 (s, 1H, Ar). M/Z (M+H)⁺: 226.0 Mp: 109-112° C.

### Example 21: 3-(Benzothiophen-3-yl)pyridine-2,6-diamine (hydrochloride)

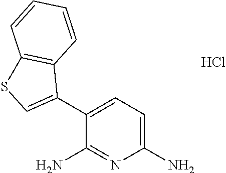 [SMILES: Nc1ccc(-c2csc3ccccc23)c(N)n1 ; Cl]

Example 21 was prepared according to method 4 starting from 2,6-diamino-3-iodopyridine 1h (100 mg, 0.43 mmol) and benzothiophen-3-yl boronic acid (90 mg, 0.51 mmol, 1.2 eq.). The crude was purified by flash chromatography (SiO₂, DCM/MeOH: 100/0 to 95/5). The obtained foam was triturated in pentane and the collected precipitate was dissolved in a mixture of aqueous 1N HCl/ACN. The solution was freeze dried to afford Example 21 as a white solid (52 mg, 44%).

¹H NMR (400 MHz, DMSO-d₆) δ: 6.09 (d, J 8.4 Hz, 1H, Ar); 6.87 (bs, 2H, NH₂); 7.39-7.46 (m, 3H, Ar+NH₂); 7.51-7.56 (m, 2H, Ar); 7.67 (s, 1H, Ar); 8.01-8.09 (m, 2H, Ar); 12.95 (bs, 1H, HCl salt). M/Z (M+H)⁺: 242.6 Mp: 35-38° C.

### Example 22: 3-(5-Fluoro-benzo[b]thiophen-3-yl)pyridine-2,6-diamine

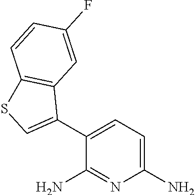 [SMILES: Nc1ccc(-c2csc3ccc(F)cc23)c(N)n1]

Example 22 was prepared according to method 3 using 5 mol % P(tBu)₃ Pd G2 instead of 7 mol %, starting from 2,6-diamino-3-iodopyridine 1h (47 mg, 0.20 mmol) and 3-(5-fluoro-benzothiophen-3-yl) boronic pinacol ester 3a (110 mg, 0.40 mmol, 2.0 eq.). The crude was purified by flash chromatography (SiO₂, CyHex/EtOAc: 100/0 to 30/70). The obtained brown oil was triturated twice in Et₂O and the collected precipitate was dried under high vacuum at 70° C. overnight to afford Example 22 as a brown powder (19 mg, 37%).

¹H NMR (400 MHz, DMSO-d₆) δ: 5.02 (s, 2H, NH₂); 5.63 (s, 2H, NH₂); 5.84 (d, J 8.0 Hz, 1H, Ar); 7.09 (d, J 8.0 Hz, 1H, Ar); 7.20 (dd, J 10.1, 2.5 Hz, 1H, Ar); 7.27 (td, J 8.9, 2.5 Hz, 1H, Ar); 7.69 (s, 1H, Ar); 8.05 (dd, J 8.9, 5.0 Hz, 1H, Ar). M/Z (M+H)⁺: 260.7 Mp: 116-118° C.

### Example 23: 3-(7-fluoro-2-methylquinolin-8-yl)pyridine-2,6-diamine (hydrochloride)

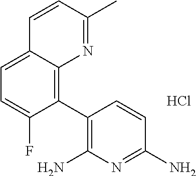 [SMILES: Cc1ccc2ccc(F)c(-c3ccc(N)nc3N)c2n1 ; Cl]

Example 23 was prepared according to method 5 starting from 2,6-diamino-3-iodopyridine 1h (100 mg, 0.43 mmol) and (7-fluoro-2-methylquinolin-8-yl)boronic acid (218 mg, 1.06 mmol, 2.5 eq.). The crude HCl was purified by flash chromatography (SiO₂, DCM/MeOH: 100/0 to 93/7). The resulting foam was further purified by flash chromatography (15 μm Interchim® SiO₂, DCM/MeOH: 100/0 to 93/7). The obtained foam was dissolved in a mixture of aqueous 1N HCl/ACN and the resulting solution was freeze dried to afford Example 23 as a white solid (58 mg, 44%).

¹H NMR (DMSO-d₆, 400 MHz) δ: 2.65 (s, 3H, CH₃); 6.10 (d, J 8.5 Hz, 1H, Ar); 6.87 (bs, 2H, NH₂); 7.48 (d, J 8.5 Hz, 1H, Ar); 7.47-7.58 (bd, 3H, Ar+NH₂); 7.62 (t, J 9.1 Hz, 1H, Ar); 8.15 (dd, J 8.8, 6.5 Hz, 1H, Ar); 8.48 (bd, J 8.8 Hz, 1H, Ar); 13.05 (bs, 1H, HCl salt). M/Z (M+H)⁺: 269.1. Mp: 130-170° C.

### Example 24: 3-(1H-Indol-4-yl)pyridine-2,6-diamine (hydrochloride)

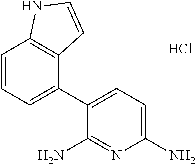 [SMILES: Nc1ccc(-c2cccc3[nH]ccc23)c(N)n1 ; Cl]

Example 24 was prepared according to method 3 starting from 2,6-diamino-3-iodopyridine 1h (100 mg, 0.43 mmol) and indole-4-boronic pinacol ester (158 mg, 0.65 mmol, 1.5 eq.). The crude was purified by flash chromatography (SiO₂, DCM/MeOH: 100/0 to 95/5). The obtained foam was triturated in pentane and the collected precipitate was dissolved in a mixture of aqueous 1N HCl/ACN. The solution was freeze dried to afford Example 24 as a white solid (48 mg, 43%).

¹H NMR (400 MHz, DMSO-d₆) δ: 6.10 (d, J 8.5 Hz, 1H, Ar); 6.20-6.23 (m, 1H, Ar); 6.69 (bs, 2H, NH₂); 6.94 (dd, J 7.3, 0.8 Hz, 1H, Ar); 7.17 (dd, J 8.0, 7.3 Hz, 1H, Ar); 7.35-7.49 (m, 4H, NH₂+2 Ar); 7.58 (d, J 8.5 Hz, 1H, Ar); 11.31 (s, 1H, HCl salt or NH); HCl salt or NH signal not observed. M/Z (M+H)⁺: 225.8. Mp: 150-154° C.

### Example 25: 3-(1H-indol-7-yl)pyridine-2,6-diamine

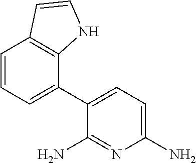 [SMILES: Nc1ccc(-c2cccc3cc[nH]c23)c(N)n1]

Example 25 was prepared according to method 3 starting from 2,6-diamino-3-iodopyridine 1h (100 mg, 0.43 mmol) and 1H-indole-7-boronic pinacol ester (158 mg, 0.65 mmol, 1.5 eq.). The crude was purified by flash chromatography (SiO₂, CyHex/EtOAc: 100/0 to 0/100). The obtained foam was further purified by flash chromatography (15 μm Interchim® SiO₂, DCM/MeOH: 100/0 to 95/5). The obtained solid was dissolved in a mixture of H₂O/ACN and the resulting solution was freeze dried to afford Example 25 as a white solid (43 mg, 45%).

¹H NMR (400 MHz, DMSO-d₆) δ: 4.92 (bs, 2H, NH₂); 5.67 (bs, 2H, NH₂); 5.89 (d, J 8.0 Hz, 1H, Ar); 5.65 (dd, J 2.9, 1.9 Hz, 1H, Ar); 7.20 (dd, J 7.5, 0.9 Hz, 1H, Ar); 7.03 (t, J 7.5 Hz, 1H, Ar); 7.14 (d, J 8.0 Hz, 1H, Ar); 7.23-7.26 (m, 1H, Ar); 7.48 (dt, J 7.5, 0.9 Hz, 1H, Ar); 10.58 (bs, 1H, NH). M/Z (M+H)⁺: 225.7. Mp: 62-70° C.

### Example 26: 3-(1-Methylindazol-7-yl)pyridine-2,6-diamine (hydrochloride)

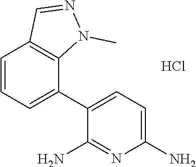 [SMILES: Cn1ncc2cccc(-c3ccc(N)nc3N)c21 ; Cl]

Example 26 was prepared according to method 3 starting from 2,6-diamino-3-iodopyridine 1h (100 mg, 0.43 mmol) and 1-methyl-1H-indazole-7-boronic acid (114 mg, 0.65 mmol, 1.5 eq.). The crude was purified by flash chromatography (SiO₂, CyHex/EtOAc: 100/0 to 0/100). The obtained foam was dissolved in a mixture of aqueous 1N HCl/ACN and the resulting solution was freeze dried to afford Example 26 as a yellow solid (52 mg, 44%).

¹H NMR (400 MHz, DMSO-d₆) δ: 3.77 (s, 3H, N—CH₃); 6.10 (d, J 8.5 Hz, 1H, Ar); 6.88 (bs, 2H, NH₂); 7.13-7.23 (m, 2H, Ar); 7.51 (d, J 8.5 Hz, 1H, Ar); 7.54 (bs, 2H, NH₂); 7.82 (dd, J 7.4, 1.7 Hz, 1H, Ar); 8.11 (s, 1H, Ar); 13.14 (s, 1H, HCl salt). M/Z (M+H)⁺: 240.8. Mp: 198-204° C.

### Example 27: 4-(2,6-Diamino-3-pyridyl)-2-methyl-isoindolin-1-one

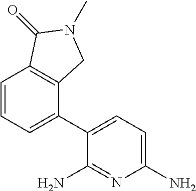 [SMILES: CN1Cc2c(cccc2-c2ccc(N)nc2N)C1=O]

Example 27 was prepared according to method 3 starting from 2,6-diamino-3-iodopyridine 1h (100 mg, 0.43 mmol) and 2-N-methyl-2,3-dihydroisoindol-1-one-4-boronic pinacol ester (178 mg, 0.65 mmol, 1.5 eq.). The crude was purified by flash chromatography (SiO₂, CyHex/EtOAc: 100/0 to 0/100). The obtained foam was triturated in pentane and the collected precipitate was dried under high vacuum at 70° C. overnight to afford Example 27 as a beige powder (69 mg, 63%).

¹H NMR (400 MHz, DMSO-d₆) δ: 3.05 (s, 3H, N—CH₃); 4.32 (bs, 2H, N—CH₂); 5.04 (bs, 2H, NH₂); 5.59 (bs, 2H, NH₂); 5.81 (d, J 7.9 Hz, 1H, Ar); 7.05 (d, J 7.9 Hz, 1H, Ar); 7.42 (dd, J 7.5, 0.9 Hz, 1H, Ar); 7.49 (t, J 7.5 Hz, 1H, Ar); 7.58 (dd, J 7.5, 0.9 Hz, 1H, Ar). M/Z (M+H)⁺: 255.7. Mp>250° C.

### Example 28: 3-(2,3-Dihydrobenzofuran-7-yl)pyridine-2,6-diamine (hydrochloride)

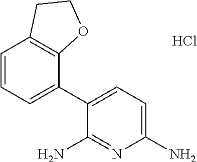 [SMILES: Nc1ccc(-c2cccc3c2OCC3)c(N)n1 ; Cl]

Example 28 was prepared according to method 3 starting from 2,6-diamino-3-iodopyridine 1h (100 mg, 0.43 mmol) and 2,3-dihydro-1-benzofuran-7-yl-boronic acid (107 mg, 0.65 mmol, 1.5 eq.). The crude was purified by flash chromatography (SiO₂, DCM/MeOH: 100/0 to 95/5). The obtained foam was triturated in pentane and the collected precipitate was dissolved in a mixture of aqueous 1N HCl/ACN. The solution was freeze dried to afford Example 28 as a white solid (88 mg, 78%).

¹H NMR (400 MHz, DMSO-d₆) δ: 3.15 (t, J 8.7 Hz, 2H, O—CH₂—CH₂); 4.48 (t, J 8.7 Hz, 2H, O—CH₂—CH₂); 5.96 (d, J 8.5 Hz, 1H, Ar); 6.74 (bs, 2H, NH₂); 6.83 (t, J 7.6 Hz, 1H, Ar); 6.93 (dd, J 7.6, 1.1 Hz, 1H, Ar); 7.16-7.20 (m, 1H, Ar); 7.31 (bs, 2H, NH₂); 7.40 (d, J 8.5 Hz, 1H, Ar); 12.86 (s, 1H, HCl). M/Z (M+H)⁺: 228.8. Mp>250° C.

### Example 29: 3-(Benzothiophen-7-yl)pyridine-2,6-diamine (hydrochloride)

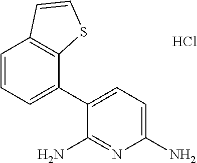 [SMILES: Nc1ccc(-c2cccc3ccsc23)c(N)n1 ; Cl]

Example 29 was prepared according to method 3 starting from 2,6-diamino-3-iodopyridine 1h (100 mg, 0.43 mmol) and 1-benzothiophen-7-yl boronic acid (116 mg, 0.65 mmol, 1.5 eq.). The crude was purified by flash chromatography (SiO₂, CyHex/EtOAc: 100/0 to 0/100). The obtained foam was triturated in pentane and the collected precipitate was dissolved in a mixture of aqueous 1N HCl/ACN. The solution was freeze dried to afford Example 29 as a white solid (74 mg, 62%).

¹H NMR (400 MHz, DMSO-d₆) δ: 6.09 (d, J 8.5 Hz, 1H, Ar); 6.91 (bs, 2H, NH₂); 7.29 (dd, J 8.1, 0.8 Hz, 1H, Ar); 7.46-7.51 (m, 1H, Ar); 7.53 (bs, 2H, NH₂); 7.54 (d, J 5.4 Hz, 1H, Ar); 7.61 (d, J 8.5 Hz, 1H, Ar); 7.78 (d, J 5.4 Hz, 1H, Ar); 7.92 (dd, J 8.1, 0.8 Hz, 1H, Ar); 13.09 (s, 1H, HCl salt). M/Z (M+H)⁺: 242.8. Mp: 100-113° C.

### Example 30: 3-(1,3-Benzothiazol-4-yl)pyridine-2,6-diamine (hydrochloride)

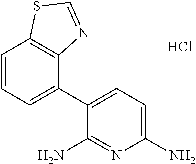 [SMILES: Nc1ccc(-c2cccc3scnc23)c(N)n1 ; Cl]

Example 30 was prepared according to method 3 starting from 2,6-diamino-3-iodopyridine 1h (100 mg, 0.43 mmol) and 1-benzothiazole-4-boronic pinacol ester (170 mg, 0.65 mmol, 1.5 eq.). The crude was purified by flash chromatography (SiO₂, CyHex/EtOAc: 100/0 to 0/100). The obtained foam was triturated in pentane and the collected precipitate was dissolved in a mixture of aqueous 1N HCl/ACN. The solution was freeze dried to afford Example 30 as a white solid (74 mg, 61%).

¹H NMR (400 MHz, DMSO-d₆) δ: 6.09 (d, J 8.5 Hz, 1H, Ar); 6.88 (bs, 2H, NH₂); 7.41-7.50 (m, 3H, Ar, NH₂); 7.56 (t, J 8.0 Hz, 1H, Ar); 7.60 (d, J 8.0 Hz, 1H, Ar); 8.21 (dd, J 8.0, 1.2 Hz, 1H, Ar); 9.38 (s, 1H, Ar); 13.03 (s, 1H, HCl salt). M/Z (M+H)⁺: 243.7. Mp>250° C.

### Example 31: 3-(8-quinolyl)pyridine-2,6-diamine (hydrochloride)

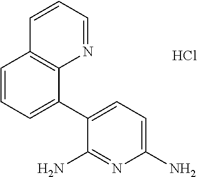 [SMILES: Nc1ccc(-c2cccc3cccnc23)c(N)n1 ; Cl]

Example 31 was prepared according to method 3 starting from 2,6-diamino-3-iodopyridine 1h (300 mg, 1.28 mmol) and 8-quinolylboronic acid (332 mg, 1.92 mmol, 1.5 eq.). The crude was purified by flash chromatography (SiO₂, CyHex/EtOAc: 100/0 to 0/100). The obtained foam was triturated in pentane and the collected precipitate dissolved in a mixture of aqueous 1N HCl/ACN. The solution was freeze dried to afford Example 31 as a white solid (297 mg, 85%).

¹H NMR (400 MHz, DMSO-d₆) δ: 6.11 (d, J 8.5 Hz, 1H, Ar); 6.84 (bs, 2H, NH₂); 7.50-7.53 (m, 3H, Ar); 7.79-7.83 (m, 3H, Ar+NH₂); 8.20 (dd, J 6.2, 2.8 Hz, 1H, Ar); 8.77 (d, J 4.6 Hz, 1H, Ar); 9.01 (dd, J 4.6, 1.2 Hz, 1H, Ar); 13.17 (bs, 1H, HCl salt). M/Z (M+H)⁺: 296.7. Mp: 182-192° C.

### Example 32: 3-Isoquinolin-8-yl-pyridine-2,6-diamine hydrochloride (hydrochloride)

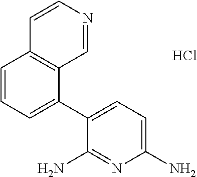 [SMILES: Nc1ccc(-c2cccc3ccncc23)c(N)n1 ; Cl]

Example 32 was prepared according to method 5 starting from 2,6-diamino-3-iodopyridine 1h (100 mg, 0.43 mmol) and 8-isoquinolylboronic acid (111 mg, 0.65 mmol, 1.5 eq.). The crude was purified by flash chromatography (SiO₂, DCM/MeOH: 100/0 to 98/2). The obtained foam was further purified by flash chromatography (15 μm Interchim® SiO₂, DCM/MeOH: 100/0 to 95/5). The resulting yellow powder was dissolved in a mixture of aqueous 1N HCl/ACN and the obtained solution was freeze dried to afford Example 32 as a white solid (58 mg, 49%).

¹H NMR (400 MHz, DMSO-d₆) δ: 6.15 (d, J 8.4 Hz, 1H, Ar); 6.99 (bs, 2H, NH₂); 7.56 (d, J 8.4 Hz, 1H, Ar); 7.67 (bs, 2H, NH₂); 7.81 (dd, J 7.2, 0.8 Hz, 1H, Ar); 8.16 (dd, J 8.4, 7.2 Hz, 1H, Ar); 8.30 (dd, J 8.4, 0.8 Hz, 1H, Ar); 8.45 (d, J 6.4 Hz, 1H, Ar); 8.68 (d, J 6.4 Hz, 1H, Ar); 9.40 (s, 1H, Ar); 13.27 (bs, 1H, HCl salt). M/Z (M+H)⁺: 237.8. Mp: 77-88° C.

### Example 33: 3-(5-Isoquinolyl)pyridine-2,6-diamine

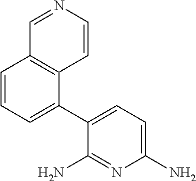 [SMILES: Nc1ccc(-c2cccc3cnccc23)c(N)n1]

Example 33 was prepared according to method 3 starting from 2,6-diamino-3-iodopyridine 1h (100 mg, 0.43 mmol) and 5-isoquinolylboronic acid (112 mg, 0.65 mmol, 1.5 eq.). The crude was purified by flash chromatography (SiO₂, DCM/MeOH, 100/0 to 95/5). The resulting foam was further purified flash chromatography (15 μm Interchim® SiO₂, DCM/MeOH: 100/0 to 95/5). The obtained powder was dried under high vacuum at 70° C. to afford Example 33 as a white solid (25 mg, 25%).

¹H NMR (400 MHz, DMSO-d₆) δ: 4.86 (bs, 2H, NH₂); 5.62 (bs, 2H, NH₂); 5.88 (d, J 8.0 Hz, 1H, Ar); 7.01 (d, J 8.0 Hz, 1H, Ar); 7.46 (dd, J 6.0, 0.9 Hz, 1H, Ar); 7.62 (dd, J 7.1, 1.1 Hz, 1H, Ar); 7.70 (t, J 7.1 Hz, 1H, Ar); 7.07 (dt, J 8.0, 1.1 Hz, 1H, Ar); 8.44 (d, J 6.0 Hz, 1H, Ar); 9.33 (d, J 0.9 Hz, 1H, Ar). M/Z (M+H)⁺: 237.7. Mp>250° C.

### Example 34: 3-Quinolin-5-yl-pyridine-2,6-diamine

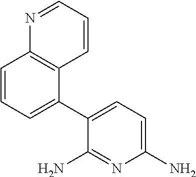 [SMILES: Nc1ccc(-c2cccc3ncccc23)c(N)n1]

Example 34 was prepared according to method 3 starting from 2,6-diamino-3-iodopyridine 1h (100 mg, 0.43 mmol) and 5-quinolylboronic acid (112 mg, 0.65 mmol, 1.5 eq.). The crude was purified by flash chromatography (SiO₂, DCM/MeOH, 100/0 to 95/5). The resulting foam was further purified flash chromatography (15 μm Interchim® SiO₂, DCM/MeOH: 100/0 to 92/8). The obtained solid was triturated in pentane and the collected precipitate was dried under high vacuum at 70° C. to afford Example 34 as a yellow powder (31 mg, 30%).

¹H NMR (400 MHz, DMSO-d₆) δ: 4.85 (bs, 2H, NH₂); 5.61 (bs, 2H, NH₂); 5.87 (d, J 8.0 Hz, 1H, Ar); 7.01 (d, J 8.0 Hz, 1H, Ar); 7.45 (dd, J 7.1, 1.1 Hz, 1H, Ar); 7.48 (dd, J 8.4, 4.2 Hz, 1H, Ar); 7.77 (dd, J 8.4, 7.1 Hz, 1H, Ar); 7.95-8.01 (m, 2H, Ar); 8.89 (dd, J 4.2, 1.8 Hz, 1H, Ar). M/Z (M+H)⁺: 237.8. Mp>250° C.

### Example 35: 3-Quinolin-4-yl-pyridine-2,6-diamine

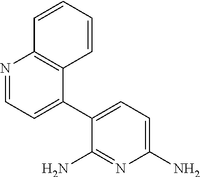 [SMILES: Nc1ccc(-c2ccnc3ccccc23)c(N)n1]

Example 35 was prepared according to method 3 starting from 2,6-diamino-3-iodopyridine 1h (100 mg, 0.43 mmol) and 4-quinolylboronic acid (112 mg, 0.65 mmol, 1.5 eq.). The crude was purified by flash chromatography (SiO₂, DCM/MeOH: 100/0 to 95/5). The resulting foam was further purified by trituration in MeOH. The obtained solid was triturated in pentane and the collected precipitate was dried under high vacuum at 70° C. to afford Example 35 as a yellow powder (55 mg, 54%).

¹H NMR (400 MHz, DMSO-d₆) δ: 5.06 (bs, 2H, NH₂); 5.72 (bs, 2H, NH₂); 5.88 (d, J 8.0 Hz, 1H, Ar); 7.05 (d, J 8.0 Hz, 1H, Ar); 7.37 (d, J 4.4 Hz, 1H, Ar); 7.50-7.57 (m, 1H, Ar); 7.70-7.77 (m, 2H, Ar); 8.02-8.06 (m, 1H, Ar); 8.85 (d, J 4.4 Hz, 1H, Ar). M/Z (M+H)⁺: 237.8. Mp>250° C.

### Example 36: 3-Isoquinolin-4-yl-pyridine-2,6-diamine

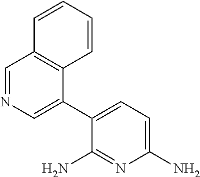 [SMILES: Nc1ccc(-c2cncc3ccccc23)c(N)n1]

Example 36 was prepared according to method 4 starting from 2,6-diamino-3-iodopyridine 1h (100 mg, 0.43 mmol) and 3-isoquinolylboronic acid (88 mg, 0.51 mmol, 1.2 eq.). The crude was purified by flash chromatography (SiO₂, DCM/MeOH: 100/0 to 90/10). The obtained solid was triturated in pentane and the collected precipitate was dried under high vacuum at 70° C. to afford Example 36 as a yellow powder (46 mg, 46%).

¹H NMR (400 MHz, DMSO-d₆) δ: 4.91 (bs, 2H, NH₂); 5.63 (bs, 2H, NH₂); 5.88 (d, J 8.0 Hz, 1H, Ar); 7.04 (d, J 8.0 Hz, 1H, Ar); 7.57-7.79 (m, 3H, Ar); 8.16 (d, J 7.6 Hz, 1H, Ar); 8.32 (s, 1H, Ar); 9.36 (s, 1H, Ar). M/Z (M+H)⁺: 237. Mp: 180-185° C.

### Example 37: 3-Chroman-8-yl-pyridine-2,6-diamine (hydrochloride)

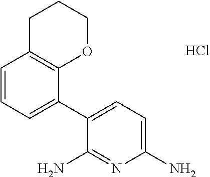 [SMILES: Nc1ccc(-c2cccc3c2OCCC3)c(N)n1 ; Cl]

Example 37 was prepared according to method 3 starting from 2,6-diamino-3-iodopyridine 1h (100 mg, 0.43 mmol) and (3,4-dihydro-2H-1-benzopyran-8-yl)boronic acid (116 mg, 0.65 mmol, 1.5 eq.). The crude was purified by flash chromatography (SiO₂, CyHex/EtOAc: 100/0 to 0/100). The obtained solid was triturated in pentane and the collected precipitate was dissolved in a mixture of aqueous 1N HCl/ACN. The resulting solution was freeze dried to afford Example 37 as a yellow powder (74 mg, 62%).

¹H NMR (400 MHz, DMSO-d₆) δ: 1.93 (tt, J 6.4, 5.0 Hz, 2H, O—CH₂—CH₂—CH₂—Ar); 2.79 (t, J 6.4 Hz, 2H, O—CH₂—CH₂—CH₂—Ar); 4.15 (t, J 5.0 Hz, 2H, O—CH₂—CH₂—CH₂—Ar); 6.01 (d, J 8.5 Hz, 1H, Ar); 6.72 (bs, 2H, NH₂); 6.88 (t, J 7.5 Hz, 1H, Ar); 6.95 (dd, J 7.5, 1.7 Hz, 1H, Ar); 7.08-7.12 (m, 1H, Ar); 7.29 (bs, 2H, NH₂); 7.37 (d, J 8.5 Hz, 1H, Ar); 12.86 (s, 1H, HCl salt). M/Z (M+H)⁺: 242.8. Mp: 110-124° C.

### Example 38: 3-(2,3-Dihydro-benzo[1,4]dioxin-5-yl)-pyridine-2,6-diamine (hydrochloride)

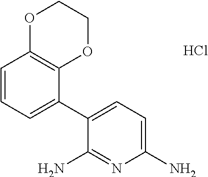 [SMILES: Nc1ccc(-c2cccc3c2OCCO3)c(N)n1 ; Cl]

Example 38 was prepared according to method 3 starting from 2,6-diamino-3-iodopyridine 1h (100 mg, 0.43 mmol) and (2,3-dihydrobenzo[b][1,4]dioxin-5-yl)boronic acid (117 mg, 0.65 mmol, 1.5 eq.). The crude was purified by flash chromatography (SiO₂, DCM/MeOH: 100/0 to 95/5). The resulting foam was further purified flash chromatography (15 μm Interchim® SiO₂, DCM/MeOH: 100/0 to 92/8). The obtained solid was triturated in pentane and the collected precipitate was dissolved in a mixture of aqueous 1N HCl/ACN. The resulting solution was freeze dried to afford Example 38 as a yellow solid (31 mg, 30%).

¹H NMR (400 MHz, DMSO-d₆) δ: 4.26 (s, 4H, 2 O—CH₂); 6.01 (d, J 8.5 Hz, 1H, Ar); 6.71 (dd, J 6.0, 3.2 Hz, 1H, Ar); 6.82 (bs, 2H, NH₂); 6.86-6.90 (m, 2H, Ar); 7.32 (bs, 2H, NH₂); 7.42 (d, J 8.5 Hz, 1H, Ar); 12.86 (s, 1H, HCl). M/Z (M+H)⁺: 244.8. Mp: 99-110° C.

### Example 39: 3-Dibenzothiophen-4-ylpyridine-2,6-diamine (hydrochloride)

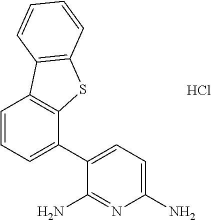 [SMILES: Nc1ccc(-c2cccc3c2sc2ccccc23)c(N)n1 ; Cl]

Example 39 was prepared according to method 3 starting from 2,6-diamino-3-iodopyridine 1h (100 mg, 0.43 mmol) and 4-dibenzothiophenylboronic acid (148 mg, 0.65 mmol, 1.5 eq.). The crude was purified by flash chromatography (SiO₂, CyHex/EtOAc: 100/0 to 0/100). The obtained solid was triturated in pentane and the collected precipitate was dissolved in a mixture of aqueous 1N HCl/ACN. The resulting solution was freeze dried to afford Example 39 as a white solid (100 mg, 71%).

¹H NMR (400 MHz, DMSO-d₆) δ: 6.11 (d, J 8.5 Hz, 1H, Ar); 6.98 (bs, 2H, NH₂); 7.44 (dd, J 7.4, 1.0 Hz, 1H, Ar); 7.49-7.68 (m, 6H, NH₂+4 Ar); 7.97-8.02 (m, 1H, Ar); 8.35-8.46 (m, 2H, Ar); 13.09 (s, 1H, HCl salt). M/Z (M+H)⁺: 292.7. Mp: 165-170° C.

### Example 40: 3-Dibenzofuran-4-ylpyridine-2,6-diamine (hydrochloride)

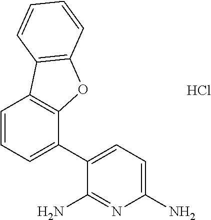 [SMILES: Nc1ccc(-c2cccc3c2oc2ccccc23)c(N)n1 ; Cl]

Example 40 was prepared according to method 3 starting from 2,6-diamino-3-iodopyridine 1h (100 mg, 0.43 mmol) and 4-dibenzofuranylboronic acid (137 mg, 0.65 mmol, 1.5 eq.). The crude was purified by flash chromatography (SiO₂, DCM/MeOH: 100/0 to 95/5). The obtained solid was triturated in pentane and the collected precipitate was dissolved in a mixture of aqueous 1N HCl/ACN. The resulting solution was freeze dried to afford Example 40 as a white solid (88 mg, 66%).

¹H NMR (400 MHz, DMSO-d₆) δ: 6.11 (d, J 8.5 Hz, 1H, Ar); 7.04 (bs, 2H, NH₂); 7.38-7.56 (m, 6H, NH₂+4 Ar); 5.65 (d, J 8.5 Hz, 1H, Ar); 7.70 (dd, J 8.2, 1.5 Hz, 1H, Ar); 8.15-8.21 (m, 2H, Ar); 13.02 (s, 1H, HCl salt). M/Z (M+H)⁺: 276.7. Mp: 135-143° C.

### Example 41: 6-Ethyl-5-(2-methylbenzothiophen-3-yl)pyridin-2-amine (hydrochloride)

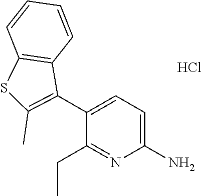 [SMILES: CCc1nc(N)ccc1-c1c(C)sc2ccccc12 ; Cl]

Example 41 was prepared according to method 6 starting from 3-bromo-2-methyl-benzothiophene 2a (100 mg, 0.44 mmol) and ethyl-5-(4,4,5,5-tetramethyl-1,3,2-dioxaborolan-2-yl)pyridin-2-amine 4a (144 mg, 0.66 mmol, 1.5 eq.). The crude was purified by preparative HPLC. The obtained solid was dissolved in a mixture of aqueous 1 N HCl/ACN and the resulting solution was freeze dried to afford Example 41 as a white solid (49 mg, 36%).

¹H NMR (400 MHz, DMSO-d₆) δ: 1.08 (t, J 7.6 Hz, 3H, CH₂—CH₃); 2.37 (s, 3H, Ar—CH₃); 2.44 (q, J 7.6 Hz, 1H, CHₐH_{b}—CH₃); 2.45 (q, J 7.6 Hz, 1H, CHₐH_{b}—CH₃); 6.98 (d, J 9.0 Hz, 1H, Ar); 7.26-7.29 (m, 1H, Ar); 7.32-7.38 (m, 3H, Ar); 7.74 (d, J 9.0 Hz, 1H, Ar); 8.09 (bs, 2H, NH₂); 14.33 (bs, 1H, HCl salt). M/Z (M+H)⁺: 269.8. Mp: 70-72° C.

### Example 42: 6-Ethyl-5-(5-methylbenzothiophen-3-yl)pyridin-2-amine (hydrochloride)

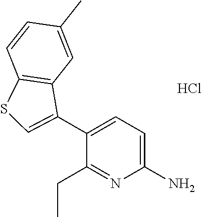 [SMILES: CCc1nc(N)ccc1-c1csc2ccc(C)cc12 ; Cl]

Example 42 was prepared according to method 6 starting from 3-bromo-5-methyl-benzothiophene 2b (100 mg, 0.44 mmol) and ethyl-5-(4,4,5,5-tetramethyl-1,3,2-dioxaborolan-2-yl)pyridin-2-amine 4a (144 mg, 0.66 mmol, s 1.5 eq.). The crude was purified by flash chromatography (SiO₂, CyHex/EtOAc: 100/0 to 50/50). The obtained solid was further purified by preparative HPLC and then was dissolved in a mixture of aqueous 1N HCl/ACN. The resulting solution was freeze dried to afford Example 42 as a white solid (71 mg, 53%).

¹H NMR (400 MHz, DMSO-d₆) δ: 1.13 (t, J 7.6 Hz, 3H, CH₂—CH₃); 2.39 (s, 3H, Ar—CH₃); 2.56 (q, J 7.6 Hz, 2H, CH₂—CH₃); 6.97 (d, J 9.0 Hz, 1H, Ar); 7.27-7.29 (m, 2H, Ar); 7.79 (s, 1H, Ar); 7.81 (d, J 9.0 Hz, 1H, Ar); 7.96 (dd, J 8.0, 0.7 Hz, 1H, Ar); 8.08 (bs, 2H, NH₂); 14.36 (bs, 1H, HCl salt). M/Z (M+H)⁺: 269.8. Mp: 64-88° C.

### Example 43: 6-Ethyl-5-(5-fluorobenzothiophen-3-yl)pyridin-2-amine (hydrochloride)

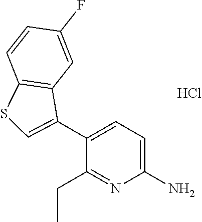 [SMILES: CCc1nc(N)ccc1-c1csc2ccc(F)cc12 ; Cl]

Example 43 was prepared according to method 7 starting from 3-bromo-5-fluoro-benzothiophene 2c (100 mg, 0.43 mmol) and ethyl-5-(4,4,5,5-tetramethyl-1,3,2-dioxaborolan-2-yl)pyridin-2-amine 4a (144 mg, 0.66 mmol, 1.5 eq.). The crude was purified by flash chromatography (SiO₂, CyHex/EtOAc: 100/0 to 50/50). The obtained solid was dissolved in a mixture of aqueous 1N HCl/ACN and the resulting solution was freeze dried to afford Example 43 as a white solid (37 mg, 27%).

¹H NMR (400 MHz, DMSO-d₆) δ: 1.12 (t, J 7.6 Hz, 3H, CH₂—CH₃); 2.55 (q, J 7.6 Hz, 2H, CH₂—CH₃); 6.95 (d, J 9.0 Hz, 1H, Ar); 7.31-7.36 (m, 2H, Ar); 7.81 (d, J 9.0 Hz, 1H, Ar); 7.96 (s, 1H, Ar); 8.06 (bs, 2H, NH₂); 8.12-8.15 (m, 1H, Ar); 14.31 (bs, 1H, HCl salt). M/Z (M+H)⁺: 273.8. Mp>250° C.

### Example 44: 6-Ethyl-5-[2-(3-pyridyl)phenyl]pyridin-2-amine (hydrochloride)

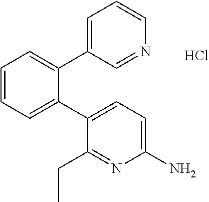 [SMILES: CCc1nc(N)ccc1-c1ccccc1-c1cccnc1 ; Cl]

Example 44 was prepared according to method 2 starting from 5-(2-chlorophenyl)-6-ethyl-pyridin-2-amine 5a (100 mg, 0.43 mmol) and 3-pyridylboronic acid (80 mg, 0.65 mmol, 1.5 eq.). The crude was purified by flash chromatography (SiO₂, DCM/MeOH: 100/0 to 95/5). The obtained solid was triturated in pentane. The collected precipitate was dissolved in a mixture of aqueous 1N HCl/ACN and the resulting solution was freeze dried to afford Example 44 as a beige solid (81.1 mg, 60%).

¹H NMR (400 MHz, DMSO-d₆) δ: 1.04 (t, J 7.6 Hz, 3H, CH₂—CH₃); 2.39 (q, J 7.6 Hz, 2H, CH₂—CH₃); 6.79 (d, J 8.8 Hz, 1H, Ar); 7.42-7.48 (m, 1H, Ar); 7.56-7.69 (m, 4H, Ar); 7.79 (dd, J 8.0, 5.6 Hz, 1H, Ar); 7.99-8.24 (m, 3H, Ar+NH₂); 8.67 (d, J 1.6 Hz, 1H, Ar); 8.72 (dd, J 5.2, 1.2 Hz, 1H, Ar); 14.32 (bs, 1H, HCl salt). M/Z (M+H)⁺: 276.8. Mp>250° C.

### Example 45: 3-[2-(3-pyridyl)phenyl]pyridine-2,6-diamine (hydrochloride)

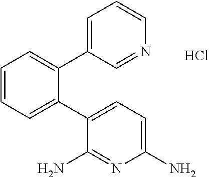 [SMILES: Nc1ccc(-c2ccccc2-c2cccnc2)c(N)n1 ; Cl]

Example 45 was prepared according to method 8 starting from 3-(2-chlorophenyl)pyridine-2,6-diamine 5b (100 mg, 0.46 mmol) and 3-pyridylboronic acid (85 mg, 0.69 mmol, 1.5 eq.). The crude was purified by flash chromatography (SiO₂, DCM/MeOH, 100/0 to 95/5). The obtained solid was dissolved in a mixture of aqueous 1N HCl/ACN and the resulting solution was freeze dried to afford to afford Example 45 as a brown solid (51 mg, 37%).

¹H NMR (400 MHz, DMSO-d₆) δ: 5.96 (d, J 8.4 Hz, 1H, Ar); 6.78 (s, 2H, NH₂); 7.33-7.52 (m, 4H, Ar+NH₂); 7.52-7.64 (m, 3H, Ar); 7.79 (dd, J 7.6, 5.6 Hz, 1H, Ar); 8.09 (d, J 8.0 Hz, 1H, Ar); 8.63-8.68 (m, 1H, Ar); 8.72 (d, J 4.8 Hz, 1H, Ar); 12.91 (bs, 1H, HCl salt). M/Z (M+H)⁺: 263.8. Mp: 100-135° C.

### Example 46: 3-[2-(6-morpholino-3-pyridyl)phenyl]pyridine-2,6-diamine (hydrochloride

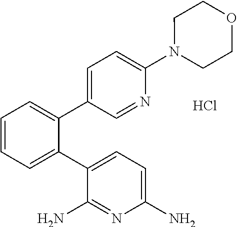 [SMILES: Nc1ccc(-c2ccccc2-c2ccc(N3CCOCC3)nc2)c(N)n1 ; Cl]

Example 46 was prepared according to method 8 starting from 3-(2-chlorophenyl)pyridine-2,6-diamine 5b (100 mg, 0.46 mmol) and 4-[5-(4,4,5,5-tetramethyl-1,3-dioxolan-2-yl)-2-pyridyl]morpholine (200 mg, 0.69 mmol, 1.5 eq.). The crude was purified by flash chromatography (SiO₂, DCM/MeOH: 100/0 to 95/5). The obtained foam was dissolved in a mixture of aqueous 1N HCl/ACN and the resulting solution was freeze dried to afford Example 46 as a yellow solid (77 mg, 43%).

¹H NMR (400 MHz, DMSO-d₆) δ: 3.54-3.62 (m, 4H, 2 N—CH₂); 3.68-3.76 (m, 4H, 2 O—CH₂); 5.97 (d, J 8.4 Hz, 1H, Ar); 6.76 (bs, 2H, NH₂); 7.03-7.15 (m, 1H, Ar); 7.29-7.55 (m, 7H, Ar+NH₂); 7.60 (d, J 8.8 Hz, 1H, Ar); 7.95 (d, J 2.0 Hz, 1H, Ar); 12.93 (bs, 1H, HCl salt). M/Z (M+H)⁺: 348.8. Mp: 180-210° C.

### Example 47: 6-ethyl-5-(quinolin-8-yl)pyridin-2-amine (hydrochloride)

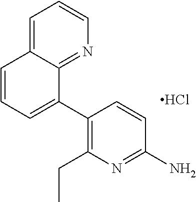 [SMILES: CCc1nc(N)ccc1-c1cccc2cccnc12 ; Cl]

Example 47 was prepared according to method 2 starting from 2-amino-5-bromo-6-ethylpyridine 1d (100 mg, 0.50 mmol) and quinolin-8-ylboronic acid (130 mg, 0.75 mmol, 1.5 eq.). The crude was purified by flash chromatography (SiO₂, DCM/MeOH: 100/0 to 95/5). The obtained solid was triturated in pentane. The collected precipitate was dissolved in a mixture of aqueous 1N HCl/ACN and the resulting solution was freeze dried to afford Example 47 as a white solid (100 mg, 70%).

¹H NMR (400 MHz, DMSO-d₆) δ: 1.07 (t, J 7.6 Hz, 3H, CH₃); 2.36-2.48 (m, 2H, CH₂); 6.96 (d, J 9.0 Hz, 1H, Ar); 7.68-7.71 (m, 1H, Ar); 7.75-7.82 (m, 3H, Ar); 8.08 (bs, 2H, NH₂); 8.17 (dd, J 7.6, 1.5 Hz, 1H, Ar); 8.61 (d, J 8.1 Hz, 1H, Ar); 8.93 (dd, J 4.3, 1.3 Hz, 1H, Ar); 14.41 (bs, 1H, HCl salt). M/Z (M+H)⁺: 250.8. Mp>250° C.

### Example 48: 3-(2-(1-methyl-1H-pyrazol-5-yl)phenyl)pyridine-2,6-diamine (hydrochloride)

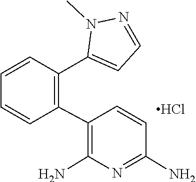 [SMILES: Cn1nccc1-c1ccccc1-c1ccc(N)nc1N ; Cl]

Example 48 was prepared according to method 8 starting from 3-(2-chlorophenyl)pyridine-2,6-diamine 5b (98 mg, 0.45 mmol) and 1-methyl-5-(4,4,5,5-tetramethyl-1,3,2-dioxaborolan-2-yl)-1H-pyrazole (230 mg, 1.10 mmol, 2.4 eq.). The crude was purified twice by flash chromatography (SiO₂, DCM/MeOH: 100/0 to 97/3 then KPNH, CycloHex/EtOAc: 100/0 to 20/80). The obtained foam was triturated in pentane and the collected precipitate was dried under high vacuum at 70° C. overnight to afford Example 48 as a beige solid (7 mg, 5%).

¹H NMR (400 MHz, DMSO-d₆) δ: 3.58 (s, 3H, N—CH₃); 5.88 (d, J 8.5 Hz, 1H, Ar); 6.05 (d, J 1.6 Hz, 1H, Ar); 6.82 (s, 2H, NH₂); 7.18 (d, J 8.5 Hz, 1H, Ar); 7.35 (bd, 3H, Ar+NH₂); 7.41 (dd, J 7.2, 1.4 Hz, 1H, Ar); 7.48 (dd, J 7.2, 1.4 Hz, 1H, Ar); 7.55 (quint d, J 7.2, 1.4 Hz, 1H, Ar); 12.73 (bs, 1H, HCl salt). M/Z (M+H)⁺: 266.0. Mp>250° C.

### Example 49: 3-(1-methyl-1H-indol-7-yl)pyridine-2,6-diamine

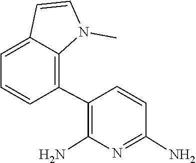 [SMILES: Cn1ccc2cccc(-c3ccc(N)nc3N)c21]

Example 49 was prepared according to method 3 starting from 2,6-diamino-3-iodopyridine 1h (100 mg, 0.43 mmol) and 1-methyl-7-(4,4,5,5-tetramethyl-1,3,2-dioxaborolan-2-yl)-1H-indole (167 mg, 0.65 mmol, 1.5 eq.). The crude was purified by flash chromatography (SiO₂, CyHex/EtOAc: 100/0 to 0/100). The obtained solid was further purified by preparative HPLC and the pure fractions were freeze dried to afford Example 49 as a white solid (21 mg, 20%).

¹H NMR (400 MHz, DMSO-d₆) δ: 3.47 (s, 3H, N—CH₃); 5.32 (bs, 2H, NH₂); 5.91 (d, J 8.0 Hz, 1H, Ar); 6.16 (bs, 2H, NH₂); 6.45 (d, J 3.0 Hz, 1H, Ar); 6.84 (dd, J 7.2, 1.0 Hz, 1H, Ar); 7.04 (dd, J 8.0, 7.2 Hz, 1H, Ar); 7.15 (d, J 8.0 Hz, 1H, Ar); 7.23 (d, J 3.0 Hz, 1H, Ar); 7.54 (dd, J 8.0, 1.0 Hz, 1H, Ar). M/Z (M+H)⁺: 239.1. Mp: 100-117° C.

### Example 50: 3-(benzofuran-7-yl)pyridine-2,6-diamine (hydrochloride)

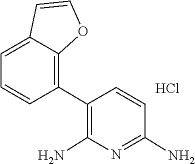 [SMILES: Nc1ccc(-c2cccc3ccoc23)c(N)n1 ; Cl]

Example 50 was prepared according to method 5 starting from 2,6-diamino-3-iodopyridine 1h (75 mg, 0.32 mmol) and 2-(benzofuran-7-yl)-4,4,5,5-tetramethyl-1,3,2-dioxaborolane (100 mg, 0.41 mmol, 1.3 eq.). The crude was purified by flash chromatography (15 μm Interchim® SiO₂, DCM/MeOH: 100/0 to 92/8). The obtained foam was further purified flash chromatography (KPNH, CyHex/EtOAc: 100/0 to 60/40). The obtained foam was dissolved in a mixture of aqueous 1N HCl/ACN and the resulting solution was freeze dried to afford Example 50 as a beige solid (40 mg, 48%).

¹H NMR (DMSO-d₆, 400 MHz) δ: 6.09 (d, J 8.5 Hz, 1H, Ar); 6.95 (bs, 2H, NH₂); 7.02 (d, J 2.2 Hz, 1H, Ar); 7.25 (dd, J 7.6, 1.2 Hz, 1H, Ar); 7.33 (t, J 7.7 Hz, 1H, Ar); 7.49 (bs, 2H, NH₂); 7.61 (d, J 8.5 Hz, 1H, Ar); 7.69 (dd, J 7.7, 1.1 Hz, 1H, Ar); 8.00 (d, J 2.2 Hz, 1H, Ar); 12.86 (bs, 1H, HCl salt). M/Z (M+H)⁺: 226.1. Mp>250° C.

### Example 51: 3-(benzo[b]thiophen-4-yl)pyridine-2,6-diamine (hydrochloride)

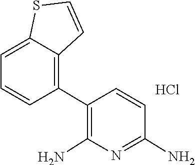 [SMILES: Nc1ccc(-c2cccc3sccc23)c(N)n1 ; Cl]

Example 51 was prepared according to method 5 starting from 2,6-diamino-3-iodopyridine 1h (75 mg, 0.32 mmol) and benzo[b]thiophen-4-ylboronic acid (74 mg, 0.41 mmol, 1.3 eq.). The crude was purified by flash chromatography (15 μm Interchim® SiO₂, DCM/MeOH: 100/0 to 95/5). The obtained foam was dissolved in a mixture of aqueous 1N HCl/ACN and the resulting solution was freeze dried to afford Example 51 as a beige solid (60 mg, 68%).

¹H NMR (DMSO-d₆, 400 MHz) δ: 6.09 (d, J 8.5 Hz, 1H, Ar); 6.79 (bs, 2H, NH₂); 7.16 (dd, J 5.6, 0.7 Hz, 1H, Ar); 7.28 (dd, J 7.3, 0.9 Hz, 1H, Ar); 7.43-7.47 (m, 3H, Ar+NH₂); 7.52 (d, J 8.5 Hz, 1H, Ar); 7.79 (d, J 5.6 Hz, 1H, Ar); 8.05 (d, J 8.1, 0.9 Hz, 1H, Ar); 12.88 (bs, 1H, HCl salt). M/Z (M+H)⁺: 242.1. Mp: 100-120° C.

### Example 52: 3-(6-fluoroquinolin-8-yl)pyridine-2,6-diamine (hydrochloride)

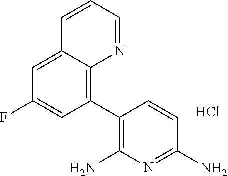 [SMILES: Nc1ccc(-c2cc(F)cc3cccnc23)c(N)n1 ; Cl]

Example 52 was prepared according to method 5 starting from 2,6-diamino-3-iodopyridine 1h (100 mg, 0.43 mmol) and (6-fluoroquinolin-8-yl)boronic acid (163 mg, 0.85 mmol, 2.0 eq.). The crude was purified by flash chromatography (SiO₂, DCM/MeOH: 100/0 to 92/8). The obtained foam was triturated in pentane and the collected precipitate was dissolved in a mixture of 1N H₂O/ACN. The suspension was freeze dried to afford Example 52 as a yellow solid (108 mg, 88%).

H NMR (DMSO-d6, 400 MHz) δ: 6.08 (d, J 8.5 Hz, 1H, Ar); 6.87 (bs, 2H, NH₂); 7.48 (bs, 2H, NH₂); 7.53 (d, J 8.5 Hz, 1H, Ar); 7.67 (dd, J 8.3, 4.4 Hz, 1H, Ar); 7.69 (dd, J 9.2, 2.9 Hz, 1H, Ar); 7.90 (dd, J 9.2, 2.9 Hz, 1H, Ar); 8.51 (dd, J 8.3, 1.6 Hz, 1H, Ar); 8.88 (dd, J 4.4, 1.6 Hz, 1H, Ar); 13.00 (bs, 1H, HCl salt). M/Z (M+H)⁺: 255.1. Mp>250° C.

### Example 53: 3-(6-methylquinolin-8-yl)pyridine-2,6-diamine (hydrochloride)

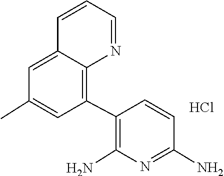 [SMILES: Cc1cc(-c2ccc(N)nc2N)c2ncccc2c1 ; Cl]

Example 53 was prepared according to method 5 starting from 2,6-diamino-3-iodopyridine 1h (100 mg, 0.43 mmol) and (6-methylquinolin-8-yl)boronic acid (159 mg, 0.85 mmol, 2.0 eq.). The crude was purified by flash chromatography (SiO₂, DCM/MeOH: 100/0 to 92/08). The obtained foam was triturated in pentane and the collected precipitate was dissolved in a mixture of 1 N H₂O/ACN. The suspension was freeze dried to afford Example 53 as a yellow solid (121 mg, 99%).

¹H NMR (DMSO-d₆, 400 MHz) δ: 2.57 (s, 3H, CH₃); 6.10 (d, J 8.5 Hz, 1H, Ar); 6.84 (bs, 2H, NH₂); 7.52 (bd, J 8.5 Hz, 3H, Ar+NH₂); 7.70 (bs, 1H, Ar); 7.77 (bs, 1H, Ar); 7.98 (bs, 1H, Ar); 8.69 (bs, 1H, Ar); 8.93 (dd, J 4.0 Hz, 1H, Ar); 13.04 (bs, 1H, HCl salt). M/Z (M+H)⁺: 251.1. Mp: 180-200° C.

### Example 54: 3-(5-(trifluoromethyl)quinolin-8-yl)pyridine-2,6-diamine (hydrochloride)

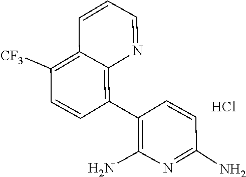 [SMILES: Nc1ccc(-c2ccc(C(F)(F)F)c3cccnc23)c(N)n1 ; Cl]

Example 54 was prepared according to method 5 starting from 2,6-diamino-3-iodopyridine 1h (75 mg, 0.32 mmol) and (5-(trifluoromethyl)quinolin-8-yl)boronic acid (116 mg, 0.48 mmol, 1.5 eq.). The crude was purified by flash chromatography (SiO₂, DCM/MeOH: 100/0 to 92/8). The obtained foam was triturated in pentane and the collected precipitate was dissolved in a mixture of 1 N H₂O/ACN. The suspension was freeze dried to afford Example 54 as a yellow solid (65 mg, 60%).

¹H NMR (DMSO-d₆, 400 MHz) δ: 6.09 (d, J 8.5 Hz, 1H, Ar); 6.87 (bs, 2H, NH₂); 7.47 (bs, 2H, NH₂); 7.53 (d, J 8.5 Hz, 1H, Ar); 7.79 (dd, J 8.7, 4.2 Hz, 1H, Ar); 7.84 (d, J 7.6 Hz, 1H, Ar); 8.14 (d, J 7.6 Hz, 1H, Ar); 8.54 (dt, J 8.7, 1.6 Hz, 1H, Ar); 9.02 (dd, J 4.2, 1.6 Hz, 1H, Ar); 13.00 (bs, 1H, HCl salt). M/Z (M+H)⁺: 305.1. Mp>250° C.

### Example 55: 3-(5-fluoroquinolin-8-yl)pyridine-2,6-diamine (hydrochloride)

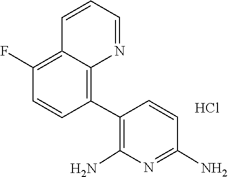 [SMILES: Nc1ccc(-c2ccc(F)c3cccnc23)c(N)n1 ; Cl]

Example 55 was prepared according to method 5 starting from 2,6-diamino-3-iodopyridine 1h (100 mg, 0.43 mmol) and (5-fluoroquinolin-8-yl)boronic acid (163 mg, 0.85 mmol, 2.0 eq.). The 4 HCl crude was purified by flash chromatography (15 μm Interchim® SiO₂, DCM/MeOH: 100/0 to 92/8). The obtained foam was triturated in Et₂O (2×5 mL) and the collected precipitate was dissolved in a mixture of 1N H₂O/ACN. The suspension was freeze dried to afford Example 55 as a yellow solid (26 mg, 21%).

¹H NMR (DMSO-d₆, 400 MHz) δ: 6.07 (d, J 8.4 Hz, 1H, Ar); 6.74 (bs, 2H, NH₂); 7.37 (bs, 2H, NH₂); 7.49 (d, J 8.4 Hz, 1H, Ar); 7.54 (dd, J 9.9, 8.1 Hz, 1H, Ar); 7.70 (dd, J 8.4, 4.1 Hz, 1H, Ar); 7.71 (dd, J 8.1, 6.1 Hz, 1H, Ar); 8.59 (dd, J 8.4, 1.7 Hz, 1H, Ar); 8.97 (dd, J 4.2, 1.7 Hz, 1H, Ar); 12.77 (bs, 1H, HCl salt). M/Z (M+H)⁺: 255.1. Mp: 73-81° C.

### Example 56: 8-(2,6-diaminopyridin-3-yl)quinolin-2(1H)-one (hydrochloride)

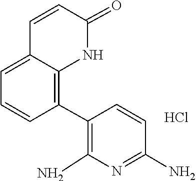 [SMILES: Nc1ccc(-c2cccc3ccc(=O)[nH]c23)c(N)n1 ; Cl]

Example 56 was prepared according to method 5 starting from 2,6-diamino-3-iodopyridine 1h (170 mg, 0.73 mmol) and 8-(4,4,5,5-tetramethyl-1,3,2-dioxaborolan-2-yl)quinolin-2(1H)-one 89 (237 mg, 0.87 mmol, 1.2 eq.). The HCl crude was purified by flash chromatography (KPNH, DCM/MeOH: 100/0 to 95/5). The resulting foam was further purified by flash chromatography (20 μm Interchim® SiO₂, DCM/MeOH: 100/0 to 95/5). The obtained foam was dissolved in a mixture of aqueous 1N HCl/ACN and the resulting solution was freeze dried to afford Example 56 as a white solid (72 mg, 34%).

¹H-NMR (DMSO-d₆, 400 MHz) δ: 6.03 (d, J 8.4 Hz, 1H, Ar); 6.51 (d, J 9.6 Hz, 1H, Ar); 6.76 (s, 2H, NH₂); 7.24 (t, J 7.6 Hz, 1H, Ar); 7.32 (dd, J 7.6, 1.5 Hz, 1H, Ar); 7.35 (d, J 8.4 Hz, 1H, Ar); 7.38 (bs, 2H, NH₂); 7.71 (dd, J 7.8, 1.6 Hz, 1H, Ar); 7.95 (d, J 9.6 Hz, 1H, Ar); 10.84 (s, 1H, NH); 12.64 (s, 1H, HCl salt). M/Z (M+H)⁺: 253.0. Mp>250° C.

### Example 57: 3-(7-fluoroquinolin-8-yl)pyridine-2,6-diamine (hydrochloride)

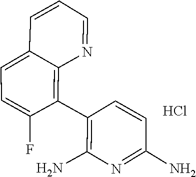 [SMILES: Nc1ccc(-c2c(F)ccc3cccnc23)c(N)n1 ; Cl]

Example 57 was prepared according to method 5 starting from 2,6-diamino-3-iodopyridine 1h (90 mg, 0.38 mmol) and (7-fluoroquinolin-8-yl)boronic acid 88 (237 mg, 0.65 mmol, 1.7 eq.). The crude was purified by flash chromatography (KPNH, DCM/MeOH: 100/0 to 95/5). The obtained foam was dissolved in a mixture of aqueous 1N HCl/ACN and the resulting solution was freeze dried to afford Example 57 as a yellow solid (53 mg, 48%).

¹H-NMR (DMSO-d₆, 400 MHz) δ: 6.10 (d, J 8.4 Hz, 1H, Ar); 6.88 (bs, 2H, NH₂); 7.49 (d, J 8.4 Hz, 1H, Ar); 7.52 (bs, 2H, NH₂); 7.65 (dd, J 8.4, 4.4 Hz, 1H, Ar); 7.71 (t, J 9.2 Hz, 1H, Ar); 8.22 (dd, J 9.2, 6.2 Hz, 1H, Ar); 8.60 (dd, J 8.4, 1.6 Hz, 1H, Ar); 13.00 (s, 1H, HCl salt). M/Z (M+H)⁺: 255.0. Mp: 190-220° C.

### Example 58: 3-(3-fluoroquinolin-8-yl)pyridine-2,6-diamine (hydrochloride)

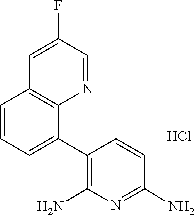 [SMILES: Nc1ccc(-c2cccc3cc(F)cnc23)c(N)n1 ; Cl]

Protected intermediate of Example 58 was prepared according to method 11 step 1 starting from 8-bromo-3-fluoroquinoline (120 mg, 0.53 mmol) and compound 9 (203 mg, 0.64 mmol, 1.2 eq.). The crude was purified by flash chromatography (SiO₂, CyHex/EtOAc: 100/0 to 20/80) to afford compound 99 (195 mg) as a yellow oil. M/Z (M+H)⁺: 339.1

Example 58 was prepared according to method 11 step 2 starting from compound 99 (195 mg). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 58 as a yellow solid (45 mg, 29% over 2 steps).

¹H-NMR (DMSO-d₆, 300 MHz) δ: 6.04 (d, J 8.4 Hz, 1H, Ar); 6.73 (bs, 2H, NH₂); 7.39 (bs, 2H, NH₂); 7.48 (d, J 8.4 Hz, 1H); 7.67 (dd, J 7.1, 1.6 Hz, 1H, Ar); 7.74 (dd, J 7.6, 7.4 Hz, 1H, Ar); 8.04 (dd, J 8.1, 1.6 Hz, 1H, Ar), 8.33 (dd, J 9.6, 2.9 Hz, 1H, Ar), 8.90 (d, J 2.9 Hz, 1H, Ar), 12.76 (bs, 1H, HCl salt). M/Z (M+H)⁺: 254.9. Mp: 114-116° C.

### Example 59: 3-(5,7-difluoroquinolin-8-yl)pyridine-2,6-diamine (hydrochloride)

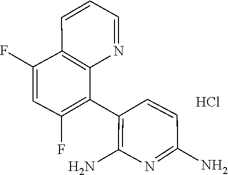 [SMILES: Nc1ccc(-c2c(F)cc(F)c3cccnc23)c(N)n1 ; Cl]

Protected intermediate of Example 59 was prepared according to method 11 step 1 starting from 8-bromo-5,7-difluoroquinoline 24 (120 mg, 0.49 mmol) and compound 9 (204 mg, 0.63 mmol, 1.3 eq.). The crude was purified by flash chromatography (SiO₂, CyHex/EtOAc: 100/0 to 20/80) to afford compound 100 (161 mg, 92%) as a light brown oil. M/Z (M+H)⁺: 357.1

Example 59 was prepared according to method 11 step 2 starting from compound 100 (161 mg, 0.45 mmol). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 59 as a yellow solid (68 mg, 49%).

¹H NMR (300 MHz, DMSO): 6.05 (d, J 8.5 Hz, 1H, Ar); 6.81 (bs, 2H, NH₂); 7.41 (bs, 2H, NH₂); 7.46 (d, J 8.5 Hz, 1H, Ar); 7.65 (dd, J 8.4, 4.3 Hz, 1H, Ar); 1H, Ar); 7.73 (t, J 10.1 Hz, 1H, Ar); 8.54 (dd, J 8.4, 1.7 Hz, 1H, Ar); 8.95 (dd, J 4.3, 1.7 Hz, 1H, Ar); 12.47 (bs, 1H, HCl salt). M/Z (M+H)⁺: 273.25.

### Example 60: 3-(3-chloro-7-fluoroquinolin-8-yl)pyridine-2,6-diamine (hydrochloride)

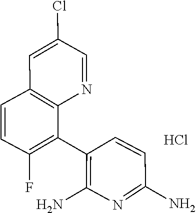 [SMILES: Nc1ccc(-c2c(F)ccc3cc(Cl)cnc23)c(N)n1 ; Cl]

Protected intermediate of Example 60 was prepared according to method 11 step 1 starting from 8-bromo-3-chloro-7-fluoroquinoline 71 (141 mg, 0.54 mmol) and compound 9 (224 mg, 0.70 mmol, 1.3 eq.). The crude was purified by flash chromatography (SiO₂, CyHex/EtOAc: 100/0 to 20/80) to afford compound 101 (131 mg, 65%) as a light brown oil. M/Z (M[³⁵Cl]+H)⁺: 373.2.

Example 60 was prepared according to method 11 step 2 starting from compound 101 (131 mg, 0.35 mmol). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 60 as a yellow solid (60 mg, 51%).

¹H-NMR (DMSO-d₆, 400 MHz) δ: 6.08 (d, J 8.5 Hz, 1H, Ar); 6.89 (bs, 2H, NH₂); 7.48 (d, J 8.5 Hz, 1H, Ar); 7.50 (bs, 2H, NH₂); 7.73 (t, J 9.1 Hz, 1H, Ar); 8.15 (dd, J 9.1, 6.1 Hz, 1H, Ar); 8.70 (d, J 2.4 Hz, 1H, Ar); 8.89 (d, J 2.4 Hz, 1H, Ar); 13.03 (bs, 1H, HCl salt). M/Z (M[³⁵Cl]+H)⁺: 365.2. Mp>250° C.

### Example 61: 3-(3,5,7-trifluoroquinolin-8-yl)pyridine-2,6-diamine (hydrochloride) F

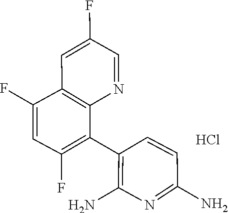 [SMILES: Nc1ccc(-c2c(F)cc(F)c3cc(F)cnc23)c(N)n1 ; Cl]

Protected intermediate of Example 61 was prepared according to method 11 step 1 starting from 8-bromo-3,5,7-trifluoroquinoline 78 (105 mg, 0.40 mmol) and compound 9 (175 mg, 0.55 mmol, 1.4 eq.). The crude was purified by flash chromatography (SiO₂, CyHex/EtOAc, 100/0 to 20/80) to afford compound 102 (110 mg, 72%) as a light brown oil. M/Z (M+H)⁺: 357.1.

Example 61 was prepared according to method 11 step 2 starting from compound 102 (110 mg, 0.29 mmol). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 61 as a yellow solid (75 mg, 79%).

¹H-NMR (DMSO-d₆, 400 MHz) δ: 6.08 (d, J 8.5 Hz, 1H, Ar); 6.89 (bs, 2H, NH₂); 7.50 (d, J 8.5 Hz, 1H, Ar); 7.50 (bs, 2H, NH₂); 7.83 (t, J 10.0 Hz, 1H, Ar); 8.44 (dd, J 9.0, 2.8 Hz, 1H, Ar); 9.04 (d, J 2.8 Hz, 1H, Ar); 12.98 (bs, 1H, HCl salt). M/Z (M+H)⁺: 291.0. Mp>250° C.

### Example 62: 8-(2,6-diaminopyridin-3-yl)-7-fluoroquinolin-2-ol (hydrochloride)

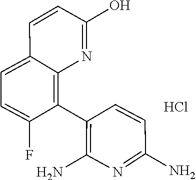 [SMILES: Nc1ccc(-c2c(F)ccc3ccc(O)nc23)c(N)n1 ; Cl]

Protected intermediate of Example 62 was prepared according to method 11 step 1 starting from 8-bromo-7-fluorophenyl-2(1H)-one 48 (175 mg, 0.72 mmol) and compound 9 (346 mg, 1.08 mmol, 1.5 eq.). The crude was purified by flash chromatography (SiO₂, DCM/MeOH: 100/0 to 97/3) to afford compound 103 (267 mg) as a light brown oil. M/Z (M+H)⁺: 355.2.

Example 62 was prepared according to method 11 step 2 starting from compound 103 (267 mg). The crude was purified by flash chromatography (KPNH, DCM/MeOH: 100/0 to 98/2). The resulting foam was further purified by flash chromatography (20 μm Interchim® SiO₂, DCM/MeOH: 100/0 to 90/10). The obtained foam was dissolved in a mixture of aqueous 1N HCl/ACN and the resulting solution was freeze dried to afford Example 62 as a white solid (34 mg, 15% over 2 steps).

¹H-NMR (DMSO-d₆, 400 MHz) δ: 6.05 (d, J 8.6 Hz, 1H, Ar); 6.46 (d, J 9.6 Hz, 1H, Ar); 6.90 (bs, 2H, NH₂); 7.14 (t, J 8.8 Hz, 1H, Ar); 7.36 (d, J 8.6 Hz, 1H, Ar); 7.47 (bs, 2H, NH₂); 7.78 (dd, J 8.6, 6.2 Hz, 2H, Ar); 7.95 (d, J 9.6 Hz, 1H, Ar); 11.02 (s, 1H, NH or OH); 12.65 (bs, 1H, HCl salt). M/Z (M+H)⁺: 271.0. Mp: 210-230° C.

### Example 63: 8-(2,6-diaminopyridin-3-yl)-7-chloroquinolin-2-ol (hydrochloride)

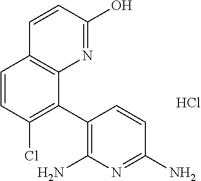 [SMILES: Nc1ccc(-c2c(Cl)ccc3ccc(O)nc23)c(N)n1 ; Cl]

Protected intermediate of Example 63 was prepared according to method 11 step 1 starting from 8-bromo-7-chlorophenyl-2(1H)-one 50 (124 mg, 0.48 mmol) and compound 9 (230 mg, 0.72 mmol, 1.5 eq.). The crude was purified by flash chromatography (SiO₂, DCM/MeOH: 100/0 to 97/3) to afford compound 104 (143 mg) as a light brown oil. M/Z (M[³⁵Cl]+H)⁺: 371.3.

Example 63 was prepared according to method 11 step 2 starting from compound 104 (143 mg), the crude was purified by flash chromatography (KPNH, DCM/MeOH: 100/0 to 96/4). The resulting foam was further purified by flash chromatography (20 μm Interchim® SiO₂, DCM/MeOH: 100/0 to 90/10). The obtained foam was dissolved in a mixture of aqueous 1N HCl/ACN and the resulting solution was freeze dried to afford Example 63 as a white solid (50 mg, 32% over 2 steps).

¹H-NMR (DMSO-d₆, 400 MHz) δ: 6.00 (d, J 8.4 Hz, 1H, Ar); 6.32 (bs, 2H, NH₂); 6.52 (d, J 9.6 Hz, 1H, Ar); 6.96 (bs, 2H, NH₂); 7.18 (d, J 8.4 Hz, 1H, Ar); 7.37 (d, J 8.4 Hz, 1H, Ar); 7.72 (d, J 8.4 Hz, 1H, Ar); 7.97 (d, J 9.6 Hz, 1H, Ar); 10.41 (s, 1H, NH or OH); HCl salt signal not observed. M/Z (M[³⁵Cl]+H)⁺: 287.0. Mp>250° C.

### Example 64: 8-(2,6-diaminopyridin-3-yl)-6,7-difluoroquinolin-2-ol (hydrochloride)

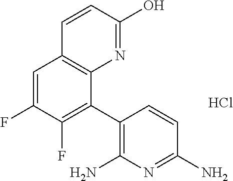 [SMILES: Nc1ccc(-c2c(F)c(F)cc3ccc(O)nc23)c(N)n1 ; Cl]

Protected intermediate of Example 64 was prepared according to method 11 step 1 starting from 8-bromo-6,7-difluorophenyl-2(1H)-one 51 (143 mg, 0.55 mmol) and compound 9 (263 mg, 0.82 mmol, 1.5 eq.). The crude was purified by flash chromatography (SiO₂, DCM/MeOH: 100/0 to 95/5) to afford compound 105 (118 mg) as a light brown solid. M/Z (M+H)⁺: 373.1.

Example 64 was prepared according to method 11 step 2 starting from compound 105 (118 mg). The crude was purified by flash chromatography (KPNH, DCM/MeOH: 100/0 to 95/5). The resulting foam was further purified by flash chromatography (20 μm Interchim® SiO₂, DCM/MeOH: 100/0 to 90/10). The obtained foam was dissolved in a mixture of aqueous 1N HCl/ACN and the resulting solution was freeze dried to afford Example 64 as a white solid (23 mg, 13% over 2 steps).

¹H-NMR (DMSO-d₆, 400 MHz) δ: 6.06 (d, J 8.6 Hz, 1H, Ar); 6.54 (d, J 9.6 Hz, 1H, Ar); 6.95 (bs, 2H, NH₂); 7.40 (d, J 8.6 Hz, 1H, Ar); 7.51 (bs, 2H, NH₂); 7.88 (dd, J 10.6, 8.6 Hz, 2H, Ar); 7.92 (d, J 9.6 Hz, 1H, Ar); 11.05 (s, 1H, NH or OH); 12.68 (bs, 1H, HCl salt). M/Z (M+H)⁺: 289.1. Mp>250° C.

### Example 65: 6-ethyl-5-(7-fluoroquinolin-8-yl)pyridin-2-amine (hydrochloride)

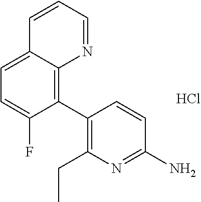 [SMILES: CCc1nc(N)ccc1-c1c(F)ccc2cccnc12 ; Cl]

Example 65 was prepared according to method 2 starting from 5-bromo-6-ethylpyridin-2-amine 1d (150 mg, 0.75 mmol) and (7-fluoroquinolin-8-yl)boronic acid 88 (171 mg, 0.90 mmol, 1.2 eq.). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 65 as a beige solid (125 mg, 55%).

¹H-NMR (DMSO-d₆, 400 MHz) δ: 1.05 (t, J 7.6 Hz, 3H, CH₂—CH₃); 2.36-2.47 (m, 2H, CH₂—CH₃); 6.99 (d, J 9.0 Hz, 1H, Ar); 7.62 (dd, J 8.2, 4.2 Hz, 1H, Ar); 7.74 (t, J 9.1 Hz, 1H, Ar); 7.82 (d, J 9.0 Hz, 1H, Ar); 8.12 (bs, 2H, NH₂); 8.25 (dd, J 9.1, 6.2 Hz, 1H, Ar); 8.55 (dd, J 8.4, 1.6 Hz, 1H, Ar); 8.91 (dd, J 4.2, 1.6 Hz, 1H, Ar); 14.43 (bs, 1H, HCl salt). M/Z (M+H)⁺: 268.1. Mp: 150-195° C.

### Example 66: 5-(chroman-8-yl)-6-ethylpyridin-2-amine (hydrochloride)

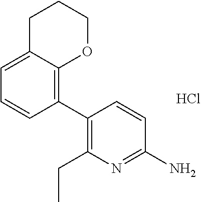 [SMILES: CCc1nc(N)ccc1-c1cccc2c1OCCC2 ; Cl]

Example 66 was prepared according to method 2 starting from 5-bromo-6-ethylpyridin-2-amine 1d (125 mg, 0.62 mmol) and chroman-8-ylboronic acid (133 mg, 0.75 mmol, 1.2 eq.). The crude was purified by flash chromatography (KPNH, DCM/MeOH: 100/0 to 98/02). The obtained foam was further purified by flash chromatography (20 μm Interchim® SiO₂, DCM/MeOH: 100/0 to 95/05). The obtained foam was dissolved in a mixture of aqueous 1N HCl/ACN and the resulting solution was freeze dried and triturated twice in Et₂O (5 mL) to afford Example 66 as a white solid (93 mg, 52%).

¹H-NMR (DMSO-d₆, 400 MHz) δ: 1.13 (t, J 7.6 Hz, 3H, CH₂—CH₃); 1.88-1.94 (m, 2H, CH₂—CH₂—CH₂); 2.52-2.55 (m, 2H, CH₂—CH₃); 2.80 (t, J 6.4 Hz, 2H, CH₂); 4.09 (t, J 5.2 Hz, 2H, O—CH₂); 6.87 (d, J 9.0 Hz, 1H, Ar); 6.90 (t, J 7.4 Hz, 1H, Ar); 6.96 (dd, J 7.4, 1.8 Hz, 1H, Ar); 7.14 (dd, J 7.4, 1.8 Hz, 1H, Ar); 7.68 (d, J 9.0 Hz, 1H, Ar); 7.88 (bs, 2H, NH₂); 14.08 (bs, 1H, HCl salt). M/Z (M+H)⁺: 254.9. Mp: 180-192° C.

### Example 67: 6-isobutyl-5-(quinolin-8-yl)pyridin-2-amine (hydrochloride)

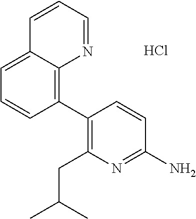 [SMILES: CC(C)Cc1nc(N)ccc1-c1cccc2cccnc12 ; Cl]

Example 67 was prepared according to method 2 starting from 5-bromo-6-isobutylpyridin-2-amine 12 (133 mg, 0.58 mmol) and quinolin-8-ylboronic acid (151 mg, 0.87 mmol, 1.5 eq.). The crude was purified by flash chromatography (KPNH, CyHex/EtOAc: 100/0 to 70/30). The resulting foam was further purified by flash chromatography (15 μm Interchim® SiO₂, DCM/MeOH: 100/0 to 80/20). The obtained foam was dissolved in a mixture of aqueous 1N HCl/ACN and the resulting solution was freeze dried to afford Example 67 as a beige solid (85 mg, 45%).

¹H-NMR (DMSO-d₆, 400 MHz) δ: 0.6 (bs, 6H, 2 CH₃); 1.78-1.88 (m, 1H, CH); 2.24 (bs, 1H, CHₐH_{b}); 2.53-2.59 (m, 1H, CH₂H_{b}); 6.97 (d, J 9.0 Hz, 1H, Ar); 7.66 (dd, J 8.4, 4.4 Hz, 1H, Ar); 7.73-7.80 (m, 2H, Ar); 7.81 (d, J 9.0 Hz, 1H, Ar); 8.06 (bs, 2H, NH₂); 8.15 (dd, J 7.6, 1.7 Hz, 1H, Ar); 8.57 (d, J 8.4, 1H, Ar); 8.91 (dd, J 4.4, 1.7 Hz, 1H, Ar); 14.42 (bs, 1H, HCl salt). M/Z (M+H)⁺: 278.2. Mp: 100-140° C.

### Example 68: 6-(cyclobutylmethyl)-5-(quinolin-8-yl)pyridin-2-amine (hydrochloride)

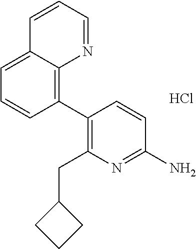 [SMILES: Nc1ccc(-c2cccc3cccnc23)c(CC2CCC2)n1 ; Cl]

Example 68 was prepared according to method 2 starting from 5-bromo-6-(cyclobutylmethyl)pyridin-2-amine 14 (100 mg, 0.42 mmol) and quinolin-8-ylboronic acid (108 mg, 0.62 mmol, 1.5 eq.). The crude residue was purified by flash chromatography (KPNH, CyHex/EtOAc: 100/0 to 70/30). The resulting foam was further purified by flash chromatography (15 μm Interchim® SiO₂, DCM/MeOH: 100/0 to 80/20). The obtained foam was dissolved in a mixture of aqueous 1N HCl/ACN and the resulting solution was freeze dried to afford Example 68 as a beige solid (40 mg, 30%).

¹H-NMR (DMSO-d₆, 400 MHz) δ: 1.34 (bs, 2H, CH₂); 1.44-1.52 (m, 1H, CHₐH_{b}); 1.53-1.65 (m, 1H, CHₐH_{b}); 1.73 (bs, 2H, CH₂); 2.39-2.49 (m, 2H, CH₂); 2.72 (bs, 1H, CH); 6.94 (d, J 9.0 Hz, 1H, Ar); 7.66 (dd, J 8.3, 4.2 Hz, 1H, Ar); 7.74-7.80 (m, 2H, Ar); 7.80 (d, J 9.0 Hz, 1H, Ar); 8.04 (bs, 2H, NH₂); 8.15 (dd, J 7.2, 2.4 Hz, 1H, Ar); 8.56 (dd, J 8.4, 1.7, 1H, Ar); 8.91 (dd, J 4.2, 1.7 Hz, 1H, Ar); 14.30 (bs, 1H, HCl salt). M/Z (M+H)⁺: 290.2. Mp: 120-180° C.

### Example 69: 5-(7-fluoroquinolin-8-yl)-6-(3,3,3-trifluoropropyl)pyridin-2-amine (hydrochloride)

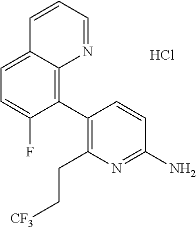 [SMILES: Nc1ccc(-c2c(F)ccc3cccnc23)c(CCC(F)(F)F)n1 ; Cl]

Example 69 was prepared according to method 2 starting from 5-bromo-6-(3,3,3-trifluoropropyl)pyridin-2-amine 16 (110 mg, 0.41 mmol) and (7-fluoroquinolin-8-yl)boronic acid 88 (156 mg, 0.82 mmol, 2.0 eq.). The crude residue was purified by flash chromatography (SiO₂, DCM/MeOH: 100/0 to 97/03). The resulting foam was further purified by flash CF₃ chromatography (SiO₂, CyHex/EtOAc: 100/0 to 0/100). The obtained foam was dissolved in a mixture of aqueous 1 N HCl/ACN and the resulting solution was freeze dried to afford Example 69 as a beige solid (27 mg, 18%).

¹H-NMR (DMSO-d₆, 400 MHz) δ: 2.59-2.68 (m, 4H, 2*CH₂); 7.03 (d, J 9.0 Hz, 1H, Ar); 7.62 (dd, J 8.4, 4.2 Hz, 1H, Ar); 7.75 (t, J 9.2 Hz, 1H, Ar); 7.87 (d, J 9.0 Hz, 1H, Ar); 8.09 (bs, 2H, NH₂); 8.26 (dd, J 9.2, 6.4 Hz, 1H, Ar); 8.55 (dd, J 8.4, 1.8 Hz, 1H, Ar); 8.92 (dd, J 4.2, 1.8 Hz, 1H, Ar); 14.56 (bs, 1H, HCl salt). M/Z (M+H)⁺: 336.0. Mp: 85-145° C.

Example 70: 5-(7-fluoroquinolin-8-yl)-6-isobutylpyridin-2-amine (hydrochloride)

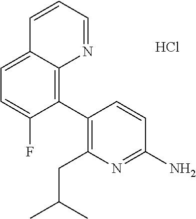 [SMILES: CC(C)Cc1nc(N)ccc1-c1c(F)ccc2cccnc12 ; Cl]

Example 70 was prepared according to method 2 starting from 5-bromo-6-isobutylpyridin-2-amine 12 (110 mg, 0.48 mmol) and (7-fluoroquinolin-8-yl)boronic acid 88 (217 mg, 1.14 mmol, 2.0 eq.). The crude residue was purified by flash chromatography (SiO₂, DCM/MeOH: 100/0 to 90/10). The resulting foam was further purified by flash chromatography (SiO₂, DCM/MeOH: 100/0 to 97/3). The obtained foam was dissolved in a mixture of aqueous 1N HCl/ACN and the resulting solution was freeze dried. The obtained solid was triturated in Et₂O (5 mL) to afford Example 70 as a beige solid (75 mg, 47%).

¹H-NMR (DMSO-d₆, 400 MHz) δ: 0.55 (d, J 6.6 Hz, 3H, CH₃); 0.63 (d, J 6.6 Hz, 3H, CH₃); 1.77-1.87 (m, 1H, CH); 2.26 (dd, J 14.0, 7.6 Hz, 1H, CHₐH_{b}); 2.40 (dd, J 14.0, 7.6 Hz, 1H, CHₐH_{b}); 6.99 (d, J 9.0 Hz, 1H, Ar); 7.60 (dd, J 8.4, 4.2 Hz, 1H, Ar); 7.73 (t, J 9.2 Hz, 1H, Ar); 7.84 (d, J 9.0 Hz, 1H, Ar); 8.05 (bs, 2H, NH₂); 8.24 (dd, J 9.2, 6.4 Hz, 1H, Ar); 8.53 (dd, J 8.4, 1.8 Hz, 1H, Ar); 8.90 (dd, J 4.2, 1.8 Hz, 1H, Ar); 14.26 (bs, 1H, HCl salt). M/Z (M+H)⁺: 296.1. Mp: 130-170° C.

### Example 71: 5-(7-fluoroquinolin-8-yl)-6-(4,4,4-trifluorobutyl)pyridin-2-amine (hydrochloride)

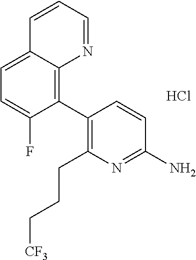 [SMILES: Nc1ccc(-c2c(F)ccc3cccnc23)c(CCCC(F)(F)F)n1 ; Cl]

Example 71 was prepared according to method 2 starting from 5-bromo-6-(4,4,4-trifluorobutyl)pyridin-2-amine 18 (110 mg, 0.39 mmol) and (7-fluoroquinolin-8-yl)boronic acid 88 (89 mg, 0.47 mmol, 1.2 eq.). The crude F residue was purified by flash chromatography (15 μm Interchim® SiO₂, DCM/MeOH: 100/0 to 90/10). The obtained foam was dissolved in a mixture of aqueous 1N HCl/ACN and the resulting solution was freeze dried to afford Example 71 as a beige solid (65 mg, 44%).

¹H-NMR (DMSO-d₆, 400 MHz) δ: 1.68-1.76 (m, 2H, CH₂); 1.95-2.08 (m, 2H, CH₂); 2.52-2.57 (m, 2H, CH₂); 7.01 (d, J 9.0 Hz, 1H, Ar); 7.61 (dd, J 8.4, 4.2 Hz, 1H, Ar); 7.74 (t, J 9.2 Hz, 1H, Ar); 7.83 (d, J 9.0 Hz, 1H, Ar); 8.08 (bs, 2H, NH₂); 8.25 (dd, J 9.2, 6.4 Hz, 1H, Ar); 8.54 (dd, J 8.4, 1.8 Hz, 1H, Ar); 8.89 (dd, J 4.2, 1.8 Hz, 1H, Ar); 14.42 (bs, 1H, HCl salt). M/Z (M+H)⁺: 349.9. Mp: 70-130° C.

### Example 72: 6-(cyclopropylmethyl)-5-(7-fluoroquinolin-8-yl)pyridin-2-amine (hydrochloride)

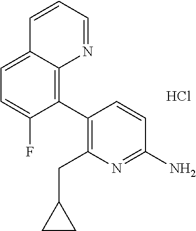 [SMILES: Nc1ccc(-c2c(F)ccc3cccnc23)c(CC2CC2)n1 ; Cl]

Example 72 was prepared according to method 2 starting from 5-bromo-6-(cyclopropylmethyl)pyridin-2-amine 20 (110 mg, 0.48 mmol) and (7-fluoroquinolin-8-yl)boronic acid 88 (111 mg, 0.58 mmol, 1.2 eq.). The crude residue was purified by flash chromatography (15 μm Interchim® SiO₂, DCM/MeOH: 100/0 to 90/10). The resulting foam was further purified by flash chromatography (KPNH, DCM/MeOH: 100/0 to 98/02). The obtained foam was dissolved in a mixture of aqueous 1 N HCl/ACN and the resulting solution was freeze dried to afford Example 72 as a beige solid (68 mg, 43%).

¹H-NMR (DMSO-d₆, 400 MHz) δ: 1.61-1.78 (m, 2H, CH₂); 1.79-1.91 (m, 2H, CH₂); 2.25-2.36 (m, 2H, CH₂); 3.34 (quint, J 9.1 Hz, 1H, CH); 6.98 (d, J 9.0 Hz, 1H, Ar); 7.61 (dd, J 8.2, 4.2 Hz, 1H, Ar); 7.72 (t, J 9.2 Hz, 1H, Ar); 7.80 (d, J 9.0 Hz, 1H, Ar); 8.23 (dd, J 9.2, 6.4 Hz, 1H, Ar); 8.34 (bs, 2H, NH₂); 8.53 (dd, J 8.4, 1.8 Hz, 1H, Ar); 8.90 (dd, J 4.2, 1.8 Hz, 1H, Ar); 13.83 (bs, 1H, HCl salt). M/Z (M+H)⁺: 294.1. Mp:170-207° C.

### Example 73: 5-(7-fluoroquinolin-8-yl)-6-isopentylpyridin-2-amine (hydrochloride)

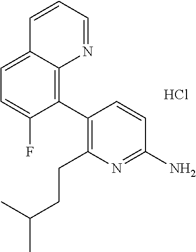 [SMILES: CC(C)CCc1nc(N)ccc1-c1c(F)ccc2cccnc12 ; Cl]

Example 73 was prepared according to method 2 starting from 5-bromo-6-isopentylpyridin-2-amine 22 (110 mg, 0.45 mmol) and (7-fluoroquinolin-8-yl)boronic acid 88 (104 mg, 0.54 mmol, 1.2 eq.). The crude residue was purified by flash chromatography (15 μm Interchim® SiO₂, DCM/MeOH: 100/0 to 95/5). The obtained foam was dissolved in a mixture of aqueous 1N HCl/ACN and the resulting solution was freeze dried to afford Example 73 as a beige solid (62 mg, 39%).

¹H-NMR (DMSO-d₆, 400 MHz) δ: 0.45 (d, J 6.6 Hz, 3H, CH₃); 0.50 (d, J 6.6 Hz, 3H, CH₃); 1.17-1.27 (m, 1H, CH); 1.29-1.39 (m, 2H, CH₂); 2.36-2.44 (m, 2H, CH₂); 6.98 (d, J 9.0 Hz, 1H, Ar); 7.61 (dd, J 8.4, 4.2 Hz, 1H, Ar); 7.73 (t, J 9.2 Hz, 1H, Ar); 7.84 (d, J 9.0 Hz, 1H, Ar); 8.03 (bs, 2H, NH₂); 8.24 (dd, J 9.2, 6.4 Hz, 1H, Ar); 8.53 (dd, J 8.4, 1.8 Hz, 1H, Ar); 8.91 (dd, J 4.2, 1.8 Hz, 1H, Ar); 14.26 (bs, 1H, HCl salt). M/Z (M+H)⁺: 310.2. Mp: 100-140° C.

### Example 74: 6-ethyl-5-(6-fluoroquinolin-8-yl)pyridin-2-amine (hydrochloride)

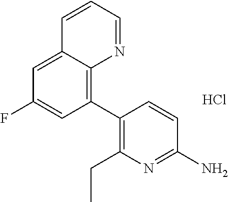 [SMILES: CCc1nc(N)ccc1-c1cc(F)cc2cccnc12 ; Cl]

Example 74 was prepared according to method 2 starting from 2-amino-5-bromo-6-ethylpyridine 1d (60 mg, 0.30 mmol) and (6-fluoroquinolin-8-yl)boronic acid (110 mg, 0.60 mmol, 2.0 eq.). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 74 as an orange solid (21 mg, 23%).

¹H-NMR (DMSO-d₆, 400 MHz) δ: 1.07 (t, J 7.6 Hz, 3H, CH₃); 2.37-2.56 (m, 2H, CH₂); 6.95 (d, J 9.0 Hz, 1H, Ar); 7.65 (dd, J 8.5, 4.2 Hz, 1H, Ar); 7.76 (dd, J 9.0, 2.8 Hz, 1H, Ar); 7.82 (d, J 9.0 Hz, 1H, Ar); 7.95 (dd, J 9.0, 2.8 Hz, 1H, Ar); 8.08 (bs, 2H, NH₂); 8.48 (dd, J 8.5, 1.6 Hz, 1H, Ar); 8.86 (dd, J 4.2, 1.6 Hz, 1H, Ar); 14.40 (bs, 1H, NH₃+). M/Z (M+H)⁺: 268.2. Mp: 50-56° C.

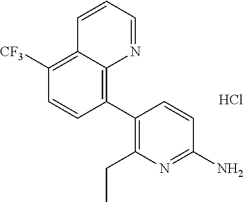 [SMILES: CCc1nc(N)ccc1-c1ccc(C(F)(F)F)c2cccnc12 ; Cl]

### Example 75: 6-ethyl-5-(5-(trifluoromethyl)quinolin-8-yl)pyridin-2-amine (hydrochloride)

Example 75 was prepared according to method 2 starting from 2-amino-5-bromo-6-ethylpyridine 1d (60 mg, 0.30 mmol) and (5-(trifluoromethyl)quinolin-8-yl)boronic acid (110 mg, 0.45 mmol, 1.5 eq.). The crude was purified by flash chromatography (SiO₂, DCM/MeOH: 100/0 to 93/7). The obtained foam was dissolved in a mixture of aqueous 1N HCl/ACN and the resulting solution was freeze dried to afford Example 75 as a beige solid (42 mg, 40%).

¹H-NMR (DMSO-d₆, 400 MHz) δ: 1.08 (t, J 7.6 Hz, 3H, CH₃); 2.36-2.44 (m, 1H, CH₂); 2.50-2.57 (m, 1H, CH₂); 6.97 (d, J 9.0 Hz, 1H, Ar); 7.82 (dd, J 8.5, 4.2 Hz, 1H, Ar); 7.84 (d, J 9.0 Hz, 1H, Ar); 7.91 (d, J 7.5 Hz, 1H, Ar); 8.08 (bs, 2H, NH₂); 8.20 (d, J 7.5 Hz, 1H, Ar); 8.56-8.62 (m, 1H, Ar); 9.04 (dd, J 4.2, 1.5 Hz, 1H, Ar); 14.30 (bs, 1H, HCl salt). M/Z (M+H)⁺: 318.2. Mp: 120-137° C.

### Example 76: 6-ethyl-5-(7-fluoro-2-methylquinolin-8-yl)pyridin-2-amine (hydrochloride)

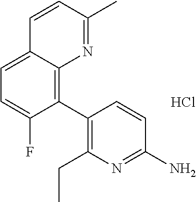 [SMILES: CCc1nc(N)ccc1-c1c(F)ccc2ccc(C)nc12 ; Cl]

Example 76 was prepared according to method 2 starting from 2-amino-5-bromo-6-ethylpyridine Id (100 mg, 0.50 mmol) and (7-fluoro-2-methylquinolin-8-yl)boronic acid (255 mg, 1.24 mmol, 2.5 eq.). The crude was purified by flash chromatography (SiO₂, DCM/MeOH: 100/0 to 96/04). The obtained foam was dissolved in a mixture of aqueous 1N HCl/ACN and the resulting solution was freeze dried to afford Example 76 as a beige solid (99 mg, 62%).

¹H-NMR (DMSO-d₆, 400 MHz) δ: 1.09 (t, J 7.6 Hz, 3H, CH₂—CH₃); 2.4 (q, J 7.6 Hz, 2H, CH₂—CH₃); 2.57 (s, 3H, CH₃); 6.98 (d, J 9.0 Hz, 1H, Ar); 7.49 (d, J 8.4 Hz, 1H, Ar); 7.63 (t, J 9.1 Hz, 1H, Ar); 7.81 (d, J 9.1 Hz, 1H, Ar); 8.08 (bs, 2H, NH₂); 8.16 (dd, J 9.0, 6.5 Hz, 1H, Ar); 8.40 (d, J 8.4 Hz, 1H, Ar); 14.27 (bs, 1H, HCl salt). M/Z (M+H)⁺: 282.1. Mp: 95-120° C.

### Example 77: 6-ethyl-5-(6-methylquinolin-8-yl)pyridin-2-amine (hydrochloride)

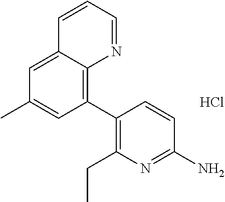 [SMILES: CCc1nc(N)ccc1-c1cc(C)cc2cccnc12 ; Cl]

Example 77 was prepared according to method 2 starting from 2-amino-5-bromo-6-ethylpyridine 1d (65 mg, 0.32 mmol) and (6-methylquinolin-8-yl)boronic acid (91 mg, 0.48 mmol, 1.5 eq.). The crude was purified by flash chromatography (SiO₂, DCM/MeOH, 100/0 to 92/8). The obtained foam was dissolved in a mixture of aqueous 1 N HCl/ACN and the resulting solution was freeze dried to afford Example 77 as a beige solid (68 mg, 70%).

¹H-NMR (DMSO-d₆, 400 MHz) δ: 1.06 (t, J 7.6 Hz, 3H, CH₂—CH₃); 2.36-2.48 (m, 2H, CH₂—CH₃); 2.56 (s, 3H, CH₃); 6.94 (d, J 9.0 Hz, 1H, Ar); 7.60-7.63 (m, 2H, Ar); 7.80 (d, J 9.0 Hz, 1H, Ar); 7.91 (s, 1H, Ar); 8.01 (bs, 2H, NH₂); 8.16 (d, J 8.4 Hz, 1H, Ar); 8.83 (dd, J 4.3, 1.7 Hz, 1H, Ar); 14.24 (bs, 1H, HCl salt). M/Z (M+H)⁺: 264.2. Mp: 100-140° C.

### Example 78: 5-(benzo[b]thiophen-4-yl)-6-ethylpyridin-2-amine (hydrochloride)

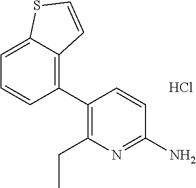 [SMILES: CCc1nc(N)ccc1-c1cccc2sccc12 ; Cl]

Example 78 was prepared according to method 2 starting from 2-amino-5-bromo-6-ethylpyridine 1d (75 mg, 0.37 mmol) and benzo[b]thiophen-4-ylboronic acid (86 mg, 0.48 mmol, 1.3 eq.). The crude was purified by flash chromatography (15 μm Interchim® SiO₂, DCM/MeOH: 100/0 to 95/5). The obtained foam was dissolved in a mixture of aqueous 1N HCl/ACN and the resulting solution was freeze dried to afford Example 78 as a beige solid (74 mg, 69%).

¹H-NMR (DMSO-d₆, 400 MHz) δ: 1.07 (t, J 7.6 Hz, 3H, CH₂—CH₃); 2.40-2.46 (m, 1H, CHₐH_{b}—CH₃); 2.54-2.59 (m, 1H, CHₐH_{b}—CH₃); 6.94 (d, J 9.0 Hz, 1H, Ar); 7.14 (dd, J 5.6, 0.7 Hz, 1H, Ar); 7.29 (dd, J 7.2, 0.9 Hz, 1H, Ar); 7.47 (t, J 7.7 Hz, 1H, Ar); 7.81 (d, J 9.0 Hz, 1H, Ar); 7.83 (d, J 5.6 Hz, 1H, Ar); 7.96 (bs, 2H, NH₂); 8.11 (dt, J 8.1, 0.9 Hz, 1H, Ar); 14.09 (bs, 1H, HCl salt). M/Z (M+H)⁺: 251.1. Mp: 80-140° C.

### Example 79: 5-(benzofuran-7-yl)-6-ethylpyridin-2-amine (hydrochloride)

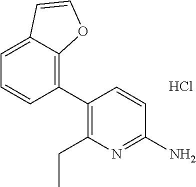 [SMILES: CCc1nc(N)ccc1-c1cccc2ccoc12 ; Cl]

Example 79 was prepared according to method 2 starting from 2-amino-5-bromo-6-ethylpyridine 1d (75 mg, 0.37 mmol) and 2-(benzofuran-7-yl)-4,4,5,5-tetramethyl-1,3,2-dioxaborolane (180 mg, 0.75 mmol, 2.0 eq.). The crude was purified by flash chromatography (15 μm Interchim® SiO₂, DCM/MeOH, 100/0 to 90/10). The resulting product was triturated in Et₂O (2×2 mL). The obtained foam was dissolved in a mixture of aqueous 1N HCl/ACN and the resulting solution was freeze dried to afford Example 79 as a beige solid (12 mg, 12%).

¹H-NMR (DMSO-d₆, 400 MHz) δ: 1.14 (t, J 7.6 Hz, 3H, CH₃); 2.57 (q, J 7.6 Hz, 2H, CH₂); 6.96 (d, J 9.0 Hz, 1H, Ar); 7.06 (d, J 2.1 Hz, 1H, Ar); 7.26 (dd, J 7.7, 1.1 Hz, 1H, Ar); 7.37 (t, J 7.7 Hz, 1H, Ar); 7.75 (dd, J 7.7, 1.1 Hz, 1H, Ar); 7.89 (d, J 9.0 Hz, 1H, Ar); 7.89-8.19 (bs, 2H, NH₂); 8.02 (d, J 2.1 Hz, 1H, Ar); 14.24 (bs, 1H, HCl salt). M/Z (M+H)⁺: 239.1.

### Example 80: 6-ethyl-5-(2-(6-(piperidin-1-yl)pyridin-3-yl)phenyl)pyridin-2-amine (hydrochloride)

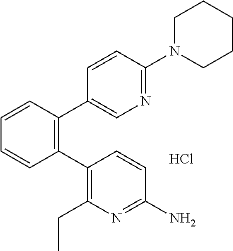 [SMILES: CCc1nc(N)ccc1-c1ccccc1-c1ccc(N2CCCCC2)nc1 ; Cl]

Example 80 was prepared according to method 2 starting from 5-(2-chlorophenyl)-6-ethyl-pyridin-2-amine 5a (100 mg, 0.43 mmol) and 2-(piperidin-1-yl)-5-(4,4,5,5-tetramethyl-1,3,2-dioxaborolan-2-yl)pyridine (186 mg, 0.65 mmol, 1.5 eq.). The crude was purified by flash chromatography (SiO₂, DCM/MeOH: 100/0 to 96/4). The obtained foam was further purified by flash chromatography (KPNH, CyHex/EtOAc: 100/0 to 70/30). The obtained foam was dissolved in a mixture of aqueous 1N HCl/ACN and the resulting solution was freeze dried to afford Example 80 as a white solid (77 mg, 45%).

¹H-NMR (DMSO-d₆, 400 MHz) δ: 1.05 (t, J 7.6 Hz, 3H, CH₂—CH₃); 1.55-1.67 (m, 6H, 3 CH₂); 2.41 (q, J 7.6 Hz, 2H, CH₂—CH₃); 3.61-3.64 (m, 4H, 2 CH₂); 6.84 (d, J 9.0 Hz, 1H, Ar); 7.16 (bs, 1H, Ar); 7.36-7.38 (m, 1H, Ar); 7.5-7.59 (m, 4H, Ar); 7.68 (t, J 9.0 Hz, 1H, Ar); 7.80 (d, J 2.2 Hz, 1H, Ar); 8.09 (bs, 2H, NH₂); 14.26 (bs, 1H, HCl salt). M/Z (M+H)⁺: 359.3. Mp: 50-80° C.

### Example 81: 6-ethyl-5-(2-(6-(trifluoromethyl)pyridin-3-yl)phenyl)pyridin-2-amine (hydrochloride)

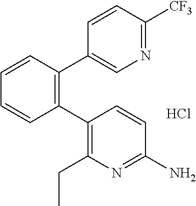 [SMILES: CCc1nc(N)ccc1-c1ccccc1-c1ccc(C(F)(F)F)nc1 ; Cl]

Example 81 was prepared according to method 2 starting from 5-(2-chlorophenyl)-6-ethyl-pyridin-2-amine 5a (100 mg, 0.43 mmol) and (6-(trifluoromethyl)pyridin-3-yl)boronic acid (123 mg, 0.65 mmol, 1.5 eq.). The crude was purified by flash chromatography (SiO₂, DCM/MeOH: 100/0 to 97/3). The obtained foam was further purified by flash chromatography (KPNH, CyHex/EtOAc: 100/0 to 70/30). The obtained foam was dissolved in a mixture of aqueous 1N HCl/ACN and the resulting solution was freeze dried to afford Example 81 as a white solid (68 mg, 42%).

¹H-NMR (DMSO-d₆, 400 MHz) δ: 0.98 (t, J 7.6 Hz, 3H, CH₂—CH₃); 2.38 (q, J 7.6 Hz, 2H, CH₂—CH₃); 6.77 (d, J 9.0 Hz, 1H, Ar); 7.42-7.45 (m, 1H, Ar); 7.59-7.66 (m, 4H, Ar); 7.85-7.94 (m, 4H, Ar+NH₂); 8.58 (d, J 1.7 Hz, 1H, Ar); 13.90 (bs, 1H, HCl salt). M/Z (M+H)⁺: 344.2. Mp>250° C.

### Example 82: 6-ethyl-5-(4-fluoro-2-(6-morpholinopyridin-3-yl)phenyl)pyridin-2-amine (hydrochloride)

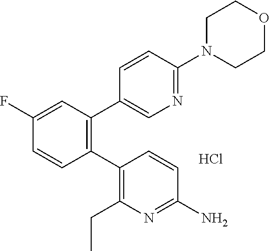 [SMILES: CCc1nc(N)ccc1-c1ccc(F)cc1-c1ccc(N2CCOCC2)nc1 ; Cl]

Example 82 was prepared according to method 2 starting from 5-(2-chloro-4-fluorophenyl)-6-ethylpyridin-2-amine 90 (97 mg, 0.39 mmol) and 4-(5-(4,4,5,5-tetramethyl-1,3,2-dioxaborolan-2-yl)pyridin-2-yl)morpholine (170 mg, 0.58 mmol, 1.5 eq.). The crude was purified by flash chromatography (KPNH, CyHex/EtOAc: 100/0 to 0/100). The obtained foam was dissolved in a mixture of aqueous 1N HCl/ACN and the resulting solution was freeze dried to afford Example 82 as a beige solid (53 mg, 31%).

¹H-NMR (DMSO-d₆, 400 MHz) δ: 1.02 (t, J 7.6 Hz, 3H, CH₂—CH₃); 2.38 (q, J 7.6 Hz, 2H, CH₂—CH₃); 3.52-3.54 (m, 4H, 2 N—CH₂); 3.69-3.71 (m, 4H, 2 O—CH₂); 6.83 (d, J 9.0 Hz, 1H, Ar); 6.98 (d, J 9.1 Hz, 1H, Ar); 7.32-7.43 (m, 3H, Ar); 7.48 (dd, J 9.1, 2.4 Hz, 1H, Ar); 7.66 (d, J 9.0 Hz, 1H, Ar); 7.92 (d, J 2.4 Hz, 1H, Ar); 8.09 (bs, 2H, NH₂); 14.25 (bs, 1H, HCl salt). M/Z (M+H)⁺: 379.2. Mp: 54-70° C.

### Example 83: 6-ethyl-5-(5-fluoro-2-(6-morpholinopyridin-3-yl)phenyl)pyridin-2-amine (hydrochloride)

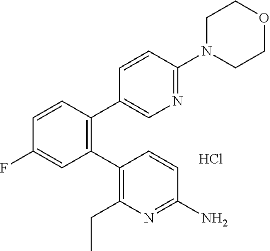 [SMILES: CCc1nc(N)ccc1-c1cc(F)ccc1-c1ccc(N2CCOCC2)nc1 ; Cl]

Example 83 was prepared according to method 2 starting from 5-(2-chloro-5-fluorophenyl)-6-ethylpyridin-2-amine 91 (100 mg, 0.40 mmol) and 4-(5-(4,4,5,5-tetramethyl-1,3,2-dioxaborolan-2-yl)pyridin-2-yl)morpholine (174 mg, 0.60 mmol, 1.5 eq.). The crude was purified by flash chromatography (KPNH, CyHex/EtOAc: 100/0 to 70/30). The obtained foam was dissolved in a mixture of aqueous 1 N HCl/ACN and the resulting solution was freeze dried to afford Example 83 as a beige solid (35 mg, 21%).

¹H-NMR (DMSO-d₆, 400 MHz) δ: 1.02 (t, J 7.6 Hz, 3H, CH₂—CH₃); 2.39 (q, J 7.6 Hz, 2H, CH₂—CH₃); 3.49-3.51 (m, 4H, 2 N—CH₂); 3.68-3.71 (m, 4H, 2O—CH₂); 6.83 (d, J 9.0 Hz, 1H, Ar); 6.95 (d, J 9.1 Hz, 1H, Ar); 7.28 (dd, J 9.4, 2.6 Hz, 1H, Ar); 7.38-7.44 (m, 2H, Ar); 7.28 (dd, J 8.6, 5.9 Hz, 1H, Ar); 7.69 (d, J 9.0 Hz, 1H, Ar); 7.92 (d, J 2.6 Hz, 1H, Ar); 8.07 (bs, 2H, NH₂); 14.16 (bs, 1H, HCl salt). M/Z (M+H)⁺: 379.3. Mp: 85-100° C.

### Example 84: 6-ethyl-5-(2-(6-morpholinopyridin-3-yl)phenyl)pyridin-2-amine (hydrochloride)

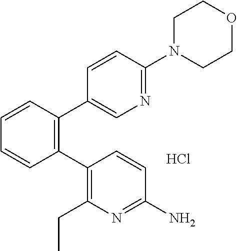 [SMILES: CCc1nc(N)ccc1-c1ccccc1-c1ccc(N2CCOCC2)nc1 ; Cl]

Example 84 was prepared according to method 2 starting from 5-(2-chlorophenyl)-6-ethylpyridin-2-amine 5a (100 mg, 0.43 mmol) and 4-(5-(4,4,5,5-tetramethyl-1,3,2-dioxaborolan-2-yl)pyridin-2-yl)morpholine (187 mg, 0.64 mmol, 1.5 eq.). The crude was purified by flash chromatography (KPNH, CyHex/EtOAc: 100/0 to 50/50). The obtained foam was further purified by (15 μm Interchim® SiO₂, DCM/MeOH: 100/0 to 97/3). The obtained foam was triturated with Et₂O (2×5 mL) and then dissolved in a mixture of aqueous 1 N HCl/ACN and the resulting solution was freeze dried to afford Example 84 as a beige solid (26 mg, 15%).

¹H-NMR (DMSO-d₆, 400 MHz) δ:1.01 (t, J 7.6 Hz, 3H, CH₂—CH₃); 2.39 (q, J 7.6 Hz, 2H, CH₂—CH₃); 3.49-3.50 (m, 4H, CH₂); 3.68-3.71 (m, 4H, CH₂); 6.82 (d, J 9.1 Hz, 1H, Ar); 6.93 (d, J 8.9 Hz, 1H, Ar); 7.34-7.36 (m, 1H, Ar); 7.43 (dd, J 8.4, 2.4 Hz, 1H, Ar); 7.47-7.57 (m, 3H, Ar); 7.67 (d, J 9.1 Hz, 1H, Ar); 7.90 (d, J 2.4 Hz, 1H, Ar); 7.99 (bs, 2H, NH₂); 14.03 (bs, 1H, HCl salt). M/Z (M+H)⁺: 361.2. Mp: 95-110° C.

### Example 85: 6-ethyl-5-(2-(5-methylpyridin-3-yl)phenyl)pyridin-2-amine (hydrochloride)

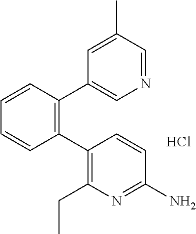 [SMILES: CCc1nc(N)ccc1-c1ccccc1-c1cncc(C)c1 ; Cl]

Example 85 was prepared according to method 2 starting from 5-(2-chlorophenyl)-6-ethylpyridin-2-amine 5a (100 mg, 0.43 mmol) and (5-methylpyridin-3-yl)boronic acid (88 mg, 0.64 mmol, 1.5 eq.). The crude was purified by flash chromatography (KPNH, CyHex/EtOAc: 100/0 to 50/50). The obtained foam was further purified by (15 μm Interchim® SiO₂, DCM/MeOH: 100/0 to 90/10). The obtained foam was dissolved in a mixture of aqueous 1N HCl/ACN and the resulting solution was freeze dried to afford Example 85 as a beige solid (34 mg, 24%).

¹H-NMR (DMSO-d₆, 400 MHz) δ: 1.01 (t, J 7.6 Hz, 3H, CH₂—CH₃); 2.32-2.39 (m, 5H, CH₂—CH₃+CH₃); 6.80 (d, J 9.0 Hz, 1H, Ar); 7.41-7.44 (m, 1H, Ar); 7.57-7.65 (m, 3H, Ar); 7.67 (d, J 9.0 Hz, 1H, Ar); 7.93 (bs, 1H, Ar); 8.07 (bs, 2H, NH₂); 8.35 (bs, 1H, Ar); 8.55 (bs, 1H, Ar); 14.18 (bs, 1H, HCl salt). M/Z (M+H)⁺: 290.2. Mp: 120-150° C.

### Example 86: 6-ethyl-5-(2-(5-methylpyridin-3-yl)phenyl)pyridin-2-amine (hydrochloride)

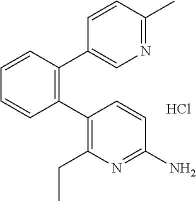 [SMILES: CCc1nc(N)ccc1-c1ccccc1-c1ccc(C)nc1 ; Cl]

Example 86 was prepared according to method 2 starting from 5-(2-chlorophenyl)-6-ethylpyridin-2-amine 5a (100 mg, 0.43 mmol) and (6-methylpyridin-3-yl)boronic acid hydrate (100 mg, 0.64 mmol, 1.5 eq.). The crude was purified by flash chromatography (KPNH, CyHex/EtOAc: 100/0 to 50/50). The obtained foam was further purified by (15 μm Interchim® SiO₂, DCM/MeOH: 100/0 to 90/10). The obtained foam was dissolved in a mixture of aqueous 1N HCl/ACN and the resulting solution was freeze dried to afford Example 86 as a beige solid (32 mg, 23%).

¹H-NMR (DMSO-d₆, 400 MHz) δ: 1.03 (t, J 7.6 Hz, 3H, CH₂—CH₃); 2.38 (q, J 7.6 Hz, 2H, CH₂—CH₃); 2.64 (s, 3H, CH₃); 6.81 (d, J 9.0 Hz, 1H, Ar); 7.42-7.44 (m, 1H, Ar); 7.58-7.65 (m, 4H, Ar); 7.67 (d, J 9.0 Hz, 1H, Ar); 7.93-7.96 (m, 1H, Ar); 8.12 (bs, 2H, NH₂); 8.54 (bs, 1H, Ar); 14.29 (bs, 1H, HCl salt). M/Z (M+H)⁺: 290.2. Mp: 90-110° C.

### Example 87: 6-ethyl-5-(2-(6-fluoropyridin-3-yl)phenyl)pyridin-2-amine (hydrochloride)

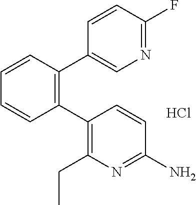 [SMILES: CCc1nc(N)ccc1-c1ccccc1-c1ccc(F)nc1 ; Cl]

Example 87 was prepared according to method 2 starting from 5-(2-chlorophenyl)-6-ethylpyridin-2-amine 5a (100 mg, 0.43 mmol) and (6-fluoropyridin-3-yl)boronic acid (91 mg, 0.64 mmol, 1.5 eq.). The crude was purified by flash chromatography (SiO₂, DCM/MeOH: 100/0 to 97/3). The obtained foam was dissolved in a mixture of aqueous 1N HCl/ACN and the resulting solution was freeze dried to afford Example 87 as a beige solid (24 mg, 17%).

¹H-NMR (DMSO-d₆, 400 MHz) δ: 0.99 (t, J 7.6 Hz, 3H, CH₂—CH₃); 2.37 (q, J 7.6 Hz, 2H, CH₂—CH₃); 6.79 (d, J 9.0 Hz, 1H, Ar); 7.16 (ddd, J 8.4, 2.8, 0.6 Hz, 1H, Ar); 7.39-7.41 (m, 1H, Ar); 7.35-7.64 (m, 4H, Ar); 7.75 (td, J 8.2, 2.6 Hz, 1H, Ar); 7.96 (bs, 2H, NH₂); 8.04 (d, J 2.6 Hz, 1H, Ar); 13.95 (bs, 1H, HCl salt). M/Z (M+H)⁺: 294.1. Mp>250° C.

### Example 88: 5-(2-(6-amino-2-ethylpyridin-3-yl)phenyl)pyridin-2-ol (hydrochloride)

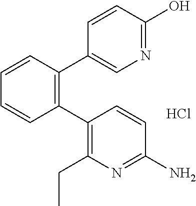 [SMILES: CCc1nc(N)ccc1-c1ccccc1-c1ccc(O)nc1 ; Cl]

Example 88 was prepared according to method 2 starting from 5-(2-chlorophenyl)-6-ethylpyridin-2-amine 5a (100 mg, 0.43 mmol) and (6-methoxypyridin-3-yl)boronic acid (99 mg, 0.64 mmol, 1.5 eq.). The crude was purified by flash chromatography (15 μm Interchim® SiO₂, DCM/MeOH: 100/0 to 96/4). The obtained product was further purified by flash chromatography (KPNH, CyHex/EtOAc: 100/0 to 70/30). The obtained foam was dissolved in a mixture of aqueous 1N HCl/ACN and the resulting solution was freeze dried. Two products were observed by UPLCMS, expected product and pyridinol suspected product. The mixture was dissolved in HCl 1M and was stirred at 25° C. for 2 days. The reaction mixture was subjected microwave irradiation at 150° C. for 5 min and was freeze dried. To finish conversion, the product was dissolved in HCl 1M and was subjected microwave irradiation at 150° C. for 5 min. The obtained foam was dissolved in a mixture of aqueous 1 N HCl/ACN and the resulting solution was freeze dried to afford Example 88 as a beige solid (56 mg, 40%).

¹H-NMR (DMSO-d₆, 400 MHz) δ: 1.05 (t, J 7.6 Hz, 3H, CH₂—CH₃); 2.42 (q, J 7.6 Hz, 2H, CH₂—CH₃); 6.27 (d, J 9.4 Hz, 1H, Ar); 6.85 (d, J 9.0 Hz, 1H, Ar); 7.16 (dd, J 9.4, 2.7 Hz, 1H, Ar); 7.21 (d, J 2.7 Hz, 1H, Ar); 7.43-7.53 (m, 3H, Ar); 7.66 (d, J 9.0 Hz, 1H, Ar); 8.04 (bs, 2H, NH₂); 14.12 (bs, 1H, HCl salt). M/Z (M+H)⁺: 292.1. Mp: 95-120° C.

### Example 89: 6-ethyl-5-(2-(6-methoxypyridin-3-yl)phenyl)pyridin-2-amine

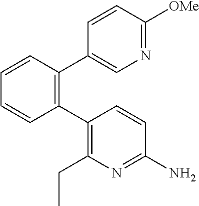 [SMILES: CCc1nc(N)ccc1-c1ccccc1-c1ccc(OC)nc1]

Example 89 was prepared according to method 2 starting from 5-(2-chlorophenyl)-6-ethylpyridin-2-amine 5a (100 mg, 0.43 mmol) and ((6-methoxypyridin-3-yl)boronic acid (99 mg, 0.64 mmol, 1.5 eq.). The crude was purified by flash chromatography (KPNH, CyHex/EtOAc: 100/0 to 70/30). The obtained foam co-evaporated with EtOAc (2×20 mL) to afford Example 89 as a white solid (30 mg, 23%).

¹H-NMR (DMSO-d₆, 400 MHz) δ: 0.85 (t, J 7.5 Hz, 3H, CH₂—CH₃); 2.01-2.10 (m, 1H, CHₐH_{b}—CH₃); 2.14-2.23 (m, 1H, CHₐH_{b}—CH₃); 3.81 (s, 3H, CH₃); 5.87 (bs, 2H, NH₂); 6.26 (d, J 8.4 Hz, 1H, Ar); 6.69 (dd, J 8.6, 0.6 Hz, 1H, Ar); 7.08 (d, J 8.4 Hz, 1H, Ar); 7.24-7.26 (m, 1H, Ar); 7.37 (d, J 2.5 Hz, 1H, Ar); 7.39-7.46 (m, 3H, Ar); 7.91 (dd, J 2.5, 0.6 Hz, 1H, Ar). M/Z (M+H)⁺: 306.2. Mp: 165-180° C.

### Example 90: 6-ethyl-5-(2-methylquinolin-8-yl)pyridin-2-amine (hydrochloride)

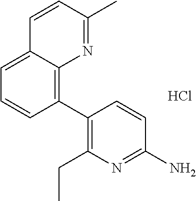 [SMILES: CCc1nc(N)ccc1-c1cccc2ccc(C)nc12 ; Cl]

Protected intermediate of Example 90 was prepared according to method 9 step 1 starting from 8-bromo-2-methylquinoline (100 mg, 0.45 mmol) and compound 7 (165 mg, 0.51 mmol, 1.1 eq.). The crude was purified by flash chromatography (SiO₂, CyHex/EtOAc: 100/0 to 90/10) to afford compound 106 (88 mg, 59%) as a white solid. M/Z (M+H)⁺: 342.2.

Example 90 was prepared according to method 9 step 2 starting from compound 106 (88 mg, 0.26 mmol). The crude was purified by flash chromatography (SiO₂, DCM/MeOH: 100/0 to 80/20). The obtained foam was dissolved in a mixture of aqueous 1N HCl/ACN and the resulting solution was freeze dried to afford Example 90 as a beige solid (93 mg, 58%).

¹H-NMR (DMSO-d₆, 400 MHz) δ: 1.11 (t, J 7.6 Hz, 3H, CH₂—CH₃); 2.41-2.46 (m, 2H, CH₂—CH₃); 2.61 (s, 3H, CH₃); 6.95 (d, J 9.0 Hz, 1H, Ar); 7.53 (d, J 8.4 Hz, 1H, Ar); 7.64-7.71 (m, 2H, Ar); 7.80 (d, J 9.0 Hz, 1H, Ar); 8.07 (dd, J 7.7, 1.8 Hz, 1H, Ar); 8.08 (bs, 2H, NH₂); 8.42 (d, J 8.4 Hz, 1H, Ar); 14.38 (bs, 1H, HCl salt). M/Z (M+H)⁺: 264.2. Mp: 110-150° C.

### Example 91: 6-ethyl-5-(4-methylquinolin-8-yl)pyridin-2-amine (hydrochloride)

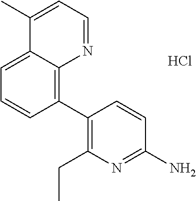 [SMILES: CCc1nc(N)ccc1-c1cccc2c(C)ccnc12 ; Cl]

Protected intermediate of Example 91 was prepared according to method 9 step 1 starting from 8-bromo-4-methylquinoline (100 mg, 0.45 mmol) and compound 7 (176 mg, 0.54 mmol, 1.2 eq.). The crude was purified by flash chromatography (SiO₂, CyHex/EtOAc: 100/0 to 85/15) to afford compound 107 (90 mg, 59%) as a white solid. M/Z (M+H)⁺: 342.2.

Example 91 was prepared according to method 9 step 2 starting from compound 107 (90 mg, 0.26 mmol). The crude was purified by flash chromatography (KPNH, CyHex/EtOAc: 100/0 to 40/60) and was triturated with Et₂O (5 mL). The obtained foam was dissolved in a mixture of aqueous 1N HCl/ACN and the resulting solution was freeze dried to afford Example 91 as a beige solid (37 mg, 47%).

¹H-NMR (DMSO-d₆, 400 MHz) δ: 1.05 (t, J 7.6 Hz, 3H, CH₂—CH₃); 2.35-2.39 (m, 1H, CHₐH_{b}—CH₃); 2.44-2.47 (m, 1H, CHₐH_{b}—CH₃); 2.84 (s, 3H, CH₃); 6.96 (d, J 9.0 Hz, 1H, Ar); 7.64-7.65 (m, 1H, Ar); 7.78 (d, J 9.0 Hz, 1H, Ar); 7.82-7.85 (m, 2H, Ar); 8.10 (bs, 2H, NH₂), 8.34 (t, J 4.8 Hz, 1H, Ar); 8.83 (d, J 4.8 Hz, 1H, Ar); 14.45 (bs, 1H, HCl salt). M/Z (M+H)⁺: 264.1. Mp>250° C.

### Example 92: 8-(6-amino-2-ethylpyridin-3-yl)quinolin-2-amine (dihydrochloride)

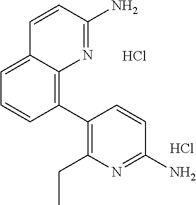 [SMILES: CCc1nc(N)ccc1-c1cccc2ccc(N)nc12 ; Cl ; Cl]

Protected intermediate of Example 92 was prepared according to method 9 step 1 starting from 8-bromoquinolin-2-amine (100 mg, 0.45 mmol) and compound 7 (219 mg, 0.67 mmol, 1.5 eq.). The crude was purified by flash chromatography (SiO₂, CyHex/EtOAc, 100/0 to 70/30) to afford compound 108 (79 mg, 51%) as a white solid. M/Z (M+H)⁺: 343.2.

Example 92 was prepared according to method 9 step 2 starting from compound 108 (79 mg, 0.23 mmol). The crude was purified by flash chromatography (KPNH, CyHex/EtOAc: 100/0 to 40/60). The resulting foam was triturated with Et₂O (10 mL). The obtained foam was dissolved in a mixture of aqueous 1N HCl/ACN and the resulting solution was freeze dried to afford Example 92 as a beige solid (38 mg, 49%).

¹H-NMR (DMSO-d₆, 400 MHz) δ: 1.07 (t, J 7.6 Hz, 3H, CH₂—CH₃); 2.28-2.37 (m, 1H, CHₐH_{b}—CH₃); 2.52-2.57 (m, 1H, CHₐH_{b}—CH₃); 6.96 (d, J 9.0 Hz, 1H, Ar); 7.15 (d, J 9.3 Hz, 1H, Ar); 7.57 (t, J 7.4 Hz, 1H, Ar); 7.65 (d, J 7.1 Hz, 1H, Ar); 7.72 (d, J 9.0 Hz, 1H, Ar); 8.02 (d, J 7.4, 1H, Ar); 8.05 (bs, 2H, NH₂); 8.44 (d, J 9.3 Hz, 1H, Ar); 9.01 (bs, 1H, NHₐNH_{b}); 9.35 (bs, 1H, NHₐNH); 12.92 (bs, 1H, HCl salt); 14.22 (bs, 1H, HCl salt). M/Z (M+H)⁺: 265.1. Mp: 200-250° C.

### Example 93: 6-ethyl-5-(7-methylquinolin-8-yl)pyridin-2-amine (hydrochloride)

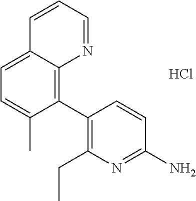 [SMILES: CCc1nc(N)ccc1-c1c(C)ccc2cccnc12 ; Cl]

Protected intermediate of Example 93 was prepared according to method 9 step 1 starting from 8-bromo-7-methylquinoline (150 mg, 0.68 mmol) and compound 7 (331 mg, 1.01 mmol, 1.5 eq.). The crude was purified by flash chromatography (SiO₂, CyHex/EtOAc, 100/0 to 70/30) to afford compound 109 (175 mg, 75%) as a yellow oil. M/Z (M+H)⁺: 342.2.

Example 93 was prepared according to method 9 step 2 starting from compound 109 (175 mg, 0.51 mmol). The crude was purified by flash chromatography (KPNH, CyHex/EtOAc: 100/0 to 50/50). The obtained foam was triturated with Et₂O (10 mL). The obtained foam was dissolved in a mixture of aqueous 1N HCl/ACN and the resulting solution was freeze dried to afford Example 93 as a beige solid (52 mg, 54%).

¹H-NMR (DMSO-d₆, 400 MHz) δ: 0.98 (t, J 7.6 Hz, 3H, CH₂—CH₃); 2.24-2.30 (m, 2H, CH₂—CH₃); 2.32 (s, 3H, CH₃); 6.99 (d, J 9.0 Hz, 1H, Ar); 7.65-7.69 (m, 1H, Ar); 7.69 (d, J 9.0 Hz, 1H, Ar); 7.73 (d, J 8.4 Hz, 1H, Ar); 8.06 (bs, 2H, NH₂); 8.11 (d, J 8.4 Hz, 1H, Ar); 8.62 (d, J 8.0 Hz, 1H, Ar); 8.88 (dd, J 4.4, 1.6 Hz, 1H, Ar); 14.35 (bs, 1H, HCl salt). M/Z (M+H)⁺: 264.1. Mp: 90-130° C.

### Example 94: 5-(2-ethoxyquinolin-8-yl)-6-ethylpyridin-2-amine (hydrochloride)

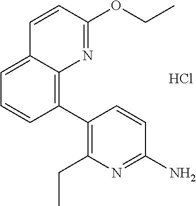 [SMILES: CCOc1ccc2cccc(-c3ccc(N)nc3CC)c2n1 ; Cl]

Protected intermediate of Example 94 was prepared according to method 9 step 1 starting from 8-bromo-2-ethoxyquinoline (150 mg, 0.60 mmol) and compound 7 (291 mg, 0.89 mmol, 1.5 eq.). The crude was purified by HCl flash chromatography (SiO₂, CyHex/EtOAc: 100/0 to 85/15). The obtained foam was further purified by flash chromatography (KPNH, CyHex/EtOAc: 100/0 to 90/10) to afford compound 110 (131 mg, 59%) as a white solid. M/Z (M+H)⁺: 372.3 Example 94 was prepared according to method 9 step 2 starting from compound 110 (131 mg, 0.35 mmol). The crude was purified by flash chromatography (KPNH, CyHex/EtOAc: 100/0 to 70/30). The obtained foam was further purified by flash chromatography (15 μm Interchim® SiO₂, DCM/MeOH: 100/0 to 95/5). The resulting compound was triturated with Et₂O (5 mL). The obtained foam was dissolved in a mixture of aqueous 1N HCl/ACN and the resulting solution was freeze dried to afford Example 94 as a white solid (17 mg, 15%).

¹H-NMR (DMSO-d₆, 400 MHz) δ: 1.05 (t, J 7.6 Hz, 3H, CH₂—CH₃); 1.25 (t, J 7.1 Hz, 3H, O—CH₂—CH₃); 2.52-2.56 (m, 2H, CH₂—CH₃); 4.16-4.22 (m, 2H, O—CH₂—CH₃); 6.92 (d, J 9.0 Hz, 1H, Ar); 7.03 (d, J 8.9 Hz, 1H, Ar); 7.52 (dd, J 8.0, 7.2 Hz, 1H, Ar); 7.63 (dd, J 7.2, 1.5 Hz, 1H, Ar); 7.85 (d, J 9.0 Hz, 1H, Ar); 7.84-7.90 (bs, 2H, NH₂); 7.98 (dd, J 8.0, 1.5 Hz, 1H, Ar); 8.31 (d, J 8.9 Hz, 1H, Ar); 14.02 (bs, 1H, HCl salt). M/Z (M+H)⁺: 294.0. Mp: 35-70° C.

### Example 95: 6-ethyl-5-(3-methylquinolin-8-yl)pyridin-2-amine (hydrochloride)

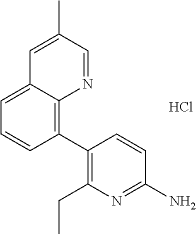 [SMILES: CCc1nc(N)ccc1-c1cccc2cc(C)cnc12 ; Cl]

Protected intermediate of Example 95 was prepared according to method 9 step 1 starting from 8-bromo-3-methylquinoline (150 mg, 0.68 mmol) and compound 7 (331 mg, 1.01 mmol, 1.5 eq.). The crude was purified by flash chromatography (SiO₂, CyHex/EtOAc: 100/0 to 80/20) to compound 111 (193 mg, 84%) as a beige solid. M/Z (M+H)⁺: 342.2

Example 95 was prepared according to method 9 step 2 starting from compound 111 (193 mg, 0.57 mmol). The crude was purified by flash chromatography (KPNH, DCM/MeOH: 100/0 to 95/5) and was triturated with Et₂O (5 mL). The obtained foam was dissolved in a mixture of aqueous 1N HCl/ACN and the resulting solution was freeze dried to afford Example 95 as a beige solid (128 mg, 76%).

¹H-NMR (DMSO-d₆, 400 MHz) δ: 1.05 (t, J 7.6 Hz, 3H, CH₂—CH₃); 2.35-2.48 (m, 2H, CH₂—CH₃); 2.52 (s, 3H, CH₃); 6.95 (d, J 9.0 Hz, 1H, Ar); 7.68-7.74 (m, 2H, Ar); 7.79 (d, J 9.0 Hz, 1H, Ar); 8.05 (m, 3H, Ar+NH₂); 8.35 (bs, 1H, Ar); 8.79 (d, J 2.2 Hz, 1H, Ar); 14.33 (bs, 1H, HCl salt). M/Z (M+H)⁺: 264.0. Mp>250° C.

### Example 96: 6-ethyl-5-(5-methylquinolin-8-yl)pyridin-2-amine (hydrochloride)

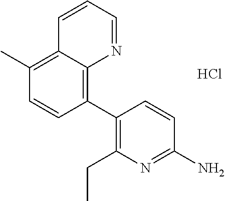 [SMILES: CCc1nc(N)ccc1-c1ccc(C)c2cccnc12 ; Cl]

Protected intermediate of Example 96 was prepared according to method 9 step 1 starting from 8-bromo-5-methylquinoline (150 mg, 0.68 mmol) and compound 7 (331 mg, 1.01 mmol, 1.5 eq.). The crude was purified by flash chromatography (SiO₂, CyHex/EtOAc: 100/0 to 80/20) to afford compound 112 (216 mg, 94%) as a white solid. M/Z (M+H)⁺: 342.2

Example 96 was prepared according to method 9 step 2 starting from compound 112 (216 mg, 0.63 mmol). The crude was purified by flash chromatography (KPNH, DCM/MeOH: 100/0 to 95/5) and was triturated with Et₂O (5 mL). The obtained foam was dissolved in a mixture of aqueous 1N HCl/ACN and the resulting solution was freeze dried to afford Example 96 as a beige solid (161 mg, 85%).

¹H-NMR (DMSO-d₆, 400 MHz) δ: 1.06 (t, J 7.6 Hz, 3H, CH₂—CH₃); 2.34-2.48 (m, 2H, CH₂—CH₃); 2.75 (s, 3H, CH₃); 6.94 (d, J 9.0 Hz, 1H, Ar); 7.61 (d, J 7.3 Hz, 1H, Ar); 7.68 (d, J 7.3 Hz, 1H, Ar); 7.71 (dd, J 8.5, 4.4 Hz, 1H, Ar); 7.77 (d, J 9.0 Hz, 1H, Ar); 8.04 (bs, 2H, NH₂); 8.68 (dd, J 8.6, 1.5 Hz, 1H, Ar); 8.92 (dd, J 4.4, 1.5 Hz, 1H, Ar); 14.35 (bs, 1H, HCl salt). M/Z (M+H)⁺: 264.0. Mp>250° C.

### Example 97: 6-ethyl-5-(3-fluoroquinolin-8-yl)pyridin-2-amine (hydrochloride)

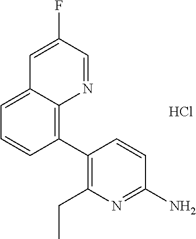 [SMILES: CCc1nc(N)ccc1-c1cccc2cc(F)cnc12 ; Cl]

Protected intermediate of Example 97 was prepared according to method 9 step 1 starting from 8-bromo-3-fluoroquinoline (150 mg, 0.66 mmol) and compound 7 (325 mg, 1.00 mmol, 1.5 eq.). The crude was purified by flash chromatography (SiO₂, CyHex/EtOAc: 100/0 to 80/20) to afford compound 113 (280 mg) as a yellow oil. M/Z (M+H)⁺: 346.2

Example 97 was prepared according to method 9 step 2 starting from compound 113 (280 mg). The crude was purified by flash chromatography (KPNH, DCM/MeOH: 100/0 to 95/5).

The obtained foam was further purified by flash chromatography (15 μm Interchim® SiO₂, DCM/MeOH: 100/0 to 90/10) and was triturated with Et₂O (5 mL). The obtained foam was dissolved in a mixture of aqueous 1N HCl/ACN and the resulting solution was freeze dried to afford Example 97 as a beige solid (116 mg, 58% over 2 steps).

¹H-NMR (DMSO-d₆, 400 MHz) δ: 1.05 (t, J 7.6 Hz, 3H, CH₂—CH₃); 2.36-2.48 (m, 2H, CH₂—CH₃); 6.94 (d, J 9.0 Hz, 1H, Ar); 7.73 (dd, J 7.1, 1.6 Hz, 1H, Ar); 7.77 (d, J 8.0 Hz, 1H, Ar); 7.80 (d, J 9.0 Hz, 1H, Ar); 7.99 (bs, 2H, NH₂); 8.12 (dd, J 8.0, 1.6 Hz, 1H, Ar); 8.38 (dd, J 9.4, 2.9 Hz, 1H, Ar); 8.92 (d, J 2.9 Hz, 1H, Ar); 14.18 (bs, 1H, HCl salt). M/Z (M+H)⁺: 267.9. Mp: 210-245° C.

### Example 98: 6-ethyl-5-(7-methoxyquinolin-8-yl)pyridin-2-amine (hydrochloride)

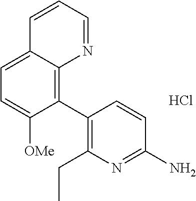 [SMILES: CCc1nc(N)ccc1-c1c(OC)ccc2cccnc12 ; Cl]

Protected intermediate of Example 98 was prepared according to method 9 step 1 starting from 8-bromo-7-methoxyquinoline (125 mg, 0.53 mmol) and compound 7 (257 mg, 0.79 mmol, 1.5 eq.). The crude was purified by flash chromatography (SiO₂, CyHex/EtOAc: 100/0 to 80/20) to afford compound 114 (164 mg) as a light yellow oil. M/Z (M+H)⁺: 358.3.

Example 98 was prepared according to method 9 step 2 starting from compound 114 (164 mg). The crude was purified by flash chromatography (KPNH, DCM/MeOH: 100/0 to 95/5) and was triturated with Et₂O (5 mL). The obtained foam was dissolved in a mixture of aqueous 1N HCl/ACN and the resulting solution was freeze dried to afford Example 98 as a white solid (92 mg, 55% over 2 steps).

¹H-NMR (DMSO-d₆, 400 MHz) δ: 1.02 (t, J 7.6 Hz, 3H, CH₂—CH₃); 2.26-2.44 (m, 2H, CH₂—CH₃); 3.96 (s, 3H, CH₃); 6.96 (d, J 9.0 Hz, 1H, Ar); 7.61-7.65 (m, 1H, Ar); 7.68 (d, J 9.0 Hz, 1H, Ar); 7.83 (d, J 9.2 Hz, 1H, Ar); 8.07 (bs, 2H, NH₂); 8.32 (d, J 9.2 Hz, 1H, Ar); 8.71-8.74 (m, 1H, Ar); 8.88 (dd, J 4.6, 1.4 Hz, 1H, Ar); 14.40 (bs, 1H, HCl salt). M/Z (M+H)⁺: 280.2. Mp: 230-250° C.

### Example 99: 6-ethyl-5-(2-(trifluoromethyl)quinolin-8-yl)pyridin-2-amine (hydrochloride)

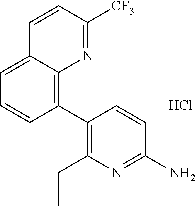 [SMILES: CCc1nc(N)ccc1-c1cccc2ccc(C(F)(F)F)nc12 ; Cl]

Protected intermediate of Example 99 was prepared according to method 9 step 1 starting from 8-bromo-2-(trifluoromethyl)quinoline (125 mg, 0.45 mmol) and compound 7 (222 mg, 0.68 mmol, 1.5 eq.). The crude was purified by flash chromatography (SiO₂, CyHex/EtOAc: 100/0 to 80/20) to afford 8-(6-(2,5-dimethyl-1H-pyrrol-1-yl)-2-ethylpyridin-3-yl)-2-(trifluoromethyl)quinoline 115 (169 mg, 94%) as a yellow oil. M/Z (M+H)⁺: 396.2.

Example 99 was prepared according to method 9 step 2 starting from compound 115 (169 mg, 0.43 mmol). The crude was purified by flash chromatography (KPNH, DCM/MeOH: 100/0 to 95/5). The obtained foam was further purified by flash chromatography (KPNH, CyHex/EtOAc: 100/0 to 50/50). The obtained foam was dissolved in a mixture of aqueous 1N HCl/ACN and the resulting solution was freeze dried to afford Example 99 as a white solid (70 mg, 46%).

¹H-NMR (DMSO-d₆, 400 MHz) δ: 1.07 (t, J 7.6 Hz, 3H, CH₂—CH₃); 2.44 (bs, 2H, CH₂—CH₃); 6.95 (d, J 9.0 Hz, 1H, Ar); 7.84 (d, J 9.0 Hz, 1H, Ar); 7.88-7.93 (m, 2H, Ar); 7.99 (bs, 2H, NH₂); 8.05 (d, J 8.6 Hz, 1H, Ar); 8.26-8.30 (m, 1H, Ar); 8.84 (d, J 8.6 Hz, 1H, Ar); 14.14 (bs, 1H, HCl salt). M/Z (M+H)⁺: 318.0. Mp: 90-130° C.

### Example 100: 6-ethyl-5-(1,7-naphthyridin-8-yl)pyridin-2-amine (hydrochloride)

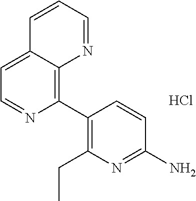 [SMILES: CCc1nc(N)ccc1-c1nccc2cccnc12 ; Cl]

Protected intermediate of Example 100 was prepared according to method 9 step 1 starting from 8-chloro-1,7-naphthyridine (125 mg, 0.76 mmol) and compound 7 (372 mg, 1.14 mmol, 1.5 eq.). The crude was purified by flash N chromatography (SiO₂, CyHex/EtOAc: 100/0 to 70/30) to compound 116 (170 N mg, 68%) as a yellow oil. M/Z (M+H)⁺: 396.2.

Example 100 was prepared according to method 9 step 2 starting from compound 116 (170 mg, 0.76 mmol). The crude was purified by flash chromatography (KPNH, DCM/MeOH: 100/0 to 95/5). The obtained foam was dissolved in a mixture of aqueous 1 N HCl/ACN and the resulting solution was freeze dried to afford Example 100 as an orange solid (124 mg, 83%). ¹H-NMR (DMSO-d₆, 400 MHz) δ: 1.11 (t, J 7.6 Hz, 3H, CH₂—CH₃); 2.58 (q, J 7.6 Hz, 2H, CH₂—CH₃); 6.97 (d, J 9.0 Hz, 1H, Ar); 7.85 (dd, J 8.4, 4.2 Hz, 1H, Ar); 8.00 (d, J 9.0 Hz, 1H, Ar); 8.04 (d, J 5.6 Hz, 1H, Ar); 8.09 (bs, 2H, NH₂); 8.55 (dd, J 8.4, 1.8 Hz, 1H, Ar); 8.73 (d, J 5.6 Hz, 1H, Ar); 9.03 (dd, J 4.2, 1.8 Hz, 1H, Ar); 14.22 (bs, 1H, HCl salt). M/Z (M+H)⁺: 251.1. Mp: 160-200° C.

### Example 101: 6-ethyl-5-(quinoxalin-5-yl)pyridin-2-amine (hydrochloride)

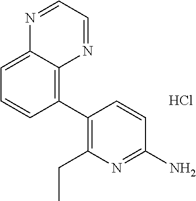 [SMILES: CCc1nc(N)ccc1-c1cccc2nccnc12 ; Cl]

Protected intermediate of Example 101 was prepared according to method 9 step 1 starting from 5-bromoquinoxaline (115 mg, 0.55 mmol, 1.2 eq.) and compound 7 (150 mg, 0.46 mmol). The crude was purified by flash chromatography (SiO₂, CyHex/EtOAc: 100/0 to 80/20) to afford compound 117 (104 mg, 69%) as an orange solid. M/Z (M+H)⁺: 329.1.

Example 101 was prepared according to method 9 step 2 starting from compound 117 (104 mg, 0.32 mmol). The crude was purified by flash chromatography (KPNH, CyHex/EtOAc: 100/0 to 20/80). The obtained foam was dissolved in a mixture of aqueous 1N HCl/ACN and the resulting solution was freeze dried to afford Example 101 as a pink solid (47 mg, 52%).

1H-NMR (DMSO-d6, 400 MHz) δ:1.06 (t, J 7.6 Hz, 3H, CH₃); 2.39-2.45 (m, 2H, CH₂); 6.94 (d, J 9.0 Hz, 1H, Ar); 7.82 (d, J 9.0 Hz, 1H, Ar); 7.86 (dd, J 7.1, 1.3 Hz, 1H, Ar); 7.96-8.00 (m, 3H, Ar+NH₂); 8.22 (dd, J 8.3, 1.3 Hz, 1H, Ar); 8.93 (d, J 1.8 Hz, 1H, Ar); 9.02 (d, J 1.8 Hz, 1H, Ar); 14.19 (bs, 1H, HCl salt). M/Z (M+H)⁺: 251.0. Mp>250° C.

### Example 102: 6-ethyl-5-(imidazo[1,2-a]pyridin-8-yl)pyridin-2-amine (hydrochloride)

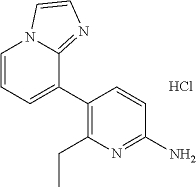 [SMILES: CCc1nc(N)ccc1-c1cccn2ccnc12 ; Cl]

Protected intermediate of Example 102 was prepared according to method 9 step 1 starting from 8-bromoimidazo[1,2-a]pyridine (125 mg, 0.63 mmol) and compound 7 (202 mg, 0.63 mmol, 1.0 eq.). The crude was purified by flash chromatography (15 μm Interchim® SiO₂, DCM/MeOH: 100/0 to 95/5) to afford compound 118 (126 mg, 63%) as a light brown solid. M/Z (M+H)⁺: 316.9.

Example 102 was prepared according to method 9 step 2 starting from compound 118 (126 mg, 0.40 mmol). The crude was purified by flash chromatography (KPNH, DCM/MeOH: 100/0 to 96/4). The obtained foam was dissolved in a mixture of aqueous 1N HCl/ACN and the resulting solution was freeze dried to afford Example 102 as a beige solid (91 mg, 84%).

¹H-NMR (DMSO-d₆, 400 MHz) δ: 1.12 (t, J 7.6 Hz, 3H, CH₂—CH₃); 2.52-2.61 (m, 2H, CH₂—CH₃); 7.01 (d, J 9.0 Hz, 1H, Ar); 7.59 (t, J 7.0 Hz, 1H, Ar); 7.82 (d, J 9.0 Hz, 1H, Ar); 7.89 (d, J 7.0 Hz, 1H, Ar); 8.22 (d, J 1.9 Hz, 1H, Ar); 8.32 (bs, 2H, NH₂); 8.48 (d, J 2.0 Hz, 1H, Ar); 8.99 (dd, J 7.0, 0.8 Hz, 1H, Ar); 14.78 (bs, 1H, HCl salt). M/Z (M+H)⁺: 239.0. Mp: 95-135° C.

### Example 103: 6-ethyl-5-(imidazo[1,2-a]pyridin-5-yl)pyridin-2-amine (hydrochloride)

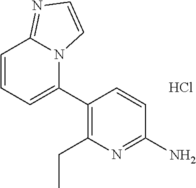 [SMILES: CCc1nc(N)ccc1-c1cccc2nccn12 ; Cl]

Protected intermediate of Example 103: Under Argon, to a solution of 5-bromoimidazo[1,2-a]pyridine (250 mg, 1.28 mmol) in DMF (6.5 mL), compound 7 (620 mg, 1.90 mmol, 1.5 eq.) and K₂CO₃ (703 mg, 4.0 Eq, 5.10 mmol) were added. The reaction mixture was sparged with argon for 10 min before addition of Pd(dppf)Cl₂ (46.5 mg, 63.5 μmol, 0.05 eq.). The reaction mixture was heated at 110° C. for 2 h. The reaction mixture was filtered through a pad of Celite® and the cake was washed with DCM (70 mL). The organic layer was hydrolyzed with NH₄Cl sat. (70 mL) and extracted twice with DCM (70 mL). The organic layers were washed with brine (50 mL), dried over magnesium sulfate and concentrated. The crude was purified twice by flash chromatography (SiO₂, DCM/MeOH: 100/0 to 95/5) to obtain compound 119 (190 mg, 47%) as a light brown oil. M/Z (M+H)⁺: 317.1.

Example 103 was prepared according to method 9 step 2 starting from compound 119 (190 mg, 0.60 mmol). The crude was purified by flash chromatography (KPNH, DCM/MeOH: 100/0 to 98/2). The obtained foam was dissolved in a mixture of aqueous 1N HCl/ACN and the resulting solution was freeze dried to afford Example 103 as a beige solid (127 mg, 77%).

¹H-NMR (DMSO-d₆, 400 MHz) δ: 1.15 (t, J 7.6 Hz, 3H, CH₂—CH₃); 2.32-2.46 (m, 1H, CHₐH_{b}—CH₃); 2.54-2.63 (m, 1H, CHₐH_{b}—CH₃); 7.04 (d, J 9.1 Hz, 1H, Ar); 7.52 (dd, J 7.0, 1.2 Hz, 1H, Ar); 7.87 (d, J 9.1 Hz, 1H, Ar); 8.03 (dd, J 9.1, 7.0 Hz, 1H, Ar); 8.10 (t, J 9.1 Hz, 1H, Ar); 8.13 (dd, J 2.2, 0.5 Hz, 1H, Ar); 8.27 (d, J 2.2 Hz, 1H, Ar); 8.49 (bs, 2H, NH₂); 14.96 (bs, 1H, HCl salt). M/Z (M+H)⁺: 239.0. Mp: 115-155° C.

### Example 104: 6-ethyl-5-(pyrazolo[1,5-a]pyridin-7-yl)pyridin-2-amine (hydrochloride)

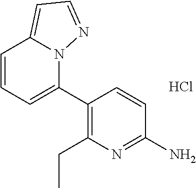 [SMILES: CCc1nc(N)ccc1-c1cccc2ccnn12 ; Cl]

Protected intermediate of Example 104 was prepared according to method 9 step 1 starting from 7-bromopyrazolo[1,5-a]pyridine (150 mg, 0.76 mmol) and compound 7 (298 mg, 0.91 mmol, 1.2 eq.). The crude was purified by flash chromatography (SiO₂, CyHex/EtOAc: 100/0 to 85/15) to afford compound 120 (140 mg, 58%) as yellow oil. M/Z (M+H)⁺: 317.1.

Example 104 was prepared according to method 9 step 2 starting from compound 120 (140 mg, 0.44 mmol). The crude was purified by flash chromatography (SiO₂, DCM/MeOH: 100/0 to 97/3). The obtained product was triturated in Et₂O (2×2 mL). The obtained foam was dissolved in a mixture of aqueous 1N HCl/ACN and the resulting solution was freeze dried to afford Example 104 as an orange solid (73 mg, 60%).

1H-NMR (DMSO-d6, 400 MHz) δ: 1.11 (t, J 7.6 Hz, 3H, CH₃); 2.52-2.53 (m, 2H, CH₂); 6.76 (d, J 2.3 Hz, 1H, Ar); 6.96 (dd, J 6.7, 1.4 Hz, 1H, Ar); 6.99 (d, J 9.1 Hz, 1H Ar); 7.32 (dd, J 8.8, 6.8 Hz, 1H, Ar); 7.82 (dd, J 8.8, 1.3 Hz, 1H, Ar); 7.93 (d, J 9.1 Hz, 1H, Ar); 7.98 (d, J 2.3 Hz, 1H, Ar); 8.25 (bs, 2H, NH₂); 14.52 (bs, 1H, HCl salt). M/Z (M+H)⁺: 238.9. Mp: 234-243° C.

### Example 105: 5-(7-(difluoromethoxy)quinolin-8-yl)-6-ethylpyridin-2-amine (hydrochloride)

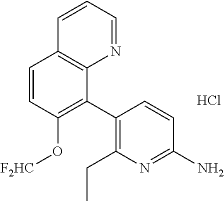 [SMILES: CCc1nc(N)ccc1-c1c(OC(F)F)ccc2cccnc12 ; Cl]

Protected intermediate of Example 105 was prepared according to method 9 step 1 starting from 8-bromo-7-(difluoromethoxy)quinoline 64 (129 mg, 0.47 mmol) and compound 7 (230 mg, 0.71 mmol, 1.5 eq.). The crude was purified by flash chromatography (15 μm Interchim® SiO₂, DCM/MeOH: 100/0 to 97/3) to afford compound 121 (82 mg) as yellow oil. M/Z (M+H)⁺: 394.2

Example 105 was prepared according to method 9 step 2 starting from compound 121 (82 mg). The crude was purified by flash chromatography (KPNH, DCM/MeOH: 100/0 to 97/3). The obtained foam was dissolved in a mixture of aqueous 1N HCl/ACN and the resulting solution was freeze dried to afford Example 105 as a beige solid (35 mg, 21% over 2 steps).

¹H-NMR (DMSO-d₆, 400 MHz) δ: 1.02 (t, J 7.6 Hz, 3H, CH₂—CH₃); 2.29-2.40 (m, 2H, CH₂—CH₃); 6.97 (d, J 9.0 Hz, 1H, Ar); 7.38 (t, J 73.3 Hz, 1H, CHF₂); 7.61 (dd, J 8.3, 4.2 Hz, 1H, Ar); 7.72 (d, J 9.2 Hz, 1H, Ar); 7.74 (d, J 9.0 Hz, 1H, Ar); 8.04 (bs, 2H, NH₂); 8.26 (d, J 9.2 Hz, 1H, Ar); 8.53 (dd, J 8.3, 1.6 Hz, 1H, Ar); 8.90 (dd, J 4.2, 1.6 Hz, 1H, Ar); 14.24 (bs, 1H, HCl salt). M/Z (M+H)⁺: 316.0. Mp: 120-160° C.

### Example 106: 6-ethyl-5-(1,2,3,4-tetrahydroquinolin-8-yl)pyridin-2-amine (hydrochloride)

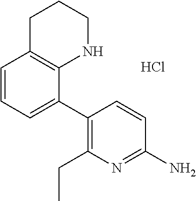 [SMILES: CCc1nc(N)ccc1-c1cccc2c1NCCC2 ; Cl]

Protected intermediate of Example 106 was prepared according to method 9 step 1 starting from 8-bromo-1,2,3,4-tetrahydroquinoline hydrochloride (165 mg, 0.67 mmol) and compound 7 (262 mg, 0.80 mmol, 1.2 eq.) and using 3.0 eq. of K₂CO₃. The crude was purified by flash chromatography (SiO₂, CyHex/EtOAc: 100/0 to 90/10) to afford compound 122 (218 mg) as a colorless oil. M/Z (M+H)⁺: 332.2

Example 106 was prepared according to method 9 step 2 starting from compound 122 (218 mg). The crude was purified by flash chromatography (KPNH, CyHex/EtOAc: 100/0 to 50/50).

The product was triturated in Et₂O (4 mL) and pentane (4 mL). The obtained foam was dissolved in a mixture of aqueous 1N HCl/ACN and the resulting solution was freeze dried to afford Example 106 as a white solid (51 mg, 27% over 2 steps).

¹H-NMR (D20, 400 MHz) δ: 1.17 (t, J 7.6 Hz, 3H, CH₃); 2.06-2.16 (m, 2H, CH₂); 2.46-2.55 (m, 1H, CH—H); 2.61-2.70 (m, 1H, CH—H); 3.00 (t, J 6.7 Hz, 2H, CH₂); 3.33-3.39 (m, 1H, CH—H); 3.44-3.50 (m, 1H, CH—H); 6.99 (d, J 9.0 Hz, 1H, Ar); 7.20 (d, J 7.5 Hz, 1H, Ar); 7.30 (t, J 7.5 Hz, 1H, Ar); 7.41 (d, J 7.7 Hz, 1H, Ar); 7.75 (d, J 9.0 Hz, 1H, Ar). M/Z (M+H)⁺: 254.0. Mp: 147-160° C.

### Example 107: 6-ethyl-5-(7-fluoro-3-phenylquinolin-8-yl)pyridin-2-amine (hydrochloride)

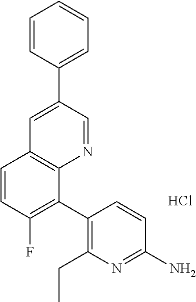 [SMILES: CCc1nc(N)ccc1-c1c(F)ccc2cc(-c3ccccc3)cnc12 ; Cl]

Protected intermediate of Example 107 was prepared according to method 9 step 1 starting from 8-bromo-7-fluoro-3-phenylquinoline 66 (105 mg, 0.35 mmol) and compound 7 (170 mg, 0.52 mmol, 1.5 eq.). The crude was purified by flash chromatography (15 μm Interchim® SiO₂, CyHex/EtOAc: 100/0 to 75/25) to afford compound 123 (129 mg) as a colorless oil. M/Z (M+H)⁺: 422.2

Example 107 was prepared according to method 9 step 2 starting from compound 123 (129 mg). The crude was purified by flash chromatography (15 μm Interchim® SiO₂, DCM/MeOH: 100/0 to 96/4). The obtained product was further purified by preparative HPLC (H₂O (0.5 wt. % HCOOH)/CH₃CN (0.5 wt. % HCOOH): 80/20 to 40/60). Then, volatiles were removed under vacuum. The obtained solution was diluted in a mixture of aqueous 1N HCl/ACN and the resulting solution was freeze dried to afford Example 107 as a white solid (67 mg, 51% over 2 steps).

1H-NMR (DMSO-d6, 400 MHz) δ: 1.08 (t, J 7.5 Hz, 3H, CH₂—CH₃); 2.37-2.48 (m, 2H, CH₂—CH₃); 7.01 (d, J 9.0 Hz, 1H, Ar); 7.45-7.50 (m, 1H, Ar), 7.54-7.60 (m, 2H, Ar); 7.77 (t, J 9.1 Hz, 1H, Ar); 7.80-7.90 (m, 3H, Ar); 8.13 (bs, 2H, NH₂); 8.30 (dd, J 9.1, 6.4 Hz, 1H, Ar); 8.82 (d, J 2.4 Hz, 1H, Ar); 9.26 (d, J 2.4 Hz, 1H, Ar); 14.43 (bs, 1H, HCl salt). M/Z (M+H)⁺: 344.1. Mp>250° C.

### Example 108: 5-(5,7-difluoroquinolin-8-yl)-6-ethylpyridin-2-amine (hydrochloride)

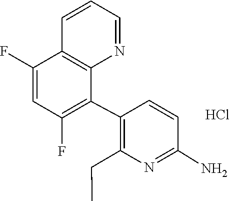 [SMILES: CCc1nc(N)ccc1-c1c(F)cc(F)c2cccnc12 ; Cl]

Protected intermediate of Example 108 was prepared according to method 9 step 1 starting from 8-bromo-5,7-difluoroquinoline 24 (150 mg, 0.62 mmol) and compound 7 (301 mg, 0.92 mmol, 1.5 eq.). The crude was purified by flash chromatography (15 μm Interchim® SiO₂, CyHex/EtOAc: 100/0 to 50/50) to afford compound 124 (196 mg, 88%) as a white solid. M/Z (M+H)⁺: 364.2.

Example 108 was prepared according to method 9 step 2 starting from compound 124 (190 mg, 0.52 mmol). The crude was purified by flash chromatography (15 μm Interchim® SiO₂, DCM/MeOH: 100/0 to 95/5). The obtained foam was dissolved in a mixture of aqueous 1N HCl/ACN and the resulting solution was freeze dried to afford Example 108 as a beige solid (90 mg, 53%).

¹H-NMR (DMSO-d₆, 400 MHz) δ:1.06 (t, J 7.5 Hz, 3H, CH₂—CH₃); 2.36-2.47 (m, 2H, CH₂—CH₃); 6.98 (d, J 8.7 Hz, 1H, Ar); 7.70 (dd, 8.4, J 4.1 Hz 1H, Ar); 7.78-7.85 (m, 2H, Ar); 8.02 (bs, 2H, NH₂); 8.60 (dd, J 8.4, 1.7 Hz, 1H, Ar); 8.98 (dd, J 4.1, 1.7 Hz, 1H, Ar); 14.10 (bs, 1H, HCl salt). M/Z (M+H)⁺: 285.9. Mp: 126-136° C.

### Example 109: 6-ethyl-5-(7-(trifluoromethyl)quinolin-8-yl)pyridin-2-amine (hydrochloride)

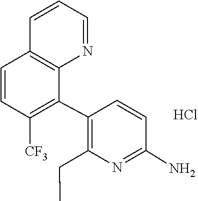 [SMILES: CCc1nc(N)ccc1-c1c(C(F)(F)F)ccc2cccnc12 ; Cl]

Protected intermediate of Example 109 was prepared according to method 9 step 1 starting from 8-bromo-7-(trifluoromethyl)quinoline 25 (125 mg, 0.45 mmol) and compound 7 (222 mg, 0.68 mmol, 1.5 eq.). The crude was purified by flash chromatography (SiO₂, CyHex/EtOAc: 100/0 to 40/60) to afford compound 125 (145 mg, 81%) as a yellow oil. M/Z (M+H)⁺: 396.2.

Example 109 was prepared according to method 9 step 2 starting from compound 125 (145 mg, 0.37 mmol). The crude was purified by flash chromatography (KPNH, CyHex/EtOAc: 100/0 to 70/30). The obtained foam was dissolved in a mixture of aqueous 1N HCl/ACN and the resulting solution was freeze dried to afford Example 109 as a light yellow solid (100 mg, 77%).

¹H-NMR (DMSO-d₆, 400 MHz) δ: 0.98 (t, J 7.6 Hz, 3H, CH₂—CH₃); 2.13-2.32 (m, 2H, CH₂—CH₃); 6.97 (d, J 9.0 Hz, 1H, Ar); 7.74-7.78 (m, 2H, Ar); 8.06 (d, J 8.8 Hz, 1H, Ar); 8.15 (bs, 2H, NH₂); 8.38 (d, J 8.8 Hz, 1H, Ar); 8.62 (dd, J 8.4, 1.8 Hz, 1H, Ar); 8.99 (dd, J 4.2, 1.8 Hz, 1H, Ar); 14.47 (bs, 1H, HCl salt). M/Z (M+H)⁺: 318.1. Mp: 150-180° C.

### Example 110: 5-(7-chloroquinolin-8-yl)-6-ethylpyridin-2-amine (hydrochloride)

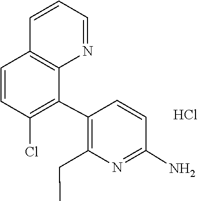 [SMILES: CCc1nc(N)ccc1-c1c(Cl)ccc2cccnc12 ; Cl]

Protected intermediate of Example 110 was prepared according to method 9 step 1 starting from 8-bromo-7-chloroquinoline 26 (435 mg, 1.79 mmol) and compound 7 (644 mg, 1.97 mmol, 1.1 eq.). The crude was purified by flash chromatography (SiO₂, DCM/MeOH: 100/0 to 98/2). The obtained foam was further purified by flash chromatography (15 μm Interchim® SiO₂, DCM/MeOH: 100/0 to 98/2) to afford compound 126 (395 mg, 61%) as a yellow oil. M/Z (M[³⁵Cl]+H)⁺: 362.1.

Example 110 was prepared according to method 9 step 2 starting from compound 126 (145 mg, 0.37 mmol). The crude was purified by flash chromatography (KPNH, DCM/MeOH: 100/0 to 98/2). The obtained foam was dissolved in a mixture of aqueous 1N HCl/ACN and the resulting solution was freeze dried to afford Example 110 as a beige solid (35 mg, 32%).

¹H-NMR (DMSO-d₆, 400 MHz) δ: 1.00 (t, J 7.6 Hz, 3H, CH₂—CH₃); 2.30-2.36 (m, 2H, CH₂—CH₃); 7.00 (d, J 9.0 Hz, 1H, Ar); 7.64 (dd, J 8.2, 4.2 Hz, 1H, Ar); 7.75 (d, J 9.0 Hz, 1H, Ar); 7.86 (d, J 8.8 Hz, 1H, Ar); 8.08 (bs, 2H, NH₂); 8.19 (d, J 8.8 Hz, 1H, Ar); 8.54 (dd, J 8.4, 1.8 Hz, 1H, Ar); 8.90 (dd, J 4.2, 1.8 Hz, 1H, Ar); 14.38 (bs, 1H, HCl salt). M/Z (M[³⁵Cl]+H)⁺: 284.6. Mp: 140-180° C.

### Example 111: 5-(6,7-difluoroquinolin-8-yl)-6-ethylpyridin-2-amine (hydrochloride)

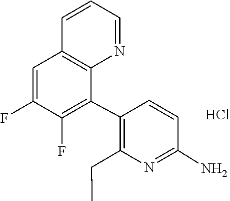 [SMILES: CCc1nc(N)ccc1-c1c(F)c(F)cc2cccnc12 ; Cl]

Protected intermediate of Example 111 was prepared according to N method 9 step 1 starting from 8-bromo-6,7-difluoroquinoline 27 (125 mg, 0.51 mmol) and compound 7 (251 mg, 0.77 mmol, 1.5 eq.). The crude was purified by flash chromatography (SiO₂, CyHex/EtOAc: 100/0 to 60/40) to afford compound 127 (140 mg, 75%) as a yellow oil. M/Z (M+H)⁺: 364.1.

Example 111 was prepared according to method 9 step 2 starting from compound 127 (140 mg, 0.39 mmol). The crude was purified by flash chromatography (KPNH, DCM/MeOH: 100/0 to 97/3). The obtained foam was dissolved in a mixture of aqueous 1N HCl/ACN and the resulting solution was freeze dried to afford Example 111 as a beige solid (62 mg, 50%).

¹H-NMR (DMSO-d₆, 400 MHz) δ: 1.07 (t, J 7.6 Hz, 3H, CH₂—CH₃); 2.36-2.47 (m, 2H, CH₂—CH₃); 7.00 (d, J 9.0 Hz, 1H, Ar); 7.65 (dd, J 8.3, 4.2 Hz, 1H, Ar); 7.84 (d, J 9.0 Hz, 1H, Ar); 8.19 (bs, 2H, NH₂); 8.24 (dd, J 10.8, 9.0 Hz, 1H, Ar); 8.50 (dd, J 8.3, 1.6 Hz, 1H, Ar); 8.89 (dd, J 4.3, 1.6 Hz, 1H, Ar); 14.52 (bs, 1H, HCl salt). M/Z (M+H)⁺: 286.0. Mp: 150-190° C.

### Example 112: 8-(6-amino-2-ethylpyridin-3-yl)-6,7-difluoroquinolin-3-ol (hydrochloride)

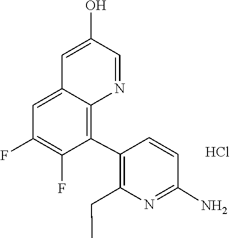 [SMILES: CCc1nc(N)ccc1-c1c(F)c(F)cc2cc(O)cnc12 ; Cl]

Protected intermediate of Example 112 was prepared according to method 9 step 1 starting from 8-bromo-6,7-difluoroquinolin-3-ol 81 (170 mg, 0.65 mmol) and compound 7 (320 mg, 0.98 mmol, 1.5 eq.). The crude was purified by flash chromatography (20 μm Interchim® SiO₂, CyHex/EtOAc: 100/0 to 50/50) to afford compound 128 (115 mg) as an orange solid. M/Z (M+H)⁺: 380.2.

Example 112 was prepared according to method 9 step 2 starting from compound 128 (115 mg). The crude was purified by flash chromatography (20 μm Interchim® SiO₂, DCM/MeOH: 100/0 to 95/5). The obtained foam was dissolved in a mixture of aqueous 1 N HCl/ACN and the resulting solution was freeze dried to afford Example 112 as a white solid (30 mg, 14% over 2 steps).

¹H-NMR (DMSO-d₆, 400 MHz) δ: 1.06 (t, J 7.6 Hz, 3H, CH₂—CH₃); 2.39-2.46 (m, 2H, CH₂—CH₃); 6.98 (d, J 9.1 Hz, 1H, Ar); 7.66 (d, J 2.8 Hz, 1H, Ar); 7.82 (d, J 9.1 Hz, 1H, Ar); 8.04 (dd, J 11.4, 8.8 Hz, 1H, Ar); 8.15 (bs, 2H, NH₂); 8.55 (d, J 2.8 Hz, 1H, Ar); 10.72 (bs, 1H, OH); 14.44 (bs, 1H, HCl salt). M/Z (M+H)⁺: 302.1. Mp: 204-210° C.

### Example 113: 6-ethyl-5-(5,6,7,8-tetrahydroacridin-4-yl)pyridin-2-amine (hydrochloride)

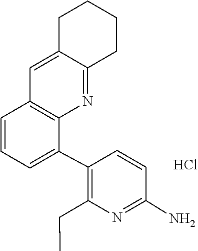 [SMILES: CCc1nc(N)ccc1-c1cccc2cc3c(nc12)CCCC3 ; Cl]

Protected intermediate of Example 113 was prepared according to method 9 step 1 starting from 5-bromo-1,2,3,4-tetrahydroacridine 28 (83 mg, 0.32 mmol) and compound 7 (150 mg, 0.47 mmol, 1.5 eq.). The crude was purified by flash chromatography (15 μm Interchim® SiO₂, CyHex/EtOAc: 100/0 to HCl 80/20) to afford compound 129 (112 mg) as a colorless oil. M/Z (M+H)⁺: 382.2

Example 113 was prepared according to method 9 step 2 starting from compound 129 (112 mg). The crude was purified by flash chromatography (15 μm Interchim® SiO₂, DCM/MeOH: 100/0 to 90/10). The obtained foam was dissolved in a mixture of aqueous 1N HCl/ACN and the resulting solution was freeze dried to afford Example 113 as a light yellow solid (65 mg, 60% over 2 steps).

¹H-NMR (DMSO-d₆, 400 MHz) δ: 1.11 (t, J 7.6 Hz, 3H, CH₃), 1.78-1.94 (m, 4H, 2*CH₂), 2.36-2.47 (m, 2H, CH₂—CH₃), 2.90-3.04 (m, 4H, 2*CH₂), 6.94 (d, J 9.1 Hz, 1H, Ar), 7.60-7.68 (m, 2H, Ar), 7.79 (d, J 9.1 Hz, 1H, Ar), 8.00 (t, J 4.5 Hz, 1H, Ar), 8.04 (bs, 2H, NH₂), 8.23 (bs, 1H, Ar), 14.28 (bs, 1H, HCl salt). M/Z (M+H)⁺: 304.1. Mp: 160-172° C.

### Example 114: 6-ethyl-5-(2-methyl-1,2,3,4-tetrahydrobenzo[b][1,6]naphthyridin-6-yl)pyridin-2-amine (dihydrochloride)

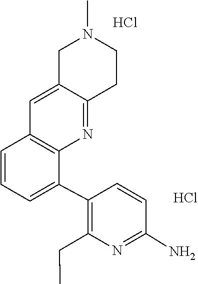 [SMILES: CCc1nc(N)ccc1-c1cccc2cc3c(nc12)CCN(C)C3 ; Cl ; Cl]

Protected intermediate of Example 114 was prepared according to method 9 step 1 starting from 6-bromo-2-methyl-1,2,3,4-tetrahydrobenzo[b][1,6]naphthyridine 29 (110 mg, 0.40 mmol) and compound 7 (194 mg, 0.60 mmol, 1.5 eq.). The crude was purified by flash chromatography (15 μm Interchim® SiO₂, DCM/MeOH: 100/0 to 90/10) to afford compound 130 (150 mg) as a colorless oil. M/Z (M+H)⁺: 397.3

Example 114 was prepared according to method 9 step 2 starting from compound 130 (150 mg). The crude was purified by flash chromatography (15 μm Interchim® SiO₂, DCM/MeOH: 100/0 to 80/20). The obtained foam was dissolved in a mixture of aqueous 1N HCl/ACN and the resulting solution was freeze dried to afford Example 114 as a yellow solid (102 mg, 44% over 2 steps).

¹H-NMR (DMSO-d₆, 400 MHz) δ: 1.12 (t, J 7.5 Hz, 3H, CH₂—CH₃); 2.36-2.46 (m, 2H, CH₂—CH₃), 2.95 (d, J 4.4 Hz, 3H, N—CH₃), 3.17 (dt, J 17.5, 3.2 Hz, 1H, CH₂), 3.38-3.60 (m, 2H, CH₂), 3.72-3.81 (m, 1H, CH₂), 4.53 (dd, J 15.6, 8.4 Hz, 1H, N—CH₂), 4.73 (d, J 15.6 Hz, 1H, N—CH₂), 6.95 (d, J 9.0 Hz, 1H, Ar), 7.65-7.74 (m, 2H, Ar), 7.80 (d, J 9.0 Hz, 1H, Ar), 8.07 (dd, J 7.7, 1.9 Hz, 1H, Ar), 8.12 (bs, 2H, NH₂), 8.34 (s, 1H, Ar), 11.56-11.94 (m, 1H, HCl salt), 14.52 (bs, 1H, HCl salt). M/Z (M+H)⁺: 319.1. Mp: 197-211° C.

### Example 115: 5-(2,3-dihydro-1H-cyclopenta[b]quinolin-5-yl)-6-ethylpyridin-2-amine (hydrochloride)

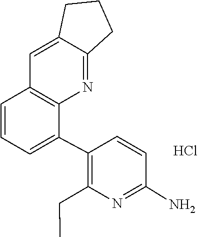 [SMILES: CCc1nc(N)ccc1-c1cccc2cc3c(nc12)CCC3 ; Cl]

Protected intermediate of Example 115 was prepared according to method 9 step 1 starting from 5-bromo-2,3-dihydro-1H-cyclopenta[b]quinoline 30 (130 mg, 0.52 mmol) and compound 7 (256 mg, 0.79 mmol, 1.5 eq.). The crude was purified by flash chromatography (15 μm Interchim® SiO₂, CyHex/EtOAc: 100/0 to 75/25) to afford compound 131 (215 mg) as a colorless oil. M/Z (M+H)⁺: 368.2.

Example 115 was prepared according to method 9 step 2 starting from compound 131 (215 mg). The crude was purified by flash chromatography (15 μm Interchim® SiO₂, DCM/MeOH: 100/0 to 90/10). The obtained foam was dissolved in a mixture of aqueous 1N HCl/ACN and the resulting solution was freeze dried to afford Example 115 as a yellow solid (127 mg, 74% over 2 steps).

¹H-NMR (DMSO-d₆, 400 MHz) δ: 1.10 (t, J 7.5 Hz, 3H, CH₂—CH₃); 2.13 (qt, J 7.5 Hz, 2H, CH₂—CH₂—CH₂); 2.35-2.47 (m, 2H, CH₂—CH₃); 3.00 (t, J 7.5 Hz, 2H, Ar—CH₂—CH₂); 3.08 (t, J 7.5 Hz, 2H, Ar—CH₂—CH₂); 6.94 (d, J 9.0 Hz, 1H, Ar); 7.59-7.66 (m, 2H, Ar); 7.79 (d, J 9.0 Hz, 1H, Ar); 7.99 (bs, 2H, NH₂); 8.01 (dd, J 6.5, 2.9 Hz, 1H, Ar); 8.25 (s, 1H, Ar); 14.17 (bs, 1H, HCl salt). M/Z (M+H)⁺: 290.1. Mp: 153-169° C.

### Example 116: 6-ethyl-5-(2-phenylquinolin-8-yl)pyridin-2-amine (hydrochloride)

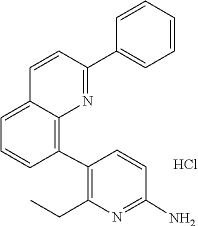 [SMILES: CCc1nc(N)ccc1-c1cccc2ccc(-c3ccccc3)nc12 ; Cl]

Protected intermediate of Example 116 was prepared according to method 9 step 1 starting from 8-bromo-2-phenylquinoline 31 (162 mg, 0.57 mmol) and compound 7 (279 mg, 0.85 mmol, 1.5 eq.). The crude was purified by flash chromatography (SiO₂, CyHex/DCM: 100/0 to 0/100) to afford compound 132 (140 mg, 61%) as a colorless oil. M/Z (M+H)⁺: 404.1

Example 116 was prepared according to method 9 step 2 starting from compound 132 (140 mg, 0.35 mmol). The crude was purified by flash chromatography (SiO₂, DCM/MeOH: 100/0 to 90/10). The obtained foam was dissolved in a mixture of aqueous 1N HCl/ACN and the resulting solution was freeze dried to afford Example 116 as a beige solid (40 mg, 35%).

¹H-NMR (DMSO-d₆, 300 MHz) δ: 1.07 (t, J 7.6 Hz, 3H, CH₂—CH₃); 2.53-2.60 (m, 2H, CH₂—CH₃); 7.00 (d, J 9.0 Hz, 1H, Ar); 7.44-7.54 (m, 3H, Ar); 7.70 (dd, J 7.6, 7.2 Hz, 1H, Ar); 7.76 (dd, J 7.2, 1.5 Hz, 1H, Ar); 7.94 (d, J 9.0 Hz, 1H, Ar); 8.05 (bs, 2H, NH₂); 8.08-8.14 (m, 3H, Ar), 8.23 (d, J 8.6 Hz, 1H, Ar); 8.26 (d, J 8.6 Hz, 1H, Ar); 14.30 (bs, 1H, HCl salt). M/Z (M+H)⁺: 326.2.

### Example 117: 6-ethyl-5-(2-(pyridin-3-yl)quinolin-8-yl)pyridin-2-amine (hydrochloride)

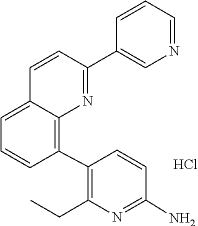 [SMILES: CCc1nc(N)ccc1-c1cccc2ccc(-c3cccnc3)nc12 ; Cl]

Protected intermediate of Example 117 was prepared according to method 9 step 1 starting from 8-bromo-2-(pyridin-3-yl)quinoline 32 (156 mg, 0.55 mmol) and compound 7 (268 mg, 0.82 mmol, 1.5 eq.). The crude was purified by flash chromatography (SiO₂, CyHex/DCM: 100/0 to 0/100 then DCM/EtOAc: 100/0 to 25/75) to afford compound 133 (50 mg) as a colorless oil. M/Z (M+H)⁺: 405.1

Example 117 was prepared according to method 9 step 2 starting from compound 133 (50 mg). The crude was purified by flash chromatography (SiO₂, DCM/MeOH: 100/0 to 90/10). The obtained foam was dissolved in a mixture of aqueous 1 N HCl/ACN and the resulting solution was freeze dried to afford Example 117 as a beige solid (20 mg, 10% over 2 steps).

¹H-NMR (DMSO-d₆, 300 MHz) δ: 1.07 (t, J 7.6 Hz, 3H, CH₂—CH₃); 2.53-2.60 (m, 2H, CH₂—CH₃); 7.00 (d, J 9.0 Hz, 1H, Ar); 7.72-7.79 (m, 2H, Ar); 7.81 (dd, J 7.1, 1.6 Hz, 1H, Ar); 7.96 (d, J 9.0 Hz, 1H, Ar); 8.05 (bs, 2H, NH₂); 8.17 (dd, J 8.0, 1.5 Hz, 1H, Ar); 8.35 (d, J 8.7 Hz, 1H, Ar); 8.63-8.70 (m, 2H, Ar); 8.77 (dd, J 5.0, 1.6 Hz, 1H, Ar); 9.33 (d, J 1.6 Hz, 1H, Ar); 14.20 (bs, 1H, HCl salt). M/Z (M+H)⁺: 327.2.

### Example 118: 5-(2-cyclohexylquinolin-8-yl)-6-ethylpyridin-2-amine (hydrochloride)

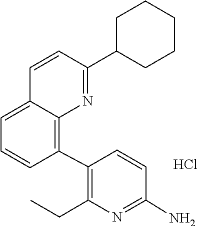 [SMILES: CCc1nc(N)ccc1-c1cccc2ccc(C3CCCCC3)nc12 ; Cl]

Protected intermediate of Example 118 was prepared according to method 9 step 1 starting from 8-bromo-2-cyclohexylquinoline 33 (160 mg, 0.55 mmol) and compound 7 (271 mg, 0.83 mmol, 1.5 eq.). The crude was purified by flash chromatography (SiO₂, DCM/EtOAc: 100/0 to 25/75) to afford compound 134 (140 mg, 62%) as a colorless oil. M/Z (M+H)⁺: 410.3 N NH₂

Example 118 was prepared according to method 9 step 2 starting from compound 134 (140 mg, 0.34 mmol). The crude was purified by flash chromatography (SiO₂, DCM/MeOH: 100/0 to 90/10). The obtained product was further purified by preparative HPLC (H₂O (0.5 wt. % HCOOH)/CH₃CN (0.5 wt. % HCOOH): 80/20 to 40/60). The obtained foam was dissolved in a mixture of aqueous 1N HCl/ACN and the resulting solution was freeze dried to afford Example 118 as a light yellow solid (40 mg, 32%).

¹H-NMR (DMSO-d₆, 400 MHz) δ: 1.06 (t, J 7.6 Hz, 3H, CH₃); 1.14-1.27 (m, 1H, CHaHb); 1.30-1.53 (m, 4H, 2*CH₂); 1.64-1.72 (m, 1H, CHaHb); 1.72-1.80 (m, 2H, CH₂); 1.81-1.89 (m, 2H, CH₂); 2.53-2.62 (m, 2H, CH₂—CH₃); 2.75-2.84 (m, 1H, CH); 6.90 (d, J 9.0 Hz, 1H, Ar); 7.54 (d, J 8.6 Hz, 1H, Ar); 7.60-7.71 (m, 2H, Ar); 7.84 (d, J 9.0 Hz, 1H, Ar); 7.97 (bs, 2H, NH₂); 8.04 (dd, J 7.8, 1.5 Hz, 1H, Ar); 8.37 (d, J 8.6 Hz, 1H, Ar); 14.14 (bs, 1H, HCl salt). M/Z (M+H)⁺: 332.3. Mp: 137-142° C.

### Example 119: 6-ethyl-5-(2-(pyridin-2-yl)quinolin-8-yl)pyridin-2-amine (hydrochloride)

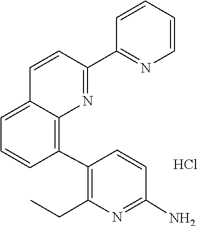 [SMILES: CCc1nc(N)ccc1-c1cccc2ccc(-c3ccccn3)nc12 ; Cl]

Protected intermediate of Example 119 was prepared according to method 9 step 1 starting from 8-bromo-2-(pyridin-2-yl)quinoline 35 (195 mg, 0.68 mmol) and compound 7 (385 mg, 1.03 mmol, 1.5 eq.). The crude was purified by flash chromatography (SiO₂, CyHex/DCM: 100/0 to 0/100, then DCM/EtOAc: 100/0 to 50/50) to afford compound 135 (110 mg, 40%) as a yellow oil. M/Z (M+H)⁺: 405.3

Example 119 was prepared according to method 9 step 2 starting from compound 135 (110 mg, 0.27 mmol). The crude was purified by flash chromatography (SiO₂, DCM/MeOH: 100/0 to 90/10). The obtained product was further purified by preparative HPLC (H₂O (0.5 wt. % HCOOH)/CH₃CN (0.5 wt. % HCOOH): 90/10 to 50/50). The obtained foam was dissolved in a mixture of aqueous 1 N HCl/ACN and the resulting solution was freeze dried to afford Example 119 as a light yellow solid (25 mg, 28%).

¹H-NMR (DMSO-d₆, 400 MHz) δ: 1.08 (t, J 7.5 Hz, 3H, CH₃); 2.53-2.62 (m, 2H, CH₂); 7.01 (d, J 9.0 Hz, 1H, Ar); 7.51 (ddd, J 7.6, 4.9, 1.2 Hz, 1H, Ar); 7.73-7.82 (m, 2H, Ar); 7.93-8.01 (m, 2H, Ar); 8.05 (bs, 2H, NH₂); 8.16 (dd, J 8.0, 1.6 Hz, 1H, Ar); 8.20 (d, J 8.0 Hz, 1H, Ar); 8.58-865 (m, 2H, Ar); 8.73-8.77 (m, 1H, Ar); 14.24 (bs, 1H, HCl salt). M/Z (M+H)⁺: 327.2. Mp: 111-116° C.

### Example 120: 6-ethyl-5-(2-(1-methylcyclopropyl)quinolin-8-yl)pyridin-2-amine (hydrochloride)

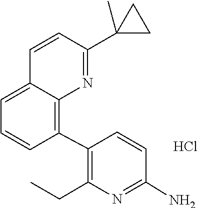 [SMILES: CCc1nc(N)ccc1-c1cccc2ccc(C3(C)CC3)nc12 ; Cl]

Protected intermediate of Example 120 was prepared according to method 9 step 1 starting from 8-bromo-2-(1-methylcyclopropyl)quinoline 34 (140 mg, 0.53 mmol) and compound 7 (261 mg, 0.80 mmol, 1.5 eq.). The crude was purified by flash chromatography (SiO₂, CyHex/DCM: 100/0 to 0/100) to afford compound 136 (160 mg, 79%) as a colorless oil. M/Z (M+H)⁺: 382.2

Example 120 was prepared according to method 9 step 2 starting from compound 136 (160 mg, 0.42 mmol). The crude was purified by flash chromatography (SiO₂, DCM/MeOH: 100/0 to 90110). The obtained product was further purified by preparative HPLC (H₂O (0.5 wt. % HCOOH)/CH₃CN (0.5 wt. % HCOOH): 80/20 to 40/60). The obtained foam was dissolved in a mixture of aqueous 1N HCl/ACN and the resulting solution was freeze dried to afford Example 120 as a light yellow solid (28 mg, 53%).

¹H-NMR (DMSO-d₆, 400 MHz) δ: 0.84 (d, J 2.8 Hz, 2H, CH₂—CH₂); 1.03 (t, J 7.6 Hz, 3H, CH₂—CH₃); 1.06-1.15 (m, 2H, CH₂—CH₂); 1.48 (s, 3H, CH₃); 2.53-2.62 (m, 2H, CH₂—CH₃); 6.93 (d, J 9.0 Hz, 1H, Ar); 7.53 (d, J 8.7 Hz, 1H, Ar); 7.60 (dd, J 7.9, 7.3 Hz, 1H, Ar); 7.68 (dd, J 6.9, 1.5 Hz, 1H, Ar); 7.82 (d, J 9.0 Hz, 1H, Ar); 7.94 (bs, 2H, NH₂); 8.02 (dd, J 8.2, 1.5 Hz, 1H, Ar); 8.33 (d, J 8.7 Hz, 1H, Ar); 14.11 (bs, 1H, HCl salt). M/Z (M+H)⁺: 304.2. Mp: 90-95° C.

### Example 121: 6-ethyl-5-(2-(tetrahydro-2H-pyran-4-yl)quinolin-8-yl)pyridin-2-amine (hydrochloride)

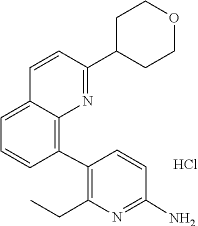 [SMILES: CCc1nc(N)ccc1-c1cccc2ccc(C3CCOCC3)nc12 ; Cl]

Protected intermediate of Example 121 was prepared according to method 9 step 1 starting from 8-bromo-2-(tetrahydro-2H-pyran-4-yl)quinoline 36 (100 mg, 0.34 mmol) and compound 7 (148 mg, 0.51 mmol, 1.5 eq.). The crude was purified by flash chromatography (SiO₂, CyHex/EtOAc: 100/0 to 0/100) to afford compound 137 (70 mg, 50%) as a colorless oil. M/Z (M+H)⁺: 382.2

Example 121 was prepared according to method 9 step 2 starting from compound 137 (70 mg, 0.17 mmol). The crude was purified by flash chromatography (SiO₂, DCM/MeOH: 100/0 to 90/10). The obtained product was further purified by preparative HPLC (H₂O (0.5 wt. % HCOOH)/CH₃CN (0.5 wt. % HCOOH): 90/10 to 50/50). The obtained foam was dissolved in a mixture of aqueous 1 N HCl/ACN and the resulting solution was freeze dried to afford Example 121 as a light yellow solid (25 mg, 39%).

¹H-NMR (DMSO-d₆, 400 MHz) δ: 1.06 (t, J 7.7 Hz, 3H, CH₂—CH₃); 1.64-1.79 (m, 4H, 2*CH₂—CH); 2.40-2.48 (m, 2H, CH₂—CH₃); 3.02-3.12 (m, 1H, CH); 3.44 (td, J 11.2, 3.0 Hz, 2H, 2*CHaHb-O); 3.87-3.95 (m, 2H, 2*CHaHb-O); 6.95 (d, J 9.0 Hz, 1H, Ar); 7.57 (d, J 8.5 Hz, 1H, Ar); 7.61-7.73 (m, 2H, Ar); 7.84 (d, J 9.0 Hz, 1H, Ar); 8.05 (dd, J 7.8, 1.6 Hz, 1H, Ar); 8.07 (bs, 2H, NH₂); 8.40 (d, J 8.6 Hz, 1H, Ar); 14.36 (bs, 1H, HCl salt). M/Z (M+H)⁺: 334.2. Mp: 175-185.

### Example 122: 6-ethyl-5-(2-(pyridin-4-yl)quinolin-8-yl)pyridin-2-amine (hydrochloride)

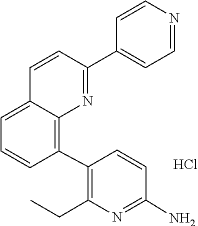 [SMILES: CCc1nc(N)ccc1-c1cccc2ccc(-c3ccncc3)nc12 ; Cl]

Protected intermediate of Example 122 was prepared according to method 9 step 1 starting from 8-bromo-2-(pyridin-4-yl)quinoline 37 (130 mg, 0.46 mmol) and compound 7 (223 mg, 0.68 mmol, 1.5 eq.). The crude was purified by flash chromatography (SiO₂, CyHex/EtOAc: 100/0 to 50/50) to afford compound 138 (230 mg) as a yellow oil. M/Z (M+H)⁺: 405.3.

Example 122 was prepared according to method 9 step 2 starting from compound 138 (230 mg). The crude was purified by flash chromatography (20 μm Interchim® SiO₂, DCM/MeOH: 100/0 to 90/10). The obtained foam was dissolved in a mixture of aqueous 1N HCl/ACN and the resulting solution was freeze dried to afford Example 122 as a light yellow solid (130 mg, 79% over 2 steps).

¹H-NMR (DMSO-d₆, 400 MHz) δ: 1.08 (t, J 7.6 Hz, 3H, CH₂—CH₃); 2.55-2.62 (m, 2H, CH₂—CH₃); 7.00 (d, J 9.0 Hz, 1H, Ar); 7.81-7.87 (m, 2H, 2*Ar); 7.94 (d, J 9.0 Hz, 1H, Ar); 8.15 (bs, 2H, NH₂); 8.22 (dd, J 7.7, 2.0 Hz, 1H, Ar); 8.44 (d, J 6.4 Hz, 2H, 2*Ar); 8.50 (d, J 8.7 Hz, 1H, Ar); 8.76 (d, J 8.7 Hz, 1H, Ar); 8.95 (d, J 6.4 Hz, 2H, 2*Ar); 14.47 (bs, 1H, HCl salt). M/Z (M+H)⁺: 327.2. Mp>250° C.

### Example 123: 6-ethyl-5-(2-(imidazo[1,2-a]pyridin-6-yl)quinolin-8-yl)pyridin-2-amine (hydrochloride)

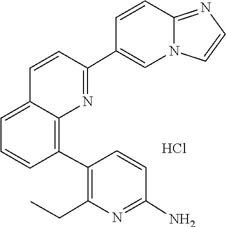 [SMILES: CCc1nc(N)ccc1-c1cccc2ccc(-c3ccc4nccn4c3)nc12 ; Cl]

Protected intermediate of Example 123 was prepared according to method 9 step 1 starting from 8-bromo-2-(imidazo[1,2-a]pyridin-6-yl)quinoline 38 (100 mg, 0.31 mmol) and compound 7 (151 mg, 0.46 mmol, 1.5 eq.). The N crude was purified by flash chromatography (SiO₂, DCM/MeOH: 100/0 to 90/10) to afford compound 139 (135 mg) as a light yellow solid. M/Z (M+H)⁺: 444.3.

Example 123 was prepared according to method 9 step 2 starting from compound 139 (135 mg). The crude was purified by flash chromatography (20 μm Interchim® SiO₂, DCM/MeOH: 100/0 to 90/10). The obtain solid was triturated in H₂O (6 mL) and filtered. The obtained solid was dissolved in a mixture of aqueous 1N HCl/ACN and the resulting solution was freeze dried and then triturated in CyHex (15 mL) to afford Example 123 as a light yellow solid (50 mg, 39% over 2 steps).

¹H-NMR (DMSO-d₆, 400 MHz) δ: 1.09 (t, J 7.5 Hz, 3H, CH₂—CH₃); 2.56-2.64 (m, 2H, CH₂—CH₃); 7.02 (d, J 9.0 Hz, 1H, Ar); 7.77-7.84 (m, 2H, 2*Ar); 7.95 (d, J 9.0 Hz, 1H, Ar); 8.09 (d, J 9.5 Hz, 1H, Ar); 8.13 (bs, 2H, NH₂); 8.18 (d, J 8.0 Hz, 1H, Ar); 8.21 (d, J 1.1 Hz, 1H, Ar); 8.30 (d, J 8.6 Hz, 1H, Ar); 8.39 (d, J 1.1 Hz, 1H, Ar); 8.46 (d, J 9.6 Hz, 1H, Ar); 8.72 (d, J 8.6 Hz, 1H, Ar); 9.71 (s, 1H, Ar); 14.48 (bs, 1H, HCl salt). M/Z (M+H)⁺: 366.2. Mp: 70-75° C.

### Example 124: 6-ethyl-5-(2-(pyrimidin-5-yl)quinolin-8-yl)pyridin-2-amine (hydrochloride) & Example 125: 6-ethyl-5-(2-(isoxazol-4-yl)quinolin-8-yl)pyridin-2-amine

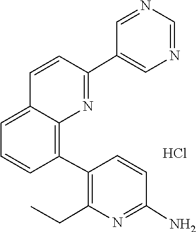 [SMILES: CCc1nc(N)ccc1-c1cccc2ccc(-c3cncnc3)nc12 ; Cl]

Protected intermediate of Example 124 was prepared according to method 9 step 1 starting from 8-bromo-2-(pyrimidin-5-yl)quinoline 39 (120 mg, 0.42 mmol) and compound 7 (205 mg, 0.63 mmol, 1.5 eq.). The crude was purified by flash chromatography (20 μm Interchim® SiO₂, CyHex/EtOAc: 100/0 to 50/50) to afford compound 140 (150 mg) as a light yellow solid. M/Z (M+H)⁺: 406.3.

Example 124 & Example 125 was prepared according to method 9 step 2 starting from compound 140 (150 mg). The crude was purified by flash chromatography (20 μm Interchim® SiO₂, DCM/MeOH: 100/0 to 90/10). The first obtained compound was triturated in CyHex (15 mL), then dissolved in a mixture of aqueous 1 N HCl/ACN and the resulting solution was freeze dried to afford Example 124 as a yellow solid (20 mg, 13% over 2 steps). The second obtained product was dissolved in a mixture of aqueous 1N HCl/ACN and the resulting solution was freeze dried. The desired was unstable in this condition. The product was further purified by preparative HPLC (H₂O (0.5 wt. % HCOOH)/CH₃CN (0.5 wt. % HCOOH): 90/10 to 50/50). Combined clean fractions were basified with NaHCO₃ sat. until pH ˜8. The aqueous layer was extracted with EtOAc (2×50 mL). Combined organic layers were dried over sodium sulfate and concentrated to afford Example 125 as a white solid (20 mg, 15% over 2 steps).

Example 124: ¹H-NMR (DMSO-d₆, 400 MHz) δ: 1.07 (t, J 7.6 Hz, 3H, CH₂—CH₃); 2.55-2.63 (m, 2H, CH₂—CH₃); 7.00 (d, J 9.0 Hz, 1H, Ar); 7.77 (t, J 7.5 Hz, 1H, Ar); 7.83 (dd, J 7.5, 1.5 Hz, 1H, Ar); 7.97 (d, J 9.0 Hz, 1H, Ar); 8.01 (bs, 2H, NH₂); 8.17 (dd, J 7.5, 1.5 Hz, 1H, Ar); 8.38 (d, J 8.7 Hz, 1H, Ar); 8.68 (d, J 8.7 Hz, 1H, Ar); 9.28 (s, 1H, Ar); 9.45 (s, 2H, 2*Ar); 14.10 (bs, 1H, HCl salt). M/Z (M+H)⁺: 328.2. Mp: 115-120° C.

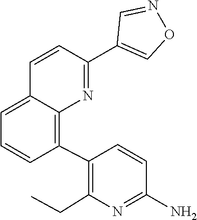 [SMILES: CCc1nc(N)ccc1-c1cccc2ccc(-c3cnoc3)nc12]

Example 125: ¹H-NMR (DMSO-d₆, 400 MHz) δ: 0.97 (t, J 7.5 Hz, 3H, CH₂—CH₃); 2.27-2.31 (m, 2H, CH₂—CH₃); 5.86 (bs, 2H, NH₂); 6.40 (d, J 8.3 Hz, 1H, Ar); 7.28 (d, J 8.3 Hz, 1H, Ar); 7.62-7.64 (m, 2H, 2*Ar); 7.94-7.97 (m, 2H, 2*Ar); 8.48 (d, J 8.6 Hz, 1H, Ar); 8.81 (s, 1H, Ar); 9.55 (s, 1H, Ar). M/Z (M+H)⁺: 317.1. Mp: 178-182° C.

### Example 126: 6-ethyl-5-(2-(pyrazin-2-yl)quinolin-8-yl)pyridin-2-amine (hydrochloride)

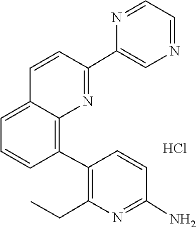 [SMILES: CCc1nc(N)ccc1-c1cccc2ccc(-c3cnccn3)nc12 ; Cl]

Protected intermediate of Example 126 was prepared according to method 9 step 1 starting from 8-bromo-2-(pyrazin-2-yl)quinoline 40 (120 mg, 0.42 mmol) and compound 7 (205 mg, 0.63 mmol, 1.5 eq.). The crude was purified by flash chromatography (20 μm Interchim® SiO₂, CyHex/EtOAc: 100/0 to HCl 80/20) to afford compound 141 (180 mg) as a light yellow solid. M/Z (M+H)⁺: 406.3.

Example 126 was prepared according to method 9 step 2 starting from compound 141 (180 mg). The crude was purified by flash chromatography (20 μm Interchim® SiO₂, DCM/MeOH: 100/0 to 90/10). The product was further purified by preparative HPLC (H₂O (0.5 wt. % HCOOH)/CH₃CN (0.5 wt. % HCOOH): 90/10 to 50/50). 1M HCl (3 mL) was added to the combined clean fractions and freeze dried to afford Example 126 as a white solid (65 mg, 43% over 2 steps).

¹H-NMR (DMSO-d₆, 400 MHz) δ: 1.08 (t, J 7.6 Hz, 3H, CH₂—CH₃); 2.55-2.63 (m, 2H, CH₂—CH₃); 7.01 (d, J 9.0 Hz, 1H, Ar); 7.79 (t, J 7.5 Hz, 1H, Ar); 7.84 (dd, J 7.5, 1.6 Hz, 1H, Ar); 7.97 (d, J 9.0 Hz, 1H, Ar); 7.99 (bs, 2H, NH₂); 8.19 (dd, J 7.5, 1.6 Hz, 1H, Ar); 8.54 (d, J 8.6 Hz, 1H, Ar); 8.68 (d, J 8.6 Hz, 1H, Ar); 8.75 (d, J 2.5 Hz, 1H, Ar); 8.80 (dd, J 2.6, 1.5 Hz, 1H, Ar); 9.34 (d, J 1.5 Hz, 1H, Ar); 14.08 (bs, 1H, HCl salt). M/Z (M+H)⁺: 328.2. Mp>250° C.

### Example 127: 6-ethyl-5-(2-(4-methylpyridin-3-yl)quinolin-8-yl)pyridin-2-amine (hydrochloride)

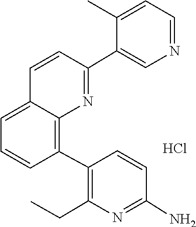 [SMILES: CCc1nc(N)ccc1-c1cccc2ccc(-c3cnccc3C)nc12 ; Cl]

Protected intermediate of Example 127 was prepared according to method 9 step 1 starting from 8-bromo-2-(4-methylpyridin-3-yl)quinoline 41 (120 mg, 0.40 mmol) and compound 7 (196 mg, 0.60 mmol, 1.5 eq.). The crude was purified by flash chromatography (20 μm Interchim® SiO₂, CyHex/EtOAc: 100/0 to 50/50) to afford compound 142 (170 mg) as a light yellow oil. M/Z (M+H)⁺: 419.3.

Example 127 was prepared according to method 9 step 2 starting from compound 142 (170 mg). The crude was purified by flash chromatography (20 μm Interchim® SiO₂, DCM/MeOH: 100/0 to 95/05). The obtained compound was triturated in H₂O (15 mL), then dissolved in a mixture of aqueous 1 N HCl/ACN and the resulting solution was freeze dried to afford Example 127 as a white solid (65 mg, 42% over 2 steps).

¹H-NMR (DMSO-d₆, 400 MHz) δ: 1.04 (t, J 7.7 Hz, 3H, CH₂—CH₃); 2.47 (s, 3H, CH₃); 2.53-2.64 (m, 2H, CH₂—CH₃); 6.93 (d, J 9.0 Hz, 1H, Ar); 7.78-7.84 (m, 3H, 3*Ar); 7.89 (d, J 9.0 Hz, 1H, Ar); 8.00 (d, J 8.6 Hz, 1H, Ar); 8.03 (bs, 2H, NH₂); 8.22 (dd, J 7.7, 1.8 Hz, 1H, Ar); 8.69 (d, J 8.6 Hz, 1H, Ar); 8.73 (d, J 5.7 Hz, 1H, Ar); 8.92 (s, 1H, Ar); 14.33 (bs, 1H, HCl salt). M/Z (M+H)⁺: 341.2. Mp: 180-188° C.

### Example 128: 6-ethyl-5-(2-(2-methylpyridin-3-yl)quinolin-8-yl)pyridin-2-amine (hydrochloride)

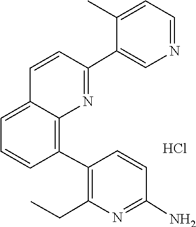 [SMILES: CCc1nc(N)ccc1-c1cccc2ccc(-c3cnccc3C)nc12 ; Cl]

Protected intermediate of Example 128 was prepared according to method 9 step 1 starting from 8-bromo-2-(2-methylpyridin-3-yl)quinoline 42 (120 mg, 0.40 mmol) and compound 7 (196 mg, 0.60 mmol, 1.5 eq.). The crude was purified by flash chromatography (20 μm Interchim® SiO₂, CyHex/EtOAc: 100/0 to 50/50) to afford compound 143 (210 mg) as a yellow oil. M/Z (M+H)⁺: 419.3.

Example 128 was prepared according to method 9 step 2 starting from compound 143 (210 mg). The crude was purified by flash chromatography (20 μm Interchim® SiO₂, DCM/MeOH: 100/0 to 95/5). The obtained compound was triturated in CyHex (10 mL), then dissolved in a mixture of aqueous 1 N HCl/ACN and the resulting solution was freeze dried to afford Example 128 as a white solid (85 mg, 57% over 2 steps).

¹H-NMR (DMSO-d₆, 400 MHz) δ: 1.04 (t, J 7.6 Hz, CH₂—CH₃); 2.54-2.60 (m, 2H, CH₂—CH₃); 2.62 (s, 3H, CH₃); 6.94 (d, J 9.0 Hz, 1H, Ar); 7.78-7.84 (m, 3H, 3*Ar); 7.88 (d, J 9.0 Hz, 1H, Ar); 7.98 (d, J 8.6 Hz, 1H, Ar); 8.02 (bs, 2H, NH₂); 8.21 (dd, J 8.0, 1.9 Hz, 1H, Ar); 8.45-8.47 (m, 1H, Ar); 8.69 (d, J 8.6 Hz, 1H, Ar); 8.73 (dd, J 5.3, 1.0 Hz, 1H, Ar); 14.28 (bs, 1H, HCl salt). M/Z (M+H)⁺: 341.2. Mp: 200-205° C.

### Example 129: 6-ethyl-5-(2-morpholinoquinolin-8-yl)pyridin-2-amine (hydrochloride)

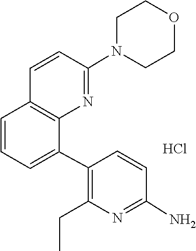 [SMILES: CCc1nc(N)ccc1-c1cccc2ccc(N3CCOCC3)nc12 ; Cl]

Protected intermediate of Example 129 was prepared according to method 9 step 1 starting from 4-(8-bromoquinolin-2-yl)morpholine 43 (150 mg, 0.51 mmol) and compound 7 (250 mg, 0.77 mmol, 1.5 eq.). XPhos PdG2 was used instead of SPhos PdG2.

The crude was purified by flash chromatography (SiO₂, CyHex/EtOAc: 100/0 to 70/30) to afford compound 144 (159 mg, 75%) as an orange solid. M/Z (M+H)⁺: 413.2 N

Example 129 was prepared according to method 9 step 2 starting from compound 144 (159 mg, 0.39 mmol). The crude was purified by flash chromatography (KPNH, CyHex/EtOAc: 100/0 to 60/40). The obtained foam was dissolved in a mixture of aqueous 1 N HCl/ACN and the resulting solution was freeze dried to afford Example 129 as a white solid (53 mg, 37%).

¹H-NMR (DMSO-d₆, 400 MHz) δ: 1.07 (t, J 7.6 Hz, 3H, CH₂—CH₃); 2.52-2.55 (m, 2H, CH₂—CH₃); 3.49 (t, J 4.8 Hz, 4H, 2*N—CH₂); 3.65 (t, J 4.8 Hz, 4H, 2*O—CH₂); 6.90 (d, J 9.0 Hz, 1H, Ar); 7.28 (d, J 9.2 Hz, 1H, Ar); 7.33 (dd, J 8.0, 7.2 Hz, 1H, Ar); 7.50 (dd, J 7.2, 1.5 Hz, 1H, Ar); 7.82 (dd, J 8.0, 1.5 Hz, 1H, Ar); 7.83 (d, J 9.0 Hz, 1H, Ar); 7.87 (bs, 2H, NH₂); 8.15 (d, J 9.2 Hz, 1H, Ar); 13.91 (bs, 1H, HCl salt). M/Z (M+H)⁺: 335.1. Mp: 100-150° C.

### Example 130: 6-ethyl-5-(2-(2-morpholinoethoxy)quinolin-8-yl)pyridin-2-amine (dihydrochloride)

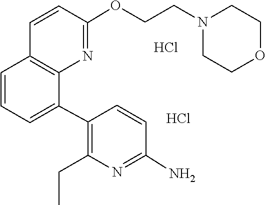 [SMILES: CCc1nc(N)ccc1-c1cccc2ccc(OCCN3CCOCC3)nc12 ; Cl ; Cl]

Protected intermediate of Example 130 was prepared according to method 9 step 1 starting from 4-(2-((8-bromoquinolin-2-yl)oxy)ethyl)morpholine 44 (150 mg, 0.45 mmol) and compound 7 (218 mg, 0.67 mmol, 1.5 eq.). The crude was purified by flash chromatography (SiO₂, CyHex/EtOAc: 100/0 to 10/90) to afford compound 145 (123 mg, 61%) as a yellow oil. M/Z (M+H)⁺: 457.3.

Example 130 was prepared according to method 9 step 2 starting from compound 145 (123 mg, 0.27 mmol). The crude was purified by flash chromatography (KPNH, DCM/MeOH: 100/0 to 95/5). The obtained product was dissolved in a mixture of aqueous 1N HCl/ACN and resulting solutions were freeze dried to afford Example 130 as a beige solid (5 mg, 4%).

¹H-NMR (DMSO-d₆, 400 MHz) δ: 1.09 (t, J 7.6 Hz, 3H, CH₂—CH₃); 2.52-2.60 (m, 2H, CH₂—CH₃); 3.09-3.19 (m, 2H, O—CH₂—CH₂); 3.42-3.49 (m, 4H, 2*N—CH₂); 3.75-3.82 (m, 2H, O—CH₂); 3.93-3.96 (m, 2H, O—CH₂); 4.55-4.59 (m, 2H, O—CH₂—CH₂); 6.95 (d, J 9.0 Hz, 1H, Ar); 7.13 (d, J 8.8 Hz, 1H, Ar); 7.57 (dd, J 8.0, 7.2 Hz, 1H, Ar); 7.67 (dd, J 7.2, 1.5 Hz, 1H, Ar); 7.87 (d, J 9.0 Hz, 1H, Ar); 7.96-8.11 (bs, 2H, NH₂); 8.03 (dd, J 8.0, 1.5 Hz, 1H, Ar); 8.40 (d, J 8.8 Hz, 1H, Ar); 10.98 (bs, 1H, HCl salt). M/Z (M+H)⁺: 379.1.

### Example 131: 6-ethyl-5-(2-(pyrrolidin-1-yl)quinolin-8-yl)pyridin-2-amine (hydrochloride)

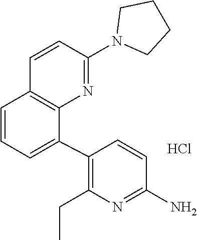 [SMILES: CCc1nc(N)ccc1-c1cccc2ccc(N3CCCC3)nc12 ; Cl]

Protected intermediate of Example 131 was prepared according to method 9 step 1 starting from 8-bromo-2-(pyrrolidin-1-yl)quinoline 45 (150 mg, 0.54 mmol) and compound 7 (265 mg, 0.81 mmol, 1.5 eq.). The crude was purified by flash chromatography (SiO₂, CyHex/EtOAc: 100/0 to 80/20) to afford compound 146 (236 mg) as a colorless oil. M/Z (M+H)⁺: 397.3.

Example 131 was prepared according to method 9 step 2 starting from compound 146 (236 mg). The crude was purified by flash chromatography (KPNH, DCM/MeOH: 100/0 to 95/5). The obtained product was dissolved in EtOAc (30 mL) and was washed with H₂O, dried over magnesium sulfate and concentrated. The obtained foam was triturated in Et₂O (5 mL). The obtained product was dissolved in a mixture of aqueous 1 N HCl/ACN and the resulting solution was freeze dried to afford Example 131 as a white solid (99 mg, 52% over 2 steps).

¹H-NMR (DMSO-d₆, 400 MHz, 80° C.) δ: 1.09 (t, J 7.6 Hz, 3H, CH₂—CH₃); 1.93-1.96 (m, 4H, CH₂); 2.59 (q, J 7.6 Hz, 2H, CH₂—CH₃); 3.40-3.43 (m, 4H, CH₂); 6.91 (d, J 9.0 Hz, 1H, Ar); 6.93 (d, J 9.0 Hz, 1H, Ar); 7.26 (dd, J 8.0, 7.2 Hz, 1H, Ar); 7.46 (dd, J 7.2, 1.5 Hz, 1H, Ar); 7.77 (dd, J 8.0, 1.4 Hz, 1H, Ar); 7.82 (d, J 9.0 Hz, 1H, Ar); 7.68-7.97 (m, 2H, NH₂); 8.05 (d, J 9.0 Hz, 1H, Ar). HCl salt signal not observed. M/Z (M+H)⁺: 319.0. Mp>250° C.

### Example 132: 5-(2-(4,4-difluoropiperidin-1-yl)quinolin-8-yl)-6-ethylpyridin-2-amine (hydrochloride)

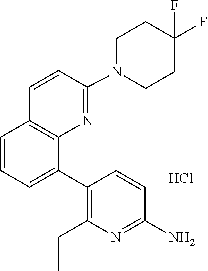 [SMILES: CCc1nc(N)ccc1-c1cccc2ccc(N3CCC(F)(F)CC3)nc12 ; Cl]

Protected intermediate of Example 132 was prepared according to method 9 step 1 starting from 8-bromo-2-(4,4-difluoropiperidin-1-yl)quinoline 46 (150 mg, 0.46 mmol) and compound 7 (224 mg, 0.69 mmol, 1.5 eq.). The crude was purified by flash chromatography (SiO₂, CyHex/EtOAc: 100/0 to 80/20). The obtain foam was further purified by flash chromatography (KPNH, CyHex/EtOAc: 100/0 to 80/20) to afford compound 147 (143 mg, 70%) as a transparent oil. M/Z (M+H)⁺: 447.3.

Example 132 was prepared according to method 9 step 2 starting from compound 147 (143 mg, 0.32 mmol). The crude was purified by flash chromatography (KPNH, CyHex/EtOAc: 100/0 to 50/50). The obtained foam was triturated with Et₂O (10 mL). The obtained product was dissolved in a mixture of aqueous 1 N HCl/ACN and the resulting solution was freeze dried to afford Example 132 as a white solid (85 mg, 66%).

¹H-NMR (DMSO-d₆, 400 MHz) δ: 1.06 (t, J 7.6 Hz, 3H, CH₂—CH₃); 1.91-1.98 (m, 4H, 2*CH₂); 2.54-2.61 (m, 2H, CH₂—CH₃); 3.71 (t, J 5.4 Hz, 4H, 2*N—CH₂); 6.91 (d, J 9.0 Hz, 1H, Ar); 7.35 (t, J 7.6 Hz, 1H, Ar); 7.39 (d, J 9.2 Hz, 1H, Ar); 7.52 (dd, J 7.2, 1.4 Hz, 1H, Ar); 7.82-7.89 (m, 4H, Ar+NH₂); 8.17 (d, J 9.2 Hz, 1H, Ar); 13.99 (bs, 1H, HCl salt). M/Z (M+H)⁺: 369.1. Mp: 120-141° C.

### Example 133: 5-(2-(1,4-oxazepan-4-yl)quinolin-8-yl)-6-ethylpyridin-2-amine (hydrochloride)

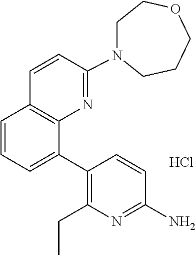 [SMILES: CCc1nc(N)ccc1-c1cccc2ccc(N3CCCOCC3)nc12 ; Cl]

Protected intermediate of Example 133 was prepared according to method 9 step 1 starting from 4-(8-bromoquinolin-2-yl)-1,4-oxazepane 47 (125 mg, 0.41 mmol) and compound 7 (199 mg, 0.61 mmol, 1.5 eq.). The crude was purified by flash chromatography (SiO₂, CyHex/EtOAc: 100/0 to 70/30) to afford compound 148 (138 mg, 79%) as a pink oil. M/Z (M+H)⁺: 427.2.

Example 133 was prepared according to method 9 step 2 starting from compound 148 (138 mg, 0.32 mmol). The crude was purified by flash chromatography (KPNH, CyHex/EtOAc: 100/0 to 50/50). The obtain compound was further purified by flash chromatography (15 μm Interchim® SiO₂, DCM/MeOH: 100/0 to 95/5). The obtained foam was triturated in Et₂O (10 mL). The obtained product was dissolved in a mixture of aqueous 1N HCl/ACN and the resulting solution was freeze dried to afford Example 133 as a beige solid (28 mg, 23%). ¹H-NMR (DMSO-d₆, 400 MHz, 80° C.) δ: 1.04 (t, J 7.6 Hz, 3H, CH₂—CH₃); 1.77-1.80 (m, 2H, CH₂); 3.51-3.55 (m, 4H, 2*N—CH₂); 3.61-3.63 (m, 2H, CH₂); 3.68-3.71 (m, 4H, 2*O—CH₂); 6.89 (d, J 9.0 Hz, 1H, Ar); 7.17 (d, J 9.2 Hz, 1H, Ar); 7.27 (dd, J 8.0, 7.2 Hz, 1H, Ar); 7.48 (dd, J 7.2, 1.5 Hz, 1H, Ar); 7.78 (dd, J 8.0, 1.5 Hz, 1H, Ar); 7.82 (d, J 9.0 Hz, 1H, Ar); 7.89 (bs, 2H, NH₂); 8.09 (d, J 9.2 Hz, 1H, Ar); 14.09 (bs, 1H, HCl salt). M/Z (M+H)⁺: 349.2. Mp: 50-90° C.

### Example 134: 6-ethyl-5-(7-fluoro-2-(1,4-oxazepan-4-yl)quinolin-8-yl)pyridin-2-amine (hydrochloride)

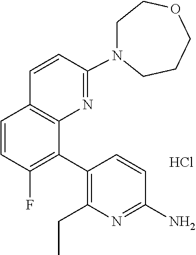 [SMILES: CCc1nc(N)ccc1-c1c(F)ccc2ccc(N3CCCOCC3)nc12 ; Cl]

Protected intermediate of Example 134 was prepared according to method 9 step 1 starting from 4-(8-bromo-7-fluoroquinolin-2-yl)-1,4-oxazepane 53 (125 mg, 0.38 mmol) and compound 7 (199 mg, 0.61 mmol, 1.6 eq.). The crude was purified by flash chromatography (15 μm Interchim® SiO₂, DCM/MeOH: 100/0 to 98/2) to afford 149 (107 mg, 63%) as an orange oil. M/Z (M+H)⁺: 445.2.

Example 134 was prepared according to method 9 step 2 starting from compound 149 (107 mg, 0.24 mmol). The crude was purified by flash chromatography (KPNH, DCM/MeOH: 100/0 to 97/03). The obtain compound was further purified by flash chromatography (15 μm Interchim® SiO₂, DCM/MeOH: 100/0 to 95/5). The product was solubilized in EtOAc (20 mL), washed with H₂O (2×30 mL), brine (30 mL) and dried over magnesium sulfate and concentrated. The obtained product was dissolved in a mixture of aqueous 1N HCl/ACN and the resulting solution was freeze dried to afford Example 134 as a beige solid (24 mg, 25%). ¹H-NMR (DMSO-d₆, 400 MHz) δ: 1.02 (t, J 7.6 Hz, 3H, CH₂—CH₃); 1.74-1.79 (m, 2H, CH₂); 2.42-2.48 (m, 2H, CH₂—CH₃); 3.48-3.55 (m, 2H, N—CH₂); 3.61-3.65 (m, 2H, N—CH₂); 3.67-3.76 (m, 4H, 2*O—CH₂); 6.94 (d, J 9.0 Hz, 1H, Ar); 7.14 (d, J 9.2 Hz, 1H, Ar); 7.20 (t, J 9.2 Hz, 1H, Ar); 7.80 (d, J 9.0 Hz, 1H, Ar); 7.86 (dd, J 8.9, 6.6 Hz, 1H, Ar); 8.00 (bs, 2H, NH₂); 8.10 (d, J 9.2 Hz, 1H, Ar); 14.26 (bs, 1H, HCl salt). M/Z (M+H)⁺: 367.1. Mp: 100-150° C.

### Example 135: 6-ethyl-5-(7-fluoro-2-morpholinoquinolin-8-yl)pyridin-2-amine (hydrochloride)

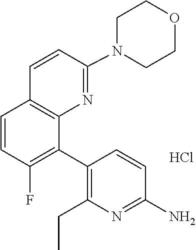 [SMILES: CCc1nc(N)ccc1-c1c(F)ccc2ccc(N3CCOCC3)nc12 ; Cl]

Protected intermediate of Example 135 was prepared according to method 9 step 1 starting from 4-(8-bromo-7-fluoroquinolin-2-yl)morpholine 54 (125 mg, 0.40 mmol) and compound 7 (197 mg, 0.60 mmol, 1.5 eq.). The crude was purified by flash chromatography (15 μm Interchim® SiO₂, CyHex/EtOAc: 100/0 to 70/30) to afford compound 150 (201 mg) as a transparent oil. M/Z (M+H)⁺: 431.2.

Example 135 was prepared according to method 9 step 2 starting from compound 150 (201 mg). The crude was purified by flash chromatography (KPNH, DCM/MeOH: 100/0 to 97/3). The obtained product was dissolved in a mixture of aqueous 1N HCl/ACN and the resulting solution was freeze dried to afford Example 135 as a white solid (83 mg, 53% over 2 steps).

¹H-NMR (DMSO-d₆, 400 MHz) δ: 1.05 (t, J 7.6 Hz, 3H, CH₂—CH₃); 2.42-2.48 (m, 2H, CH₂—CH₃); 3.49-3.52 (m, 4H, 2*N—CH₂); 3.63-3.65 (m, 4H, 2*O—CH₂); 6.94 (d, J 9.0 Hz, 1H, Ar); 7.23-7.29 (m, 2H, Ar); 7.79 (d, J 9.0 Hz, 1H, Ar); 7.90 (dd, J 8.8, 6.5 Hz, 1H, Ar); 7.98 (bs, 2H, NH₂); 8.16 (d, J 9.2 Hz, 1H, Ar); 14.13 (bs, 1H, HCl salt). M/Z (M+H)⁺: 353.2. Mp: 100-146° C.

### Example 136: 5-(2-(8-oxa-3-azabicyclo[3.2.1]octan-3-yl)-7-fluoroquinolin-8-yl)-6-ethylpyridin-2-amine (hydrochloride)

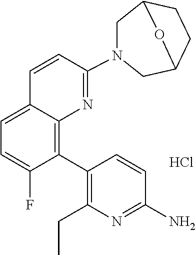 [SMILES: CCc1nc(N)ccc1-c1c(F)ccc2ccc(N3CC4CCC(C3)O4)nc12 ; Cl]

Protected intermediate of Example 136 was prepared according to method 9 step 1 starting from 3-(8-bromo-7-fluoroquinolin-2-yl)-8-oxa-3-azabicyclo[3.2.1]octane

55 (125 mg, 0.37 mmol) and compound 7 (181 mg, 0.56 mmol, 1.5 eq.). The crude was purified by flash chromatography (SiO₂, CyHex/EtOAc: 100/0 to 70/30) to afford compound 151 (192 mg) as a transparent oil. M/Z (M+H)⁺: 457.3.

Example 136 was prepared according to method 9 step 2 starting from compound 151 (192 mg). The crude was purified by flash chromatography (KPNH, DCM/MeOH: 100/0 to 99/1). The product was dissolved in HCl 1M (30 mL) and was washed with Et₂O (2×40 mL). The aqueous layer was basified with NaOH 6M and was extracted with Et₂O (2×40 mL), dried over magnesium sulfate and concentrated. The obtained product was dissolved in a mixture of aqueous 1N HCl/ACN and the resulting solution was freeze dried and triturated twice in Et₂O (2 mL) to afford Example 136 as a white solid (41 mg, 27% over 2 steps).

¹H-NMR (DMSO-d₆, 400 MHz) δ: 1.04 (t, J 7.6 Hz, 3H, CH₂—CH₃); 1.58-1.67 (m, 2H, CH₂); 1.77-1.81 (m, 2H, CH₂); 2.45 (q, J 7.6 Hz, 2H, CH₂—CH₃); 2.97 (dt, J 12.6, 3.0 Hz, 2H, 2*N—CHₐH_{b}); 3.88 (d, J 12.6 Hz, 2H, 2*N—CHₐH_{b}); 4.39 (d, J 3.1 Hz, 2H, 2*CH); 6.94 (d, J 9.0 Hz, 1H, Ar); 7.15 (d, J 9.2 Hz, 1H, Ar); 7.24 (t, J 9.0 Hz, 1H, Ar); 7.80 (d, J 9.0 Hz, 1H, Ar); 7.88 (dd, J 8.9, 6.6 Hz, 1H, Ar); 7.97 (bs, 2H, NH₂); 8.13 (d, J 9.2 Hz, 1H, Ar); 14.07 (bs, 1H, HCl salt). M/Z (M+H)⁺: 379.2. Mp: 140-172° C.

### Example 137: 5-(2-(azepan-1-yl)-7-fluoroquinolin-8-yl)-6-ethylpyridin-2-amine (hydrochloride)

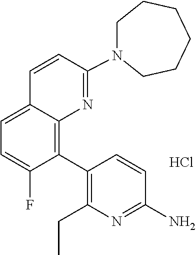 [SMILES: CCc1nc(N)ccc1-c1c(F)ccc2ccc(N3CCCCCC3)nc12 ; Cl]

Protected intermediate of Example 137 was prepared according to method 9 step 1 starting from 2-(azepan-1-yl)-8-bromo-7-fluoroquinoline 56 (125 mg, 0.39 mmol) and compound 7 (189 mg, 0.58 mmol, 1.5 eq.). The crude was purified by flash chromatography (SiO₂, CyHex/EtOAc: 100/0 to 80/20) to afford compound 152 (165 mg) as a transparent oil. M/Z (M+H)⁺: 443.2

Example 137 was prepared according to method 9 step 2 starting from compound 152 (165 mg). The crude was purified by flash chromatography (KPNH, DCM/MeOH: 100/0 to 99/01). The obtain foam was further purified by flash chromatography (20 μm Interchim® SiO₂, DCM/MeOH: 100/0 to 95/5). The obtained product was dissolved in a mixture of aqueous 1N HCl/ACN and the resulting solution was freeze dried to afford Example 137 as a white solid (75 mg, 48% over 2 steps).

¹H-NMR (DMSO-d₆, 400 MHz) δ: 1.01 (t, J 7.6 Hz, 3H, CH₂—CH₃); 1.41 (m, 4H, 2*CH₂); 1.61 (m, 4H, 2*CH₂); 2.46 (q, J 7.6 Hz, 2H, CH₂—CH₃); 3.55-3.59 (m, 4H, 2*N—CH₂); 6.94 (d, J 9.0 Hz, 1H, Ar); 7.06 (d, J 9.2 Hz, 1H, Ar); 7.17 (t, J 9.0 Hz, 1H, Ar); 7.80 (d, J 9.0 Hz, 1H, Ar); 7.83 (dd, J 9.0, 6.7 Hz, 1H, Ar); 7.96 (bs, 2H, NH₂); 8.06 (d, J 9.2 Hz, 1H, Ar); 14.18 (bs, 1H, HCl salt). M/Z (M+H)⁺: 365.2. Mp: 110-145° C.

### Example 138: 8-(6-amino-2-ethylpyridin-3-yl)-N-cyclohexyl-N-ethyl-7-fluoroquinolin-2-amine (hydrochloride)

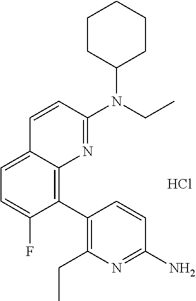 [SMILES: CCc1nc(N)ccc1-c1c(F)ccc2ccc(N(CC)C3CCCCC3)nc12 ; Cl]

Protected intermediate of Example 138 was prepared according to method 9 step 1 starting from 8-bromo-N-cyclohexyl-N-ethyl-7-fluoroquinolin-2-amine 57 (132 mg, 0.38 mmol) and compound 7 (184 mg, 0.56 mmol, 1.5 eq.). The crude was purified by flash chromatography (SiO₂, CyHex/EtOAc: 100/0 to 90/10) to afford compound 153 (173 mg) as an orange oil. M/Z (M+H)⁺: 471.3 N NH₂

Example 138 was prepared according to method 9 step 2 starting from compound 153 (173 mg). The crude was purified by flash chromatography (SiO₂, DCM/MeOH: 100/0 to 98/2). The obtained product was dissolved in a mixture of aqueous 1 N HCl/ACN and the resulting solution was freeze dried to afford Example 138 as a white solid (71 mg, 44% over 2 steps).

¹H-NMR (DMSO-d₆, 400 MHz, 80° C.) δ: 1.05 (t, J 7.6 Hz, 3H, CH₂—CH₃); 1.08 (t, J 6.9 Hz, 3H, N—CH₂—CH₃); 1.12-1.24 (m, 2H, CH₂); 1.45-1.54 (m, 2H, CH₂); 1.59-1.64 (m, 2H, CH₂); 1.75-1.78 (m, 2H, CH₂); 2.45-2.47 (m, 1H, CHₐH_{b}—CH₃); 2.53-2.55 (m, 1H, CHₐH_{b}—CH₃); 3.38-3.42 (m, 4H, 2*CH₂); 4.15 (tt, J 11.8, 3.6 Hz, 1H, CH₂—CH—CH₂); 6.98 (dd, J 9.0, 2.2 Hz, 2H, Ar); 7.13 (t, J 9.0 Hz, 1H, Ar); 7.76 (d, J 9.0 Hz, 1H, Ar); 7.81 (dd, J 8.8, 6.6 Hz, 1H, Ar); 7.91 (bs, 2H, NH₂); 8.03 (d, J 9.2 Hz, 1H, Ar); HCl salt signal not observed. M/Z (M+H)⁺: 393.3. Mp: 160-210° C.

### Example 139: 8-(6-amino-2-ethylpyridin-3-yl)-N-ethyl-7-fluoro-N-isopropylquinolin-2-amine (hydrochloride)

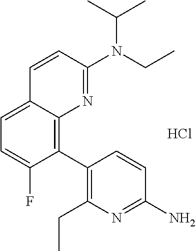 [SMILES: CCc1nc(N)ccc1-c1c(F)ccc2ccc(N(CC)C(C)C)nc12 ; Cl]

Protected intermediate of Example 139 was prepared according to method 9 step 1 starting from 8-bromo-N-ethyl-7-fluoro-N-isopropylquinolin-2-amine 58 (132 mg, 0.42 mmol) and compound 7 (208 mg, 0.64 mmol, 1.5 eq.). The crude was purified by flash chromatography (SiO₂, CyHex/EtOAc: 100/0 to 85/15) to afford compound 154 (159 mg) as an orange oil. M/Z (M+H)⁺: 431.2

Example 139 was prepared according to method 9 step 2 starting from compound 154 (159 mg). The crude was purified by flash chromatography (20 μm Interchim® SiO₂, DCM/MeOH: 100/0 to 97/3). The obtain product was further purified by preparative HPLC (H₂O (0.5 wt. % HCOOH)/CH₃CN (0.5 wt. % HCOOH): 80/20 to 40/60). The obtained product was dissolved in a mixture of aqueous 1N HCl/ACN and the resulting solution was freeze dried to afford Example 139 as a white solid (68 mg, 41% over 2 steps).

¹H-NMR (DMSO-d₆, 400 MHz) δ: 1.03 (m, 6H, 2*CH₂—CH₃); 1.10 (dd, J 6.7, 1.8 Hz, 6H, 2*CH₃); 2.42-2.48 (m, 2H, CH₂—CH₃); 3.35 (qd, J 7.0, 1.2 Hz, 2H); 4.50 (quint, J 6.7 Hz, 1H, CH₃—CH—CH₃); 6.94 (d, J 9.0 Hz, 1H, Ar); 7.04 (d, J 9.4 Hz, 1H, Ar); 7.17 (t, J 9.0 Hz, 1H, Ar); 7.80 (d, J 9.0 Hz, 1H, Ar); 7.83 (dd, J 8.9, 6.6 Hz, 1H, Ar); 8.01 (bs, 2H, NH₂); 8.08 (d, J 9.2 Hz, 1H, Ar); 14.33 (bs, 1H, HCl salt). M/Z (M+H)⁺: 353.2. Mp: 214-229° C.

### Example 140: 8-(6-amino-2-ethylpyridin-3-yl)-N,N-dimethylquinoline-2-carboxamide (hydrochloride)

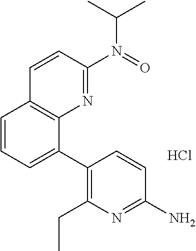

Protected intermediate of Example 140 were prepared according to method 9 step 1 starting from 8-bromo-N,N-dimethylquinoline-2-carboxamide 59 (150 mg, 0.54 mmol) and compound 7 (263 mg, 0.81 mmol, 1.5 eq.). The crude was purified by flash chromatography (15 μm Interchim® SiO₂, CyHex/EtOAc: 100/0 to 65/35) to afford compound 155 (200 mg, 93%) as a colorless oil. M/Z (M+H)⁺: 399.3

Example 140 was prepared according to method 9 step 2 starting from compound 155 (201 mg). The crude was purified by flash chromatography (15 μm Interchim® SiO₂, DCM/MeOH: 100/0 to 95/5). The obtained product was dissolved in a mixture of aqueous 1N HCl/ACN and resulting solutions were freeze dried to afford Example 140 as a beige solid (79 mg, 45%).

¹H-NMR (DMSO-d₆, 400 MHz) δ:1.05 (t, J 7.5 Hz, 3H, CH₂—CH₃); 2.35-2.47 (m, 2H, CH₂—CH₃); 2.89 (s, 3H, N—(CH₃)₂); 3.00 (s, 3H, N—(CH₃)₂); 6.94 (d, J 8.9 Hz, 1H, Ar); 7.74 (d, J 8.1 Hz, 1H, Ar); 7.76-7.81 (m, 2H, Ar); 7.83 (d, J 9.0 Hz, 1H, Ar); 7.97 (bs, 2H, NH₂); 8.16 (dd, J 7.1, 2.4 Hz, 1H, Ar); 8.59 (d, J 8.2 Hz, 1H, Ar); 14.10 (bs, 1H, HCl salt). M/Z (M+H)⁺: 321.0. Mp: 103-113° C.

### Example 141: (8-(6-amino-2-ethylpyridin-3-yl)quinolin-2-yl)(pyrrolidin-1-yl)methanone (hydrochloride)

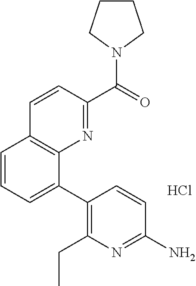 [SMILES: CCc1nc(N)ccc1-c1cccc2ccc(C(=O)N3CCCC3)nc12 ; Cl]

Protected intermediate of Example 141 was prepared according to method 9 step 1: starting from (8-bromoquinolin-2-yl)(pyrrolidin-1-yl)methanone 60 (125 mg, 0.41 mmol) and compound 7 (200 mg, 0.61 mmol, 1.5 eq.). The crude was purified by flash chromatography (SiO₂, CyHex/EtOAc: 100/0 to 50/50) to afford compound 156 (150 mg) as a transparent oil. M/Z (M+H)⁺: 425.3.

Example 141 was prepared according to method 9 step 2 starting from compound 156 (110 mg). The crude was purified by flash chromatography (KPNH DCM/MeOH: 100/0 to 98/2). The obtain foam was triturated in Et₂O (10 mL). The obtained product was dissolved in a mixture of aqueous 1N HCl/ACN and the resulting solution was freeze dried to afford Example 141 as a beige solid (79 mg, 50% over 2 steps).

¹H-NMR (DMSO-d₆, 400 MHz, 80° C.) δ: 1.03 (t, J 7.6 Hz, 3H, CH₂—CH₃); 1.75-1.82 (m, 4H, CH₂); 2.35-2.46 (m, 1H, CHₐH_{b}—CH₃); 2.52-2.54 (m, 1H, CHₐH_{b}—CH₃); 3.45 (t, J 7.6 Hz, 2H, N—CH₂), 3.50 (t, J 7.6 Hz, 2H, N—CH₂); 6.95 (d, J 9.0 Hz, 1H, Ar); 7.77-7.85 (m, 3H, Ar); 7.96 (d, J 8.6 Hz, 1H, Ar); 8.00 (bs, 2H, NH₂); 8.16 (dd, J 7.5, 2.1 Hz, 1H, Ar); 8.59 (d, J 8.6 Hz, 1H, Ar); 14.27 (bs, 1H, HCl salt). M/Z (M+H)⁺: 347.1. Mp: 100-125° C.

### Example 142: 6-ethyl-5-(2-(methoxymethyl)quinolin-8-yl)pyridin-2-amine (hydrochloride)

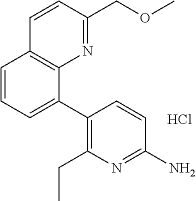 [SMILES: CCc1nc(N)ccc1-c1cccc2ccc(COC)nc12 ; Cl]

Protected intermediate of Example 142 was prepared according to method 9 step 1 starting from 8-bromo-2-(methoxymethyl)quinoline 63 (130 mg, 0.52 mmol) and compound 7 (202 mg, 0.62 mmol, 1.2 eq.). The crude was purified by flash chromatography (SiO₂, CyHex/EtOAc: 100/0 to 50/50) to afford compound 157 (113 mg, 59%) as a colorless oil. M/Z (M+H)⁺: 372.2

Example 142 was prepared according to method 9 step 2 starting from compound 157 (113 mg, 0.30 mmol). The crude was purified by flash chromatography (15 μm Interchim® SiO₂, DCM/MeOH: 100/0 to 90/10). The obtained product was dissolved in a mixture of aqueous 1N HCl/ACN and the resulting solution was freeze dried to afford Example 142 as a white solid (83 mg, 85%).

¹H-NMR (DMSO-d₆, 400 MHz) δ: 1.10 (t, J 7.7 Hz, 3H, CH₂—CH₃); 2.37-2.47 (m, 2H, CH₂—CH₃); 3.36 (s, 3H, O—CH₃); 4.56 (s, 2H, CH₂-0); 6.94 (d, J 9.2 Hz, 1H, Ar); 7.64 (d, J 8.2 Hz, 1H, Ar); 7.67-7.75 (m, 2H, Ar); 7.82 (d, J 8.9 Hz, 1H, Ar); 8.04 (bs, 2H, NH₂); 8.09 (dd, J 7.5, 2.1 Hz, 1H, Ar); 8.48 (d, J 8.3 Hz, 1H, Ar); 14.30 (bs, 1H, HCl salt). M/Z (M+H)⁺: 294.1. Mp: 210-225° C.

### Example 143: 5-(3,7-difluoroquinolin-8-yl)-6-ethylpyridin-2-amine (hydrochloride)

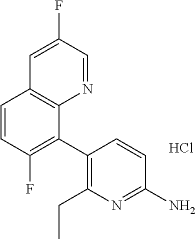 [SMILES: CCc1nc(N)ccc1-c1c(F)ccc2cc(F)cnc12 ; Cl]

Protected intermediate of Example 143 was prepared according to method 9 step 1 starting from 8-bromo-3,7-difluoroquinoline 70 (110 mg, 0.45 mmol) and compound 7 (221 mg, 0.68 mmol, 1.5 eq.). The crude was purified by flash chromatography (15 μm Interchim® SiO₂, CyHex/EtOAc: 100/0 to 80/20) to afford compound 158 (144 mg) as a colorless oil. M/Z (M+H)⁺: 364.2

Example 143 was prepared according to method 9 step 2 starting from compound 158 (144 mg). The crude was purified by flash chromatography (15 μm Interchim® SiO₂, DCM/MeOH: 100/0 to 92/8). The obtained product was dissolved in a mixture of aqueous 1N HCl/ACN and the resulting solution was freeze dried to afford Example 143 as a white solid (94 mg, 64% over 2 steps).

¹H-NMR (DMSO-d₆, 400 MHz) δ: 1.04 (t, J 7.5 Hz, 3H, CH₂—CH₃); 2.34-2.46 (m, 2H, CH₂—CH₃), 6.98 (d, J 8.9 Hz, 1H, Ar), 7.76-7.83 (m, 2H, Ar), 8.06 (bs, 2H, NH₂), 8.23 (dd, J 9.2, 6.2 Hz, 1H, Ar), 8.44 (dd, J 9.2, 2.7 Hz, 1H, Ar), 8.96 (d, J 2.7 Hz, 1H, Ar), 14.31 (bs, 1H, HCl salt). M/Z (M+H)⁺: 286.0. Mp: 245-250° C.

### Example 144: 5-(7-chloro-3-fluoroquinolin-8-yl)-6-ethylpyridin-2-amine (hydrochloride)

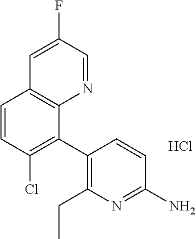 [SMILES: CCc1nc(N)ccc1-c1c(Cl)ccc2cc(F)cnc12 ; Cl]

Protected intermediate of Example 144 was prepared according to method 9 step 1 starting from 8-bromo-7-chloro-3-fluoroquinoline 74 (125 mg, 0.48 mmol) and compound 7 (204 mg, 0.62 mmol, 1.3 eq.). The crude was purified by flash chromatography (15 μm Interchim® SiO₂, CyHex/EtOAc: 100/0 to 80/20) to afford compound 159 (134 mg) as a colorless oil. M/Z (M[³⁷Cl]+H)⁺: 382.1

Example 144 was prepared according to method 9 step 2 starting from compound 159 (134 mg). The crude was purified by flash chromatography (15 μm Interchim® SiO₂, DCM/MeOH: 100/0 to 92/8). The obtained product was dissolved in a mixture of aqueous 1N HCl/ACN and the resulting solution was freeze dried to afford Example 144 as a white solid (76 mg, 46% over 2 steps).

¹H-NMR (DMSO-d₆, 400 MHz) δ: 0.99 (t, J 7.5 Hz, 3H, CH₂—CH₃); 2.28-2.36 (m, 2H, CH₂—CH₃); 6.98 (d, J 9.0 Hz, 1H, Ar); 7.74 (d, J 9.0 Hz, 1H, Ar); 7.92 (d, J 8.8 Hz, 1H, Ar); 7.99 (bs, 2H, NH₂); 8.18 (d, J 8.8 Hz, 1H, Ar); 8.44 (dd, J 9.1, 2.8 Hz, 1H, Ar); 8.95 (d, J 2.9 Hz, 1H, Ar); 14.15 (bs, 1H, HCl salt). M/Z [(M[³⁵Cl]+H)⁺]: 302.0. Mp>250° C.

### Example 145: 6-ethyl-5-(3,5,7-trifluoroquinolin-8-yl)pyridin-2-amine (hydrochloride)

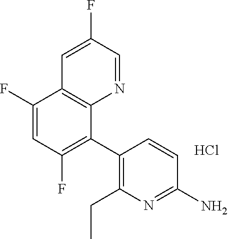 [SMILES: CCc1nc(N)ccc1-c1c(F)cc(F)c2cc(F)cnc12 ; Cl]

Protected intermediate of Example 145 was prepared according to method 9 step 1 starting from 8-bromo-3,5,7-trifluoroquinoline 78 (110 mg, 0.42 mmol) and compound 7 (178 mg, 0.55 mmol, 1.3 eq.). The crude was purified by flash chromatography (15 μm Interchim® SiO₂, CyHex/EtOAc: 100/0 to 75/25) to afford compound 160 (140 mg) as a colorless oil. M/Z (M+H)⁺: 382.2

Example 145 was prepared according to method 9 step 2 starting from compound 160 (140 mg), the crude was purified by flash chromatography (15 μm Interchim® SiO₂, DCM/MeOH: 100/0 to 94/6). The obtained product was dissolved in a mixture of aqueous 1 N HCl/ACN and the resulting solution was freeze dried to afford Example 145 as a white solid (101 mg, 71% over 2 steps).

¹H-NMR (DMSO-d₆, 400 MHz) δ: 1.04 (t, J 7.5 Hz, 3H, CH₂—CH₃); 2.35-2.45 (m, 2H, CH₂—CH₃); 6.96 (d, J 9.0 Hz, 1H, Ar); 7.78 (d, J 9.0 Hz, 1H, Ar); 7.91 (t, J 10.0 Hz, 1H, Ar); 7.99 (bs, 2H, NH₂); 8.48 (dd, J 8.8, 2.9 Hz, 1H, Ar); 9.04 (d, J 2.9 Hz, 1H, Ar); 14.12 (bs, 1H, HCl salt). M/Z (M+H)⁺: 304.0. Mp: 110-120° C.

### Example 146: 5-(3-chloro-7-fluoroquinolin-8-yl)-6-ethylpyridin-2-amine (hydrochloride)

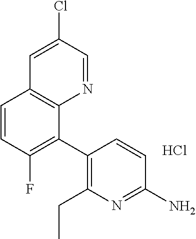 [SMILES: CCc1nc(N)ccc1-c1c(F)ccc2cc(Cl)cnc12 ; Cl]

Protected intermediate of Example 146 was prepared according to modified method 12 step 1 from compound 7 (175 mg, 0.54 mmol, 1.1 eq.) and 8-bromo-3-chloro-7-fluoroquinoline 71 (125 mg, 0.48 mmol). The crude residue was purified by flash chromatography (SiO₂, CyHex/EtOAc: 100/0 to 70/30) to afford compound 161 (103 mg, 57%) as a yellow oil. M/Z (M[³⁵Cl]+H)⁺: 380.1.

Example 146 was prepared according to method 12 step 2 starting from compound 161 (103 mg, 0.27 mmol). The crude was purified by flash chromatography (KPNH, DCM/MeOH: 100/0 to 97/3). The obtained foam was triturated in Et₂O (5 mL) and pentane (5 mL). The obtained product was dissolved in a mixture of aqueous 1N HCl/ACN and the resulting solution was freeze dried to afford Example 146 as a white solid (23 mg, 25%).

¹H-NMR (DMSO-d₆, 400 MHz) δ: 1.04 (t, J 7.6 Hz, 3H, CH₂—CH₃); 2.35-2.46 (m, 2H, CH₂—CH₃); 6.99 (d, J 9.0 Hz, 1H, Ar); 7.79-7.83 (m, 2H, Ar); 8.03 (bs, 2H, NH₂); 8.22 (dd, J 9.2, 6.2 Hz, 1H, Ar); 8.75 (d, J 2.5 Hz, 1H, Ar); 8.91 (d, J 2.5 Hz, 1H, Ar); 14.22 (bs, 1H, HCl salt). M/Z (M[³⁵Cl]+H)⁺: 302.0. Mp: 145-170° C.

### Example 147: 5-(3,7-dichloroquinolin-8-yl)-6-ethylpyridin-2-amine (hydrochloride)

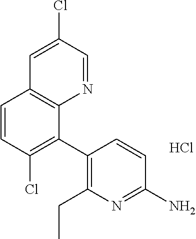 [SMILES: CCc1nc(N)ccc1-c1c(Cl)ccc2cc(Cl)cnc12 ; Cl]

Protected intermediate of Example 147 was prepared according to modified method 12 step I from compound 7 (147 mg, 0.45 mmol, 1.0 eq.) and 8-bromo-3,7-dichloroquinoline 75 (125 mg, 0.45 mmol, 1.0 eq.). The crude residue was purified by flash chromatography (20 μm Interchim® SiO₂, CyHex/EtOAc: 100/0 to 70/30) to afford compound 162 (111 mg) as a white solid. M/Z (M[³⁷Cl]₂+H)⁺: 399.2

Example 147 was prepared according to method 12 step 2 starting from compound 162 (111 mg). The crude was purified by flash chromatography (15 μm Interchim® SiO₂, DCM/MeOH: 100/0 to 92/8). The obtained compound was solubilized in HCl 1N (50 mL) and extracted twice with Et₂O (50 mL). The organic layer was discarded. The aqueous layer was basified with NaOH 6M (25 mL) and extracted thrice with DCM (30 mL). Combined organic layers were dried over sodium sulfate and concentrated. The obtained product was dissolved in a mixture of aqueous 1N HCl/ACN and the resulting solution was freeze dried to afford Example 147 as a white solid (61 mg, 38% over 2 steps).

¹H-NMR (DMSO-d₆, 400 MHz) δ: 1.00 (t, J 7.5 Hz, 3H, CH₂—CH₃); 2.28-2.37 (m, 2H, CH₂—CH₃); 6.99 (d, 9.0 Hz, 1H, Ar); 7.75 (d, 9.0 Hz, 1H, Ar); 7.93 (d, 9.0 Hz, 1H, Ar); 8.04 (bs, 2H, NH₂); 8.16 (d, 9.0 Hz, 1H, Ar); 8.75 (d, 2.5 Hz, 1H, Ar); 8.90 (d, 2.5 Hz, 1H, Ar); 14.25 (bs, 1H, HCl salt). M/Z [(M[³⁵Cl]₂+H)⁺: 318.0. Mp: 145-160° C.

### Example 148: 5-(3-chloro-5,7-difluoroquinolin-8-yl)-6-ethylpyridin-2-amine (hydrochloride)

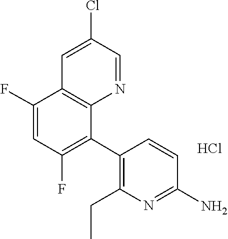 [SMILES: CCc1nc(N)ccc1-c1c(F)cc(F)c2cc(Cl)cnc12 ; Cl]

Protected intermediate of Example 148 was prepared according to modified method 12 step 1 from 8-bromo-3-chloro-5,7-difluoroquinoline 79 (110 mg, 0.40 mmol, 1.0 eq.) and compound 7 (142 mg 0.44 mmol, 1.1 eq.) The crude residue was purified by flash chromatography (20 μm Interchim® SiO₂, CyHex/DCM: 100/0 to 0/100) to obtain compound 163 (80 mg) as a colorless oil. M/Z (M[³⁷Cl]+H)⁺: 400.2

Example 148 was prepared according to method 12 step 2 starting from compound 163 (80 mg). The crude was purified by flash chromatography (15 μm Interchim® SiO₂, DCM/MeOH: 100/0 to 92:08). The obtained compound was solubilized in HCl 1N (50 mL), extracted twice with Et₂O (50 mL). The organic layer was discarded. The aqueous layer was basified with NaOH 6M (25 mL) and extracted thrice with DCM (30 mL). Combined organic layers were dried over sodium sulfate and concentrated. The obtained product was dissolved in a mixture of aqueous 1N HCl/ACN and the resulting solution was freeze dried to afford 148 as a white solid (58 mg, 40% over 2 steps).

¹H-NMR (DMSO-d₆, 400 MHz) δ: 1.04 (t, J 7.5 Hz, 3H, CH₂—CH₃); 2.35-2.45 (m, 2H, CH₂—CH₃); 6.95 (d, J 9.0 Hz, 1H, Ar); 7.78 (d, J 9.0 Hz, 1H, Ar); 7.91 (t, J 10.0 Hz, 1H, Ar); 8.00 (bs, 2H, NH₂); 8.73 (d, J 2.5 Hz, 1H, Ar); 8.99 (d, J 2.5 Hz, 1H, Ar); 14.11 (bs, 1H, HCl salt). M/Z [(M[³⁵Cl]+H)⁺]: 320.0. Mp:112-130° C.

### Example 149: 5-(3-chloro-6,7-difluoroquinolin-8-yl)-6-ethylpyridin-2-amine hydrochloride

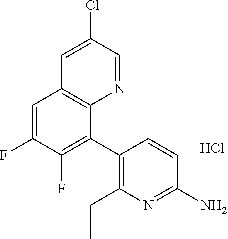 [SMILES: CCc1nc(N)ccc1-c1c(F)c(F)cc2cc(Cl)cnc12 ; Cl]

Protected intermediate of Example 149 was prepared according to modified method 12 step 1 from 8-bromo-3-chloro-6,7-difluoroquinoline 84 (110 mg, 0.40 mmol, 1.0 eq.) and compound 7 (142 mg, 0.44 mmol, 1.1 eq.). The crude was purified by flash chromatography (20 μm Interchim® SiO₂, CyHex/EtOAc: 100/0 to 70/30) to obtain compound 164 (110 mg) as a colorless oil. M/Z (M[³⁵Cl]+H)⁺: 398.2.

Example 149 was prepared according to method 12 step 2 starting from compound 164 (110 mg). The crude was purified by flash chromatography (20 μm Interchim® SiO₂, DCM/MeOH: 100/0 to 95/5). The obtained product was dissolved in a mixture of aqueous 1 N HCL/ACN and the resulting solution was freeze dried to afford Example 149 as a white solid (55 mg, 38% over 2 steps).

¹H-NMR (DMSO-d₆, 400 MHz) δ: 1.06 (t, J 7.6 Hz, 3H, CH₂—CH₃); 2.37-2.47 (m, 2H, CH₂—CH₃); 6.99 (d, J 9.0 Hz, 1H, Ar); 7.83 (d, J 9.0 Hz, 1H, Ar); 8.12 (bs, 2H, NH₂); 8.20 (dd, J 10.8, 8.9 Hz, 1H, Ar); 8.69 (d, J 2.4 Hz, 1H, Ar); 8.90 (d, J 2.4 Hz, 1H, Ar); 14.30 (bs, 1H, HCl salt).

M/Z (M[³⁵Cl]+H)⁺: 320.0. Mp: 140-150° C.

### Example 150: 6-ethyl-5-(3,6,7-trifluoroquinolin-8-yl)pyridin-2-amine (hydrochloride)

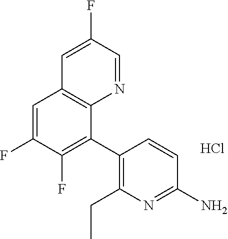 [SMILES: CCc1nc(N)ccc1-c1c(F)c(F)cc2cc(F)cnc12 ; Cl]

Protected intermediate of Example 150 was prepared according to method 9 step 1 starting from 8-bromo-3,6,7-trifluoroquinoline 83 (120 mg, 0.46 mmol) and compound 7 (224 mg, 0.69 mmol, 1.5 eq.). The crude was purified by flash chromatography (20 μm Interchim® SiO₂, CyHex/EtOAc: 100/0 to 75/25) to afford compound 165 (153 mg) as a colorless oil. M/Z (M+H)⁺: 382.2

Example 150 was prepared according to method 9 step 2 starting from compound 165 (153 mg). The crude was purified by flash chromatography (15 μm Interchim® SiO₂, DCM/MeOH: 100/0 to 94/6). The obtained foam was further purified by preparative HPLC (H₂O (0.5 wt. % HCOOH)/CH₃CN (0.5 wt. % HCOOH): 90/10 to 50/50). The obtained product was dissolved in a mixture of aqueous 1N HCl/ACN and the resulting solution was freeze dried to afford Example 150 as a white solid (95 mg, 61% over 2 steps).

¹H-NMR (DMSO-d₆, 400 MHz) δ: 1.06 (t, J 7.6 Hz, 3H, CH₂—CH₃); 2.37-2.47 (m, 2H, CH₂—CH₃); 6.98 (d, J 9.0 Hz, 1H, Ar); 7.81 (d, J 9.0 Hz, 1H, Ar); 8.08 (bs, 2H, NH₂); 8.21 (dd, J 10.8, 8.9 Hz, 1H, Ar); 8.40 (dd, J 9.3, 2.8 Hz, 1H, Ar); 8.95 (d, J 2.8 Hz, 1H, Ar); 14.32 (bs, 1H, HCl salt). M/Z (M+H)*304.0. Mp: 120-130° C.

### Example 151: 5-(3-bromo-7-fluoroquinolin-8-yl)-6-ethylpyridin-2-amine (hydrochloride)

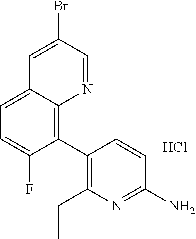 [SMILES: CCc1nc(N)ccc1-c1c(F)ccc2cc(Br)cnc12 ; Cl]

Example 151 was prepared according to method 9 step 2 starting from 3-bromo-8-(6-(2,5-dimethyl-1H-pyrrol-1-yl)-2-ethylpyridin-3-yl)-7-fluoroquinoline 86 (47 mg, 0.11 mmol, 1.0 eq.). The crude was purified by flash chromatography (20 μm Interchim® SiO₂, DCM/MeOH: 100/0 to 95/5). The obtained product was purified by preparative HPLC (H₂O (0.5 wt. % HCOOH)/CH₃CN (0.5 wt. % HCOOH): 90/10 to 50/50). The obtain product was suspended in ACN (2 mL)/H₂O (6 mL) and HCl 1M (2 mL) was added. The obtained product was dissolved in a mixture of aqueous 1N HCl/ACN and the resulting solution was freeze dried to afford Example 151 (25 mg, 59%) as a light yellow solid.

¹H-NMR (DMSO-d₆, 400 MHz) δ: 1.04 (t, J 7.5 Hz, 3H, CH₂—CH₃); 2.35-2.44 (m, 2H, CH₂—CH₃); 6.98 (d, J 9.0 Hz, 1H, Ar); 7.77-7.84 (m, 2H, Ar); 8.04 (bs, 2H, NH₂); 8.21 (dd, J 9.2, 6.2 Hz, 1H, Ar); 8.90 (d, J 2.4 Hz, 1H, Ar); 8.96 (d, J 2.4 Hz, 1H, Ar); 14.14 (bs, 1H, HCl salt). M/Z (M[⁸¹Br]+H)⁺: 348.0.

### Example 152: 8-(6-amino-2-ethylpyridin-3-yl)quinoline-7-carboxamide (hydrochloride)

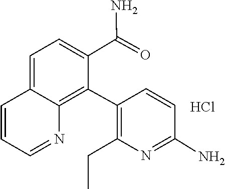 [SMILES: CCc1nc(N)ccc1-c1c(C(N)=O)ccc2cccnc12 ; Cl]

Protected intermediate of Example 152: In a MW tube under Argon, to a solution of 7-chloro-8-(6-(2,5-dimethyl-1H-pyrrol-1-yl)-2-ethylpyridin-3-yl)quinoline 126 (100 mg, 0.28 mmol) in DMA (2.1 mL), zinc cyanide (49 N mg, 0.42 mmol, 1.5 eq.) was added. The reaction mixture was sparged with argon for 10 min and Pd(PtBu₃)₂ (14 mg, 0.028 mmol, 0.1 eq.) was added. The reaction mixture was subjected to microwave irradiation at 150° C. for 15 min. The mixture was filtered through a pad of Celite® and the cake was washed with EtOAc (40 mL). The filtrate was hydrolyzed with NaHCO₃ sat. (40 mL) and extracted thrice with EtOAc (40 mL). The organic layers were washed with brine (40 mL), dried over magnesium sulfate and concentrated. The crude was purified by flash chromatography (SiO₂, CyHex/EtOAc: 100/0 to 70/30) to afford compound 166 (90 mg) as a yellow oil. M/Z (M+H)⁺: 353.2.

Example 152 was prepared according to method 9 step 2 starting from compound 166 (90.0 mg). The crude was purified by flash chromatography (KPNH, DCM/MeOH: 100/0 to 90/10) to obtain a white solid. The obtained product was dissolved in a mixture of aqueous 1 N HCl/ACN and the resulting solution was freeze dried to afford Example 152 as a beige solid (18 mg, 19% over 2 steps).

¹H-NMR (DMSO-d₆, 400 MHz) δ: 0.98 (t, J 7.6 Hz, 3H, CH₃); 2.21-2.40 (m, 2H, CH₂); 6.91 (d, J 9.0 Hz, 1H, Ar); 7.49 (bs, 1H, CO—N—H—H); 7.64 (dd, J 8.2, 4.2 Hz, 1H, Ar); 7.70 (d, J 9.0 Hz, 1H, Ar); 7.76 (d, J 8.3 Hz, 1H, Ar); 7.90 (bs, 1H, CO—N—H—H); 7.95 (bs, 2H, NH₂); 8.18 (d, J 8.5 Hz, 1H, Ar); 8.52 (dd, J 8.3, 1.8 Hz, 1H, Ar); 8.90 (dd, J 5.9, 1.8 Hz, 1H, Ar); 14.11 (bs, 1H, HCl salt). M/Z (M+H)⁺: 293.1. Mp: 150-164° C.

### Example 153: 8-(6-amino-2-ethylpyridin-3-yl)quinoline-7-carbonitrile

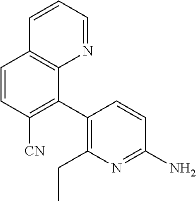 [SMILES: CCc1nc(N)ccc1-c1c(C#N)ccc2cccnc12]

Intermediate of Example 153 was prepared according to method 9 step 2 starting from compound 7-chloro-8-(6-(2,5-dimethyl-1H-pyrrol-1-yl)-2-ethylpyridin-3-yl)quinoline 126 (150 mg, 0.42 mmol). The crude was purified by flash chromatography (KPNH, CyHex/EtOAc: 100/0 to 20/80) to obtain 5-(7-chloroquinolin-8-yl)-6-ethylpyridin-2-amine 167 (60 mg, 51%) as a beige solid. M/Z (M[³⁵Cl]+H)⁺: 284.1.

Example 153: In a MW vial under Argon, to a solution of compound 167 (50 mg, 0.18 mmol) in DMA (1.4 mL) was added zinc cyanide (31 mg, 0.26 mmol, 1.5 eq.). The reaction mixture was sparged with argon for 10 min and Pd(PtBu₃)₂ (9 mg, 0.02 mmol, 0.1 eq.) was added. The reaction mixture was subjected twice to microwave irradiation at 180° C. for 15 min. Pd(PtBu₃)₂ (9.0 mg, 17.6 μmol, 0.1 eq.) was added and the reaction mixture was subjected to microwave irradiation at 150° C. for 15 min. The reaction mixture was filtered through a pad of Celite® and the cake was washed with EtOAc (40 mL). The filtrate was hydrolyzed with NaHCO₃ sat. (40 mL) and extracted thrice with EtOAc (40 mL). The organic layers were washed with brine (40 mL), dried over magnesium sulfate and concentrated. The organic layer was washed with NaHCO₃ sat. (40 mL), brine (40 mL), dried over magnesium sulfate and concentrated. The crude was purified by flash chromatography (KPNH, DCM/MeOH: 100/0 to 95/5). The obtain foam was further purified by flash chromatography (KPNH, CyHex/EtOAc: 100/0 to 20/80) to obtain a yellow solid. The obtained solid was triturated twice in Et₂O (3 mL) to afford Example 153 as a yellow solid (18 mg, 16% over 2 steps).

¹H-NMR (DMSO-d₆, 400 MHz) δ: 0.92 (t, J 7.6 Hz, 3H, CH₃); 2.06-2.20 (m, 2H, CH₂); 6.04 (s, 2H, NH₂); 6.41 (d, J 8.3 Hz, 1H, Ar); 7.21 (d, J 8.3 Hz, 1H, Ar); 7.70 (dd, J 8.3, 4.0 Hz, 1H, Ar); 7.97 (d, J 8.6 Hz, 1H, Ar); 8.17 (d, J 8.6 Hz, 1H, Ar); 8.53 (dd, J 8.3, 1.6 Hz, 1H, Ar); 8.96 (dd, J 4.2, 1.8 Hz, 1H, Ar). M/Z (M+H)⁺: 275.1. Mp: 226-228° C.

### Example 154: 8-(6-amino-2-ethylpyridin-3-yl)quinolin-2(1H)-one (hydrochloride)

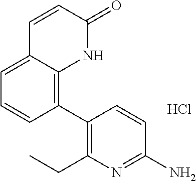 [SMILES: CCc1nc(N)ccc1-c1cccc2ccc(=O)[nH]c12 ; Cl]

Protected intermediate of 154 was prepared according to method 9 step 1 starting from 8-bromoquinolin-2(1H)-one (125 mg, 0.56 mmol) and compound 7 (273 mg, 0.84 mmol, 1.5 eq.). The crude was purified by flash chromatography (SiO₂, CyHex/EtOAc: 100/0 to 50/50) to afford compound 168 (207 mg) as a light yellow oil. M/Z (M+H)⁺: 344.2

Example 154 was prepared according to method 9 step 2 starting from compound 168 (207 mg). The crude was purified by flash chromatography (KPNH, DCM/MeOH: 100/0 to 98/2).

The obtained product was triturated twice in Et₂O (3 mL), then dissolved in a mixture of aqueous 1N HCl/ACN and the resulting solution was freeze dried to afford Example 154 as a white solid (96 mg, 57% over 2 steps).

¹H-NMR (DMSO-d₆, 400 MHz) δ: 1.08 (t, J 7.6 Hz, 3H, CH₂—CH₃); 2.27-2.36 (m, 1H, CHₐH_{b}—CH₃); 2.42-2.48 (m, 1H, CHₐH—CH₃); 6.53 (d, J 9.6 Hz, 1H, Ar); 6.91 (d, J 9.0 Hz, 1H, Ar); 7.27 (t, J 7.5 Hz, 1H, Ar); 7.37 (dd, J 7.5, 1.4 Hz, 1H, Ar); 7.66 (d, J 9.0 Hz, 1H, Ar); 7.76 (dd, J 7.8, 1.4 Hz, 1H, Ar); 7.98 (d, J 9.6 Hz, 1H, Ar); 7.99 (brs, 2H, NH₂); 10.91 (s, 1H, NH); 14.16 (bs, 1H, HCl salt). M/Z (M+H)⁺: 266.0. Mp>250° C.

### Example 155: 8-(6-amino-2-ethylpyridin-3-yl)-3,4-dihydroquinolin-2(1H)-one (hydrochloride)

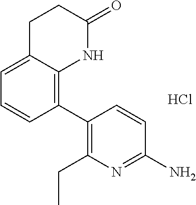 [SMILES: CCc1nc(N)ccc1-c1cccc2c1NC(=O)CC2 ; Cl]

In a sealed vial under Argon, a solution of Example 154 (free base, 30 mg, 0.11 mmol) in MeOH (1.0 mL) was sparged with Ar during 10 min before addition of Pd/C (10 wt. %, 10 mg). The reaction mixture was stirred under H₂ atmosphere at 25° C. for 4 days. The mixture was filtered through a pad of Celite® and the cake was washed with MeOH (50 mL). The filtrate was concentrated. The crude was purified by flash chromatography (SiO₂, DCM/MeOH: 100/0 to 95/05) The obtained product was dissolved in a mixture of aqueous 1N HCl/ACN and the resulting solution was freeze dried to afford Example 155 as a white solid (11 mg, 33%).

¹H-NMR (D₂O, 400 MHz) δ: 1.13 (t, J 7.5 Hz, 3H, CH₃); 2.49-2.66 (m, 2H, CH₂); 2.67-2.71 (m, 2H, CH₂); 3.09 (t, J 7.7 Hz, 2H, CH₂); 6.96 (d, J 9.0 Hz, 1H, Ar); 7.18-7.25 (m, 2H, Ar); 7.41-7.43 (m, 1H, Ar); 7.72 (d, J 9.0 Hz, 1H, Ar). M/Z (M+H)⁺: 268.1. Mp: 233-237° C.

### Example 156: 8-(6-amino-2-ethylpyridin-3-yl)-1-methylquinolin-2(1H)-one (hydrochloride)

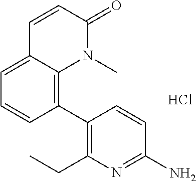 [SMILES: CCc1nc(N)ccc1-c1cccc2ccc(=O)n(C)c12 ; Cl]

Protected intermediate of Example 156: Under Argon, to a solution of compound 168 (354 mg, 1.03 mmol, 1 eq.) in THF (15 mL) potassium 2-methylpropan-2-olate (200 mg, 1.78 mmol, 1.7 eq.) and iodomethane (252 mg, 1.78 mmol, 1.7 eq.) were added. The reaction mixture was stirred at 25° C. for 5 h. The reaction mixture was hydrolyzed with water (125 mL) and extracted twice with EtOAc (100 mL). The organic layers were washed with brine (100 mL), dried over magnesium sulfate and concentrated. The crude was purified by flash chromatography (SiO₂, CyHex/EtOAc: 100/0 to 60/40) to obtain compound 169 (313 mg, 85%) as a yellow oil. M/Z (M+H)⁺: 458.2

Example 156 was prepared according to method 9 step 2 starting from compound 169 (313 mg, 0.88 mmol, 1 eq.). The crude was purified by flash chromatography (SiO₂, DCM/MeOH: 100/0 to 96/4) to obtain compound 170 (217 mg, 88%) as white solid. M/Z (M+H)⁺: 280.0.

50 mg of compound 170 were dissolved in a mixture of aqueous 1N HCl/ACN and the resulting solution was freeze dried to afford Example 156 as a white solid. M/Z (M+H)⁺: 280.0.

¹H-NMR (D₂O, 400 MHz) δ: 1.05 (t, J 7.6 Hz, 3H, CH₂—CH₃); 2.40-2.60 (m, 2H, CH₂—CH₃); 3.27 (s, 3H, N—CH₃); 6.83 (d, J 9.2 Hz, 1H, CH); 7.03 (d, J 9.2 Hz, 1H, CH); 7.49-7.57 (m, 2H, Ar); 7.89 (dd, J 7.5, 1.9 Hz, 1H, Ar); 7.92 (d, J 9.3 Hz, 1H, Ar); 8.12 (d, J 9.3 Hz, 1H, Ar). NH₂ and HCl salt signals not observed. M/Z (M+H)⁺: 280.0. Mp: 132-145° C.

### Example 157: 8-(6-amino-2-ethylpyridin-3-yl)-1-methyl-3,4-dihydroquinolin-2(1H)-one (hydrochloride)

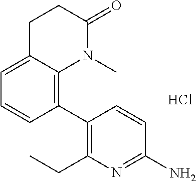 [SMILES: CCc1nc(N)ccc1-c1cccc2c1N(C)C(=O)CC2 ; Cl]

Under Argon, to a solution of 8-(6-amino-2-ethylpyridin-3-yl)-1-methylquinolin-2(1H)-one 170 (167 mg, 0.60 mmol, 1 eq.) in methanol (5 mL) was sparged sparged with argon for 10 min before addition of palladium on charcoal 10 wt. % (63.6 mg, 0.06 mmol, 0.1 eq.). The reaction mixture was stirred under H₂ atmosphere at 25° C. for 16 h. The mixture was filtered through a pad of Celite® and the cake was washed with MeOH (50 mL). The filtrate was concentrated. The crude was purified by flash chromatography (SiO₂, DCM/MeOH: 100/0 to 96/4). The obtained product was in a mixture of aqueous 1N HCl/ACN and the resulting solution was freeze dried to afford Example 157 (142 mg, 75%) as a white solid.

¹H-NMR (DMSO, 400 MHz) δ: 1.11 (t, J 7.6 Hz, 3H, CH₂—CH₃); 2.44-2.47 (m, 2H, CH₂—CH₃); 2.52-2.61 (m, 2H, Ar—CH₂); 2.66 (s, 3H, N—CH₃); 2.88 (t, J 6.9 Hz, 2H, C═O—CH₂); 6.93 (d, J 9.1 Hz, 1H, Ar); 7.08 (dd, J 7.7, 1.6 Hz, 1H, Ar); 7.16 (t, J 7.6 Hz, 1H, Ar); 7.33 (dd, J 7.3, 1.4 Hz, 1H, Ar); 7.81 (d, J 9.1 Hz, 1H, Ar); 8.00 (bs, 2H, NH₂); 13.92 (bs, 1H, HCl salt). M/Z (M+H)⁺: 282.1.

### Example 158: 8-(6-amino-2-ethylpyridin-3-yl)-7-fluoroquinolin-2(1H)-one (hydrochloride)

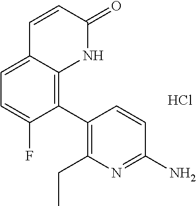 [SMILES: CCc1nc(N)ccc1-c1c(F)ccc2ccc(=O)[nH]c12 ; Cl]

Protected intermediate of Example 158 was prepared according to method 9 step 1 starting from 8-bromo-7-fluoroquinolin-2(1H)-one 48 (125 mg, 0.52 mmol) and compound 7 (253 mg, 0.76 mmol, 1.5 eq.). The crude was purified by flash chromatography (SiO₂, CyHex/EtOAc: 100/0 to 70/30) to afford compound 171 (185 mg) as a colorless oil. M/Z (M+H)⁺: 382.2

Example 158 was prepared according to method 9 step 2 starting from compound 171 (185 mg). The crude was purified by flash chromatography (20 μm Interchim® SiO₂, DCM/MeOH: 100/0 to 95/5). The obtained product was dissolved in a mixture of aqueous 1N HCl/ACN and the resulting solution was freeze dried to afford Example 158 as a white solid (65 mg, 39% over 2 steps).

¹H-NMR (DMSO-d₆, 400 MHz) δ: 1.08 (t, J 7.6 Hz, 3H, CH₂—CH₃); 2.29-2.38 (m, 1H, CHₐH_{b}—CH₃); 2.40-2.48 (m, 1H CHₐH_{b}—CH): 6.49 (d, J 9.6 Hz, 1H, Ar); 6.94 (d, J 9.0 Hz, 1H, Ar); 7.21 (t, J 8.9 Hz, 1H, Ar); 7.69 (d, J 9.0 Hz, 1H, Ar); 7.85 (dd, J 8.9, 6.2 Hz, 1H, Ar); 7.98 (d, J 9.6 Hz, 1H, Ar); 8.06 (bs, 2H, NH₂); 11.07 (s, 1H, NH or OH); 14.27 (bs, 1H, HCl salt). M/Z (M+H)⁺: 284.1. Mp: 172-208° C.

### Example 159: 8-(6-amino-2-ethylpyridin-3-yl)-5,7-difluoroquinolin-2(1H)-one (hydrochloride)

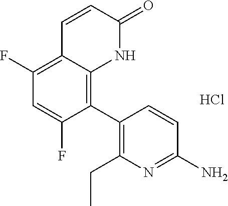 [SMILES: CCc1nc(N)ccc1-c1c(F)cc(F)c2ccc(=O)[nH]c12 ; Cl]

Protected intermediate of Example 159 was prepared according to method 9 step 1 starting from 8-bromo-5,7-difluoroquinolin-2(1H)-one 49 (157 mg, 0.60 mmol) and compound 7 (295 mg, 0.91 mmol, 1.5 eq.). The crude was purified by flash chromatography (SiO₂, CyHex/EtOAc: 100/0 to 70/30) to afford compound 172 (106 mg, 46%) as a colorless oil. M/Z (M+H)⁺: 382.2

Example 159 was prepared according to method 9 step 2 starting from compound 172 (106 mg, 0.28 mmol). The crude was purified by flash chromatography (SiO₂, DCM/MeOH: 100/0 to 95/05). The obtained product was solubilized in HCl 1 N (20 mL) and extracted twice with Et₂O (40 mL). The aqueous layer was basified with NaOH 6N and was extracted thrice with DCM (40 mL). The DCM layers were dried over magnesium sulfate and concentrated. The obtained product was dissolved in a mixture of aqueous 1N HCl/ACN and the resulting solution was freeze dried to afford Example 159 as a white solid (48 mg, 51%).

¹H-NMR (DMSO-d₆, 400 MHz) δ: 1.09 (t, J 7.6 Hz, 3H, CH₂—CH₃); 2.31-2.40 (m, 1H, CHₐH_{b}—CH₃); 2.41-2.47 (m, 1H, CHₐH_{b}—CH₃); 6.55 (d, J 9.6 Hz, 1H, Ar); 6.93 (d, J 9.0 Hz, 1H, Ar); 7.31 (t, J 9.8 Hz, 1H, Ar); 7.68 (d, J 9.0 Hz, 1H, Ar); 8.02 (d, J 9.6 Hz, 1H, Ar); 8.08 (bs, 2H, NH₂); 11.29 (s, 1H, NH or OH); 14.22 (bs, 1H, HCl salt). M/Z (M+H)⁺: 302.0. Mp>250° C.

### Example 160: 8-(6-amino-2-ethylpyridin-3-yl)-7-chloroquinolin-2(1H)-one (hydrochloride)

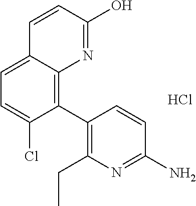 [SMILES: CCc1nc(N)ccc1-c1c(Cl)ccc2ccc(O)nc12 ; Cl]

Protected intermediate of Example 160 was prepared according to method 9 step 1 starting from 8-bromo-7-chloroquinolin-2(1H)-one 50 (125 mg, 0.48 mmol) and compound 7 (174 mg, 0.53 mmol, 1.1 eq.). The crude was purified by flash chromatography (SiO₂, CyHex/EtOAc: 100/0 to 50/50) to afford compound 173 (114 mg) as a yellow oil. M/Z (M[³⁵Cl]+H)⁺: 378.2.

Example 160 was prepared according to method 9 step 2 starting from compound 173 (114 mg). The crude was purified by flash chromatography (SiO₂, DCM/MeOH: 100/0 to 96/4). The obtained product was solubilized in HCl 1N (20 mL) and extracted twice with Et₂O (40 mL). The aqueous layer was basified with NaOH 6N and was extracted thrice with DCM (40 mL). The DCM layers were dried over magnesium sulfate and concentrated. dried over magnesium sulfate and concentrated. The obtained product was dissolved in a mixture of aqueous 1N HCl/ACN and the resulting solution was freeze dried to afford Example 160 as a white solid (25 mg, 16% over 2 steps).

¹H-NMR (DMSO-d₆, 400 MHz) δ: 1.08 (t, J 7.6 Hz, 3H, CH₂—CH₃); 2.25-2.31 (m, 1H, CHₐH_{b}—CH₃); 2.34-2.44 (m, 1H, CHₐH_{b}—CH₃); 6.54 (dd, J 9.6, 1.7 Hz, 1H, Ar); 6.93 (d, J 9.0 Hz, 1H, Ar); 7.43 (d, J 8.4 Hz, 1H, Ar); 7.63 (d, J 9.0 Hz, 1H, Ar); 7.80 (d, J 8.4 Hz, 1H, Ar); 7.99 (d, J 9.6 Hz, 1H, Ar); 8.04 (bs, 2H, NH₂); 11.00 (s, 1H, NH or OH); 14.16 (bs, 1H, HCl salt). M/Z (M[³⁵Cl]+H)⁺: 300.0. Mp>250° C.

### Example 161: 8-(6-amino-2-ethylpyridin-3-yl)-6,7-difluoroquinolin-2(1H)-one (hydrochloride)

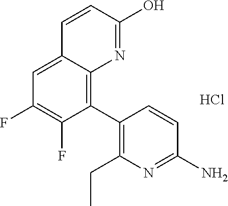 [SMILES: CCc1nc(N)ccc1-c1c(F)c(F)cc2ccc(O)nc12 ; Cl]

Protected intermediate of Example 161 was prepared according to method 9 step 1 starting from 8-bromo-6,7-difluoroquinolin-2(1H)-one 51 (125 mg, 0.48 mmol) and compound 7 (235 mg, 0.72 mmol, 1.5 eq.). The crude was purified by flash chromatography (SiO₂, CyHex/EtOAc: 100/0 to 40/60) to afford compound 174 (120 mg) as a brown oil. M/Z (M+H)⁺: 380.1.

Example 161 was prepared according to method 9 step 2 starting from compound 174 (120 mg). The crude was purified by flash chromatography (SiO₂, DCM/MeOH: 100/0 to 91/9). (20 μm Interchim® SiO₂, DCM/MeOH: 100/0 to 95/5). The obtained product was further purified by flash chromatography (20 μm Interchim® SiO₂, DCM/MeOH: 100/0 to 91/9). The obtained product was triturated twice in Et₂O (5 mL). The obtained product was dissolved in a mixture of aqueous 1N HCl/ACN and the resulting solution was freeze dried to afford Example 161 as a white solid (31 mg, 19% over 2 steps).

¹H-NMR (DMSO-d₆, 400 MHz) δ: 1.10 (t, J 7.6 Hz, 3H, CH₂—CH₃); 2.32-2.41 (m, 1H, CHₐH_{b}—CH₃); 2.42-2.48 (m, 1H, CHₐH_{b}—CH₃); 6.57 (d, J 9.6 Hz, 1H, Ar); 6.95 (d, J 9.0 Hz, 1H, Ar); 7.72 (d, J 9.0 Hz, 1H, Ar); 7.93-7.98 (m, 2H, Ar); 8.11 (bs, 2H, NH₂); 11.13 (s, 1H, NH or OH); 14.27 (bs, 1H, HCl salt). M/Z (M+H)⁺: 302.0. Mp>250° C.

### Example 162: 6-ethyl-5-(1-methylindolin-7-yl)pyridin-2-amine (hydrochloride)

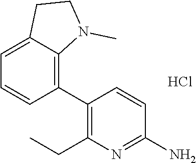 [SMILES: CCc1nc(N)ccc1-c1cccc2c1N(C)CC2 ; Cl]

Protected intermediate of Example 162 was prepared according to method 9 step 1 starting from 7-bromo-1-methylindoline 67 (105 mg, 0.50 mmol) and compound 7 (242 mg, 0.74 mmol, 1.5 eq.). The crude was purified by flash chromatography (SiO₂, CyHex/EtOAc: 100/0 to 95/5) to afford compound 175 (125 mg) as a transparent oil. M/Z (M+H)⁺: 332.2.

Example 162 was prepared according to method 9 step 2 starting from compound 175 (125 mg). The crude was purified by flash chromatography (SiO₂, DCM/MeOH: 100/0 to 95/5). The obtained product was dissolved in a mixture of aqueous 1N HCl/ACN and the resulting solution was freeze dried to afford Example 162 as a pink solid (80 mg, 57% over 2 steps).

¹H-NMR (DMSO-d₆, 400 MHz) δ: 1.11 (t, J 7.6 Hz, 3H, CH₂—CH₃); 2.36 (s, 3H, N—CH₃); 2.54-2.61 (m, 2H, CH₂—CH₃); 2.97 (t, J 8.3 Hz, 2H, Ar—CH₂); 3.30-3.40 (m, 2H, N—CH₂); 6.79-6.83 (m, 2H, 2 Ar); 6.90 (d, J 9.0 Hz, 1H, Ar); 7.17 (d, J 6.8 Hz, 1H, Ar); 7.76 (d, J 9.0 Hz, 1H, Ar); 8.05 (bs, 2H, NH₂); 14.90 (bs, 1H, HCl salt). M/Z (M+H)⁺: 254.1. Mp>250° C.

### Example 163: 7-(6-amino-2-ethylpyridin-3-yl)indolin-2-one (hydrochloride)

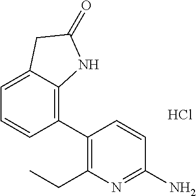 [SMILES: CCc1nc(N)ccc1-c1cccc2c1NC(=O)C2 ; Cl]

Protected intermediate of Example 163 was prepared according to method 9 step 1 starting from 7-bromoindolin-2-one (150 mg, 0.71 mmol) and compound 7 (346 mg, 1.06 mmol, 1.5 eq.). The crude was purified by flash chromatography (SiO₂, CyHex/EtOAc: 90/10 to 70/30) to afford compound 176 (200 mg, 85%) as a yellow solid. M/Z (M+H)⁺: 332.2.

Example 163 was prepared according to method 9 step 2 starting from compound 176 (100 mg, 0.30 mmol). The crude was purified by flash chromatography (SiO₂, DCM/MeOH: 100/0 to 90/10). The obtained product was triturated in H₂O (5 mL). The obtained solid was dissolved in a mixture of aqueous 1N HCl/ACN and the resulting solution was freeze dried to afford Example 163 as a white solid (43 mg, 49%).

¹H-NMR (DMSO-d₆, 400 MHz) δ: 1.07 (t, J 7.6 Hz, 3H, CH₂—CH₃); 2.39-2.47 (m, 1H, CHₐH_{b}—CH₃); 2.54-2.60 (m, 1H, CHₐH_{b}—CH₃); 3.49-3.62 (m, 2H, CH₂—CO); 6.88 (d, J 9.0 Hz, 1H, Ar); 7.01-7.06 (m, 2H, 2*Ar); 7.26-7.28 (m, 1H, Ar); 7.66 (d, J 9.0 Hz, 1H, Ar); 7.93 (bs, 2H, NH₂); 10.25 (s, 1H, NH); 14.17 (bs, 1H, HCl salt). M/Z (M+H)⁺: 254.0. Mp>250° C.

### Example 164: 6-ethyl-5-(indolin-7-yl)pyridin-2-amine (hydrochloride)

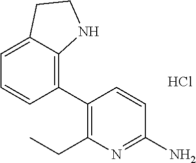 [SMILES: CCc1nc(N)ccc1-c1cccc2c1NCC2 ; Cl]

Protected intermediate of Example 164 was prepared according to method 9 step 1 starting from 7-bromoindoline (120 mg, 0.61 mmol) and compound 7 (297 mg, 0.91 mmol, 1.5 eq.). The crude was purified by flash chromatography (20 μm Interchim® SiO₂, CyHex/EtOAc: 100/0 to 90/10) to afford compound 177 (165 mg) as a light yellow solid. M/Z (M+H)⁺: 318.2

Example 164 was prepared according to method 9 step 2 starting from compound 177 (165 mg). The crude was purified by flash chromatography (20 μm Interchim® SiO₂, DCM/MeOH: 100/0 to 95/5). The obtained product was further purified by preparative HPLC (H₂O (0.5 wt. % HCOOH)/CH₃CN (0.5 wt. % HCOOH): 95/05 to 55/45). The obtained foam was dissolved in a mixture of aqueous 1 N HCl/ACN and the resulting solution was freeze dried to afford Example 164 as a white solid (75 mg, 45% over 2 steps).

¹H-NMR (D₂O, 400 MHz) δ: 1.19 (t, J 7.7 Hz, 3H, CH₂—CH₃); 2.57-2.66 (m, 2H, CH₂—Ar); 3.39 (t, J 7.8 H, 2H, CH₂—CH₃); 3.83 (m, 2H, CH₂—NH); 6.98 (d, J 9.1 Hz, 1H, Ar); 7.33 (d, J 7.1 Hz, 1H, Ar); 7.50 (t, J 7.7 Hz, 1H, Ar); 7.59 (dd, J 7.7, 0.9 Hz, 1H, Ar); 7.78 (d, J 9.1 Hz, 1H, Ar). M/Z (M+H)⁺: 240.0. Mp: 175-183° C.

### Example 165: 6-ethyl-5-(1-methyl-1,2,3,4-tetrahydroquinolin-8-yl)pyridin-2-amine (hydrochloride)

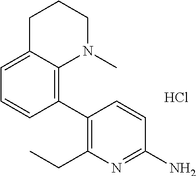 [SMILES: CCc1nc(N)ccc1-c1cccc2c1N(C)CCC2 ; Cl]

Protected intermediate of Example 165 was prepared according to method 9 step 1 starting from 8-bromo-1-methyl-1,2,3,4-tetrahydroquinoline 87 (150 mg, 0.66 mmol) and compound 7 (260 mg, 0.80 mmol, 1.2 eq.). The crude was purified by flash chromatography (SiO₂, CyHex/EtOAc: 100/0 to 90/10) to afford compound 178 (203 mg) as a yellow oil. M/Z (M+H)⁺: 346.2

Example 165 was prepared according to method 9 step 2 starting from compound 178 (203 mg). The crude was purified by flash chromatography (SiO₂, DCM/MeOH: 100/0 to 96/04). The obtained product was further purified by preparative HPLC (H₂O (0.5 wt. % HCOOH)/CH₃CN (0.5 wt. % HCOOH): 90/10 to 50/40). Clean fractions were combined, HCl 1M in H₂O was added and the obtained solution was freeze dried to afford Example 165 as a yellow solid (87 mg, 44% over 2 steps).

¹H-NMR (D₂O, 400 MHz) δ: 1.16 (t, J 7.7 Hz, 3H, CH₂—CH₃); 2.26-2.36 (m, 1H, Ar—CH—H); 2.39-2.49 (m, 2H, CH₂—CH₂); 2.64 (m, 1H, Ar—CH—H); 3.16-3.20 (m, 2H, CH₂—CH₂—CH₂); 3.66-3.69 (m, 2H, CH₂—NMe); 7.04 (d, 1H, J 9.1 Hz, Ar); 7.29 (dd, J 6.9, 2.0 Hz, 1H, Ar); 7.54-7.61 (m, 2H, Ar); 7.93 (d, 1H, J 9.1 Hz, Ar). NH₂ and HCl salt signals not observed. M/Z (M+H)⁺: 268.1. Mp: 155-165° C.

### Example 166: (8-(6-amino-2-ethylpyridin-3-yl)quinolin-2-yl)(morpholino)methanone (hydrochloride)

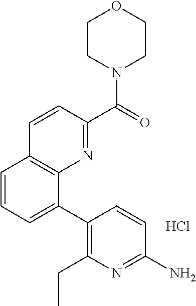 [SMILES: CCc1nc(N)ccc1-c1cccc2ccc(C(=O)N3CCOCC3)nc12 ; Cl]

Protected intermediate of Example 166 was prepared according to method 10 step 1 starting from compound 10 (150 mg, 0.40 mmol) and morpholine (39 mg, 0.44 mmol, 1.1 eq.). The crude was purified by flash chromatography (SiO₂, CyHex/EtOAc: 100/0 to 50/50) to afford compound 179 (131 mg, 74%) as a yellow oil. M/Z (M+H)⁺: 441.2.

Example 166 was prepared according to method 10 step 2 starting from compound 179 (131 mg, 0.30 mmol). The crude was purified by flash chromatography (SiO₂, DCM/MeOH: 100/0 to 95/5). The obtained solid was triturated in Et₂O (2×2 mL). The obtained product was dissolved in a mixture of aqueous 1N HCl/ACN and the resulting solution was freeze dried to afford Example 166 as a white solid (51 mg, 43%).

¹H-NMR (D₂O, 400 MHz) δ: 1.07 (t, J 7.6 Hz, 3H, CH₃); 2.46-2.67 (m, 2H, CH₂); 3.45-3.58 (m, 2H, CH₂); 3.63-3.65 (m, 2H, CH₂); 3.80-3.89 (m, 4H, 2*CH₂); 7.01 (d, J 9.0 Hz, 1H, Ar); 7.77 (d, J 8.5 Hz, 1H, Ar); 7.82-7.89 (m, 3H, Ar); 8.18 (dd, J 7.8, 1.9 Hz, 1H, Ar); 8.65 (d, J 8.5 Hz, 1H, Ar). NH₂ and HCl salt not observed. M/Z (M+H)⁺: 363.2. Mp: 130-135° C.

### Example 167: (8-(6-amino-2-ethylpyridin-3-yl)quinolin-2-yl)(1,4-oxazepan-4-yl)methanone (hydrochloride)

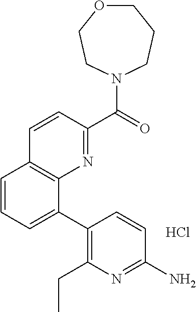 [SMILES: CCc1nc(N)ccc1-c1cccc2ccc(C(=O)N3CCCOCC3)nc12 ; Cl]

Protected intermediate of Example 167 was prepared according to method 10 step 1 starting from compound 10 (150 mg, 0.40 mmol) and 1,4-oxazepane (45 mg, 0.44 mmol, 1.1 eq.). The crude was purified by flash chromatography (SiO₂, CyHex/EtOAc: 100/0 to 50/50) to afford compound 180 (109 mg, 59%) as a yellow solid. M/Z (M+H)⁺: 455.2.

Example 167 was prepared according to method 10 step 2 starting from compound 180 (109 mg, 0.24 mmol). The crude was purified by flash chromatography (SiO₂, DCM/MeOH: 100/0 to 95/5). The obtained foam was triturated twice in Et₂O (2 mL). The obtained product was dissolved in a mixture of aqueous 1N HCl/ACN and the resulting solution was freeze dried to afford Example 167 as a white solid (69 mg, 70%).

¹H-NMR (D₂O, 400 MHz) δ:1.05 (t, J 7.7 Hz, 3H, CH₃); 1.65-1.71 (m, 1H, C—H—H); 2.04-2.10 (m, 1H, C—H—H); 2.45-2.64 (m, 2H, CH₂); 3.49-3.54 (m, 2H, CH₂); 3.60-3.62 (m, 1H, C—H—H); 3.78 (t, J 5.4 Hz, 1H, C—H—H); 3.81-3.96 (m, 4H, 2*CH₂); 7.00 (dd, J 9.1, 7.1 Hz, 1H, Ar); 7.75 (d, J 8.5 Hz, 1H, Ar); 7.81-7.88 (m, 3H, Ar); 8.16-8.18 (m, 1H, Ar); 8.62 (d, J 8.5 Hz, 1H, Ar). NH₂ and HCl salt not observed. M/Z (M+H)⁺: 377.2. Mp: 120-131° C.

### Example 168: 8-(6-amino-2-ethylpyridin-3-yl)-N-cyclohexyl-N-ethylquinoline-2-carboxamide (hydrochloride)

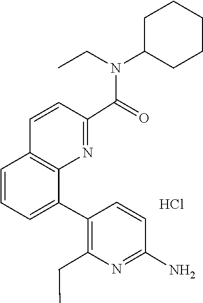 [SMILES: CCc1nc(N)ccc1-c1cccc2ccc(C(=O)N(CC)C3CCCCC3)nc12 ; Cl]

Protected intermediate of Example 168 was prepared according to method 10 step I starting from compound 10 (180 mg, 0.49 mmol) and N-ethylcyclohexanamine (68 mg, 0.53 mmol, 1.1 eq.). The crude was purified by flash chromatography (SiO₂, CyHex/EtOAc: 100/0 to 50/50) to afford compound 181 (155 mg, 67%) as a yellow oil. M/Z (M+H)⁺: 481.3

Example 168 was prepared according to method 10 step 2 starting from compound 181 (155 mg, 0.33 mmol). The crude was purified by flash chromatography (SiO₂, DCM/MeOH: 100/0 to 97/3). The obtained foam was triturated twice in Et₂O (2 mL). The obtained product was dissolved in a mixture of aqueous 1N HCl/ACN and the resulting solution was freeze dried to afford Example 168 as a white solid (58 mg, 41%).

¹H-NMR (DMSO-d₆+D₂O, 400 MHz) presence of rotamer δ: 0.65-0.71 (m, 1.4H, CH₂); 0.76 (t, J 7.1 Hz, 0.6H, CH₃); 0.88-0.96 (m, 0.6H, CH₂); 0.99 (t, J 7.7 Hz, 0.6H, CH₃); 1.02 (t, J 7.7 Hz, 2.4H, CH₃); 1.11 (t, J 7.1 Hz, 2.4H, CH₃); 1.25-1.78 (m, 8H, 4*CH₂); 2.36-2.42 (m, 2H, CH₂); 3.09 (m, 0.6H, CH); 3.30-3.37 (m, 2.4H, CH+CH₂); 6.87-6.90 (m, 1H, Ar); 7.61-7.65 (m, 1H, Ar); 7.74-7.78 (m, 3H, Ar); 8.06-8.12 (m, 1H, Ar); 8.51-8.55 (m, 1H, Ar). NH₂ and HCl salt not observed. M/Z (M+H)⁺: 403.3. Mp: 142-148° C.

### Example 169: (8-(6-amino-2-ethylpyridin-3-yl)quinolin-2-yl)(azepan-1-yl)methanone (hydrochloride)

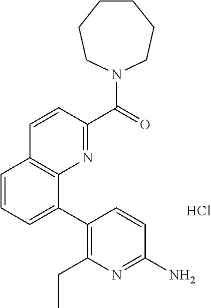 [SMILES: CCc1nc(N)ccc1-c1cccc2ccc(C(=O)N3CCCCCC3)nc12 ; Cl]

Protected intermediate of Example 169 was prepared according to method 10 step 1 starting from compound 10 (180 mg, 0.49 mmol) and azepane (53 mg, 0.53 mmol, 1.1 eq.). The crude was purified by flash chromatography (SiO₂, CyHex/EtOAc: 100/0 to 60/40) to afford compound 182 (144 mg, 66%) as an orange solid. M/Z (M+H)⁺: 481.3

Example 169 was prepared according to method 10 step 2 starting from compound 182 (144 mg, 0.32 mmol). The crude was purified by flash chromatography (KPNH, CyHex/EtOAc: 100/0 to 30/70). The obtained foam was triturated twice in Et₂O (2 mL). The obtained product was dissolved in a mixture of aqueous 1 N HCl/ACN and the resulting solution was freeze dried to afford Example 169 as a white solid (54 mg, 41%).

¹H-NMR (D₂O, 400 MHz) δ: 1.05 (t, J 7.6 Hz, 3H, CH₃); 1.47-1.54 (m, 4H, 2*CH₂); 1.61-1.67 (m, 2H, CH₂); 1.78-1.84 (m, 2H, CH₂); 2.43-2.64 (m, 2H, CH₂); 3.32-3.34 (m, 2H, CH₂); 3.61-3.74 (m, 2H, CH₂); 6.98 (d, J 9.0, 1 H, Ar); 7.70 (d, J 8.5 Hz, 1H, Ar); 7.80-7.87 (m, 3H, Ar); 8.15 (dd, J 8.1, 1.6 Hz, 1H, Ar); 8.60 (d, J 8.5 Hz, 1H, Ar). NH₂ and HCl salt not observed. M/Z (M+H)⁺: 375.2. Mp: 140-144° C.

### Example 170: 8-(6-amino-2-ethylpyridin-3-yl)-N-ethyl-N-isopropylquinoline-2-carboxamide (hydrochloride)

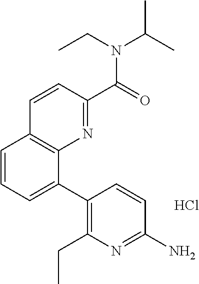 [SMILES: CCc1nc(N)ccc1-c1cccc2ccc(C(=O)N(CC)C(C)C)nc12 ; Cl]

Protected intermediate of Example 170 was prepared according to method 10 step 1 starting from compound 10 (180 mg, 0.49 mmol) and ethylpropan-2-amine (47 mg, 0.53 mmol, 1.1 eq.). The crude was purified by flash chromatography (SiO₂, CyHex/EtOAc: 100/0 to 50/50) to afford compound 183 (126 mg, 59%) as a yellow oil. M/Z (M+H)⁺: 441.3

Example 170 was prepared according to method 10 step 2 starting from compound 183 (126 mg, 0.29 mmol). The crude was purified by flash chromatography (SiO₂, DCM/MeOH: 100/0 to 95/5). The obtained foam was triturated twice in Et₂O (2 mL). The obtained product was dissolved in a mixture of aqueous 1N HCl/ACN and the resulting solution was freeze dried to afford Example 170 as a white solid (54 mg, 47%).

¹H-NMR (D₂O, 400 MHz) presence of rotamer δ: 0.94-0.97 (m, 0.75H, CH₃); 1.03-1.14 (m, 7.25H, CH₃); 1.28-1.35 (m, 4H, CH₃); 2.46-2.64 (m, 2H, CH₂); 3.18-3.34 (m, 0.5H, CH); 3.46-3.51 (m, 1.5H, CH+CH₂); 3.89-3.95 (m, 0.75H, CH₂); 4.51-4.57 (m, 0.25H, CH); 6.97 (d, J 9.0 Hz, 1H, Ar); 7.67-7.71 (m, 1H, Ar); 7.80-7.86 (m, 3H, Ar); 8.17 (dd, J 7.8, 2.0 Hz, 1H, Ar); 8.62 (d, J 8.5 Hz, 1H, Ar). NH₂ and HCl salt not observed. M/Z (M+H)⁺: 363.3. Mp: 121-125° C.

### Example 171: (8-(6-amino-2-ethylpyridin-3-yl)quinolin-2-yl)(8-oxa-3-azabicyclo[3.2.1]octan-3-yl)methanone (hydrochloride)

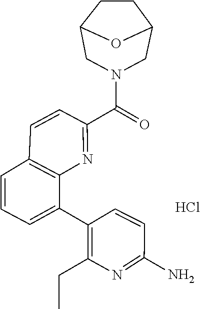 [SMILES: CCc1nc(N)ccc1-c1cccc2ccc(C(=O)N3CC4CCC(C3)O4)nc12 ; Cl]

Protected intermediate of Example 171 was prepared according to method 10 step 1 starting from compound 10 (145 mg, 0.39 mmol) and 8-oxa-3-azabicyclo[3.2.1]octane hydrochloride (64 mg, 0.43 mmol, 1.1 eq.). The crude was purified by flash chromatography (SiO₂, CyHex/EtOAc: 100/0 to 40/60) to afford compound 184 (139 mg, 76%) as a yellow oil. M/Z (M+H)⁺: 467.3

Example 171 was prepared according to method 10 step 2 starting from compound 184 (139 mg, 0.30 mmol). The crude was purified by flash chromatography (SiO₂, DCM/MeOH: 100/0 to 96/4). The obtained foam was triturated twice in Et₂O (2 mL). The obtained product was dissolved in a mixture of aqueous 1N HCl/ACN and the resulting solution was freeze dried to afford Example 171 as a white solid (72 mg, 57%).

¹H-NMR (D₂O, 400 MHz) presence of rotamer δ: 1.00 (t, J 7.5, 1.5H, CH₃); 1.07 (t, J 7.5, 1.5H, CH₃); 1.69-1.88 (m, 3H, CH₂+CH—H); 1.98-2.07 (m, 1H, CH—H); 2.45-2.70 (m, 2H, CH₂); 3.23 (d, J 13.1 Hz, 1H, CH—H); 3.29 (d, J 13.1 Hz, 0.5H, CH—H); 3.37 (d, J 13.1 Hz, 0.5H, CH—H); 3.58 (d, J 12.8 Hz, 0.5H, CH—H); 3.77 (d, J 12.8 Hz, 0.5H, CH—H); 4.21 (dd, J 13.1, 6.8 Hz, 1H, CH); 4.32 (t, J 7.3 Hz, 1H, CH—H); 4.61 (d, J 7.3 Hz, 1H, CH—H); 7.00 (d, J 9.2 Hz, 1H, Ar); 7.75 (d, J 8.2 Hz, 1H, Ar); 7.78-7.89 (m, 4H, Ar); 8.17 (d, J 8.0 Hz, 1H, Ar); 8.62 (t, J 7.3 Hz, 1H, Ar). M/Z (M+H)⁺: 389.2. Mp: 150-160° C.

### Example 172: (8-(6-amino-2-ethylpyridin-3-yl)quinolin-2-yl)(4-phenylpiperidin-1-yl)methanone (hydrochloride)

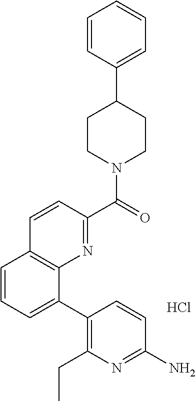 [SMILES: CCc1nc(N)ccc1-c1cccc2ccc(C(=O)N3CCC(c4ccccc4)CC3)nc12 ; Cl]

Protected intermediate of Example 172 was prepared according to method 10 step 1 starting from compound 10 (145 mg, 0.39 mmol) and 4-phenylpiperidine (69 mg, 0.43 mmol, 1.1 eq.). The crude was purified by flash chromatography (SiO₂, CyHex/EtOAc: 100/0 to 40/60) to afford compound 185 (177 mg, 88%) as a yellow oil. M/Z (M+H)⁺: 515.3

Example 172 was prepared according to method 10 step 2 starting from compound 185 (177 mg, 0.34 mmol), the crude was purified by flash chromatography (SiO₂, DCM/MeOH: 100/0 to 97/3). The obtained foam was triturated twice in pentane (2 mL). The obtained product was dissolved in a mixture of aqueous 1N HCl/ACN and the resulting solution was freeze dried to afford Example 172 as a white solid (85 mg, 52%).

¹H-NMR (DMSO-d₆, 400 MHz) presence of rotamer δ: 0.98 (m, 1.5H, CH₃); 1.14 (m, 1.5H, CH₃); 1.36-1.58 (m, 3H, CH₂+CH—H); 1.88 (d, J 12.5 Hz, 1H, CH—H); 2.38-2.45 (m, 2H, CH₂—CH₃); 2.77-2.90 (m, 2H, CH); 2.97-3.06 (m, 1H, CH); 3.90 (d, J 13.0 Hz, 1H, CH—H); 4.63 (d, J 13.0 Hz, 1H, CH—H); 6.83-7.01 (m, 1H, Ar); 7.17 (d, J 7.2 Hz, 2H, Ar); 7.23 (t, J 7.2 Hz, 1H, Ar); 7.34-7.42 (m, 2H, Ar); 7.78-7.81 (m, 4H, Ar); 7.86 (bs, 2H, NH₂); 8.15-8.18 (m, 1H, Ar); 8.61 (d, J 8.5 Hz, 1H, Ar); 13.97-14.14 (m, 1H, HCl salt). M/Z (M+H)⁺: 437.2. Mp>250° C.

### Example 173: 8-(6-amino-2-ethylpyridin-3-yl)-N-(tetrahydro-2H-pyran-4-yl)quinoline-2-carboxamide (hydrochloride)

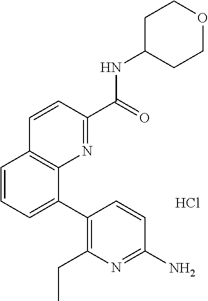 [SMILES: CCc1nc(N)ccc1-c1cccc2ccc(C(=O)NC3CCOCC3)nc12 ; Cl]

Protected intermediate of Example 173 was prepared according to method 10 step 1 starting from compound 10 (145 mg, 0.39 mmol) and tetrahydro-2H-pyran-4-amine (43 mg, 0.43 mmol, 1.1 eq.). The crude was purified by flash chromatography (SiO₂, CyHex/EtOAc: 100/0 to 50/50) to afford compound 186 (152 mg, 86%) as a yellow oil. M/Z (M+H)⁺: 455.2

Example 173 was prepared according to method 10 step 2 starting from compound 186 (152 mg, 0.33 mmol), the crude was purified by flash chromatography (SiO₂, DCM/MeOH: 100/0 to 95/5). The obtained foam was triturated twice in pentane (2 mL). The obtained product was dissolved in a mixture of aqueous 1 N HCl/ACN and the resulting solution was freeze dried to afford Example 173 as a white solid (88 mg, 64%).

¹H-NMR (DMSO-d₆, 400 MHz) δ: 1.11 (t, J 7.6 Hz, 3H, CH₃); 1.43-1.52 (m, 2H, CH₂); 1.81-1.85 (m, 2H, CH₂); 2.52-2.54 (m, 2H, CH₂); 3.44-3.51 (m, 2H, CH₂); 3.77 (td, J 11.6, 4.2 Hz, 2H, CH₂); 3.95-4.05 (m, 1H, CH); 6.98 (d, J 9.1 Hz, 1H, Ar); 7.91-8.06 (m, 6H, NH+NH₂+Ar); 8.17-8.21 (m, 2H, Ar); 8.69 (d, J 8.6 Hz, 1H, Ar); 14.13 (s, 1H, HCl salt). M/Z (M+H)⁺: 377.2. Mp: 112-125° C.

### Example 174: 8-(6-amino-2-ethylpyridin-3-yl)-N-benzylquinoline-2-carboxamide (hydrochloride)

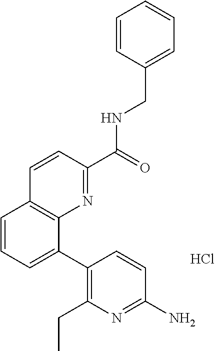 [SMILES: CCc1nc(N)ccc1-c1cccc2ccc(C(=O)NCc3ccccc3)nc12 ; Cl]

Protected intermediate of Example 174 was prepared according to method 10 step 1 starting from compound 10 (145 mg, 0.39 mmol) and phenylmethanamine (46 mg, 0.43 mmol, 1.1 eq.). The crude was purified by flash chromatography (SiO₂, CyHex/EtOAc: 100/0 to 70/30) to afford compound 187 (140 mg, 78%) as an orange oil. M/Z (M+H)⁺: 461.3

Example 174 was prepared according to method 10 step 2 starting from compound 187 (140 mg, 0.30 mmol). The crude was purified by flash chromatography (SiO₂, DCM/MeOH: 100/0 to 96/4). The obtained foam was triturated in Et₂O (2 mL) and in pentane (2 mL). The obtained product was dissolved in a mixture of aqueous 1 N HCl/ACN and the resulting solution was freeze dried to afford Example 174 as a white solid (74 mg, 58%).

1H-NMR (D₂O, 400 MHz) δ: 0.91 (t, J 7.7 Hz, 3H, CH₃); 2.35-2.42 (m, 2H, CH₂); 4.46-4.57 (m, 2H, CH₂); 6.71 (d, J 9.0 Hz, 1H, Ar); 7.29-7.31 (m, 2H, Ar); 7.42-7.48 (m, 3H, Ar); 7.54 (d, J 9.0 Hz, 1H, Ar); 7.73-7.78 (m, 2H, Ar); 8.02-8.07 (m, 2H, Ar); 8.47 (d, J 8.6 Hz, 1H, Ar). M/Z (M+H)⁺: 383.2. Mp: 102-114° C.

### Example 175: 8-(6-amino-2-ethylpyridin-3-yl)-N-(oxetan-3-yl)quinoline-2-carboxamide

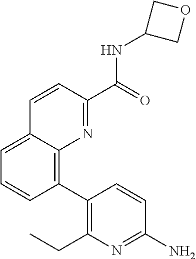 [SMILES: CCc1nc(N)ccc1-c1cccc2ccc(C(=O)NC3COC3)nc12]

Protected intermediate of Example 175 was prepared according to method 9 step 1 starting from 8-bromo-N-(oxetan-3-yl)quinoline-2-carboxamide 61 (96 mg, 0.31 mmol) and compound 7 (112 mg, 0.34 mmol, 1.1 eq.). The crude was purified by flash chromatography (SiO₂, CyHex/EtOAc: 100/0 to 50/50) to afford compound 188 (111 mg, 83%) as a yellow oil. M/Z (M+H)⁺: 427.2.

Example 175 was prepared according to method 9 step 2 starting from compound 188 (111 mg, 0.26 mmol), the crude was purified by flash chromatography (SiO₂, DCM/MeOH: 100/0 to 95/5). The obtained product was triturated in pentane (5 mL) and filtered to afford Example 175 as a beige solid (32 mg, 35%).

¹H-NMR (DMSO-d₆, 400 MHz) δ: 1.01 (t, J 7.5 Hz, 3H, CH₃); 2.28-2.33 (m, 2H, CH₂—CH₃); 4.38 (t, J 6.4 Hz, 2H, 2*CHₐH_{b}—O); 4.81 (t, J 7.1 Hz, 2H, 2*CHₐH_{b}—O); 4.94-5.02 (m, 1H, CH); 5.96 (bs, 2H, NH₂); 6.45 (d, J 8.3 Hz, 1H, Ar); 7.38 (d, J 8.3 Hz, 1H, Ar); 7.74-7.79 (m, 2H, Ar); 8.05-8.09 (m, 1H, Ar); 8.09 (d, J 8.6 Hz, 1H, Ar); 8.38 (d, J 7.3 Hz, 1H, Ar); 8.62 (d, J 8.6 Hz, 1H, Ar). M/Z (M+H)⁺: 349.2. Mp: 156-162° C.

### Example 176: 6-ethyl-5-(7-fluorochroman-8-yl)pyridin-2-amine (hydrochloride)

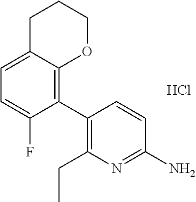 [SMILES: CCc1nc(N)ccc1-c1c(F)ccc2c1OCCC2 ; Cl]

Protected intermediate of Example 176 was prepared according to method 9 step 1 starting from 8-bromo-7-fluorochromane 95 (163 mg, 0.71 mmol) and compound 7 (345 mg, 1.06 mmol, 1.5 eq.). The crude was purified by flash chromatography (SiO₂, DCM/MeOH: 100/0 to 98/02). The obtained foam was further purified by flash chromatography (SiO₂, CyHex/EtOAc: 100/0 to 80/20) to afford compound 189 (281 mg) as a colorless oil. M/Z (M+H)⁺: 351.2.

Example 176 was prepared according to method 9 step 2 starting from compound 189 (281 mg). The crude was purified by flash chromatography (SiO₂, DCM/MeOH: 100/0 to 95/5). The obtained foam was further purified by flash chromatography (20 μm Interchim® SiO₂, CyHex/EtOAc: 100/0 to 30/70). The obtained foam was triturated twice in Et₂O (5 mL), then dissolved in a mixture of aqueous 1 N HCl/ACN and the resulting solution was freeze dried to afford Example 176 as a white solid (138 mg, 63% over 2 steps).

¹H-NMR (DMSO-d₆, 400 MHz) δ: 1.11 (t, J 7.6 Hz, 3H, CH₂—CH₃); 1.67-1.92 (m, 2H, O—CH₂—CH₂); 2.47 (q, J 7.6 Hz, 2H, CH₂—CH₃); 2.77 (t, J 6.4 Hz, 2H, Ph-CH₂); 4.10 (dd, J 5.6, 4.4 Hz, 2H, O—CH₂—CH₂); 6.82 (dd, J, 9.2, 8.4 Hz, 1H, Ar); 6.90 (d, J 9.2 Hz, 1H, Ar); 7.19 (dd, J 8.4, 6.8 Hz, 1H, Ar); 7.69 (d, J 9.2 Hz, 1H, Ar); 7.96 (bs, 2H, NH₂); 14.16 (bs, 1H, HCl salt). M/Z (M+H)⁺: 273.1. Mp: 85-95° C.

### Example 177: 6-ethyl-5-(7-fluoro-2,2-dimethylchroman-8-yl)pyridin-2-amine (hydrochloride)

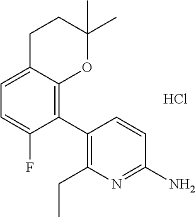 [SMILES: CCc1nc(N)ccc1-c1c(F)ccc2c1OC(C)(C)CC2 ; Cl]

Protected intermediate of Example 177 was prepared according to method 9 step 1 starting from 8-bromo-7-fluoro-2,2-dimethylchromane 96 (85 mg, 0.33 mmol) and compound 7 (160 mg, 0.49 mmol, 1.5 eq.). The crude was purified by flash chromatography (SiO₂, CyHex/EtOAc: 100/0 to 90/10). The obtained F product was further purified by flash chromatography (20 μm Interchim® SiO₂, CyHex/EtOAc: 100/0 to 90/10) to afford compound 190 (99 mg) as a yellow oil. M/Z (M+H)⁺: 379.3.

Example 177 was prepared according to method 9 step 2 starting from compound 190 (99 mg). The crude was purified by flash chromatography (SiO₂, CyHex/EtOAc: 100/0 to 30/70). The obtained foam was triturated twice in Et₂O (3 mL), then dissolved in a mixture of aqueous 1N HCl/ACN and the resulting solution was freeze dried to afford Example 177 as a white solid (35 mg, 31% over 2 steps).

¹H-NMR (DMSO-d₆, 400 MHz) δ: 1.10 (t, J 7.6 Hz, 1H, CH₂—CH₃); 1.20 (s, 6H, 2*CH₃); 1.76 (t, J 6.8 Hz, 2H, Ph-CH₂—CH₂); 2.45 (q, J 7.6 Hz, 2H, CH₂—CH₃); 2.77 (t, J 6.8 Hz, 2H, Ph-CH₂); 6.80 (dd, J, 9.2, 8.4 Hz, 1H, Ar); 6.90 (d, J 8.8 Hz, 1H, Ar); 7.22 (dd, J 8.4, 6.8 Hz, 1H, Ar); 7.65 (d, J 8.8 Hz, 1H, Ar); 7.94 (bs, 2H, NH₂); 14.11 (bs, 1H, HCl salt). M/Z (M+H)⁺: 301.1. Mp: 80-90° C.

### Example 178: 6-ethyl-5-(8-fluoro-2,5-dihydrobenzo[b]oxepin-9-yl)pyridin-2-amine (hydrochloride)

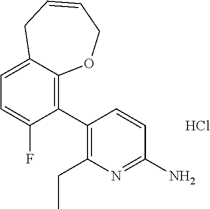 [SMILES: CCc1nc(N)ccc1-c1c(F)ccc2c1OCC=CC2 ; Cl]

Protected intermediate of Example 178 was prepared according to method 9 step 1 starting from 9-bromo-8-fluoro-2,5-dihydrobenzo[b]oxepine 98 (380 mg, 1.56 mmol) and compound 7 (765 mg, 2.34 mmol, 1.5 eq.). The crude was purified by flash chromatography (SiO₂, CyHex/EtOAc: 100/0 to 80/20). The obtained product was further purified by flash chromatography (20 μm Interchim® SiO₂, CyHex/EtOAc: 100/0 to 80/20) to afford compound 191 (265 mg, 47%) as a yellow oil. M/Z (M+H)⁺: 363.3.

Example 178 was prepared according to method 9 step 2 starting from compound 191 (262 mg, 0.72 mmol). The crude was purified by flash chromatography (20 μm Interchim® SiO₂, DCM/MeOH: 100/0 to 98/2) to afford 6-ethyl-5-(8-fluoro-2,5-dihydrobenzo[b]oxepin-9-yl)pyridin-2-amine 192 (203 mg, 99%) as a white solid. 40 mg of 192 were dissolved in a mixture of aqueous 1N HCl/ACN and the resulting solution was freeze dried to afford Example 178 as a white solid (38 mg).

¹H-NMR (DMSO-d₆, 400 MHz) δ: 1.13 (t, J 7.6 Hz, 1.5 H one rotamer of CH₂—CH₃); 1.14 (t, J 7.6 Hz, 1.5 H other rotamer of CH₂—CH₃); 2.52-2.56 (m, 2H, CH₂—CH₃); 2.97-3.05 (m, 1H, Ph-CH₂); 3.41-3.48 (m, 1H, Ph-CH₂); 5.21 (ddt, J 10.4, 6.8, 1.2 Hz, 1H, O—CH₂); 5.27-5.37 (m, 2H, O—CH₂+CH═CH); 6.02 (dddd, J 17.2, 10.4, 6.4; 2.0 Hz, 1H, CH═CH); 6.81 (dd, J 10.0, 8.4 Hz, 1H, Ar); 6.91-6.92 (m, 1H, Ar); 7.27-7.30 (m, 1H, Ar); 7.74 (d, J 9.2 Hz, 1H, Ar); 7.97 (bs, 2H, NH₂); 14.13 (bs, 1H, HCl salt). M/Z (M+H)⁺: 285.0. Mp: 43-52° C.

### Example 179: 6-ethyl-5-(8-fluoro-2,3,4,5-tetrahydrobenzo[b]oxepin-9-yl)pyridin-2-amine (hydrochloride)

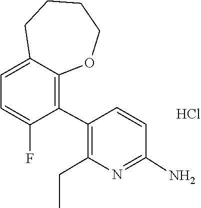 [SMILES: CCc1nc(N)ccc1-c1c(F)ccc2c1OCCCC2 ; Cl]

Under Argon, to a solution of 6-ethyl-5-(8-fluoro-2,5-dihydrobenzo[b]oxepin-9-yl)pyridin-2-amine 192 (163 mg, 0.57 mmol, 1.0 eq.) in EtOH (2.9 mL) was added Pd/C 10 wt. % (61 mg, 0.06 mmol, 0.1 eq.). The reaction mixture was sparged with H₂ for 5 min, then stirred under H₂ atmosphere at 25° C. for 22 h. The reaction mixture was filtered through a pad of Celite® and the cake was washed with EtOH (20 mL). The filtrate was concentrated to dryness. The crude was purified by flash chromatography (SiO₂, CyHex/EtOAc: 100/0 to 20/80). The obtained foam was dissolved in a mixture of aqueous 1 N HCl/ACN and the resulting solution was freeze dried to afford Example 179 as a white solid (58 mg, 31%).

¹H-NMR (DMSO-d₆, 400 MHz) δ: 0.89 (t, J 7.4 Hz, 1.5 H, one rotamer of CH₂—CH₃); 0.89 (t, J 7.4 Hz, 1.5 H, other rotamer of CH₂—CH₃); 1.10-1.17 (m, 3H, O—CH₂—CH₂—CH₂+O—CH₂—CH₂—CH₂); 1.62-1.75 (m, 2H, O—CH₂—CH₂—CH₂+Ph-CH₂); 2.52-2.60 (m, 2H, CH₂—CH₃); 2.84-2.94 (m, 1H, Ph-CH₂); 3.28-3.32 (m, 1H, O—CH₂); 4.78-4.86 (m, 1H, O—CH₂); 6.77 (dd, J 10.0, 8.4 Hz, 1H, Ar); 6.91-6.92 (m, 1H, Ar); 7.26 (m, 1H, Ar); 7.73 (d, J 9.2 Hz, 1H, Ar); 7.97 (bs, 2H, NH₂); 14.15 (bs, 1H, HCl salt). M/Z (M+H)⁺: 287.1. Mp: 47-65° C.

### III. Biological Experiments

### Example 180: Human NPFFR1 Evaluation Using BRET Biosensors (IC₅₀)

Compounds of the present invention were tested successively for their agonist and antagonist activities on human NPFFR1 (hNPFFR1) receptor transiently over-expressed in HEK-293 T cells. Compounds exert agonist activity if, by themselves in absence of neuropeptide RFRP-3 (also named NPVF) they activate hNPFFR1; and they exert antagonist activity if they decrease the action of RFRP-3 on the receptor.

The assay used to measure compound activity is based on BRET (Bioluminescence Resonance Energy Transfer) biosensors and is designed to monitor the plasma membrane translocation of protein that interacts with specific Ga subunit. The specific effector (luciferase tagged: BRET donor) recruited at the membrane will be in close proximity to a plasma membrane anchor (GFP tagged: BRET acceptor) to induce a BRET signal. Biosensors are described in the patent application WO 2016/041093 A1 (Biosensors for monitoring biomolecule localization and trafficking in cells).

### Cell Culture and Transfection

HEK-293 T cells are maintained in Dulbecco's Modified Eagle's Medium supplemented with 10% Foetal Calf Serum, 1% Penicillin/Streptomycin at 37° C./5% CO₂.

Cells are co-transfected using polyethylenimine (25 kDa linear) with four DNA plasmids encoding: hNPFFR1, GαoB, a Gi family specific intracellular effector fused to luciferase (BRET donor), a plasma membrane effector fused to GFP (BRET acceptor). After transfection, cells are cultured for 48 h at 37° C./5% CO₂.

### BRET Assay

Receptor activity is detected by changes in BRET signal.

On the day of the assay, cells are detached using trypsin 0.05%, resuspended in assay buffer (1.8 mM CaCl₂, 1 mM MgCl₂, 2.7 mM KCl, 137 mM NaCl, 0.4 mM NaH₂PO₄, 5.5 mM D-Glucose, 11.9 mM NaHCO₃, 25 mM Hepes) and seeded in 384 well plate at a density of 20,000 cells per well. Then, plates are equilibrated 3.5 hours at 37° C. before adding compounds.

Compounds and luciferase substrate are added to the cells using an automated device (Freedom Evo®, Tecan) and BRET readings are collected on EnVision (PerkinElmer) with specific filters (410 nm BW 80 nm, 515 nm BW 30 nm).

Agonist and antagonist activities of compounds are consecutively evaluated on the same cell plate. Agonist activity is first measured after 10 minutes incubation with compound alone on the cells. Then, cells are stimulated by an EC80 RFRP-3 concentration and luminescence is recorded for additional 10 minutes. EC80 RFRP-3 concentration is the concentration giving 80% of the maximal RFRP-3 response. Agonist or antagonist activities are evaluated in comparison to basal signals evoked by assay buffer or EC80 RFRP-3 alone, respectively.

### IC₅₀ Determination

For IC₅₀ determination, a dose-response test is performed using 20 concentrations (ranging over 6 logs) of each compound. Dose-response curves are fitted using the sigmoidal dose-response (variable slope) analysis in GraphPad Prism software (GraphPad Software) and IC₅₀ of antagonist activity is calculated. Dose-response experiments are performed in duplicate, in two independent experiments.

According to the biological test procedure, the following compounds showed IC₅₀ values in the ranges as detailed below:

IC₅₀>1000 nM: Examples 2, 5, 16, 17, 18, 19, 27, 35, 36, 48, 54, 75, 100, 102, 103, 152, 156, 157

IC₅₀ between 100 nM and 1000 nM: Examples 3, 4, 7, 10, 12, 13, 14, 15, 20, 21, 22, 24, 26, 28, 29, 30, 33, 34, 38, 41, 42, 43, 44, 45, 50, 69, 78, 79, 80, 81, 82, 83, 85, 86, 87, 88, 89, 91, 92, 96, 101, 104, 106, 107, 112, 114, 120, 130, 131, 140, 153, 154, 155, 158, 159, 161, 162, 163, 164, 165, 172

IC₅₀<100 nM: Examples 1, 6, 8, 9, 11, 23, 25, 31, 32, 37, 39, 40, 46, 47, 49, 51, 52, 53, 55, 56, 57, 58, 59, 60, 61, 62, 63, 64, 65, 66, 67, 68, 70, 71, 72, 73, 74, 76, 77, 84, 90, 93, 94, 95, 97, 98, 99, 105, 108, 109, 110, 111, 113, 115, 116, 117, 118, 119, 121, 122, 123, 124, 125, 126, 127, 128, 129, 132, 133, 134, 135, 136, 137, 141, 142, 143, 144, 145, 146, 147, 148, 149, 150, 151, 160, 166, 167, 168, 169, 170, 171, 173, 174, 176, 177, 178, 179

### Example 181: In Vivo Evaluation on Morphine-Induced Hyperalgesia Model in the Mouse

This method, which detects opioid-induced hyperalgesia, follows those well-known by one skilled in the art and described in the literature (Elhabazi K et al., J Vis Exp. 2014; (89):e51264). The procedure applied to the compounds of the invention is as follows:

Nociceptive thresholds are assessed using the tail immersion test (TIT). After TIT, mice are treated daily during 8 consecutive days with morphine (10 mg/kg, sub-cutaneous s.c.) +/− tested compounds (10 mg/kg, s.c.) 20 min before morphine injection for example 31 and 32 and twice a day for examples 56 and 143 (20 min before morphine injection, and in the afternoon).

Mice used were male C57/B16N 8 weeks old at the beginning of the experiment. During TIT the tail of the animal is immersed two-thirds in a water bath at 47° C. and the withdrawal latency is recorded in seconds. The cutoff is 25 seconds, to avoid tissue damage. After TIT mice received an injection depending on their group then are replaced to their home cages.

### Data Analysis

FIGS. 1A and 1B show the mean time of tail withdrawal latency in each group of animals. The anti-hyperalgesia effect of the tested compounds was compared to vehicle-treated group using ANOVA test followed by the Bonferroni's test. The insert at the bottom shows the comparison between groups of the global Area Under Curve (AUC) over D0 to D8 period.

### Results

As illustrated in FIGS. 1A and 1B, examples 31, 32, 56 and 143 administered at 10 mg/kg s.c. showed a significant blockade of morphine-induced hyperalgesia.

These results demonstrate that the compounds of formula (I) can be used in the therapeutic or prophylactic treatment of opioid-induced hyperalgesia, including in particular morphine-induced hyperalgesia, and further that the therapeutic use of an opioid analgesic (such as morphine) in combination with a compound of formula (I) according to the invention is advantageous as it allows to prevent or reduce the development of opioid-induced hyperalgesia.

1. A compound of the following formula (I) 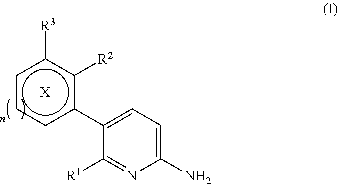
wherein:
R¹ is selected from C₁₋₅ alkyl, C₂₋₅ alkenyl, C₂₋₅ alkynyl, —Br, —I, C₁₋₅ haloalkyl, —CN, —NH₂, —NH(C₁₋₅ alkyl), —N(C₁₋₅ alkyl)(C₁₋₅ alkyl), —(C₀₋₃ alkylene)-cycloalkyl, and —(C₀₋₃ alkylene)-heterocycloalkyl, wherein the cycloalkyl moiety in said —(C₀₋₃ alkylene)-cycloalkyl and the heterocycloalkyl moiety in said —(C₀₋₃ alkylene)-heterocycloalkyl are each optionally substituted with one or more groups R^{A};
ring X is phenyl or a monocyclic heteroaryl having 5+n ring members, wherein said phenyl or said heteroaryl is optionally substituted with one or more groups R^{X};
n is 0 or 1;
R² and R³ are mutually joined to form, together with ring X, a bicyclic or tricyclic heteroaryl, wherein said heteroaryl is optionally substituted with one or more groups R^{X}, and wherein said heteroaryl is not 1H-indazol-4-yl or benzimidazolyl; or alternatively, R² is ring Y, and R³ is hydrogen or R^{X};
ring Y is phenyl or a monocyclic heteroaryl, wherein said phenyl or said monocyclic heteroaryl is optionally substituted with one or more groups R^{Y}, and further wherein ring X and ring Y are not both phenyl; and
each R^{A}, each R^{X}, and each R^{Y} is independently selected from C₁₋₅ alkyl, C₂₋₅ alkenyl, C₂₋₅ alkynyl, —(C₀₋₃ alkylene)-O—R^{B}, —(C₀₋₃ alkylene)-O—(C₁₋₅ alkylene)-O—R^{B}, —(C₀₋₃ alkylene)-S—R^{B}, —(C₀₋₃ alkylene)-S—(C₁₋₅ alkylene)-S—R^{B}, —(C₀₋₃ alkylene)-N(R^{B})—R^{B}, —(C₀₋₃ alkylene)-N(R^{B})—O—R^{B}, halogen, C₁₋₅ haloalkyl, —(C₀₋₃ alkylene)-O—(C₁₋₅ haloalkyl), —(C₀₋₃ alkylene)-CN, —(C₀₋₃ alkylene)-CO—R^{B}, —(C₁₋₃ alkylene)-COOH, —(C₀₋₃ alkylene)-CO—O—(C₁₋₅ alkyl), —(C₀₋₃ alkylene)-CO—O—(C₁₋₅ haloalkyl), —(C₀₋₃ alkylene)-O—CO—R^{B}, —(C₀₋₃ alkylene)-CO—N(R^{B})—R^{B}, —(C₀₋₃ alkylene)-N(R^{B})—CO—R^{B}, —(C₀₋₃ alkylene)-N(R^{B})—CO—O—R^{B}, —(C₀₋₃ alkylene)-O—CO—N(R^{B})—R^{B}, —(C₀₋₃ alkylene)-SO₂—N(R^{B})—R^{B}, —(C₀₋₃ alkylene)-N(R^{B})—SO₂—(C₁₋₅ alkyl), —(C₀₋₃ alkylene)-SO₂—(C₁₋₅ alkyl), —(C₀₋₃ alkylene)-SO—(C₁₋₅ alkyl), -L-carbocyclyl, and -L-heterocyclyl, wherein the carbocyclyl moiety in said -L-carbocyclyl and the heterocyclyl moiety in said -L-heterocyclyl are each optionally substituted with one or more groups independently selected from C₁₋₅ alkyl, C₂₋₅ alkenyl, C₂₋₅ alkynyl, —O—R^{B}, —(C₁₋₅ alkylene)-O—R^{B}, —S—R^{B}, —S—(C₁₋₅ alkylene)-S—R^{B}, —N(R^{B})—R^{B}, —N(R^{B})—O—R^{B}, halogen, C₁₋₅ haloalkyl, —O—(CO₁₋₅ haloalkyl), —CN, —CO—R^{B}, —CO—O—R^{B}, —O—CO—R^{B}, —CO—N(R^{B})—R^{B}, —N(R^{B})CO—R^{B}, —N(R^{B})—CO—R^{B}, —O—CO—N(R^{B})—R^{B}, —SO₂—N(R^{B})—R^{B}, —N(R^{B})—SO₂˜(C₁₋₅ alkyl), —SO₂—(C₁₋₅ alkyl), and —SO—(C₁₋₅ alkyl), wherein each L is independently a covalent bond or C₁₋₅ alkylene, wherein one or more —CH₂— units comprised in said C₁₋₅ alkylene are each optionally replaced by a group independently selected from —O—, —N(R^{B})—, —CO—, —S—, —SO—, and —SO₂—, and further wherein each R^{B} is independently hydrogen, C₁₋₅ alkyl or C₁₋₅ haloalkyl;
or a pharmaceutically acceptable salt or solvate thereof.

2. The compound of claim 1, wherein R¹ is selected from C₁₋₅ alkyl, C₁₋₃ haloalkyl, —NH₂, —NH(C₁₋₃ alkyl), —N(C₁₋₃ alkyl)(C₁₋₃ alkyl), and —(C₀₋₃ alkylene)-cyclopropyl.

3. The compound of claim 1 or 2, wherein R¹ is —NH₂ or C₁₋₅ alkyl.

4. The compound of any one of claims 1 to 3, wherein R² and R³ are mutually joined to form, together with ring X, a bicyclic or tricyclic heteroaryl, wherein said bicyclic or tricyclic heteroaryl is optionally substituted with one or more groups R^{X}, and further wherein said heteroaryl is not 1H-indazol-4-yl or benzimidazolyl.

5. The compound of any one of claims 1 to 4, wherein R² and R³ are mutually joined to form, together with ring X, a bicyclic heteroaryl, wherein said bicyclic heteroaryl is optionally substituted with one or more groups R^{X}, and further wherein said bicyclic heteroaryl is not 1H-indazol-4-yl or benzimidazolyl.

6. The compound of claim 5, wherein R² and R³ are mutually joined to form, together with ring X, a bicyclic heteroaryl selected from quinolin-4-yl, quinolin-5-yl, quinolin-8-yl, isoquinolin-4-yl, isoquinolin-5-yl, isoquinolin-8-yl, benzo[b]thiophen-3-yl, benzo[b]thiophen-4-yl, benzo[b]thiophen-7-yl, pyrazolo[1,5-a]pyridin-3-yl, pyrazolo[1,5-a]pyridin-4-yl, pyrazolo[1,5-a]pyridin-7-yl, benzofuran-3-yl, benzofuran-4-yl, benzofuran-7-yl, 2,3-dihydrobenzofuran-4-yl, 2,3-dihydrobenzofuran-7-yl, 1H-indol-1-yl, 1H-indol-3-yl, 1H-indol-4-yl, 1H-indol-7-yl, 2H-isoindol-1-yl, 2H-isoindol-7-yl, 1H-indazol-1-yl, 1H-indazol-3-yl, 1H-indazol-7-yl, 1,3-benzothiazol-4-yl, 1,3-benzothiazol-7-yl, chroman-5-yl, chroman-8-yl, and 1,4-benzodioxan-5-yl, wherein said bicyclic heteroaryl is optionally substituted with one or more groups R^{X}.

7. The compound of claim 5 or 6, wherein R² and R³ are mutually joined to form, together with ring X, a quinolin-8-yl which is optionally substituted with one or more groups R^{X}, such that the compound of formula (I) has the following structure: 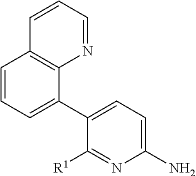
wherein the quinolin-8-yl group comprised in this compound is optionally substituted with one or more groups R^{X}.

8. The compound of any one of claims 1 to 7, wherein the number of substituents R^{X} in the compound of formula (I) is 1, 2 or 3, and further wherein each R^{X} is independently selected from C₁₋₅ alkyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, and —CN.

9. The compound of any one of claims 1 to 8, wherein the number of substituents R^{X} in the compound of formula (I) is 2 or 3, and further wherein each R^{X} is independently selected from C₁₋₅ alkyl, —OH, and halogen.

10. The compound of claim 1, wherein said compound is selected from:
6-ethyl-5-(5-fluoroquinolin-8-yl)pyridin-2-amine;
6-methyl-5-quinolin-8-yl-pyridin-2-ylamine;
5-benzo[b]thiophen-3-yl-6-ethyl-pyridin-2-ylamine;
6-ethyl-5-(6-methoxybenzothiophen-3-yl)pyridin-2-amine;
6-ethyl-5-(8-isoquinolyl)pyridin-2-amine;
5-benzo[b]thiophen-3-yl-6-propyl-pyridin-2-ylamine;
6-propyl-5-(8-quinolyl)pyridin-2-amine;
5-(8-isoquinolyl)-6-propyl-pyridin-2-amine;
5-benzo[b]thiophen-3-yl-6-isopropyl-pyridin-2-ylamine;
6-isopropyl-5-(8-quinolyl)pyridin-2-amine;
6-isopropyl-5-(8-isoquinolyl)pyridin-2-amine;
5-benzo[b]thiophen-3-yl-6-cyclopropyl-pyridin-2-ylamine;
6-cyclopropyl-5-(8-quinolyl)pyridin-2-amine;
6-cyclopropyl-5-(8-isoquinolyl)pyridin-2-amine;
3-(1-methylindol-3-yl)pyridine-2,6-diamine;
tert-butyl 3-(2,6-diamino-3-pyridyl)indole-1-carboxylate;
3-(1H-indol-3-yl)pyridine-2,6-diamine;
3-pyrazolo[1,5-a]pyridin-3-ylpyridine-2,6-diamine;
3-(benzofuran-3-yl)pyridine-2,6-diamine;
3-(benzothiophen-3-yl)pyridine-2,6-diamine;
3-(5-fluoro-benzo[b]thiophen-3-yl)pyridine-2,6-diamine;
3-(7-fluoro-2-methylquinolin-8-yl)pyridine-2,6-diamine;
3-(1H-indol-4-yl)pyridine-2,6-diamine;
3-(1H-indol-7-yl)pyridine-2,6-diamine;
3-(1-methylindazol-7-yl)pyridine-2,6-diamine;
4-(2,6-diamino-3-pyridyl)-2-methyl-isoindolin-1-one;
3-(2,3-dihydrobenzofuran-7-yl)pyridine-2,6-diamine;
3-(benzothiophen-7-yl)pyridine-2,6-diamine;
3-(1,3-benzothiazol-4-yl)pyridine-2,6-diamine;
3-(8-quinolyl)pyridine-2,6-diamine;
3-isoquinolin-8-yl-pyridine-2,6-diamine;
3-(5-isoquinolyl)pyridine-2,6-diamine;
3-quinolin-5-yl-pyridine-2,6-diamine;
3-quinolin-4-yl-pyridine-2,6-diamine;
3-isoquinolin-4-yl-pyridine-2,6-diamine;
3-chroman-8-yl-pyridine-2,6-diamine;
3-(2,3-dihydro-benzo[1,4]dioxin-5-yl)-pyridine-2,6-diamine;
3-dibenzothiophen-4-ylpyridine-2,6-diamine;
3-dibenzofuran-4-ylpyridine-2,6-diamine;
6-ethyl-5-(2-methylbenzothiophen-3-yl)pyridin-2-amine;
6-ethyl-5-(5-methylbenzothiophen-3-yl)pyridin-2-amine;
6-ethyl-5-(5-fluorobenzothiophen-3-yl)pyridin-2-amine;
6-ethyl-5-[2-(3-pyridyl)phenyl]pyridin-2-amine;
3-[2-(3-pyridyl)phenyl]pyridine-2,6-diamine;
3-[2-(6-morpholino-3-pyridyl)phenyl]pyridine-2,6-diamine;
6-ethyl-5-(quinolin-8-yl)pyridin-2-amine;
3-(2-(1-methyl-1H-pyrazol-5-yl)phenyl)pyridine-2,6-diamine;
3-(1-methyl-1H-indol-7-yl)pyridine-2,6-diamine;
3-(benzofuran-7-yl)pyridine-2,6-diamine;
3-(benzo[b]thiophen-4-yl)pyridine-2,6-diamine;
3-(6-fluoroquinolin-8-yl)pyridine-2,6-diamine;
3-(6-methylquinolin-8-yl)pyridine-2,6-diamine;
3-(5-(trifluoromethyl)quinolin-8-yl)pyridine-2,6-diamine;
3-(5-fluoroquinolin-8-yl)pyridine-2,6-diamine;
8-(2,6-diaminopyridin-3-yl)quinolin-2(1H)-one;
3-(7-fluoroquinolin-8-yl)pyridine-2,6-diamine;
3-(3-fluoroquinolin-8-yl)pyridine-2,6-diamine;
3-(5,7-difluoroquinolin-8-yl)pyridine-2,6-diamine;
3-(3-chloro-7-fluoroquinolin-8-yl)pyridine-2,6-diamine;
3-(3,5,7-trifluoroquinolin-8-yl)pyridine-2,6-diamine;
8-(2,6-diaminopyridin-3-yl)-7-fluoroquinolin-2-ol;
8-(2,6-diaminopyridin-3-yl)-7-chloroquinolin-2-ol;
8-(2,6-diaminopyridin-3-yl)-6,7-difluoroquinolin-2-ol;
6-ethyl-5-(7-fluoroquinolin-8-yl)pyridin-2-amine;
5-(chroman-8-yl)-6-ethylpyridin-2-amine;
6-isobutyl-5-(quinolin-8-yl)pyridin-2-amine;
6-(cyclobutylmethyl)-5-(quinolin-8-yl)pyridin-2-amine;
5-(7-fluoroquinolin-8-yl)-6-(3,3,3-trifluoropropyl)pyridin-2-amine;
5-(7-fluoroquinolin-8-yl)-6-isobutylpyridin-2-amine;
5-(7-fluoroquinolin-8-yl)-6-(4,4,4-trifluorobutyl)pyridin-2-amine;
6-(cyclopropylmethyl)-5-(7-fluoroquinolin-8-yl)pyridin-2-amine;
5-(7-fluoroquinolin-8-yl)-6-isopentylpyridin-2-amine;
6-ethyl-5-(6-fluoroquinolin-8-yl)pyridin-2-amine;
6-ethyl-5-(5-(trifluoromethyl)quinolin-8-yl)pyridin-2-amine;
6-ethyl-5-(7-fluoro-2-methylquinolin-8-yl)pyridin-2-amine;
6-ethyl-5-(6-methylquinolin-8-yl)pyridin-2-amine;
5-(benzo[b]thiophen-4-yl)-6-ethylpyridin-2-amine;
5-(benzofuran-7-yl)-6-ethylpyridin-2-amine;
6-ethyl-5-(2-(6-(piperidin-1-yl)pyridin-3-yl)phenyl)pyridin-2-amine;
6-ethyl-5-(2-(6-(trifluoromethyl)pyridin-3-yl)phenyl)pyridin-2-amine;
6-ethyl-5-(4-fluoro-2-(6-morpholinopyridin-3-yl)phenyl)pyridin-2-amine;
6-ethyl-5-(5-fluoro-2-(6-morpholinopyridin-3-yl)phenyl)pyridin-2-amine;
6-ethyl-5-(2-(6-morpholinopyridin-3-yl)phenyl)pyridin-2-amine;
6-ethyl-5-(2-(5-methylpyridin-3-yl)phenyl)pyridin-2-amine;
6-ethyl-5-(2-(5-methylpyridin-3-yl)phenyl)pyridin-2-amine;
6-ethyl-5-(2-(6-fluoropyridin-3-yl)phenyl)pyridin-2-amine;
5-(2-(6-amino-2-ethylpyridin-3-yl)phenyl)pyridin-2-ol;
6-ethyl-5-(2-(6-methoxypyridin-3-yl)phenyl)pyridin-2-amine;
6-ethyl-5-(2-methylquinolin-8-yl)pyridin-2-amine;
6-ethyl-5-(4-methylquinolin-8-yl)pyridin-2-amine;
8-(6-amino-2-ethylpyridin-3-yl)quinolin-2-amine;
6-ethyl-5-(7-methylquinolin-8-yl)pyridin-2-amine;
5-(2-ethoxyquinolin-8-yl)-6-ethylpyridin-2-amine;
6-ethyl-5-(3-methylquinolin-8-yl)pyridin-2-amine;
6-ethyl-5-(5-methylquinolin-8-yl)pyridin-2-amine;
6-ethyl-5-(3-fluoroquinolin-8-yl)pyridin-2-amine;
6-ethyl-5-(7-methoxyquinolin-8-yl)pyridin-2-amine;
6-ethyl-5-(2-(trifluoromethyl)quinolin-8-yl)pyridin-2-amine;
6-ethyl-5-(1,7-naphthyridin-8-yl)pyridin-2-amine;
6-ethyl-5-(quinoxalin-5-yl)pyridin-2-amine;
6-ethyl-5-(imidazo[1,2-a]pyridin-8-yl)pyridin-2-amine;
6-ethyl-5-(imidazo[1,2-a]pyridin-5-yl)pyridin-2-amine;
6-ethyl-5-(pyrazolo[1,5-a]pyridin-7-yl)pyridin-2-amine;
5-(7-(difluoromethoxy)quinolin-8-yl)-6-ethylpyridin-2-amine;
6-ethyl-5-(1,2,3,4-tetrahydroquinolin-8-yl)pyridin-2-amine;
6-ethyl-5-(7-fluoro-3-phenylquinolin-8-yl)pyridin-2-amine;
5-(5,7-difluoroquinolin-8-yl)-6-ethylpyridin-2-amine;
6-ethyl-5-(7-(trifluoromethyl)quinolin-8-yl)pyridin-2-amine;
5-(7-chloroquinolin-8-yl)-6-ethylpyridin-2-amine;
5-(6,7-difluoroquinolin-8-yl)-6-ethylpyridin-2-amine;
8-(6-amino-2-ethylpyridin-3-yl)-6,7-difluoroquinolin-3-ol;
6-ethyl-5-(5,6,7,8-tetrahydroacridin-4-yl)pyridin-2-amine;
6-ethyl-5-(2-methyl-1,2,3,4-tetrahydrobenzo[b][1,6]naphthyridin-6-yl)pyridin-2-amine,
5-(2,3-dihydro-1H-cyclopenta[b]quinolin-5-yl)-6-ethylpyridin-2-amine;
6-ethyl-5-(2-phenylquinolin-8-yl)pyridin-2-amine;
6-ethyl-5-(2-(pyridin-3-yl)quinolin-8-yl)pyridin-2-amine;
5-(2-cyclohexylquinolin-8-yl)-6-ethylpyridin-2-amine;
6-ethyl-5-(2-(pyridin-2-yl)quinolin-8-yl)pyridin-2-amine;
6-ethyl-5-(2-(1-methylcyclopropyl)quinolin-8-yl)pyridin-2-amine;
6-ethyl-5-(2-(tetrahydro-2H-pyran-4-yl)quinolin-8-yl)pyridin-2-amine;
6-ethyl-5-(2-(pyridin-4-yl)quinolin-8-yl)pyridin-2-amine;
6-ethyl-5-(2-(imidazo[1,2-a]pyridin-6-yl)quinolin-8-yl)pyridin-2-amine;
6-ethyl-5-(2-(pyrimidin-5-yl)quinolin-8-yl)pyridin-2-amine;
6-ethyl-5-(2-(isoxazol-4-yl)quinolin-8-yl)pyridin-2-amine;
6-ethyl-5-(2-(pyrazin-2-yl)quinolin-8-yl)pyridin-2-amine;
6-ethyl-5-(2-(4-methylpyridin-3-yl)quinolin-8-yl)pyridin-2-amine;
6-ethyl-5-(2-(2-methylpyridin-3-yl)quinolin-8-yl)pyridin-2-amine;
6-ethyl-5-(2-morpholinoquinolin-8-yl)pyridin-2-amine;
6-ethyl-5-(2-(2-morpholinoethoxy)quinolin-8-yl)pyridin-2-amine;
6-ethyl-5-(2-(pyrrolidin-1-yl)quinolin-8-yl)pyridin-2-amine;
5-(2-(4,4-difluoropiperidin-1-yl)quinolin-8-yl)-6-ethylpyridin-2-amine;
5-(2-(1,4-oxazepan-4-yl)quinolin-8-yl)-6-ethylpyridin-2-amine;
6-ethyl-5-(7-fluoro-2-(1,4-oxazepan-4-yl)quinolin-8-yl)pyridin-2-amine;
6-ethyl-5-(7-fluoro-2-morpholinoquinolin-8-yl)pyridin-2-amine;
5-(2-(8-oxa-3-azabicyclo[3.2.1]octan-3-yl)-7-fluoroquinolin-8-yl)-6-ethylpyridin-2-amine;
5-(2-(azepan-1-yl)-7-fluoroquinolin-8-yl)-6-ethylpyridin-2-amine;
8-(6-amino-2-ethylpyridin-3-yl)-N-cyclohexyl-N-ethyl-7-fluoroquinolin-2-amine;
8-(6-amino-2-ethylpyridin-3-yl)-N-ethyl-7-fluoro-N-isopropylquinolin-2-amine;
8-(6-amino-2-ethylpyridin-3-yl)-N,N-dimethylquinoline-2-carboxamide;
(8-(6-amino-2-ethylpyridin-3-yl)quinolin-2-yl)(pyrrolidin-1-yl)methanone;
6-ethyl-5-(2-(methoxymethyl)quinolin-8-yl)pyridin-2-amine;
5-(3,7-difluoroquinolin-8-yl)-6-ethylpyridin-2-amine;
5-(7-chloro-3-fluoroquinolin-8-yl)-6-ethylpyridin-2-amine;
6-ethyl-5-(3,5,7-trifluoroquinolin-8-yl)pyridin-2-amine;
5-(3-chloro-7-fluoroquinolin-8-yl)-6-ethylpyridin-2-amine;
5-(3,7-dichloroquinolin-8-yl)-6-ethylpyridin-2-amine;
5-(3-chloro-5,7-difluoroquinolin-8-yl)-6-ethylpyridin-2-amine;
5-(3-chloro-6,7-difluoroquinolin-8-yl)-6-ethylpyridin-2-amine;
6-ethyl-5-(3,6,7-trifluoroquinolin-8-yl)pyridin-2-amine;
5-(3-bromo-7-fluoroquinolin-8-yl)-6-ethylpyridin-2-amine;
8-(6-amino-2-ethylpyridin-3-yl)quinoline-7-carboxamide;
8-(6-amino-2-ethylpyridin-3-yl)quinoline-7-carbonitrile;
8-(6-amino-2-ethylpyridin-3-yl)quinolin-2(1H)-one;
8-(6-amino-2-ethylpyridin-3-yl)-3,4-dihydroquinolin-2(1H)-one;
8-(6-amino-2-ethylpyridin-3-yl)-1-methylquinolin-2(1H)-one;
8-(6-amino-2-ethylpyridin-3-yl)-1-methyl-3,4-dihydroquinolin-2(1H)-one;
8-(6-amino-2-ethylpyridin-3-yl)-7-fluoroquinolin-2(1H)-one;
8-(6-amino-2-ethylpyridin-3-yl)-5,7-difluoroquinolin-2(1H)-one;
8-(6-amino-2-ethylpyridin-3-yl)-7-chloroquinolin-2(1H)-one;
8-(6-amino-2-ethylpyridin-3-yl)-6,7-difluoroquinolin-2(1H)-one;
6-ethyl-5-(1-methylindolin-7-yl)pyridin-2-amine;
7-(6-amino-2-ethylpyridin-3-yl)indolin-2-one;
6-ethyl-5-(indolin-7-yl)pyridin-2-amine;
6-ethyl-5-(1-methyl-1,2,3,4-tetrahydroquinolin-8-yl)pyridin-2-amine;
(8-(6-amino-2-ethylpyridin-3-yl)quinolin-2-yl)(morpholino)methanone;
(8-(6-amino-2-ethylpyridin-3-yl)quinolin-2-yl)(1,4-oxazepan-4-yl)methanone;
8-(6-amino-2-ethylpyridin-3-yl)-N-cyclohexyl-N-ethylquinoline-2-carboxamide;
(8-(6-amino-2-ethylpyridin-3-yl)quinolin-2-yl)(azepan-1-yl)methanone;
8-(6-amino-2-ethylpyridin-3-yl)-N-ethyl-N-isopropylquinoline-2-carboxamide;
(8-(6-amino-2-ethylpyridin-3-yl)quinolin-2-yl)(8-oxa-3-azabicyclo[3.2.1]octan-3-yl)methanone;
(8-(6-amino-2-ethylpyridin-3-yl)quinolin-2-yl)(4-phenylpiperidin-1-yl)methanone;
8-(6-amino-2-ethylpyridin-3-yl)-N-(tetrahydro-2H-pyran-4-yl)quinoline-2-carboxamide;
8-(6-amino-2-ethylpyridin-3-yl)-N-benzylquinoline-2-carboxamide;
8-(6-amino-2-ethylpyridin-3-yl)-N-(oxetan-3-yl)quinoline-2-carboxamide;
6-ethyl-5-(7-fluorochroman-8-yl)pyridin-2-amine;
6-ethyl-5-(7-fluoro-2,2-dimethylchroman-8-yl)pyridin-2-amine;
6-ethyl-5-(8-fluoro-2,5-dihydrobenzo[b]oxepin-9-yl)pyridin-2-amine; and
6-ethyl-5-(8-fluoro-2,3,4,5-tetrahydrobenzo[b]oxepin-9-yl)pyridin-2-amine;
or a pharmaceutically acceptable salt or solvate thereof.

11. A pharmaceutical composition comprising the compound of any one of claims 1 to 10 and a pharmaceutically acceptable excipient.

12. The compound of any one of claims 1 to 10 or the pharmaceutical composition of claim 11 for use as a medicament.

13. The compound of any one of claims 1 to 10 or the pharmaceutical composition of claim 11 for use in the treatment or prevention of pain.

14. The compound for use according to claim 13 or the pharmaceutical composition for use according to claim 13, wherein said pain is selected from acute pain, chronic pain, postsurgical pain, cancer pain, inflammatory pain, rheumatoid arthritis-associated pain, neuropathic pain, and diabetes-associated pain.

15. The compound of any one of claims 1 to 10 or the pharmaceutical composition of claim 11 for use in the treatment or prevention of opioid-induced hyperalgesia.

16. The compound of any one of claims 1 to 10 or the pharmaceutical composition of claim 11 for use in the treatment or prevention of addiction.

17. The compound for use according to claim 16 or the pharmaceutical composition for use according to claim 16, wherein said addiction is a substance addiction or a behavioral addiction.

18. The compound for use according to claim 16 or the pharmaceutical composition for use according to claim 16, wherein said addiction is selected from alcohol addiction, amphetamine addiction, cocaine addiction, methamphetamine addiction, methylphenidate addiction, nicotine addiction, and opioid addiction.

19. The compound for use according to any one of claims 13 to 15 or the pharmaceutical composition for use according to any one of claims 13 to 15, wherein the compound or the pharmaceutical composition is to be administered in combination with one or more opioid analgesics.

20. In vitro use of a compound as defined in any one of claims 1 to 10 as an NPFF receptor antagonist.
